(19) United States
(12) Patent Application Publication
(10) Pub. No.: US 2010/0004277 A1
(43) Pub. Date: Jan. 7, 2010

(54) COMPOUNDS AND METHODS FOR MODULATING PROTEIN TRAFFICKING

(75) Inventors: Christine Bulawa, Arlington, MA (US); Michael DeVit, Somerville, MA (US)

Correspondence Address: FISH & RICHARDSON P.C., P.O. BOX 1022, MINNEAPOLIS, MN, 55440-1022, US

(73) Assignee: FoldRx Pharmaceuticals, Inc., Cambridge, MA (US)

(21) Appl. No.: 12/162,143

(22) PCT Filed: Jan. 26, 2007
(86) PCT No.: PCT/US07/02102

§ 371 Date: Mar. 10, 2009

Related U.S. Application Data
(60) Provisional application No. 60/762,955, filed on Jan. 26, 2006.
(60) Provisional application No. 60/857,940, filed on Nov. 9, 2006.

(51) Int. Cl. A61K 31/435; G01N 33/567; C12P 21/02; C07D 277/82; C07D 263/58; C07D 417/12; C07D 221/06; C07D 311/82; C07D 221/18; A61K 31/428; A61K 31/426; A61K 31/423; A61K 31/352; A61K 31/4015; A61P 3/10

(52) U.S. Cl. 514/284; 435/7.21; 435/71.1; 548/161; 548/222; 548/162; 546/110; 546/79; 549/392; 546/76; 514/369; 514/366; 514/375; 514/290; 514/455; 514/426; 435/69.1

(57) ABSTRACT

Disclosed are compositions and methods for modulating protein trafficking and treating or preventing disorders characterized by impaired protein trafficking. Also disclosed are methods for identification of compounds that rescue protein trafficking defects and methods of enhancing protein production.

### CROSS-REFERENCE TO RELATED APPLICATIONS

This application claims priority to U.S. application Ser. No. 60/762,955, filed on Jan. 26, 2006, and U.S. application Ser. No. 60/857,940, filed on Nov. 9, 2006, the contents of each of which are hereby incorporated by reference.

### TECHNICAL FIELD

This invention relates to compounds and methods for modulating protein trafficking and treating or preventing disorders characterized by impaired protein trafficking.

### BACKGROUND

Disorders characterized by impaired protein trafficking are numerous and include genetic diseases such as Huntington's disease, Tay-Sachs disease, familial hypercholesterolemia, and cystic fibrosis. Mutations in genes associated with these disorders often result in proteins that improperly fold and/or are retained in the endoplasmic reticulum. As a result, these proteins are often prematurely degraded.

The failure of a cell (e.g., in a tissue) to express a sufficient amount of an essential protein, e.g., an enzyme, can result in disease states, which vary in presentation and severity among protein trafficking disorders. For example, cystic fibrosis affects the entire body, causing progressive disability and early death. Difficulty breathing is the most common symptom and results from frequent lung infections, which are treated by antibiotics and other medications. A multitude of other symptoms, including sinus infections, poor growth, diarrhea, and infertility result from the effects of cystic fibrosis on other parts of the body. Cystic fibrosis, like many other disorders characterized by impaired protein trafficking, can be lethal if untreated.

### SUMMARY

The invention is based, at least in part, on the identification of compounds that rescue protein trafficking defects. These compounds can be used to treat a variety of disorders characterized by impaired protein trafficking. The invention is also based, at least in part, on the discovery that cells with defects in protein trafficking can be used to screen for compounds that rescue the protein trafficking defects.

Described herein are methods of treating or preventing a disorder characterized by impaired protein trafficking, the method comprising administering to a subject a compound of Formula Ia:

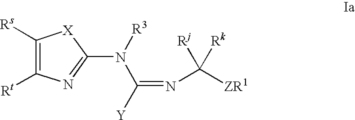

or a pharmaceutically acceptable derivative thereof. In Formula Ia, R^{j} and R^{k} are independently selected from hydrogen, alkyl, alkenyl, alkynyl, cycloalkyl, heterocyclyl, aryl, heteroaryl or aralkyl; or, R^{j} and R^{k}, together with the carbon to which they are both bonded, are —C(═O)—, —CH(OR*)—, —C(═S)—, —CH(SR*)—, —CH(N*R*′)-or —C(═NR*)—, where R* and R*′ are independently hydrogen, alkyl, alkenyl, alkynyl, cycloalkyl, heterocyclyl, aryl, heteroaryl or aralkyl, R^{s} and R^{t} are independently selected from hydrogen, alkyl, halo, pseudohalo, alkenyl, alkynyl, cycloalkyl, heterocyclyl, aryl, heteroaryl or aralkyl; or, R^{s} and R^{t}, together with the carbon-carbon double bond between them, form a 4-6 membered cycloalkenyl, aryl, heterocyclyl, or heteroaryl ring, wherein the ring formed by R^{s} and R^{t} is optionally substituted with 0-4 substituents R² defined herein below.

Also described herein are compounds represented by Formula Ia or pharmaceutically acceptable derivatives thereof, wherein R^{j} and R^{k} are independently selected from hydrogen, alkyl, alkenyl, alkynyl, cycloalkyl, heterocyclyl, aryl, heteroaryl or aralkyl; or, R^{j} and R^{k}, together with the carbon to which they are both bonded, are —C(═O)—, —CH(OR*)—, —C(═S)—, —CH(SR*)—, —CH(NR*R*′)-or —C(═NR*)—; Y is NRR″, OR′, SR′, or CRR″; where R″ is hydrogen, alkyl, alkenyl, alkynyl, cycloalkyl, heterocyclyl, aryl, heteroaryl or aralkyl, or R″, together with R³ and the atoms therebetween, is a 4-6 membered heterocyclyl or heteroaryl ring; provided that when R^{j} and R^{k}, together with the carbon to which they are both bonded, are —C(═O)—, R″, together with R³ and the atoms therebetween, is a 4-6 membered heterocyclyl or heteroaryl ring. In some embodiments, when R^{j} and R^{k}, together with the carbon to which they are both bonded, are —C(═O)—, —CH(OR*)—, —C(═S)—, —CH(SR*)—, —CH(NR*R*′)-or —C(═NR*)—, Y is NRR″ or CRR″ and R″, together with R³ and the atoms therebetween, is a 4-6 membered heterocyclyl or heteroaryl ring. In various embodiments of the compound represented by Formula Ia, R^{j} and R^{k} are independently selected from hydrogen, alkyl, alkenyl, alkynyl, cycloalkyl, heterocyclyl, aryl, heteroaryl or aralkyl, or R^{j} and R^{k}, taken together, are —CH(OR*)—, —C(═S)—, —CH(SR*)—, —CH(NR*R*′)-or —C(═NR*)—. In some embodiments, the compounds are represented by Formula Ia or pharmaceutically acceptable derivatives thereof wherein R^{j} and R^{k} are independently selected from hydrogen, alkyl, alkenyl, alkynyl, cycloalkyl, heterocyclyl, aryl, heteroaryl or aralkyl. Also described herein are pharmaceutical compositions comprising the compounds and a pharmaceutically acceptable carrier.

In some embodiments, the compound is represented by structural Formula I:

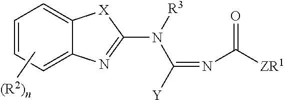

or a pharmaceutically acceptable derivative thereof.

In Formulas Ia and I:

X is O, S or NR, where R is hydrogen, alkyl, alkenyl, alkynyl, cycloalkyl, heterocyclyl, aryl, heteroaryl or aralkyl; in some embodiments, when R^{j} and R^{k} in Formula Ia are both hydrogen, X is O;

Y is NRR′ or OH; where R′ is hydrogen, alkyl, alkenyl, alkynyl, cycloalkyl, heterocyclyl, aryl, heteroaryl or aralkyl; in some embodiments, Y is NRR″, OR′, SR′, or CRR″; where R″ is hydrogen, alkyl, alkenyl, alkynyl, cycloalkyl, heterocyclyl, aryl, heteroaryl or aralkyl, or R″, together with R³ and the atoms therebetween, is a 4-6 membered heterocyclyl or heteroaryl ring, for example, the heteroaryl rings represented by rings A and B in the following compounds:

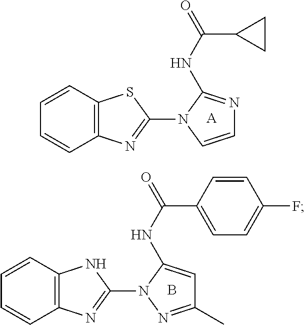

Z is a direct bond or NR;

R¹ is hydrogen, alkyl, alkenyl, alkynyl, cycloalkyl, heterocyclyl, aryl, heteroaryl, aralkyl, aralkenyl, heteroaralkyl or heteroaralkenyl; in some embodiments, when R^{j} and R^{k} in Formula Ia are both hydrogen, R^{l} is a cycloalkyl group; in some embodiments, when R^{j} and R^{k} in Formula Ia are both hydrogen, R^{l} is a cycloalkyl and Z is a direct bond; in some embodiments, when R^{j} and R^{k} in Formula Ia are both hydrogen, R^{l} is a cycloalkyl, Z is a direct bond, and X is O;

n is 0 to 4;

R² is selected from (i) or (ii) as follows:

(i) hydrogen, alkyl, alkenyl, alkynyl, aryl, heteroaryl, heteroarylium, cycloalkyl, heterocyclyl, C(A)R¹¹⁰, halo, pseudohalo, OR¹¹¹, S(D)ₐR¹¹², NR¹¹⁵R¹¹⁶ or N⁺R¹¹⁵R¹¹⁶R¹¹⁷; or

(ii) any two R² groups, which substitute adjacent atoms on the ring, together form alkylene, alkenylene, alkynylene or heteroalkylene;

A is O, S or NR¹²⁵;

R¹¹⁰ is hydrogen, alkyl, alkenyl, alkynyl, aryl, heteroaryl, heteroarylium, cycloalkyl, heterocyclyl, C(A)R¹²⁶, halo pseudohalo, OR¹²⁵, SR¹²⁵, N¹²⁷R¹²⁸ or SiR¹²²R¹²³R¹²⁴;

R¹¹¹ is hydrogen, alkyl, alkenyl, alkynyl, aryl, heteroaryl, heteroarylium, cycloalkyl, heterocyclyl, C(A)R¹²⁹, NR¹³⁰R¹³¹ or SiR¹²²R¹²³R¹²⁴;

D is O or NR¹²⁵;

a is 0, 1 or 2;

when a is 1 or 2, R¹¹² is selected from hydrogen, alkyl, alkenyl, alkynyl, aryl, heteroaryl, heteroarylium, cycloalkyl, heterocyclyl, halo, pseudohalo, OR¹²⁵, SR¹²⁵ and NR¹³²R¹³³;

when a is 0, R¹¹² is selected from hydrogen, alkyl, alkenyl, alkynyl, aryl, heteroaryl, heteroarylium, cycloalkyl, heterocyclyl, SR¹²⁵ and C(A)R¹²⁹;

R¹¹⁵, R¹¹⁶ and R¹¹⁷ are each independently selected from (a) and (b) as follows:

(a) hydrogen, alkyl, alkenyl, alkynyl, aryl, heteroaryl, heteroarylium, cycloalkyl, heterocyclyl, C(A)R¹²⁹, OR¹²⁵ or NR¹³²R¹³³; or

(b) any two of R¹¹⁵, R¹¹⁶ and R¹¹⁷ together form alkylene, alkenylene, alkynylene, heteroalkylene, and the other is selected as in (a);

R¹²², R¹²³ and R¹²⁴ are selected as in (i) or (ii) as follows:

(i) R¹²², R¹²³ and R¹²⁴ are each independently hydrogen, alkyl, alkenyl, alkynyl, aryl, heteroaryl, heteroarylium, cycloalkyl, heterocyclyl, OR¹²⁵ or NR¹³²R¹³³; or

(ii) any two of R¹²², R¹²³ and R¹²⁴ together form alkylene, alkenylene, alkynylene, heteroalkylene; and the other is selected as in (i);

R¹²⁵ is hydrogen, alkyl, alkenyl, alkynyl, aryl, heteroaryl, heteroarylium, cycloalkyl or heterocyclyl;

R¹²⁶ is hydrogen, alkyl, alkenyl, alkynyl, aryl, heteroaryl, heteroarylium, cycloalkyl, heterocyclyl, OR¹²⁵ or NR¹³⁴R¹³⁵; where R¹³⁴ and R¹³⁵ are each independently hydrogen, alkyl, alkenyl, alkynyl, aryl, heteroaryl, heteroarylium, cycloalkyl, heterocyclyl, OR¹³⁶ or NR¹³²R¹³³, or R¹³⁴ and R¹³⁵ together form alkylene, alkenylene, alkynylene, heteroalkylene, where R¹³⁶ is hydrogen, alkyl, alkenyl, alkynyl, aryl, heteroaryl, heteroarylium, cycloalkyl or heterocyclyl;

R¹²⁷ and R¹²⁸ are selected as in (i) or (ii) as follows:

(i) R¹²⁷ and R¹²⁸ are each independently hydrogen, alkyl, alkenyl, alkynyl, aryl, heteroaryl, heteroarylium, cycloalkyl, heterocyclyl, OR¹²⁵, NR¹³⁷R¹³⁸ or C(A)R¹³⁹, where R¹³⁷ and R¹³⁸ are each independently hydrogen, alkyl, alkenyl, alkynyl, aryl, heteroaryl, heteroarylium, cycloalkyl or heterocyclyl, or together form alkylene, alkenylene, alkynylene, heteroalkylene; and R¹³⁹ is hydrogen, alkyl, alkenyl, alkynyl, aryl, heteroaryl, heteroarylium, cycloalkyl, heterocyclyl, OR¹⁴⁰ or NR¹³²R¹³³, where R¹⁴⁰ is alkyl, alkenyl, alkynyl, aryl, heteroaryl, heteroarylium, cycloalkyl, heterocyclyl; or

(ii) R¹²⁷ and R¹²⁸ together form alkylene, alkenylene, alkynylene, heteroalkylene;

R¹²⁹ is hydrogen, alkyl, alkenyl, alkynyl, aryl, heteroaryl, heteroarylium, cycloalkyl, heterocyclyl, OR¹⁴⁰ or NR¹³²R¹³³;

R¹³⁰ and R¹³¹ are each independently hydrogen, alkyl, alkenyl, alkynyl, aryl, heteroaryl, heteroarylium, cycloalkyl, heterocyclyl or C(A)R¹⁴¹, where R¹⁴¹ is alkyl, alkenyl, alkynyl, aryl, heteroaryl, heteroarylium, cycloalkyl, heterocyclyl, OR¹²⁵ or NR¹³²R¹³³; or R¹³⁰ and R¹³¹ together form alkylene, alkenylene, alkynylene, heteroalkylene;

R¹³² and R¹³³ are each independently hydrogen, alkyl, alkenyl, alkynyl, aryl, heteroaryl, heteroarylium, cycloalkyl, heterocyclyl, or R¹³² and R¹³³ together form alkylene, alkenylene, alkynylene, heteroalkylene; and

R³ is hydrogen, alkyl, alkenyl, alkynyl, cycloalkyl, heterocyclyl, aryl or heteroaryl;

wherein X, Y, Z, R¹, R² and R³, or in some embodiments, X, Y, Z, R, R′, R″, R*, R¹, R² and R³, are each independently unsubstituted or substituted with one or more substituents, in one embodiment one, two or three substituents, each independently selected from Q¹, where Q¹ is halo, pseudohalo, hydroxy, oxo, thia, nitrile, nitro, formyl, mercapto, hydroxycarbonyl, hydroxycarbonylalkyl, hydroxycarbonylalkenyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, alkoxyxarbonylalkoxy, aryloxycarbonyl, aryloxycarbonylalkyl, aralkoxycarbonyl, aralkoxycarbonylalkyl, aralkoxycarbonylalkoxy, arylcarbonylalkyl, aminocarbonyl, aminocarbonylalkyl, aminocarbonylalkoxy, alkylaminocarbonyl, alkylaminocarbonylalkyl, alkylaminocarbonylalkoxy, dialkylaminocarbonyl, dialkylaminocarbonylalkyl, dialkylaminocarbonylalkoxy, arylaminocarbonyl, arylaminocarbonylalkyl, arylaminocarbonylalokoxy, diarylaminocarbonyl, diarylaminocarbonylalkyl, diarylaminocarbonyl alkoxy, arylalkylaminocarbonyl, arylalkylaminocarbonylalkyl, arylalkylaminocarbonylalkoxy, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R¹⁵¹R¹⁵²R¹⁵³, P(R¹⁵⁰)₂, P(═O)(R¹⁵⁰)₂, OP(═O)(R¹⁵⁰)₂, —NR¹⁶⁰C(═O)R¹⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; azido, tetrazolyl or two Q¹ groups, which substitute atoms in a 1,2 or 1,3 arrangement, together form alkylenedioxy (i.e., —O—(CH₂)_{y}—O—), thioalkylenoxy (i.e., —S—(CH₂)_{y}—O—)or alkylenedithioxy (i.e., —S—(CH₂)_{y}—S—) where y is 1 or 2; or two Q¹ groups, which substitute the same atom, together form alkylene; and

each Q¹ is independently unsubstituted or substituted with one or more substituents, in one embodiment one, two or three substituents, each independently selected from Q²;

each Q² is independently halo, pseudohalo, hydroxy, oxo, thia, nitrile, nitro, formyl, mercapto, hydroxycarbonyl, hydroxycarbonylalkyl, hydroxycarbonylalkenyl alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R¹⁵¹R¹⁵²R¹⁵³, P(R¹⁵⁰)₂, P(═O)(R¹⁵⁰)₂, OP(═O)(R¹⁵⁰)₂, —NR¹⁶⁰C(═O)R¹⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q² groups, which substitute atoms in a 1,2 or 1,3 arrangement, together form alkylenedioxy (i.e., —O—(CH₂)_{y}—O—), thioalkylenoxy (i.e., —S—(CH₂)_{y}—O—)or alkylenedithioxy (i.e., —S—(CH₂)_{y}—S—) where y is 1 or 2; or two Q² groups, which substitute the same atom, together form alkylene;

R¹⁵⁰ is hydroxy, alkoxy, aralkoxy, alkyl, heteroaryl, heterocyclyl, aryl or —NR¹⁷⁰R¹⁷¹, where R¹⁷⁰ and R¹⁷¹ are each independently hydrogen, alkyl, aralkyl, aryl, heteroaryl, heteroaralkyl or heterocyclyl, or R¹⁷⁰ and R¹⁷¹ together form alkylene, azaalkylene, oxaalkylene or thiaalkylene;

R¹⁵¹, R¹⁵² and R¹⁵³ are each independently hydrogen, alkyl, aryl, aralkyl, heteroaryl, heteroaralkyl, heterocyclyl or heterocyclylalkyl;

R¹⁶⁰ is hydrogen, alkyl, aryl, aralkyl, heteroaryl, heteroaralkyl, heterocyclyl or heterocyclylalkyl; and

R¹⁶³ is alkoxy, aralkoxy, alkyl, heteroaryl, heterocyclyl, aryl or —NR¹⁷⁰R¹⁷¹.

In some embodiments, R¹ is substituted with one or more substituents independently selected from aryloxy, aryl, heteroaryl, halo, pseudohalo, alkyl, alkoxy, cycloalkyl, alkoxycarbonyl, hydroxycarbonyl, alkylamino, and dialkylamino.

As one of skill in the art will recognize, Formulas Ia and I structurally set forth one tautomeric form of the compounds encompassed therein; all such tautomeric forms are contemplated herein. For example, Formulas Ia and I include a fragment represented by —NH—CH(Y)═N—, and when Y is NH₂, the fragment is a guanidine group which includes the three tautomeric forms —NH—CH(NH₂)═N—, —NH—CH(═NH)—NH—, and —N═CH(NH₂)—NH—.

In some embodiments:

X is O, S or NR, where R is hydrogen or alkyl;

Y is NRR′ or OH, where R is hydrogen or alkyl;

Z is a direct bond or NR;

R¹ is alkyl, alkenyl, alkynyl, cycloalkyl, aryl, heterocyclyl, heteroaryl, aralkyl, aralkenyl, heteroaralkyl, or heteroaralkenyl;

R² is halo, pseudohalo, alkoxy or alkyl;

n is 0 or 1;

R³ is hydrogen or alkyl;

wherein X, Y, Z, R¹, R² and R³ are each independently unsubstituted or substituted with one or more substituents, in one embodiment one, two or three substituents, each independently selected from Q¹.

In some embodiments R is hydrogen.

In some embodiments n is 0 or 1.

In some embodiments X is S, O or NH.

In some embodiments Y is NH₂.

In some embodiments Z is a direct bond or NH.

In some embodiments R¹ is alkyl, alkenyl, cycloalkyl, heterocyclyl, aryl or heteroaryl, and is unsubstituted or substituted with aryloxy, aryl, heteroaryl, halo, pseudohalo, alkyl, alkoxy, cycloalkyl, alkoxycarbonyl, hydroxycarbonyl, alkylamino, and dialkylamino.

In some embodiments R¹ is ethyl, 2-(2-furyl)ethenyl, phenyl, methyl, 2-naphthyloxymethyl, benzyl, 3-chloro-2-benzothienyl, cyclopropyl, cyclopropylmethyl, isobutyl, 4-tert-butylphenyl, 4-biphenyl, tert-butyl, 3-chlorophenyl, 2-furyl, 2,4-dichlorophenyl, 3,4-dimethoxyphenyl, 2-(4-methoxyphenyl)ethenyl, 4-methoxyphenoxymethyl, isopentyl, isopropyl, 2-cyclopentylethyl, cyclopentylmethyl, 2-phenylpropyl, 2-phenylethyl, 1-methyl-2-phenylethyl, 1-methyl-2-phenylethenyl, 2-benzylethyl, 2-phenylethenyl, 5-hexynyl, 3-butynyl, 4-pentynyl, propyl, butyl, pentyl, hexyl, t-butoxymethyl, t-butylmethyl, 1-ethylpentyl, cyclopropyl, cyclopropylmethyl, cyclopentyl, cyclohexyl, cyclobutyl, 2-cyclopentylethyl, cyclopentylmethyl, 2-fluorocyclopropyl, 2-methylcyclopropyl, 2-phenylcyclopropyl, 2,2-dimethylethenyl, 1,2-propenyl, 2-(3-trifluoromethylphenyl)ethenyl, 3,4-butenyl, 2-(2-furyl)ethyl, 2-chloroethenyl, 2-(2-chlorophenyl)ethenyl, 1-methyl-2,2-dichlorocyclopropyl, 2,2-difluorocyclopropyl, methylpropionate, proprionic acid, methylbutyrate, butyric acid, pentanoic acid, methyl-t-butylether, dimethylaminomethyl, 2-(2-tetrahydrofuryl)-ethyl, or 2-(2-tetrahydrofuryl)-methyl.

In some embodiments R² is halo or alkyl.

In some embodiments R² is chloro or methyl.

In some embodiments R³ is hydrogen.

In various embodiments, the compound is represented by one of Formulas Ib-Im.

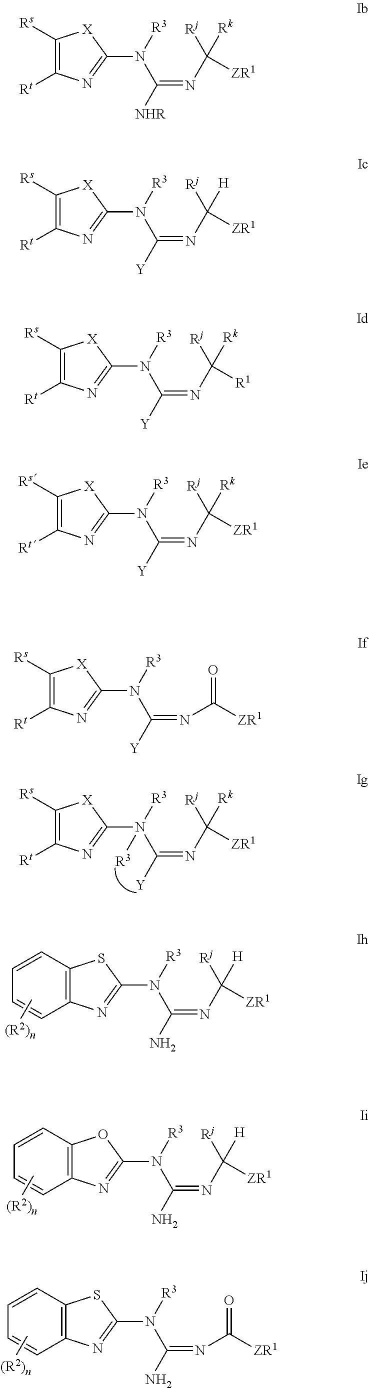

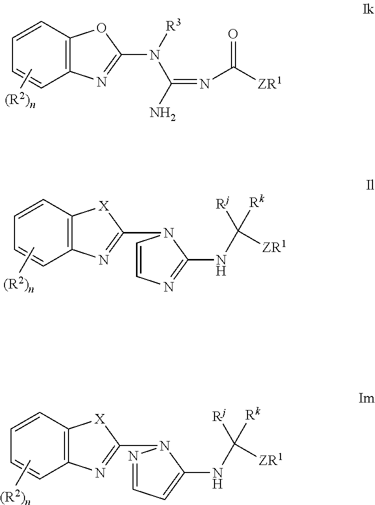

In Formulas Ib-Im, the variables have the values described herein above for Formulas I and Ia.

In various embodiments, R¹ in Formulas Ib-Im is hydrogen, alkyl, aryl, aralkyl, aralkenyl, alkynyl, heteroaryl, heteroaralkyl, heteroarylalkenyl, cycloalkyl, each of which is substituted with 0, 1 or 2 groups selected from phenyl, alkyl, cycloalkyl, alkoxy, halo, pseudohalo, amino, alkylamino, or dialkylamino. In various embodiments, R¹ in Formulas Ib-Im is phenyl, furyl, thienyl, alkynyl, alkyl, cyclopropyl, cyclobutyl or cyclopentyl; or alkyl or alkenyl substituted with phenyl, furyl, thienyl, alkynyl, alkyl, cyclopropyl, cyclobutyl or cyclopentyl; in some embodiments, R¹ is optionally substituted with 0, 1 or 2 groups selected from phenyl, alkyl, alkoxy, halo, or CN.

In some embodiments, R^{j} and R^{k} in Formulas Ib-Im are both hydrogen. In some embodiments, R³ in Formulas Ib-Im is hydrogen.

In various embodiments represented by Formula Ie, R^{s}′ and R^{t}′ are independently selected from hydrogen, alkyl, halo, pseudohalo, alkenyl, alkynyl, cycloalkyl, heterocyclyl, aryl, heteroaryl or aralkyl; in some embodiments, R^{s}′ and R^{t}′ are independently selected from hydrogen, alkyl, and halo; and in certain embodiments, R^{s}′ and R^{t}′ are independently selected from hydrogen, alkyl, and Br, wherein typically, R^{s}′ and R^{t}′ are not both hydrogen.

In some embodiments of Formulas Ih-Im, n is 0, 1 or 2 and each R² is independently selected from halogen, alkyl, alkoxy, haloalkyl, and haloalkoxy; in some embodiments, n is 0, 1 or 2 and each R² is independently selected from hydrogen, F, fluoroalkyl (e.g., CHF₂, CF₃), and fluoroalkoxy (e.g., OCHF₂, OCF₃).

In some embodiments the compound is selected from the compounds in Table I. In certain embodiments, the compound is selected from compounds I.1-I.57 in Table I; in some embodiments, the compound is selected from compounds I.1-I.35 in Table I. In some embodiments, the compound is selected from compounds I.1-I.6 and I.36-I.57 in Table I. In some embodiments, the compound is selected from compounds I.7-I.35 in Table I.

Also disclosed are methods of treating or preventing a disorder characterized by impaired protein trafficking, the method comprising administering to a subject a compound of Formula IIa:

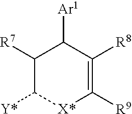

or a pharmaceutically acceptable derivative thereof. In Formula IIa, X* is selected from the group consisting of —O—, ═N—, —N(R^{o})—, ═C(R)— and —C(R^{o}R^{o}′)—, and Y* is selected from ═O, —OR^{o}, ═NR^{o}′, —NR^{o}R^{o}′, ═CR^{o}R^{o}′ and —CHR^{o}R^{o}′; where X* and Y* are selected such that one of the dashed bonds (— — —) is a single bond and the other is a double bond, or both dashed bonds are single bonds. Each R^{o}′ is independently selected from the group consisting of hydrogen, halogen, pseudohalo, amino, amido, carboxamido, sulfonamide, carboxyl, alkyl, alkenyl, alkynyl, cycloalkyl, heterocyclyl, aryl, heteroaryl, aralkyl, alkoxy, cycloalkoxy, heterocycloxy, aryloxy, heteroaryloxy, and aralkyloxy. Each R^{o} is selected from the group consisting of hydrogen, alkyl, alkenyl, alkynyl, cycloalkyl, heterocyclyl, aryl, heteroaryl and aralkyl. In some embodiments, R^{o} is independently selected from the group consisting of hydrogen, alkyl, alkenyl, alkynyl, cycloalkyl, heterocyclyl, aryl, heteroaryl or aralkyl. In certain embodiments, R^{o} is hydrogen or alkyl, typically hydrogen.

Also described herein are compounds represented by Formula IIa or pharmaceutically acceptable derivatives thereof, wherein X* is selected from the group consisting of —O—, ═N—, —N(R^{o})—, ═C(R^{o})— and —C(R^{o}R^{o}′)—; and Y* is selected from the group consisting of ═O, —OR^{°}, ═NR^{o}′, —NR^{o}R^{o}′, ═CR^{°}R^{o}′ and —CHR^{o}R^{o}′; where X* and Y* are selected such that both dashed bonds are single bonds, or one of the dashed bonds (— — —) is a single bond and the other is a double bond, provided that Y* is not ═O when X* is —N(H)—. In various embodiments of the compounds represented by by Formula IIa, X* and Y* are selected such that both dashed bonds are single bonds, or one of the dashed bonds (— — —) is a single bond and the other is a double bond, provided that Y* is not ═O when X* is —N(R^{o})—. In some embodiments of the compounds represented by by Formula IIa, X* and Y* are selected such that both dashed bonds are single bonds, or one of the dashed bonds (— — —) is a single bond and the other is a double bond, provided that Y* is not ═O, ═NR^{o}′, or ═CR^{o}R^{o}′ when X* is —N(R^{o})—. Also described herein are pharmaceutical compositions comprising the compounds of Formula IIa and a pharmaceutically acceptable carrier.

In some embodiments, the compounds of Formula IIa can also be represented by Formula II:

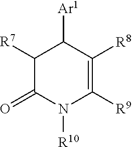

or a pharmaceutically acceptable derivative thereof.

In Formulas IIa and II:

Ar¹ is aryl, heteroaryl, or cycloalkyl;

R⁷ is hydrogen, alkyl, alkenyl, alkynyl, cycloalkyl, heterocyclyl, aryl, heteroaryl or NRR, where R is hydrogen or alkyl;

R¹⁰ is hydrogen, alkyl, alkenyl, alkynyl, cycloalkyl, heterocyclyl, aryl or heteroaryl;

R⁸ and R⁹ are each independently selected from (i) or (ii) as follows:

(i) hydrogen, alkyl, alkenyl, alkynyl, aryl, heteroaryl, heteroarylium, cycloalkyl, heterocyclyl, C(A)R¹¹⁰, halo, pseudohalo, OR¹¹¹, S(D)ₐR¹¹², NR¹¹⁵R¹¹⁶ or N⁺R¹¹⁵R¹¹⁶R¹¹⁷; or

(ii) R⁸ and R⁹ together form alkylene, alkenylene, alkynylene or heteroalkylene; for example, in some embodiments, R⁸ and R⁹ together with the atoms to which they are attached form a fused phenyl ring, which is unsubstituted or substituted with halo, pseudohalo, alkyl, alkoxy, cycloalkyl, fused cycloalkyl, fused heterocyclyl, fused heteroaryl, or fused aryl, which is unsubstituted or substituted with halo, pseudohalo, alkyl, alkoxy, aryl, cycloalkyl, heterocyclyl, fused aryl, fused heterocyclyl, and fused cycloalkyl;

A is O, S or NR¹²⁵;

R¹¹⁰ is hydrogen, alkyl, alkenyl, alkynyl, aryl, heteroaryl, heteroarylium, cycloalkyl, heterocyclyl, C(A)R¹²⁶, halo, pseudohalo, OR¹²⁵, SR¹²⁵, NR¹²⁷R¹²⁸ and SiR¹²²R¹²³R¹²⁴;

R¹¹¹ is hydrogen, alkyl, alkenyl, alkynyl, aryl, heteroaryl, heteroarylium, cycloalkyl, heterocyclyl, C(A)R¹²⁹, NR¹³⁰R¹³¹ and SiR¹²²R¹²³R¹²⁴;

D is O or NR¹²⁵;

a is 0, 1 or 2;

when a is 1 or 2, R¹¹² is selected from hydrogen, alkyl, alkenyl, alkynyl, aryl, heteroaryl, heteroarylium, cycloalkyl; heterocyclyl, halo, pseudohalo, OR¹²⁵, SR¹²⁵ and NR¹³²R¹³³;

when a is 0, R¹¹² is selected from hydrogen, alkyl, alkenyl, alkynyl, aryl, heteroaryl, heteroarylium, cycloalkyl, heterocyclyl, SR¹²⁵ and C(A)R¹²⁹;

R¹¹⁵, R¹¹⁶ and R¹¹⁷ are each independently selected from (a) and (b) as follows:

(a) hydrogen, alkyl, alkenyl, alkynyl, aryl, heteroaryl, heteroarylium, cycloalkyl, heterocyclyl, C(A)R¹²⁹, OR¹²⁵ or NR¹³²R¹³³; or

(b) any two of R¹¹⁵, R¹¹⁶ and R¹¹⁷ together form alkylene, alkenylene, alkynylene, heteroalkylene, and the other is selected as in (a);

R¹²², R¹²³ and R¹²⁴ are selected as in (i) or (ii) as follows:

(i) R¹²², R¹²³ and R¹²⁴ are each independently hydrogen, alkyl, alkenyl, alkynyl, aryl, heteroaryl, heteroarylium, cycloalkyl, heterocyclyl, OR¹²⁵ or NR¹³²R¹³³; or

(ii) any two of R¹²², R¹²³ and R¹²⁴ together form alkylene, alkenylene, alkynylene, heteroalkylene; and the other is selected as in (i);

R¹²⁵ is hydrogen, alkyl, alkenyl, alkynyl, aryl, heteroaryl, heteroarylium, cycloalkyl or heterocyclyl; in some embodiments, where R¹²⁵ is alkyl, alkenyl, or alkynyl, R¹²⁵ is optionally substituted with aryl, heteroaryl, heteroarylium, cycloalkyl or heterocyclyl;

R¹²⁶ is hydrogen, alkyl, alkenyl, alkynyl, aryl, heteroaryl, heteroarylium, cycloalkyl, heterocyclyl, OR¹²⁵ or NR¹³⁴R¹³⁵; where R¹³⁴ and R¹³⁵ are each independently hydrogen, alkyl, alkenyl, alkynyl, aryl, heteroaryl, heteroarylium, cycloalkyl, heterocyclyl, OR¹³⁶ or NR¹³²R¹³³, or R¹³⁴ and R¹³⁵ together form alkylene, alkenylene, alkynylene, heteroalkylene, where R¹³⁶ is hydrogen, alkyl, alkenyl, alkynyl, aryl, heteroaryl, heteroarylium, cycloalkyl or heterocyclyl;

R¹²⁷ and R¹²⁸ are selected as in (i) or (ii) as follows:

(i) R¹²⁷ and R¹²⁸ are each independently hydrogen, alkyl, alkenyl, alkynyl, aryl, 30 heteroaryl, heteroarylium, cycloalkyl, heterocyclyl, OR¹²⁵, NR¹³⁷R¹³⁸ or C(A)R¹³⁹, where R¹³⁷ and R¹³⁸ are each independently hydrogen, alkyl, alkenyl, alkynyl, aryl, heteroaryl, heteroarylium, cycloalkyl or heterocyclyl, or together form alkylene, alkenylene, alkynylene, heteroalkylene; and R¹³⁹ is hydrogen, alkyl, alkenyl, alkynyl, aryl, heteroaryl, heteroarylium, cycloalkyl, heterocyclyl, OR¹⁴⁰ or NR¹³²R¹³³, where R¹⁴⁰ is alkyl, alkenyl, alkynyl, aryl, heteroaryl, heteroarylium, cycloalkyl, heterocyclyl; or

(ii) R¹²⁷ and R¹²⁸ together form alkylene, alkenylene, alkynylene, heteroalkylene;

R¹²⁹ is hydrogen, alkyl, alkenyl, alkynyl, aryl, heteroaryl, heteroarylium, cycloalkyl, heterocyclyl, OR¹⁴⁰ or NR¹³²R¹³³;

R¹³⁰ and R¹³¹ are each independently hydrogen, alkyl, alkenyl, alkynyl, aryl, heteroaryl, heteroarylium, cycloalkyl, heterocyclyl or C(A)R¹⁴¹, where R¹⁴¹ is alkyl, alkenyl, alkynyl, aryl, heteroaryl, heteroarylium, cycloalkyl, heterocyclyl, OR¹²⁵ or NR¹³²R¹³³; or R¹³⁰ and R¹³¹ together form alkylene, alkenylene, alkynylene, heteroalkylene;

R¹³² and R¹³³ are each independently hydrogen, alkyl, alkenyl, alkynyl, aryl, heteroaryl, heteroarylium, cycloalkyl, heterocyclyl, or R¹³² and R¹³³ together form alkylene, alkenylene, alkynylene, heteroalkylene; and

R¹⁰ is hydrogen, alkyl, alkenyl, alkynyl, cycloalkyl, heterocyclyl, aryl or heteroaryl;

where Ar¹, R⁷, R⁸, R⁹ and R¹⁰ are each independently unsubstituted or substituted with one or more, in one embodiment one, two or three substituents, each independently selected from Q¹, where Q¹ is halo, pseudohalo, hydroxy, oxo, thia, nitrile, nitro, formyl, mercapto, hydroxycarbonyl, hydroxycarbonylalkyl, hydroxycarbonylalkenyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, alkoxyxarbonylalkoxy, aryloxycarbonyl, aryloxycarbonylalkyl, aralkoxycarbonyl, aralkoxycarbonylalkyl, aralkoxycarbonylalkoxy, arylcarbonylalkyl, aminocarbonyl, aminocarbonylalkyl, aminocarbonylalkoxy, alkylaminocarbonyl, alkylaminocarbonylalkyl, alkylaminocarbonylalkoxy, dialkylaminocarbonyl, dialkylaminocarbonylalkyl, dialkylaminocarbonylalkoxy, arylaminocarbonyl, arylaminocarbonylalkyl, arylaminocarbonylalokoxy, diarylaminocarbonyl, diarylaminocarbonylalkyl, diarylaminocarbonyl alkoxy, arylalkylaminocarbonyl, arylalkylaminocarbonylalkyl, arylalkylaminocarbonylalkoxy, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R¹⁵¹R¹⁵²R¹⁵³, P(R¹⁵⁰)₂, P(═O)(R¹⁵⁰)₂, OP(═O)(R¹⁵⁰)₂, —NR¹⁶⁰C(═O)R¹⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; azido, tetrazolyl or two Q¹ groups, which substitute atoms in a 1,2 or 1,3 arrangement, together form alkylenedioxy (i.e., —O—(CH₂)_{y}—O—), thioalkylenoxy (i.e., —S—(CH₂)_{y}—O—)or alkylenedithioxy (i.e., —S—(CH₂)_{y}—S—) where y is 1 or 2; or two Q¹ groups, which substitute the same atom, together form alkylene; and

each Q¹ is independently unsubstituted or substituted with one or more substituents, in one embodiment one, two or three substituents, each independently selected from Q²;

each Q² is independently halo, pseudohalo, hydroxy, oxo, thia, nitrile, nitro, formyl, mercapto, hydroxycarbonyl, hydroxycarbonylalkyl, hydroxycarbonylalkenyl alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R¹⁵¹R¹⁵²R¹⁵³, P(R¹⁵⁰)₂, P(═O)(R¹⁵⁰)₂, OP(═O)(R¹⁵⁰)₂, —NR¹⁶⁰(═O)R¹⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q² groups, which substitute atoms in a 1,2 or 1,3 arrangement, together form alkylenedioxy (i.e., —O—(CH₂)_{y}—O—), thioalkylenoxy (i.e., —S—(CH₂)_{y}—O—)or alkylenedithioxy (i.e., —S—(CH₂)_{y}—S—) where y is 1 or 2; or two Q² groups, which substitute the same atom, together form alkylene;

R¹⁵⁰ is hydroxy, alkoxy, aralkoxy, alkyl, heteroaryl, heterocyclyl, aryl or —NR¹⁷⁰R¹⁷¹, where R¹⁷⁰ and R¹⁷¹ are each independently hydrogen, alkyl, aralkyl, aryl, heteroaryl, heteroaralkyl or heterocyclyl, or R¹⁷⁰ and R¹⁷¹ together form alkylene, azaalkylene, oxaalkylene or thiaalkylene;

R¹⁵¹, R¹⁵² and R¹⁵³ are each independently hydrogen, alkyl, aryl, aralkyl, heteroaryl, heteroaralkyl, heterocyclyl or heterocyclylalkyl;

R¹⁶⁰ is hydrogen, alkyl, aryl, aralkyl, heteroaryl, heteroaralkyl, heterocyclyl or heterocyclylalkyl; and

R¹⁶³ is alkoxy, aralkoxy, alkyl, heteroaryl, heterocyclyl, aryl or —NR¹⁷⁰R¹⁷¹.

In some embodiments Ar¹ is aryl, heteroaryl, or cycloalkyl, and is unsubstituted or substituted with alkyl, alkenyl, alkynyl, heteroaryl, halo, pseudohalo, dialkylamino, aryloxy, aralkoxy, haloalkyl, alkoxy, haloalkoxy, cycloalkyl, or COOR, where R is hydrogen or alkyl;

R⁷ is hydrogen or NRR, where R is hydrogen or alkyl;

R⁸ and R⁹ are each independently selected from (i) and (ii) as follows:

(i) R⁸ and R⁹ together with the atoms to which they are attached form a fused phenyl ring, which is unsubstituted or substituted with halo, pseudohalo, alkyl, alkoxy, cycloalkyl, fused cycloalkyl, fused heterocyclyl, fused heteroaryl, or fused aryl, which is unsubstituted or substituted with halo, pseudohalo, alkyl, alkoxy, aryl, cycloalkyl, heterocyclyl, fused aryl, fused heterocyclyl, and fused cycloalkyl; and

(ii) R⁸ is CN or COOR²⁰⁰ where R²⁰⁰ is hydrogen, alkyl, alkenyl, alkynyl, cycloalkyl, heterocyclyl, aryl or heteroaryl; and R⁹ is hydrogen, alkyl or alkylthio; and

R¹⁰ is hydrogen;

where Ar¹, R⁷, R⁸, R⁹ and R¹⁰ are each independently unsubstituted or substituted with one or more, in one embodiment one, two or three substituents, each independently selected from Q¹.

In some embodiments Ar¹ is phenyl, naphthyl, pyridyl, furyl, or thienyl, and is unsubstituted or substituted with alkyl, alkenyl, halo, pseudohalo, dialkylamino, aryloxy, haloalkyl, alkoxy, aryloxy, cycloalkyl, heterocyclyl, fused heterocyclyl, aryl, fused aryl, heteroaryl, fused heteroaryl, or COOR, where R is hydrogen or alkyl.

In some embodiments Ar¹ is substituted with methyl, fluoro, bromo, chloro, iodo, dimethylamino, phenoxy, trifluoromethyl or methoxycarbonyl.

In some embodiments Ar¹ is phenyl, 2-thienyl, 3-thienyl, 2-furyl, 3-furyl, 5-chloro-2-thienyl, 5-bromo-2-thienyl, 3-methyl-2-thienyl, 5-methyl-2-thienyl, 5-ethyl-2-thienyl, 2-methylphenyl, 3-methylphenyl, 4-fluoro-3-bromophenyl, 2-fluorophenyl, 3,4-difluorophenyl, 2-chlorophenyl, 3-chlorophenyl, 3,4-dichlorophenyl, 3,4,5,-methoxyphenyl, 2,4-methoxyphenyl, 2-fluoro-5-bromophenyl, 4-dimethylaminophenyl, 3-trifluoromethyl, 3-bromophenyl, 2-trifluoromethyl-4-fluorophenyl, 3-trifluoromethyl-4-fluorophenyl, 2-fluoro-3-chlorophenyl, 3-bromo-4-fluorophenyl, perfluorophenyl, 3-pyridyl, 4-pyridyl, 4-bromophenyl, 4-chlorophenyl, 3-phenoxyphenyl, 2,4-dichlorophenyl, 2,3-difluorophenyl, 2-chlorophenyl, 2-fluoro-6-chlorophenyl, 1-naphthyl, 4-trifluoromethylphenyl, 2-trifluoromethylphenyl, 4-trifluoromethoxyphenyl, or 4-methoxycarbonylphenyl.

In some embodiments R⁷ is hydrogen or dialkylamino, or is hydrogen or diethylamino.

In some embodiments R⁸ and R⁹ are each independently selected from (i) and (ii) as follows:

(i) R⁸ and R⁹ together with the atoms to which they are attached form a fused phenyl ring, which is unsubstituted or substituted with methyl, chloro, methoxy, cyclopentyl, fused cyclopentyl, or another fused phenyl ring, which is unsubstituted or substituted with bromo; and

(ii) R⁸ is CN or COOR²⁰⁰, where R²⁰⁰ is methyl, benzyl, ethyl, 4-methoxybenzyl or 2-phenylethyl; and R⁹ is methyl, methylthio or phenylaminocarbonylmethylthio.

In various embodiments, the compound is represented by one of Formulas IIb-IIp:

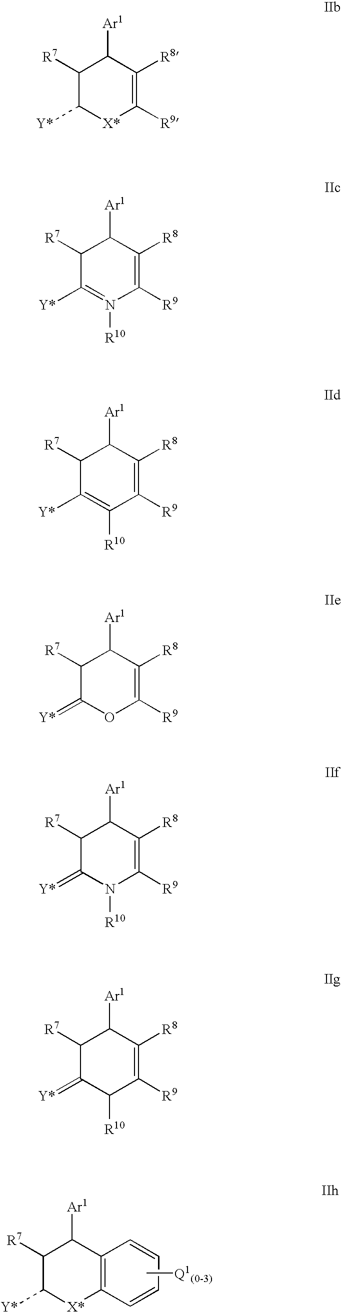

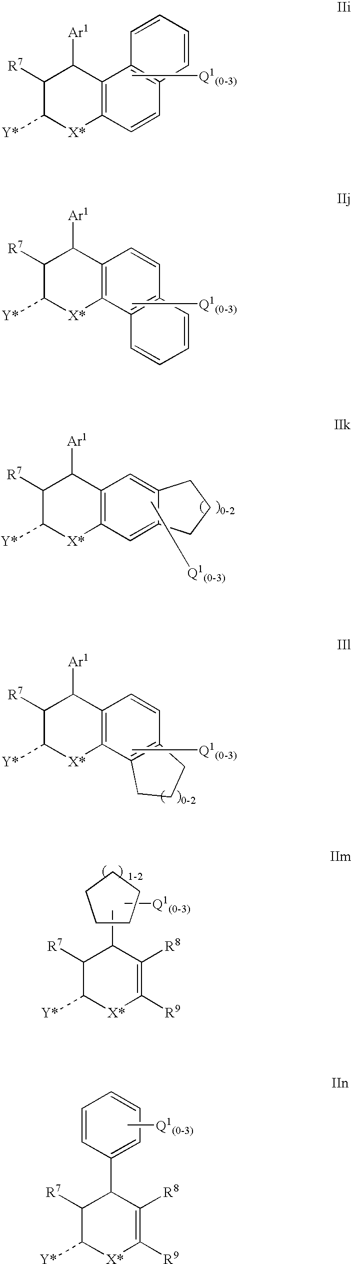

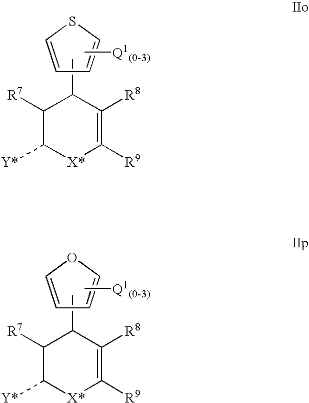

In Formulas IIb-IIp, the variables have the values described herein above for Formulas II and IIa, where X* and Y* are selected such that one of the dashed bonds (— — —) is a single bond and the other is a double bond. In various embodiments represented by Formula Ib, R⁸′ and R⁹′ are independently selected from hydrogen, alkyl, alkenyl, alkynyl, aryl, heteroaryl, heteroarylium, cycloalkyl, heterocyclyl, C(A)R¹¹⁰, halo, pseudohalo, OR¹¹¹, S(D)ₐR¹¹², NR¹¹⁵R¹¹⁶ or N⁺R¹¹⁵R¹¹⁶R¹¹⁷; in some embodiments, R^{8′} is CN or COOR²⁰⁰, where R²⁰⁰ is hydrogen, alkyl, alkenyl, alkynyl, cycloalkyl, heterocyclyl, aryl or heteroaryl; and R⁹′ is hydrogen, alkyl or alkylthio; and in some embodiments, R⁸′ is CN or COOR², where R²⁰⁰ is methyl, benzyl, ethyl, 4-methoxybenzyl or 2-phenylethyl; and R⁹′ is methyl, methylthio or phenylaminocarbonylmethylthio. In various embodiments of Formulas IIh-IIp, each Q¹ is independently selected from halogen, alkyl, alkoxy, nitro, CN, N₃, aryl, aryloxy, arylalkyloxy, alkynyl, amino, alkylamino, heterocyclyl, heteroaryl, substituted carboxyl (e.g., CO₂-alkyl, CO₂-benzyl), haloalkyl, and haloalkoxy, or two adjacent Q¹, on the same phenyl or adjacent fused phenyl rings, together form a cycloalkyl or heterocyclyl ring fused with the phenyl or adjacent fused phenyl rings. In Formulas IIh-IIp, the bond line from Q¹ indicates that each Q¹ may independently be bonded to any ring crossed by the bond line.

In some embodiments, the compound is represented by one of Formulas IIq, IIr, and IIs:

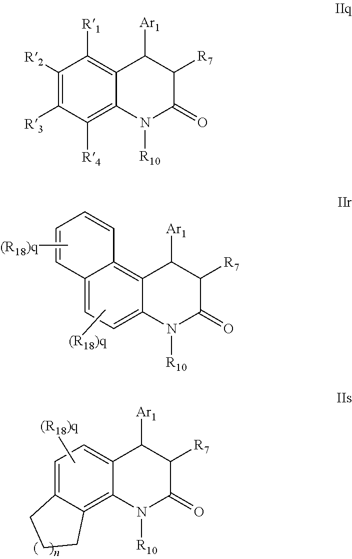

In Formulas IIq, IIr, and IIs, Ar1, R7, and R10 can have the values recited herein; and each q is independently 0, 1, or 2;

n is 0, or 2;

R′1, R′2, R′3, R′4, and each R18 are independently selected from hydrogen, alkyl, alkenyl, alkynyl, aryl, heteroaryl, heteroarylium, cycloalkyl, heterocyclyl, C(A)R¹¹⁰, halo, pseudohalo, OR¹¹¹, S(D)ₐR¹¹², NR¹¹⁵R¹¹⁶ or N⁺R¹¹⁵R¹¹⁶R¹¹⁷, wherein values for A, R¹¹⁰, R¹¹¹, D, a, R¹¹², R¹¹⁵, R¹¹⁶ and R¹¹⁷ are selected as described herein above.

In some embodiments the compound is selected from the compounds in Table II. In certain embodiments, the compound is selected from compounds II.1-II.95 in Table II; in some embodiments, the compound is selected from compounds II.1-II.69 in Table II. In some embodiments, the compound is selected from compounds II.1-II.3 and II.70-II.95 in Table II. In some embodiments, the compound is selected from compounds II.4-II.69 in Table II.

Also disclosed are methods of treating or preventing a disorder characterized by impaired protein trafficking, the method comprising administering to a subject a compound selected from the group consisting of doxorubicin, cycloheximide, hygromycin, novobiocin, aureobasidin, and tunicamycin.

Also provided are pharmaceutically-acceptable derivatives, including salts, esters, enol ethers, enol esters, solvates, hydrates and prodrugs of the compounds described herein. Pharmaceutically-acceptable salts, include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc, aluminum, and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates.

Further provided are pharmaceutical compositions containing any of the compounds described herein and a pharmaceutically acceptable carrier. In one embodiment, the pharmaceutical compositions are formulated for single dosage administration.

In some embodiments, the disorder characterized by impaired protein trafficking is a synucleinopathy. Examples of synucleinopathies include Parkinson's disease, Lewy body disease, the Lewy body variant of Alzheimer's disease, dementia with Lewy bodies, multiple system atrophy, or the Parkinsonism-dementia complex of Guam.

Synucleins are a family of small, presynaptic neuronal proteins composed of alpha-, beta-, and gamma-synucleins, of which only alpha-synuclein aggregates have been associated with several neurological diseases (Ian et al., Clinical Neurosc. Res. 1:445-455, 2001; Trojanowski and Lee, Neutrotoxicology 23:457-460, 2002). The role of synucleins (and in particular, alpha-synuclein) in the etiology of a number of neurodegenerative and/or amyloid diseases has developed from several observations. Pathologically, alpha-synuclein was identified as a major component of Lewy bodies, the hallmark inclusions of Parkinson's disease, and a fragment thereof was isolated from amyloid plaques of a different neurological disease, Alzheimer's disease. Biochemically, recombinant alpha-synuclein was shown to form amyloid-like fibrils that recapitulated the ultrastructural features of alpha-synuclein isolated from patients with dementia with Lewy bodies, Parkinson's disease and multiple system atrophy. Additionally, the identification of mutations within the alpha-synuclein gene, albeit in rare cases of familial Parkinson's disease, demonstrated an unequivocal link between synuclein pathology and neurodegenerative diseases. The common involvement of alpha-synuclein in a spectrum of diseases such as Parkinson's disease, dementia with Lewy bodies, multiple system atrophy and the Lewy body variant of Alzheimer's disease has led to the classification of these diseases under the umbrella term of “synucleinopathies.”

In some embodiments, the disorder characterized by impaired protein trafficking is not a synucleinopathy.

In some embodiments, the disorder characterized by impaired protein trafficking is a lysosomal storage disorder such as Fabry disease, Farber disease, Gaucher disease, GM₁-gangliosidosis, Tay-Sachs disease, Sandhoff disease, GM₂ activator disease, Krabbe disease, metachromatic leukodystrophy, Niemann-Pick disease (types A, B, and C), Hurler disease, Scheie disease, Hunter disease, Sanfilippo disease, Morquio disease, Maroteaux-Lamy disease, hyaluronidase deficiency, aspartylglucosaminuria, fucosidosis, mannosidosis, Schindler disease, sialidosis type 1, Pompe disease, Pycnodysostosis, ceroid lipofuscinosis, cholesterol ester storage disease, Wolman disease, Multiple sulfatase, galactosialidosis, mucolipidosis (types II, III, and IV), cystinosis, sialic acid storage disorder, chylomicron retention disease with Marinesco-Sjögren syndrome, Hermansky-Pudlak syndrome, Chediak-Higashi syndrome, Danon disease, or Geleophysic dysplasia. Lysosomal storage disorders are reviewed in, e.g., Wilcox (2004) J. Pediatr 144:S3-S14.

In some embodiments, the disorder characterized by impaired protein trafficking is characterized by an impaired delivery of cargo to a cellular compartment.

In some embodiments, the disorder characterized by impaired protein trafficking is characterized by a Rab27a mutation or a deficiency of Rab27a. The disorder can be, e.g., Griscelli syndrome.

In some embodiments,.the disorder characterized by impaired protein trafficking is cystic fibrosis.

In some embodiments, the disorder characterized by impaired protein trafficking is diabetes (e.g., diabetes mellitus).

In some embodiments, the disorder characterized by impaired protein trafficking is hereditary emphysema, hereditary hemochromatosis, oculocutaneous albinism, protein C deficiency, type I hereditary angioedema, congenital sucrase-isomaltase deficiency, Crigler-Najjar type II, Laron syndrome, hereditary Myeloperoxidase, primary hypqthyroidism, congenital long QT syndrome, tyroxine binding globulin deficiency, familial hypercholesterolemia, familial chylomicronemia, abeta-lipoproteinema, low plasma lipoprotein a levels, hereditary emphysema with liver injury, congenital hypothyroidism, osteo genesis imperfecta, hereditary hypofibrinogenemia, alpha-lantichymotrypsin deficiency, nephrogenic diabetes insipidus, neurohypophyseal diabetes, insipidus, Charcot-Marie-Tooth syndrome, Pelizaeus Merzbacher disease, von Willebrand disease type IIA, combined factors V and VIII deficiency, spondyloepiphyseal dysplasia tarda, choroideremia, I cell disease, Batten disease, ataxia telangiectasias, acute lymphoblastic leukemia, acute myeloid leukemia, myeloid leukemia, ADPKD-autosomal dominant polycystic kidney disease, microvillus inclusion disease, tuberous sclerosis, oculocerebro-renal syndrome of Lowe, amyotrophic lateral sclerosis, myelodysplastic syndrome, Bare lymphocyte syndrome, Tangier disease, familial intrahepatic cholestasis, X-linked adreno-leukodystrophy, Scott syndrome, Hermansky-Pudlak syndrome types 1 and 2, Zellweger syndrome, rhizomelic chondrodysplasia puncta, autosomal recessive primary hyperoxaluria, Mohr Tranebjaerg syndrome, spinal and bullar muscular atrophy, primary ciliary diskenesia (Kartagener's syndrome), Miller Dieker syndrome, lissencephaly, motor neuron disease, Usher's syndrome, Wiskott-Aldrich syndrome, Optiz syndrome, Huntington's disease, hereditary pancreatitis, anti-phospholipid syndrome, overlap connective tissue disease, Sjögren's syndrome, stiff-man syndrome, Brugada syndrome, congenital nephritic syndrome of the Finnish type, Dubin-Johnson syndrome, X-linked hypophosphosphatemia, Pendred syndrome, persistent hyperinsulinemic hypoglycemia of infancy, hereditary spherocytosis, aceruloplasminemia, infantile neuronal ceroid lipofuscinosis, pseudoachondroplasia and multiple epiphyseal, Stargardt-like macular dystrophy, X-linked Charcot-Marie-Tooth disease, autosomal dominant retinitis pigmentosa, Wolcott-Rallison syndrome, Cushing's disease, limb-girdle muscular dystrophy, mucoploy-saccharidosis type IV, hereditary familial amyloidosis of Finish, Anderson disease, sarcoma, chronic myelomonocytic leukemia, cardiomyopathy, faciogenital dysplasia, Torsion disease, Huntington and spinocerebellar ataxias, hereditary hyperhomosyteinemia, polyneuropathy, lower motor neuron disease, pigmented retinitis, seronegative polyarthritis, interstitial pulmonary fibrosis, Raynaud's phenomenon, Wegner's granulomatosis, preoteinuria, CDG-Ia, CDG-Ib, CDG-Ic, CDG-Id, CDG-Ie, CDG-If, CDG-IIa, CDG-IIb, CDG-IIc, CDG-IId, Ehlers-Danlos syndrome, multiple exostoses, Griscelli syndrome (type 1 or type 2), or X-linked non-specific mental retardation. Disorders characterized by impaired protein trafficking are reviewed in Aridor et al. (2000) Traffic 1:836-51 and Aridor et al. (2002) Traffic 3:781-90.

The subject treated according to the methods described herein can be a human or another mammal such as a mouse, rat, cow, pig, dog, cat, or monkey.

Also disclosed are methods of identifying a compound that rescues impaired endoplasmic reticulum-mediated transport, the method comprising: (i) providing a cell that exhibits reduced expression or activity of a protein required for endoplasmic reticulum-mediated transport; (ii) contacting the cell with a candidate agent; and (iii) determining whether growth of the cell is enhanced in the presence of the candidate agent as compared to in the absence of the candidate agent, wherein a compound that enhances growth is identified as a compound that rescues impaired endoplasmic reticulum-mediated transport. The protein can be, e.g., Ypt1, Rab1a, Rab1b, Rab2, Sar1, Sar1a, Sar1b, Sec23, Sec23a, or Sec23b.

In some embodiments, the method further comprises determining whether a compound identified as enhancing growth of the cell decreases toxicity in a second cell expressing a toxic amount or form of alpha-synuclein.

Also disclosed are methods of identifying a compound that enhances protein secretion, the method comprising: (i) providing a cell that exhibits reduced expression or activity of a protein required for endoplasmic reticulum-mediated transport; (ii) contacting the cell with a candidate agent; and (iii) determining whether protein secretion is enhanced in the presence of the candidate agent as compared to in the absence of the candidate agent, wherein a compound that enhances growth is identified as a compound that enhances protein secretion. The protein can be, e.g., Ypt1, Rab1a, Rab1b, Rab2, Sar1, Sar1a, Sar1b, Sec23, Sec23a, or Sec23b.

Also disclosed are methods of identifying a compound that rescues impaired protein trafficking, the method comprising: (i) providing a cell that exhibits reduced expression or activity of a protein required for protein trafficking; (ii) contacting the cell with a candidate agent; and (iii) determining whether the impairment in protein trafficking is mitigated in the presence of the candidate agent as compared to in the absence of the candidate agent.

Also disclosed are methods of identifying a compound that rescues impaired protein trafficking, the method comprising: (i) providing a cell with a defect in protein trafficking; (ii) contacting the cell with a candidate agent; and (iii) determining whether the impairment in protein trafficking is mitigated in the presence of the candidate agent as compared to in the absence of the candidate agent.

Also disclosed are methods of identifying a compound that rescues impaired Rab-mediated protein trafficking, the method comprising: (i) providing a cell with a defect in a Rab-mediated protein trafficking; (ii) contacting the cell with a candidate agent; and (iii) determining whether the Rab-mediated protein trafficking impairment is mitigated in the presence of the candidate agent as compared to in the absence of the candidate agent. In some embodiments, the defect in a Rab-mediated protein trafficking is defective exocytosis of a bioactive substance. In some embodiments, the defect in a Rab-mediated protein trafficking is caused by a defect in a Rab regulatory protein. In some embodiments, the Rab is Rab27a. In other embodiments, the Rab is selected from Rab1a, Rab1b, Rab8b, Rab8a, Rab10, Rab13, Rab35, Rab11b, Rab30, Rab11a, Rab3a, Rab3c, Rab3d, Rab3b, Rab2, Rab43, Rab4a, Rab2b, Rab4b, Rab25, Rab14, Rab37, Rab18, Rab5b, Rab33a, Rab26, Rab5a, Rab19b, Rab5c, Rab33b, Rab39b, Rab39, Rab31, Rab15, Rab40c, Rab27b, Rab22a, Rab6b, Rab40b, Rasef, Rab21, Rab27a, Loc286526, Rab40a, Rab6a, Rab17, Rab6c, Rab7, Rab9a, Rab711, Rab9b, Rab34, Rab7b, Rab41, Rab23, Rab32, Rab38, Rab36, Rab28, Rab20, or Rab12.

In some embodiments of the methods described herein, the cell is permeabilized.

In some embodiments of the methods described herein, the cell is a yeast cell.

Also provided is a method of identifying a compound that rescues impaired endoplasmic reticulum-mediated transport. The method can include the steps of: providing a cell lysate prepared from a cell that exhibits impaired endoplasmic reticulum-mediated transport; contacting the cell lysate with a candidate agent; and determining whether the candidate agent enhances endoplasmic reticulum-mediated transport in the cell lysate as compared to in the absence of the candidate agent, wherein a compound that enhances growth is identified as a compound that rescues impaired endoplasmic reticulum-mediated transport.

In some embodiments, the cell can exhibit an impaired ability to form COPII vesicles or exhibit impaired docking of COPII vesicles.

In some embodiments, the cell can exhibit reduced expression or activity of a protein required for endoplasmic reticulum-mediated transport. The protein can be Sec23, Sec23a, Sec23b, Sar1, YPT1, Rab1a, Rab1b, or Rab2.

Also featured is a method of identifying a compound that rescues impaired endoplasmic reticulum-mediated transport, which method includes the steps of: contacting a cell that exhibits impaired endoplasmic reticulum-mediated transport with a candidate agent; preparing a cell lysate from the cell; and determining whether endoplasmic reticulum-mediated transport in the lysate in the presence of the candidate agent is enhanced as compared to in the absence of the candidate agent, wherein a compound that enhances endoplasmic reticulum-mediated transport is identified as a compound that rescues impaired endoplasmic reticulum-mediated transport.

In some embodiments, the cell can exhibit an impaired ability to form COPII vesicles or exhibit impaired docking of COPII vesicles.

In some embodiments, the cell can exhibit reduced expression or activity of a protein required for endoplasmic reticulum-mediated transport. The protein can be Sec23, Sec23a, Sec23b, Sar1, YPT1, Rab1a, Rab1b, or Rab2.

The disclosure further provides a method of identifying a compound that rescues impaired endoplasmic reticulum-mediated transport, which method can include the steps of: contacting a cellular material that exhibits impaired formation of COPII vesicles with a candidate agent; and determining whether formation of COPII vesicles in the cellular material is enhanced in the presence of the candidate agent as compared to in the absence of the candidate agent, wherein a compound that enhances formation of COPII vesicles is identified as a compound that rescues impaired endoplasmic reticulum-mediated transport. The cellular material can be a cell or a lysate prepared from a cell (i.e., a cell lysate).

In some embodiments, the cellular material can exhibit an impaired ability to form COPII vesicles or exhibit impaired docking of COPII vesicles.

In some embodiments, the cellular material can exhibit reduced expression or activity of a protein required for docking of COPII vesicles. The protein can be Sec23, Sec23a, Sec23b, or Sar1.

Also featured is a method of identifying a compound that rescues impaired endoplasmic reticulum-mediated transport. The method can include the steps of: contacting a cellular material that exhibits impaired docking of COPII vesicles with a candidate agent; and determining whether docking of COPII vesicles in the cellular material is enhanced in the presence of the candidate agent as compared to in the absence of the candidate agent, wherein a compound that enhances docking of COPII vesicles is identified as a compound that rescues impaired endoplasmic reticulum-mediated transport. The cellular material can be a cell or a lysate prepared from a cell (i.e., a cell lysate).

In some embodiments, the cellular material can exhibits reduced expression or activity of a protein required for docking of COPII vesicles. The protein can be YPT1, Rab1a, Rab1b, or Rab2.

Also provided is a method of identifying a compound that rescues impaired endoplasmic reticulum-mediated transport, which method can include the steps of: contacting a cellular material that exhibits impaired endoplasmic reticulum-mediated transport with a candidate compound that inhibits translation, transcription, a heat shock protein, sphingolipid biosynthesis, protein glycosylation, or the proteasome; and determining whether endoplasmic reticulum-mediated transport in the cellular material is enhanced in the presence of the candidate compound as compared to in the absence of the candidate compound, wherein a candidate compound that enhances endoplasmic reticulum-mediated transport is identified as a compound that rescues impaired endoplasmic reticulum-mediated transport. The method can also include the step of before contacting the cellular material with the candidate compound, determining whether the compound inhibits translation, transcription, a heat shock protein, the proteasome, sphingolipid biosynthesis, or protein glycosylation. The cellular material can be a cell or a lysate prepared from a cell (i.e., a cell lysate).

In some embodiments, the cellular material can exhibit an impaired ability to form COPII vesicles. In some embodiments, the cellular material can exhibit impaired docking of COPII vesicles.

In some embodiments, the cellular material can exhibit reduced expression or activity of a protein required for endoplasmic reticulum-mediated transport. The protein can be Sec23, Sec23a, Sec23b, Sar1, YPT1, Rab1a, Rab1b, or Rab2.

In some embodiments, the compound can inhibit the large subunit of the ribosome, Hsp90, or inositol phosphorylceramide synthase.

The disclosure also features a method of identifying a compound that increases endoplasmic reticulum-mediated transport, which method can include the steps of: providing a cell that exhibits impaired endoplasmic reticulum-mediated transport; contacting the cell with an agent that inhibits expression or activity of Bst1, Emp24, PGAP1, TMED2, TMED10, or TMED7; and measuring endoplasmic reticulum-mediated transport in the cell in the presence of the agent, wherein an increase in endoplasmic reticulum-mediated transport in the presence of the agent as compared to endoplasmic reticulum-mediated transport in the absence of the agent identifies the agent as a compound that increases endoplasmic reticulum-mediated transport. The agent can be a synthetic compound, a naturally occurring compound, a small molecule, nucleic acid, antibody, or peptidomimetic. The cell can be a yeast cell or a mammalian cell such as a mouse cell, a rat cell, or a human cell.

Also featured is a method of identifying a compound that inhibits expression of a protein. The method can include the steps of: providing a cell expressing a protein selected from the group consisting of Bst1, Emp24, PGAP1, TMED2, TMED10, and TMED7;

contacting the cell with an agent; and measuring the expression of the protein in the presence of the agent, wherein a reduction in the expression of the protein in the presence of the agent as compared to the expression of the protein in the absence of the agent identifies the agent as a compound that inhibits the expression of the protein. The agent can be a synthetic compound, a naturally occurring compound, a small molecule, nucleic acid, antibody, or peptidomimetic. The cell can be a yeast cell or a mammalian cell such as a mouse cell, a rat cell, or a human cell.

Featured herein is a method of identifying a compound that inhibits expression of a protein, which method includes the steps of: providing a cell comprising a reporter construct comprising (i) a promoter sequence of a gene encoding a protein selected from the group consisting of Bst1, Emp24, PGAP1, TMED2, TMED10, and TMED7, and (ii) a nucleotide sequence encoding a reporter protein; contacting the cell with an agent; and measuring the expression of the reporter protein in the presence of the agent, wherein a reduction in the expression of the reporter protein in the presence of the agent as compared to the expression of the reporter protein in the absence of the agent identifies the agent as a compound that inhibits the expression of the protein. The agent can be a synthetic compound, a naturally occurring compound, a small molecule, nucleic acid, antibody, or peptidomimetic. The cell can be a yeast cell or a mammalian cell such as a mouse cell, a rat cell, or a human cell.

Also featured is a method of identifying a compound that inhibits the activity of a protein. The method can include the steps of: providing a protein selected from the group consisting of Bst1, Emp24, PGAP1, TMED2, TMED10, and TMED7; contacting the protein with an agent; and measuring the activity of the protein in the presence of the agent, wherein a reduction in the activity of the protein in the presence of the agent as compared to the activity of the protein in the absence of the agent identifies the agent as a compound that inhibits the activity the protein. The agent can be a synthetic compound, a naturally occurring compound, a small molecule, nucleic acid, antibody, or peptidomimetic. The cell can be a yeast cell or a mammalian cell such as a mouse cell, a rat cell, or a human cell.

Also provided is a method of identifying a compound that increases endoplasmic reticulum-mediated transport. The method can include the steps of: providing a cell that exhibits impaired endoplasmic reticulum-mediated transport; contacting the cell with an agent that enhances expression or activity of a protein selected from the group consisting of SEC12, Sec12, SED4, SEC16, HRD3, IRE1, STS1, SEC24, SEL1L, S20orf50, Ire1, Sec24A, Sec24B, Sec24C, and Sec24D; and measuring cell viability in the presence of the agent; wherein an increase in cell viability in the presence of the agent as compared to cell viability in the absence of the agent identifies the agent as a compound that increases endoplasmic reticulum-mediated transport. The agent can be a synthetic compound, a naturally occurring compound, a small molecule, nucleic acid, antibody, or peptidomimetic. The cell can be a yeast cell or a mammalian cell such as a mouse cell, a rat cell, or a human cell.

Also featured is a method of identifying a compound that increases endoplasmic reticulum-mediated transport, which method can include the steps of: screening to identify an agent that enhances expression or activity of a protein selected from the group consisting of SEC12, Sec12, SED4, SEC16, HRD3, IRE1, STS1, SEC24, SEL1L, S20orf50, Ire1, Sec24A, Sec24B, Sec24C, and Sec24D; providing a cell that exhibits impaired endoplasmic reticulum-mediated transport; contacting the cell with the agent; and measuring endoplasmic reticulum-mediated transport in the presence of the agent, wherein an increase in endoplasmic reticulum-mediated transport in the presence of the agent as compared to endoplasmic reticulum-mediated transport in the absence of the agent identifies the agent as a compound that rescues endoplasmic reticulum-mediated transport. The agent can be a synthetic compound, a naturally occurring compound, a small molecule, nucleic acid, antibody, or peptidomimetic. The cell can be a yeast cell or a mammalian cell such as a mouse cell, a rat cell, or a human cell.

Also provided is a method of identifying a compound that increases expression of a protein. The method can include the steps of: providing a cell expressing a protein selected from the group consisting of SEC12, Sec12, SED4, SEC16, HRD3, IRE1, STS1, SEC24, SEL1L, S20orf50, Ire1, Sec24A, Sec24B, Sec24C, and Sec24D; contacting the cell with an agent; and measuring the expression of the protein in the presence of the agent, wherein an increase in the expression of the protein in the presence of the agent as compared to the expression of the protein in the absence of the agent identifies the agent as a compound that increases the expression of the protein. The agent can be a synthetic compound, a naturally occurring compound, a small molecule, nucleic acid, antibody, or peptidomimetic. The cell can be a yeast cell or a mammalian cell such as a mouse cell, a rat cell, or a human cell.

Also featured is a method of identifying a compound that increases expression of a protein. The method can include the steps of: providing a cell comprising a reporter construct comprising (i) a promoter sequence of a gene encoding a protein selected from the group consisting of SEC12, Sec12, SED4, SEC16, HRD3, IRE1, STS1, SEC24, SEL1L, S20orf50, Ire1, Sec24A, Sec24B, Sec24C, and Sec24D, and (ii) a nucleotide sequence encoding a reporter protein; contacting the cell with an agent; and measuring the expression of the reporter protein in the presence of the agent, wherein an increase in the expression of the reporter protein in the presence of the agent as compared to the expression of the protein in the absence of the agent identifies the agent as a compound that increases the expression of the protein. The agent can be a synthetic compound, a naturally occurring compound, a small molecule, nucleic acid, antibody, or peptidomimetic. The cell can be a yeast cell or a mammalian cell such as a mouse cell, a rat cell, or a human cell.

The disclosure also provides a method of identifying a compound that increases the activity of a protein, which method can include the steps of: providing a protein selected from the group consisting of SEC12, Sec12, SED4, SEC16, HRD3, IRE1, STS1, SEC24, SEL1L, S20orf50, Ire1, Sec24A, Sec24B, Sec24C, and Sec24D; contacting the protein with an agent; and measuring the activity of the protein in the presence of the agent, wherein an increase in the activity of the protein in the presence of the agent as compared to the activity of the protein in the absence of the agent identifies the agent as a compound that increases the activity the protein. The agent can be a synthetic compound, a naturally occurring compound, a small molecule, nucleic acid, antibody, or peptidomimetic. The cell can be a yeast cell or a mammalian cell such as a mouse cell, a rat cell, or a human cell.

Also disclosed is a method of producing a protein, which method includes the steps of: culturing a cell in the presence of a compound described herein (e.g., a compound depicted in Table I or II); and purifying a protein produced by the cell, wherein the culturing of the cell in the presence of the compound results in enhanced production of the purified protein as compared to culture of the cell in the absence of the compound. The protein can be a recombinant protein encoded by a heterologous nucleic acid. In some embodiments, the protein is a secreted protein and/or a glycosylated protein. For example, the protein can be a cytokine, a lymphokine, a growth factor, or an antibody. The cell used in the protein production methods can be, e.g., an insect cell, a mammalian cell (e.g., a Chinese Hamster Ovary cell), a fungal cell, or a bacterial cell.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described below. All publications, patent applications, patents, and other references mentioned herein are incorporated by reference in their entirety. In case of conflict, the present application, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a line graph depicting the rescue of the ypt1^{ts} mutant phenotype by the proteasome inhibitor MG132. The optical density at 600 nm (OD₆₀₀) of the ypt1^{ts} yeast cells (a function of the growth of the cells) is represented on the Y-axis. The X-axis represents the concentration of MG132 the cells were exposed to ranging from 0 to 50 μM.

FIG. 2 is a bar graph depicting the rescue of the ypt1^{ts} mutant phenotype by Cpd. I.3 and Cpd. II.3. The Y-axis represents the optical density at 600 nm (OD₆₀₀) of the ypt1^{ts} yeast cells as a function of the growth of the cells. The concentrations of Cpd. I.3 and Cpd. II.3 used were 2.0 μM and 5.0 μM, respectively. Dimethylsulfoxide (DMSO), the carrier in which the compounds were dissolved, was used as a control.

FIGS. 3A and 3B are photographs of western blots depicting the stabilization of ΔF508 CFTR protein in CFBE cells by Cpd. I.3, Cpd. II.2, and VRT-325. FIG. 3A displays data for Cpd. I.3, Cpd. II.2, and DMSO control. CFBE cells at 37° C. were incubated with 10 μM compound or DMSO for 16 hours. Determinations were done in duplicate. FIG. 3B displays data for VRT-325 and a DMSO control. CFBE cells at 37° C. were incubated with 10 μM compound or DMSO for 16 hours. ΔF508 CFTR was detected using an antibody specific for CFTR. “C” and “B” represent the relative positions of the mature form and ER forms of ΔF508 CFTR on the protein gel respectively.

FIG. 4A is a photograph of a western blot depicting the dose-response effect of Cpd. I.3 on the stabilization of ΔF508 CFTR in CFBE cells. CFBE cells were cultured in the absence (the “0” lane) or presence of various concentrations of the compound (1, 2.5, 5, and 10 μM) for 16 hours at 37° C. ΔF508 CFTR was detected using an antibody specific for CFTR. “C” and “B” represent the relative positions of the mature form and ER forms of ΔF508 CFTR on the protein gel respectively.

FIG. 4B is a line graph depicting the plotted intensities of bands “B” or “C” from FIG. 4A as quantitated by densitometry. The Y-axis represents relative intensity and the X-axis represents the concentration of Cpd. I.3. The upper line (curve) (“BandB”) represents the intensity of band “B” at each concentration. The lower line (curve) (“BandC”) represents the plot of the intensity of band “C” at each concentration.

FIG. 4C is a photograph of a western blot depicting the dose-response effect of Cpd. II.2 on the stabilization of ΔF508 CFTR in CFBE cells. CFBE cells were cultured in the absence (the “0” lane) or presence of various concentrations of the compound (1, 2.5, 5, and 10 μM) for 16 hours at 37° C. ΔF508 CFTR was detected using an antibody specific for CFTR. “C” and “B” represent the relative positions of the mature form and ER forms of ΔF508 CFTR on the gel respectively.

FIG. 4D is a line graph depicting the plotted intensities of bands “B” or “C” from FIG. 4C as quantitated by densitometry. The Y-axis represents relative intensity and the X-axis represents the concentration of Cpd. II.2. The upper line (curve) (“BandB”) represents the intensity of band “B” at each concentration. The lower line (curve) (“BandC”) represents the plot of the intensity of band “C” at each concentration.

### DETAILED DESCRIPTION OF THE INVENTION

### A. Definitions

As used herein, pharmaceutically acceptable derivatives of a compound include salts, esters, enol ethers, enol esters, acetals, ketals, orthoesters, hemiacetals, hemiketals, acids, bases, solvates, hydrates or prodrugs thereof Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, nitrates, borates, methanesulfonates, benzenesulfonates, toluenesulfonates, salts of mineral acids, such as but not limited to hydrochlorides, hydrobromides, hydroiodides and sulfates; and salts of organic acids, such as but not limited to acetates, trifluoroacetates, maleates, oxalates, lactates, malates, tartrates, citrates, benzoates, salicylates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl or heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl or heterocyclyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. As used herein, amelioration of the symptoms of a particular disorder by administration of a particular compound or pharmaceutical composition refers to any lessening, whether permanent or temporary, lasting or transient that can be attributed to or associated with administration of the composition.

As used herein, IC₅₀ refers to an amount, concentration or dosage of a particular test compound that achieves a 50% inhibition of a maximal response, such as modulation of protein trafficking, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

As used herein, a prodrug is a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1 985) Medicinal Chemistry A Biochemical Approach, Oxford University Press, New York, pages 388-392).

It is to be understood that the compounds provided herein may contain chiral centers. Such chiral centers may be of either the (R) or (S) configuration, or may be a mixture thereof. Thus, the compounds provided herein may be enantiomerically pure, or be stercoisomeric or diastereomeric mixtures. In the case of amino acid residues, such residues may be of either the L- or D-form. The configuration for naturally occurring amino acid residues is generally L. When not specified the residue is the L form. As used herein, the term “amino acid” refers to α-amino acids which are racemic, or of either the D- or L-configuration. The designation “d” preceding an amino acid designation (e.g., dAla, dSer, dVal, etc.) refers to the D-isomer of the amino acid. The designation “dl” preceding an amino acid designation (e.g., dlPip) refers to a mixture of the L- and D-isomers of the amino acid. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, “alkyl,” “alkenyl” and “alkynyl” carbon chains, if not specified, contain from 1 to 20 carbons, or 1 or 2 to 16 carbons, and are straight or branched. Alkenyl carbon chains of from 2 to 20 carbons, in certain embodiments, contain 1 to 8 double bonds and alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, allyl (propenyl) and propargyl (propynyl). As used herein, lower alkyl, lower alkenyl, and lower alkynyl refer to carbon chains having from about 1 or about 2 carbons up to about 6 carbons. As used herein, “alk(en)(yn)yl” refers to an alkyl group containing at least one double bond and at least one triple bond.

As used herein, “cycloalkyl” refers to a saturated mono- or multi-cyclic ring system, in certain embodiments of 3 to 10 carbon atoms, in other embodiments of 3 to 6 carbon atoms; cycloalkenyl and cycloalkynyl refer to mono- or multicyclic ring systems that respectively include at least one double bond and at least one triple bond. Cycloalkenyl and cycloalkynyl groups may, in certain embodiments, contain 3 to 10 carbon atoms, with cycloalkenyl groups, in further embodiments, containing 4 to 7 carbon atoms and cycloalkynyl groups, in further embodiments, containing 8 to 10 carbon atoms. The ring systems of the cycloalkyl, cycloalkenyl and cycloalkynyl groups may be composed of one ring or two or more rings which may be joined together in a fused, bridged or spiro-connected fashion. “Cycloalk(en)(yn)yl” refers to a cycloalkyl group containing at least one double bond and at least one triple bond.

As used herein, “aryl” refers to aromatic monocyclic or multicyclic groups containing from 6 to 19 carbon atoms. Aryl groups include, but are not limited to groups such as unsubstituted or substituted fluorenyl, unsubstituted or substituted phenyl, and unsubstituted or substituted naphthyl.

As used herein, “heteroaryl” refers to a monocyclic or multicyclic aromatic ring system, in certain embodiments, of about 5 to about 15 members where one or more, in one embodiment 1 to 3, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl.

As used herein, a “heteroarylium” group is a heteroaryl group that is positively charged on one or more of the heteroatoms.

As used herein, “heterocyclyl” refers to a monocyclic or multicyclic non-aromatic ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen or sulfur. In embodiments where the heteroatom(s) is(are) nitrogen, the nitrogen is optionally substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidino, or the nitrogen maybe quaternized to form an ammonium group where the substituents are selected as above.

As used herein, “aralkyl” refers to an alkyl group in which one of the hydrogen atoms of the alkyl is replaced by an aryl group.

As used herein, “heteroaralkyl” refers to an alkyl group in which one of the hydrogen atoms of the alkyl is replaced by a heteroaryl group.

As used herein, “halo”, “halogen” or “halide” refers to F, Cl, Br or I.

As used herein, pseudohalides or pseudohalo groups are groups that behave substantially similar to halides. Such compounds can be used in the same manner and treated in the same manner as halides. Pseudohalides include, but are not limited to, cyanide, oyanate, thiocyanate, selenocyanate, trifluoromethoxy, and azide.

As used herein, “haloalkyl” refers to an alkyl group in which one or more of the hydrogen atoms are replaced by halogen. Such groups include, but are not limited to, chloromethyl, trifluoromethyl and 1-chloro-2-fluoroethyl.

As used herein, “haloalkoxy” refers to RO— in which R is a haloalkyl group.

As used herein, “sulfinyl” or “thionyl” refers to —S(O)—. As used herein, “sulfonyl” or “sulfuryl” refers to —S(O)₂—. As used herein, “sulfo” refers to —S(O)₂O—.

As used herein, “carboxy” refers to a divalent radical, —C(O)O—.

As used herein, “aminocarbonyl” refers to —C(O)NH₂.

As used herein, “alkylaminocarbonyl” refers to —C(O)NHR in which R is alkyl, including lower alkyl. As used herein, “dialkylaminocarbonyl” refers to —C(O)NR′R in which R′ and R are independently alkyl, including lower alkyl; “carboxamide” refers to groups of formula —NR′COR in which R′ and R are independently alkyl, including lower alkyl.

As used herein, “diarylaminocarbonyl” refers to —C(O)NRR′ in which R and R′ are independently selected from aryl, including lower aryl, such as phenyl.

As used herein, “arylalkylaminocarbonyl” refers to —C(O)NRR′ in which one of R and R′ is aryl, including lower aryl, such as phenyl, and the other of R and R′ is alkyl, including lower alkyl.

As used herein, “arylaminocarbonyl” refers to —C(O)NHR in which R is aryl, including lower aryl, such as phenyl.

As used herein, “hydroxycarbonyl” refers to —COOH.

As used herein, “alkoxycarbonyl” refers to —C(O)OR in which R is alkyl, including lower alkyl.

As used herein, “aryloxycarbonyl” refers to —C(O)OR in which R is aryl, including lower aryl, such as phenyl.

As used herein, “alkoxy” and “alkylthio” refer to RO— and RS—, in which R is alkyl, including lower alkyl.

As used herein, “aryloxy” and “arylthio” refer to RO— and RS—, in which R is aryl, including lower aryl, such as phenyl.

As used herein, “alkylene” refers to a straight, branched or cyclic, in certain embodiments straight or branched, divalent aliphatic hydrocarbon group, in one embodiment having from 1 to about 20 carbon atoms, in another embodiment having from 1 to 12 carbons. In a further embodiment alkylene includes lower alkylene. There may be optionally inserted along the alkylene group one or more oxygen, sulfur, including S(═O) and S(═O)₂ groups, or substituted or unsubstituted nitrogen atoms, including —NR— and —N⁺RR— groups, where the nitrogen substituent(s) is(are) alkyl, aryl, aralkyl, heteroaryl, heteroaralkyl or COR′, where R′ is alkyl, aryl, aralkyl, heteroaryl, heteroaralkyl, —OY or —NYY, where Y is hydrogen, alkyl, aryl, heteroaryl, cycloalkyl or heterocyclyl. Alkylene groups include, but are not limited to, methylene (—CH₂—), ethylene (—CH₂CH₂—), propylene (—(CH₂)₃—), methylenedioxy (—O—CH₂—O—) and ethylenedioxy (—O—(CH₂)₂—O—). The term “lower alkylene” refers to alkylene groups having 1 to 6 carbons. In certain embodiments, alkylene groups are lower alkylene, including alkylene of 1 to 3 carbon atoms.

As used herein, “azaalkylene” refers to —(CRR)ₙ—NR—(CRR)ₘ—, where n and m are each independently an integer from 0 to 4. As used herein, “oxaalkylene” refers to (CRR)ₙ—O—(CRR)ₘ—, where n and m are each independently an integer from 0 to 4. As used herein, “thiaalkylene” refers to —(CRR)ₙ—S—(CRR)ₘ—, —(CRR)ₙ—S(═O)—(CRR)ₘ—, and —(CRR)ₙ—S(═O)₂—(CRR)ₘ—, where n and m are each independently an integer from 0 to 4.

As used herein, “alkenylene” refers to a straight, branched or cyclic, in one embodiment straight or branched, divalent aliphatic hydrocarbon group, in certain embodiments having from 2 to about 20 carbon atoms and at least one double bond, in other embodiments 1 to 12 carbons. In further embodiments, alkenylene groups include lower alkenylene. There may be optionally inserted along the alkenylene group one or more oxygen, sulfur or substituted or unsubstituted nitrogen atoms, where the nitrogen substituent is alkyl. Alkenylene groups include, but are not limited to, —CH═CH—CH═CH— and —CH═CH—CH₂—. The term “lower alkenylene” refers to alkenylene groups having 2 to 6 carbons. In certain embodiments, alkenylene groups are lower alkenylene, including alkenylene of 3 to 4 carbon atoms.

As used herein, “alkynylene” refers to a straight, branched or cyclic, in certain embodiments straight or branched, divalent aliphatic hydrocarbon group, in one embodiment having from 2 to about 20 carbon atoms and at least one triple bond, in another embodiment 1 to 12 carbons. In a further embodiment, alkynylene includes lower alkynylene. There may be optionally inserted along the alkynylene group one or more oxygen, sulfur or substituted or unsubstituted nitrogen atoms, where the nitrogen substituent is alkyl. Alkynylene groups include, but are not limited to, —C≡C—C≡C—, —C≡C— and —C≡C—CH₂—. The term “lower alkynylene” refers to alkynylene groups having 2 to 6 carbons. In certain embodiments, alkynylene groups are lower alkynylene, including alkynylene of 3 to 4 carbon atoms.

As used herein, “alk(en)(yn)ylene” refers to a straight, branched or cyclic, in certain embodiments straight or branched, divalent aliphatic hydrocarbon group, in one embodiment having from 2 to about 20 carbon atoms and at least one triple bond, and at least one double bond; in another embodiment 1 to 12 carbons. In further embodiments, alk(en)(yn)ylene includes lower alk(en)(yn)ylene. There may be optionally inserted along the alkynylene group one or more oxygen, sulfur or substituted or unsubstituted nitrogen atoms, where the nitrogen substituent is alkyl. Alk(en)(yn)ylene groups include, but are not limited to, —C═C—(CH₂)ₙ—C≡C—, where n is 1 or 2. The term “lower alk(en)(yn)ylene” refers to alk(en)(yn)ylene groups having up to 6 carbons. In certain embodiments, alk(en)(yn)ylene groups have about 4 carbon atoms.

As used herein, “cycloalkylene” refers to a divalent saturated mono- or multicyclic ring system, in certain embodiments of 3 to 10 carbon atoms, in other embodiments 3 to 6 carbon atoms; cycloalkenylene and cycloalkynylene refer to divalent mono- or multicyclic ring systems that respectively include at least one double bond and at least one triple bond. Cycloalkenylene and cycloalkynylene groups may, in certain embodiments, contain 3 to 10 carbon atoms, with cycloalkenylene groups in certain embodiments containing 4 to 7 carbon atoms and cycloalkynylene groups in certain embodiments containing 8 to 10 carbon atoms. The ring systems of the cycloalkylene, cycloalkenylene and cycloalkynylene groups may be composed of one ring or two or more rings which may be joined together in a fused, bridged or spiro-connected fashion. “Cycloalk(en)(yn)ylene” refers to a cycloalkylene group containing at least one double bond and at least one triple bond.

As used herein, “arylene” refers to a monocyclic or polycyclic, in certain embodiments monocyclic, divalent aromatic group, in one embodiment having from 5 to about 20 carbon atoms and at least one aromatic ring, in another embodiment 5 to 12 carbons. In further embodiments, arylene includes lower arylene. Arylene groups include, but are not limited to, 1,2-, 1,3- and 1,4-phenylene. The term “lower arylene” refers to arylene groups having 6 carbons.

As used herein, “heteroarylene” refers to a divalent monocyclic or multicyclic aromatic ring system, in one embodiment of about 5 to about 15 atoms in the ring(s), where one or more, in certain embodiments 1 to 3, of the atoms in the ring system is a heteroatom, that is, an element other than carbon, including but not limited to, nitrogen, oxygen or sulfur. The term “lower heteroarylene” refers to heteroarylene groups having 5 or 6 atoms in the ring.

As used herein, “heterocyclylene” refers to a divalent monocyclic or multicyclic non-aromatic ring system, in certain embodiments of 3 to 10 members, in one embodiment 4 to 7 members, in another embodiment 5 to 6 members, where one or more, including 1 to 3, of the atoms in the ring system is a heteroatom, that is, an element other than carbon, including but not limited to, nitrogen, oxygen or sulfur.

As used herein, “substituted alkyl,” “substituted alkenyl,” “substituted alkynyl,” “substituted cycloalkyl,” “substituted cycloalkenyl,” “substituted cycloalkynyl,” “substituted aryl,” “substituted heteroaryl,” “substituted heterocyclyl,” “substituted alkylene,” “substituted alkenylene,” “substituted alkynylene,” “substituted cycloalkylene,” “substituted cycloalkenylene,” “substituted cycloalkynylene,” “substituted arylene,” “substituted heteroarylene” and “substituted heterocyclylene” refer to alkyl, alkenyl, alkynyl, cycloalkyl, cycloalkenyl, cycloalkynyl, aryl, heteroaryl, heterocyclyl, alkylene, alkenylene, alkynylene, cycloalkylene, cycloalkenylene, cycloalkynylene, arylene, heteroarylene and heterocyclylene groups, respectively, that are substituted with one or more substituents, in certain embodiments one, two, three or four substituents, where the substituents are as defined herein, in one embodiment selected from Q¹.

As used herein, “alkylidene” refers to a divalent group, such as ═CR′R″, which is attached to one atom of another group, forming a double bond. Alkylidene groups include, but are not limited to, methylidene (═CH₂) and ethylidene (═CHCH₃). As used herein, “arylalkylidene” refers to an alkylidene group in which either R′ or R″ is an aryl group. “Cycloalkylidene” groups are those where R′ and R″ are linked to form a carbocyclic ring. “Heterocyclylid-ene” groups are those where at least one of R′ and R″ contain a heteroatom in the chain, and R′ and R″ are linked to form a heterocyclic ring.

As used herein, “amido” refers to the divalent group —C(O)NH—. “Thioamido” refers to the divalent group —C(S)NH—. “Oxyamido” refers to the divalent group —OC(O)NH—. “Thiaamido” refers to the divalent group —SC(O)NH—. “Dithiaamido” refers to the divalent group —SC(S)NH—. “Ureido” refers to the divalent group —HNC(O)NH—. “Thioureido” refers to the divalent group —HNC(S)NH—.

As used herein, “semicarbazide” refers to —NHC(O)NHNH—. “Carbazate” refers to the divalent group —OC(O)NHNH—. “Isothiocarbazate” refers to the divalent group —SC(O)NHNH—. “Thiocarbazate” refers to the divalent group —OC(S)NHNH—. “Sulfonylhydrazide” refers to the divalent group —SO₂NHNH—. “Hydrazide” refers to the divalent group —C(O)NHNH—. “Azo” refers to the divalent group —N═N—. “Hydrazinyl” refers to the divalent group —NH—NH—.

Where the number of any given substituent is not specified (e.g., haloalkyl), there may be one or more substituents present. For example, “haloalkyl” may include one or more of the same or different halogens.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

### B. Compounds

The compounds disclosed herein for use in the compositions and methods provided herein rescue protein trafficking defects and can be used to treat a wide variety of disorders characterized by impaired protein trafficking.

In one embodiment, the compounds for use in the compositions and methods provided herein have the Formula Ia:

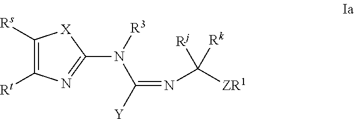

or a pharmaceutically acceptable derivative thereof. In Formula Ia, R^{j} and R^{k} are independently selected from hydrogen, alkyl, alkenyl, alkynyl, cycloalkyl, heterocyclyl, aryl, heteroaryl or aralkyl; or, R^{j} and R^{k}, together with the carbon to which they are both bonded, are —C(═O)—, —CH(OR*)—, —C(═S)—, —CH(SR*)—, —CH(NR*R*′)-or —C(═NR*)—, where R* and R*′ are independently hydrogen, alkyl, alkenyl, alkynyl, cycloalkyl, heterocyclyl, aryl, heteroaryl or aralkyl, R^{s} and R^{t} are independently selected from hydrogen, alkyl, halo, pseudohalo, alkenyl, alkynyl, cycloalkyl, heterocyclyl, aryl, heteroaryl or aralkyl; or, R^{s} and R^{t}, together with the carbon-carbon double bond between them, form a 4-6 membered cycloalkenyl, aryl, heterocyclyl, or heteroaryl ring, wherein the ring formed by R^{s} and R^{t} is optionally substituted with 0-4 substituents R² defined herein below.

Also described herein are compounds represented by Formula Ia or pharmaceutically acceptable derivatives thereof, wherein R^{j} and R^{k} are independently selected from hydrogen, alkyl, alkenyl, alkynyl, cycloalkyl, heterocyclyl, aryl, heteroaryl or aralkyl; or, R^{j} and R^{k}, together with the carbon to which they are both bonded, are —C(═O)—, —CH(OR*)—, —C(═S)—, —CH(SR*)—, —CH(NR*R*′)-or —C(═NR*)—; Y is NRR″, OR′, SR′, or CRR″; where R″ is hydrogen, alkyl, alkenyl, alkynyl, cycloalkyl, heterocyclyl, aryl, heteroaryl or aralkyl, or R″, together with R³ and the atoms therebetween, is a 4-6 membered heterocyclyl or heteroaryl ring; provided that when R^{j} and R^{k}, together with the carbon to which they are both bonded, are —C(═O)—, R″, together with R³ and the atoms therebetween, is a 4-6 membered heterocyclyl or heteroaryl ring. In some embodiments, when R^{j} and R^{k}, together with the carbon to which they are both bonded, are —C(═O)—, —CH(OR*)—, —C(═S)—, —CH(SR*)—, —CH(NR*R*′)-or —C(═NR*)—, Y is NRR″ or CRR″ and R″, together with R³ and the atoms therebetween, is a 4-6 membered heterocyclyl or heteroaryl ring. In various embodiments of the compound represented by Formula Ia, R^{j} and R^{k} are independently selected from hydrogen, alkyl, alkenyl, alkynyl, cycloalkyl, heterocyclyl, aryl, heteroaryl or aralkyl, or R^{j} and R^{k}, taken together, are —CH(OR*)—, —C(═S)—, —CH(SR*)—, —CH(NR*R*′)-or —C(═NR*)—. In some embodiments, the compounds are represented by Formula Ia or pharmaceutically acceptable derivatives thereof wherein R^{j} and R^{k} are independently selected from hydrogen, alkyl, alkenyl, alkynyl, cycloalkyl, heterocyclyl, aryl, heteroaryl or aralkyl. Also described herein are pharmaceutical compositions comprising the compounds and a pharmaceutically acceptable carrier.

In some embodiments, the compound is represented by structural Formula I:

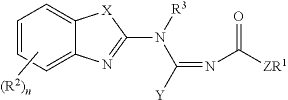

or a pharmaceutically acceptable derivative thereof.

In Formulas Ia and I:

X is O, S or NR, where R is hydrogen, alkyl, alkenyl, alkynyl, cycloalkyl, heterocyclyl, aryl, heteroaryl or aralkyl; in some embodiments, when R^{j} and R^{k} in Formula Ia are both hydrogen, X is O;

Y is NRR′ or OH; where R′ is hydrogen, alkyl, alkenyl, alkynyl, cycloalkyl, heterocyclyl, aryl, heteroaryl or aralkyl; in some embodiments, Y is NRR″, OR′, SR′, or CRR″; where R″ is hydrogen, alkyl, alkenyl, alkynyl, cycloalkyl, heterocyclyl, aryl, heteroaryl or aralkyl, or R″, together with R³ and the atoms therebetween, is a 4-6 membered heterocyclyl or heteroaryl ring, for example, the heteroaryl rings represented by rings A and B in the following compounds:

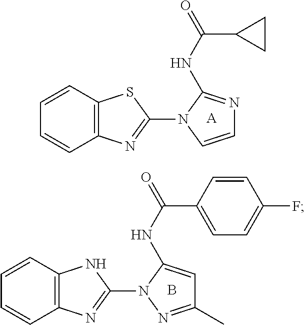

Z is a direct bond or NR;

R¹ is hydrogen, alkyl, alkenyl, alkynyl, cycloalkyl, heterocyclyl, aryl, heteroaryl, aralkyl, aralkenyl, heteroaralkyl or heteroaralkenyl; in some embodiments, when R^{j} and R^{k} in Formula Ia are both hydrogen, R¹ is a cycloalkyl group; in some embodiments, when R^{j} and R^{k} in Formula Ia are both hydrogen, R¹ is a cycloalkyl and Z is a direct bond; in some embodiments, when R^{j} and R^{k} in Formula Ia are both hydrogen, R¹ is a cycloalkyl, Z is a direct bond, and X is O;

n is 0 to 4;

R² is selected from (i) or (ii) as follows:

(i) hydrogen, alkyl, alkenyl, alkynyl, aryl, heteroaryl, heteroarylium, cycloalkyl, heterocyclyl, C(A)R¹¹⁰, halo, pseudohalo, OR¹¹¹, S(D)ₐR¹¹², NR¹¹⁵R¹¹⁶ or N⁺R¹¹⁵R¹¹⁶R¹¹⁷; or

(ii) any two R² groups, which substitute adjacent atoms on the ring, together form alkylene, alkenylene, alkynylene or heteroalkylene;

A is O, S or NR¹²⁵;

R¹¹⁰ is hydrogen, alkyl, alkenyl, alkynyl, aryl, heteroaryl, heteroarylium, cycloalkyl, heterocyclyl, C(A)R¹²⁶, halo pseudohalo, OR¹²⁵, SR¹²⁵, NR¹²⁷R¹²⁸ or SiR¹²²R¹²³R¹²⁴;

R¹¹¹ is hydrogen, alkyl, alkenyl, alkynyl, aryl, heteroaryl, heteroarylium, cycloalkyl, heterocyclyl, C(A)R¹²⁹, NR¹³⁰R¹³¹ or SiR¹²²R¹²³R¹²⁴;

D is O or NR¹²⁵;

a is 0, 1 or 2;

when a is 1 or 2, R¹¹² is selected from hydrogen, alkyl, alkenyl, alkynyl, aryl, heteroaryl, heteroarylium, cycloalkyl, heterocyclyl, halo, pseudohalo, OR¹²⁵, SR¹²⁵ and NR¹³²R¹³³;

when a is 0, R¹¹² is selected from hydrogen, alkyl, alkenyl, alkynyl, aryl, heteroaryl, heteroarylium, cycloalkyl, heterocyclyl, SR¹²⁵ and C(A)R¹²⁹;

R¹¹⁵, R¹¹⁶ and R¹¹⁷ are each independently selected from (a) and (b) as follows:

(a) hydrogen, alkyl, alkenyl, alkynyl, aryl, heteroaryl, heteroarylium, cycloalkyl, heterocyclyl, C(A)R¹²⁹, OR¹²⁵ or NR¹³²R¹³³; or

(b) any two of R¹¹⁵, R¹¹⁶ and R¹¹⁷ together form alkylene, alkenylene, alkynylene, heteroalkylene, and the other is selected as in (a);

R¹²², R¹²³ and R¹²⁴ are selected as in (i) or (ii) as follows:

(i) R¹²², R¹²³ and R¹²⁴ are each independently hydrogen, alkyl, alkenyl, alkynyl, aryl, heteroaryl, heteroarylium, cycloalkyl, heterocyclyl, OR¹²⁵ or NR¹³²R¹³³; or

(ii) any two of R¹²², R¹²³ and R¹²⁴ together form alkylene, alkenylene, alkynylene, heteroalkylene; and the other is selected as in (i);

R¹²⁵ is hydrogen, alkyl, alkenyl, alkynyl, aryl, heteroaryl, heteroarylium, cycloalkyl or heterocyclyl;

R¹²⁶ is hydrogen, alkyl, alkenyl, alkynyl, aryl, heteroaryl, heteroarylium, cycloalkyl, heterocyclyl, OR¹²⁵ or NR¹³⁴R¹³⁵; where R¹³⁴ and R¹³⁵ are each independently hydrogen, alkyl, alkenyl, alkynyl, aryl, heteroaryl, heteroarylium, cycloalkyl, heterocyclyl, OR¹³⁶ or NR¹³²R¹³³, or R¹³⁴ and R¹³⁵ together form alkylene, alkenylene, alkynylene, heteroalkylene, where R¹³⁶ is hydrogen, alkyl, alkenyl, alkynyl, aryl, heteroaryl, heteroarylium, cycloalkyl or heterocyclyl;

R¹²⁷ and R¹²⁸ are selected as in (i) or (ii) as follows:

(i) R¹²⁷ and R¹²⁸ are each independently hydrogen, alkyl, alkenyl, alkynyl, aryl, heteroaryl, heteroarylium, cycloalkyl, heterocyclyl, OR¹²⁵, NR¹³⁷R¹³⁸ or C(A)R¹³⁹, where R¹³⁷ and R¹³⁸ are each independently hydrogen, alkyl, alkenyl, alkynyl, aryl, heteroaryl, heteroarylium, cycloalkyl or heterocyclyl, or together form alkylene, alkenylene, alkynylene, heteroalkylene; and R¹³⁹ is hydrogen, alkyl, alkenyl, alkynyl, aryl, heteroaryl, heteroarylium, cycloalkyl, heterocyclyl, OR¹⁴⁰ or NR¹³²R¹³³, where R¹⁴⁰ is alkyl, alkenyl, alkynyl, aryl, heteroaryl, heteroarylium, cycloalkyl, heterocyclyl; or

(ii) R¹²⁷ and R¹²⁸ together form alkylene, alkenylene, alkynylene, heteroalkylene;

R¹²⁹ is hydrogen, alkyl, alkenyl, alkynyl, aryl, heteroaryl, heteroarylium, cycloalkyl, heterocyclyl, OR¹⁴⁰ or NR¹³²R¹³³;

R¹³⁰ and R¹³¹ are each independently hydrogen, alkyl, alkenyl, alkynyl, aryl, heteroaryl, heteroarylium, cycloalkyl, heterocyclyl or C(A)R¹⁴¹, where R¹⁴¹ is alkyl, alkenyl, alkynyl, aryl, heteroaryl, heteroarylium, cycloalkyl, heterocyclyl, OR¹²⁵ or NR¹³²R³³; or R¹³⁰ and R¹³¹ together form alkylene, alkenylene, alkynylene, heteroalkylene;

R¹³² and R¹³³ are each independently hydrogen, alkyl, alkenyl, alkynyl, aryl, heteroaryl, heteroarylium, cycloalkyl, heterocyclyl, or R¹³² and R¹³³ together form alkylene, alkenylene, alkynylene, heteroalkylene; and

R³ is hydrogen, alkyl, alkenyl, alkynyl, cycloalkyl, heterocyclyl, aryl or heteroaryl;

wherein X, Y, Z, R¹, R² and R³, or in some embodiments, X, Y, Z, R, R′, R″, R*, R¹, R² and R³, are each independently unsubstituted or substituted with one or more substituents, in one embodiment one, two or three substituents, each independently selected from Q¹, where Q¹ is halo, pseudohalo, hydroxy, oxo, thia, nitrile, nitro, formyl, mercapto, hydroxycarbonyl, hydroxycarbonylalkyl, hydroxycarbonylalkenyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, alkoxyxarbonylalkoxy, aryloxycarbonyl, aryloxycarbonylalkyl, aralkoxycarbonyl, aralkoxycarbonylalkyl, aralkoxycarbonylalkoxy, arylcarbonylalkyl, aminocarbonyl, aminocarbonylalkyl, aminocarbonylalkoxy, alkylaminocarbonyl, alkylaminocarbonylalkyl, alkylaminocarbonylalkoxy, dialkylaminocarbonyl, dialkylaminocarbonylalkyl, dialkylaminocarbonylalkoxy, arylaminocarbonyl, aryl aminocarbonylalkyl, arylaminocarbonylalokoxy, diarylaminocarbonyl, diarylaminocarbonylalkyl, diarylaminocarbonyl alkoxy, arylalkylaminocarbonyl, arylalkylaminocarbonylalkyl, arylalkylaminocarbonylalkoxy, alkoxy, aryloxy, hetero aryloxy, hetero aralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diaryl aminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylaamino, aryloxycarbonylamino, alkylsulfonylamino, arylsulfonylamino, heteroarylsulfonylamino, heterocyclylsulfonylamino, heteroarylthio, azido, —N⁺R¹⁵¹R¹⁵²R¹⁵³, P(R¹⁵⁰)₂, P(═O)(R¹⁵⁰)₂, OP(═O)(R¹⁵⁰)₂, —NR¹⁶⁰C(═O)R¹⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; azido, tetrazolyl or two Q¹ groups, which substitute atoms in a 1,2 or 1,3 arrangement, together form alkylenedioxy (i.e., —O—(CH₂)_{y}—O—), thioalkylenoxy (i.e., —S—(CH₂)_{y}—O—)or alkylenedithioxy (i.e., —S—(CH₂)_{y}—S—) where y is 1 or 2; or two Q¹ groups, which substitute the same atom, together form alkylene; and

each Q¹ is independently unsubstituted or substituted with one or more substituents, in one embodiment one, two or three substituents, each independently selected from Q²;

each Q² is independently halo, pseudohalo, hydroxy, oxo, thia, nitrile, nitro, formyl, mercapto, hydroxycarbonyl, hydroxycarbonylalkyl, hydroxycarbonylalkenyl alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R¹⁵¹R¹⁵²R¹⁵³, P(R¹⁵²)₂, P(═O)(R¹⁵⁰)₂, OP(═O)(R¹⁵⁰)₂, —NR¹⁶⁰C(═O)R¹⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q² groups, which substitute atoms in a 1,2 or 1,3 arrangement, together form alkylenedioxy (i.e. —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q² groups, which substitute the same atom, together form alkylene;

R¹⁵⁰ is hydroxy, alkoxy, aralkoxy, alkyl, heteroaryl, heterocyclyl, aryl or —NR¹⁷⁰R¹⁷¹, where R¹⁷⁰ and R¹⁷¹ are each independently hydrogen, alkyl, aralkyl, aryl, heteroaryl, heteroaralkyl or heterocyclyl, or R¹⁷⁰ and R¹⁷¹ together form alkylene, azaalkylene, oxaalkylene or thiaalkylene;

R¹⁵¹, R¹⁵² and R¹⁵³ are each independently hydrogen, alkyl, aryl, aralkyl, heteroaryl, heteroaralkyl, heterocyclyl or heterocyclylalkyl;

R¹⁶⁰ is hydrogen, alkyl, aryl, aralkyl, heteroaryl, heteroaralkyl, heterocyclyl or heterocyclylalkyl; and

R¹⁶³ is alkoxy, aralkoxy, alkyl, heteroaryl, heterocyclyl, aryl or —NR¹⁷⁰R¹⁷¹.

In some embodiments, R¹ is substituted with one or more substituents independently selected from aryloxy, aryl, heteroaryl, halo, pseudohalo, alkyl, alkoxy, cycloalkyl, alkoxycarbonyl, hydroxycarbonyl, alkylamino, and dialkylamino.

As one of skill in the art will recognize, Formulas Ia and I structurally set forth one tautomeric form of the compounds encompassed therein; all such tautomeric forms are contemplated herein. For example, Formulas Ia and I include a fragment represented by —NH—CH(Y)═N—, and when Y is NH₂, the fragment is a guanidine group which includes the three tautomeric forms —NH—CH(NH₂)═N—, —NH—CH(═NH)—NH—, and —N═CH(NH₂)—NH—.

In some embodiments:
X is O, S or NR, where R is hydrogen or alkyl;
Y is NRR′ or OH, where R is hydrogen or alkyl;
Z is a direct bond or NR;
R¹ is alkyl, alkenyl, alkynyl, cycloalkyl, aryl, heterocyclyl, heteroaryl, aralkyl, aralkenyl, heteroaralkyl, or heteroaralkenyl;
R⁷ is halo, pseudohalo, alkoxy or alkyl;
n is 0 or 1;
R³ is hydrogen or alkyl;

wherein X, Y, Z, R¹, R² and R³ are each independently unsubstituted or substituted with one or more substituents, in one embodiment one, two or three substituents, each independently selected from Q¹.

In some embodiments R is hydrogen.

In some embodiments n is 0 or 1.

In some embodiments X is S, O or NH.

In some embodiments Y is NH₂.

In some embodiments Z is a direct bond or NH.

In some embodiments R¹ is alkyl, alkenyl, cycloalkyl, heterocyclyl, aryl or heteroaryl, and is unsubstituted or substituted with aryloxy, aryl, heteroaryl, halo, pseudohalo, alkyl, alkoxy, cycloalkyl, alkoxycarbonyl, hydroxycarbonyl, alkylamino, and dialkylamino.

In some embodiments R¹ is ethyl, 2-(2-furyl)ethenyl, phenyl, methyl, 2-naphthyloxymethyl, benzyl, 3-chloro-2-benzothienyl, cyclopropyl, cyclopropylmethyl, isobutyl, 4-tert-butylphenyl, 4-biphenyl, tert-butyl, 3-chlorophenyl, 2-furyl, 2,4-dichlorophenyl, 3,4-dimethoxyphenyl, 2-(4-methoxyphenyl)ethenyl, 4-methoxyphenoxymethyl, isopentyl, isopropyl, 2-cyclopentylethyl, cyclopentylmethyl, 2-phenylpropyl, 2-phenylethyl, 1-methyl-2-phenylethyl, 1-methyl-2-phenylethenyl, 2-benzylethyl, 2-phenylethenyl, 5-hexynyl, 3-butynyl, 4-pentynyl, propyl, butyl, pentyl, hexyl, t-butoxymethyl, t-butylmethyl, 1-ethylpentyl, cyclopropyl, cyclopropylmethyl, cyclopentyl, cyclohexyl, cyclobutyl, 2-cyclopentylethyl, cyclopentylmethyl, 2-fluorocyclopropyl, 2-methylcyclopropyl, 2-phenylcyclopropyl, 2,2-dimethylethenyl, 1,2-propenyl, 2-(3-trifluoromethylphenyl)ethenyl, 3,4-butenyl, 2-(2-furyl)ethyl, 2-chloroethenyl, 2-(2-chlorophenyl)ethenyl, 1-methyl-2,2-dichlorocyclopropyl, 2,2-difluorocyclopropyl, methylpropionate, proprionic acid, methylbutyrate, butyric acid, pentanoic acid, methyl-t-butylether, dimethylaminomethyl, 2-(2-tetrahydrofuryl)-ethyl, or 2-(2-tetrahydrofuryl)-methyl.

In some embodiments R² is halo or alkyl.

In some embodiments R² is chloro or methyl.

In some embodiments R³ is hydrogen.

In various embodiments, the compound is represented by one of Formulas Ib-Im:

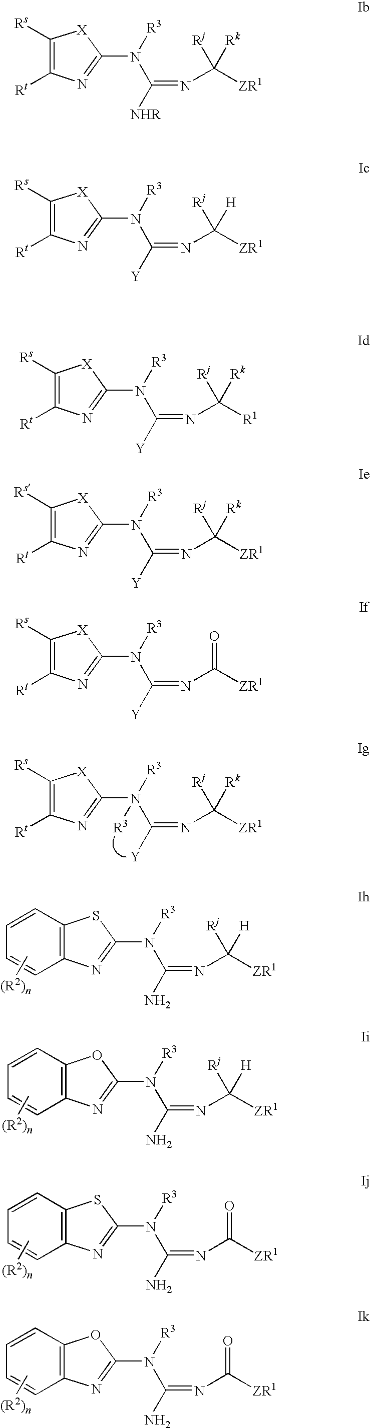

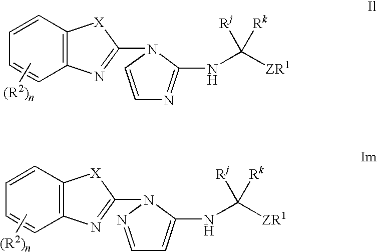

In Formulas Ib-Im, the variables have the values described herein above for Formulas I and Ia.

In various embodiments, R¹ in Formulas Ib-Im is hydrogen, alkyl, aryl, aralkyl, aralkenyl, alkynyl, heteroaryl, heteroaralkyl, heteroarylalkenyl, cycloalkyl, each of which is substituted with 0, 1 or 2 groups selected from phenyl, alkyl, cycloalkyl, alkoxy, halo, pseudohalo, amino, alkylamino, or dialkylamino. In various embodiments, R¹ in Formulas Ib-Im is phenyl, furyl, thienyl, alkynyl, alkyl, cyclopropyl, cyclobutyl or cyclopentyl; or alkyl or alkenyl substituted with phenyl, furyl, thienyl, alkynyl, alkyl, cyclopropyl, cyclobutyl or cyclopentyl; in some embodiments, R¹ is optionally substituted with 0, 1 or 2 groups selected from phenyl, alkyl, alkoxy, halo, or CN.

In some embodiments, R^{j} and R^{k} in Formulas Ib-Im are both hydrogen. In some embodiments, R³ in Formulas Ib-Im is hydrogen.

In various embodiments represented by Formula Ie, R^{s}′ and R^{t}′ are independently selected from hydrogen, alkyl, halo, pseudohalo, alkenyl, alkynyl, cycloalkyl, heterocyclyl, aryl, heteroaryl or aralkyl; in some embodiments, R^{s}′ and R^{t}′ are independently selected from hydrogen, alkyl, and halo; and in certain embodiments, R^{s}′ and R^{t}′ are independently selected from hydrogen, alkyl, and Br, wherein typically, R^{s}′ and R^{t}′ are not both hydrogen.

In some embodiments of Formulas Ih-Im, n is 0, 1 or 2 and each R² is independently selected from halogen, alkyl, alkoxy, haloalkyl, and haloalkoxy; in some embodiments, n is 0, 1 or 2 and each R² is independently selected from hydrogen, F, fluoroalkyl (e.g., CHF₂, CF₃), and fluoroalkoxy (e.g., OCHF₂, OCF₃).

In some embodiments the compound is selected from the compounds in Table I. In certain embodiments, the compound is selected from compounds I. 1-1.57 in Table I; in some embodiments, the compound is selected from compounds I.1-I.35 in Table I. In some embodiments, the compound is selected from compounds I.1-I.6 and I.36-I.57 in Table I. In some embodiments, the compound is selected from compounds I.7-I.35 in Table I.

In another embodiment, the compounds for use in the compositions and methods provided herein have Formula IIa:

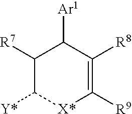

or a pharmaceutically acceptable derivative thereof. In Formula Ia, X* is selected from the group consisting of —O—, ═N—, —N(R^{o})—, ═C(R^{o})— and —C(R^{o}R^{o}′)—, and Y* is selected from ═O, —OR^{o}, ═NR^{o}′, —NR^{o}R^{o}′, —CR^{o}R^{o}′ and —CHR^{o}R^{o}′; where X* and Y* are selected such that one of the dashed bonds (- - -) is a single bond and the other is a double bond, or both dashed bonds are single bonds. Each R^{o}′ is independently selected from the group consisting of hydrogen, halogen, pseudohalo, amino, amido, carboxamido, sulfonamide, carboxyl, alkyl, alkenyl, alkynyl, cycloalkyl, heterocyclyl, aryl, heteroaryl, aralkyl, alkoxy, cycloalkoxy, heterocycloxy, aryloxy, heteroaryloxy, and aralkyloxy. Each R^{o} is selected from the group consisting of hydrogen, alkyl, alkenyl, alkynyl, cycloalkyl, heterocyclyl, aryl, heteroaryl and aralkyl. In some embodiments, R^{o}′ is independently selected from the group consisting of hydrogen, alkyl, alkenyl, alkynyl, cycloalkyl, heterocyclyl, aryl, heteroaryl or aralkyl. In certain embodiments, R^{o} is hydrogen or alkyl, typically hydrogen.

Also described herein are compounds represented by Formula Ia or pharmaceutically acceptable derivatives thereof, wherein X* is selected from the group consisting of —O—, ═N—, —N(R^{o})—, ═C(R^{o})— and —C(R^{o}R^{o}′)—; and Y* is selected from the group consisting of ═O, —OR^{o}, ═NR^{o}′, —NR^{o}R^{o}′, ═CR^{o}R^{o}′ and —CHR^{o}R^{o}′; where X* and Y* are selected such that both dashed bonds are single bonds, or one of the dashed bonds (- - -) is a single bond and the other is a double bond, provided that Y* is not ═O when X* is —N(H)—. In various embodiments of the compounds represented by by Formula IIa, X* and Y* are selected such that both dashed bonds are single bonds, or one of the dashed bonds (- - -) is a single bond and the other is a double bond, provided that Y* is not ═O when X* is —N(R^{o})—. In some embodiments of the compounds represented by by Formula IIa, X* and Y* are selected such that both dashed bonds are single bonds, or one of the dashed bonds (- - -) is a single bond and the other is a double bond, provided that Y* is not ═O, ═NR^{o}′, or ═CR^{o}R^{o}′ when X* is —N(R^{o})—. Also described herein are pharmaceutical compositions comprising the compounds of Formula IIa and a pharmaceutically acceptable carrier.

In some embodiments, the compounds of Formula IIa can also be represented by Formula II:

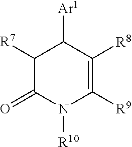

or a pharmaceutically acceptable derivative thereof.

In Formulas IIa and II:

Ar¹ is aryl, heteroaryl, or cycloalkyl;

R⁷ is hydrogen, alkyl, alkenyl, alkynyl, cycloalkyl, heterocyclyl, aryl, heteroaryl or NRR, where R is hydrogen or alkyl;

R¹⁰ is hydrogen, alkyl, alkenyl, alkynyl, cycloalkyl, heterocyclyl, aryl or heteroaryl;

R⁸ and R⁹ are each independently selected from (i) or (ii) as follows:

(i) hydrogen, alkyl, alkenyl, alkynyl, aryl, heteroaryl, heteroarylium, cycloalkyl, heterocyclyl, C(A)R¹¹⁰, halo, pseudohalo, OR¹¹¹, S(D)ₐR¹¹², NR¹¹⁵R¹¹⁶ or N⁺R¹¹⁵R¹¹⁶R¹¹⁷; or

(ii) R⁸ and R⁹ together form alkylene, alkenylene, alkynylene or heteroalkylene; for example, in some embodiments, R⁸ and R⁹ together with the atoms to which they are attached form a fused phenyl ring, which is unsubstituted or substituted with halo, pseudohalo, alkyl, alkoxy, cycloalkyl, fused cycloalkyl, fused heterocyclyl, fused heteroaryl, or fused aryl, which is unsubstituted or substituted with halo, pseudohalo, alkyl, alkoxy, aryl, cycloalkyl, heterocyclyl, fused aryl, fused heterocyclyl, and fused cycloalkyl;

A is O, S or NR¹²⁵;

R¹¹⁰ is hydrogen, alkyl, alkenyl, alkynyl, aryl, heteroaryl, heteroarylium, cycloalkyl, heterocyclyl, C(A)R¹²⁶, halo, pseudohalo, OR¹²⁵, SR¹²⁵, NR¹²⁷R¹²⁸ and SiR¹²²R¹²³R¹²⁴;

R¹¹¹ is hydrogen, alkyl, alkenyl, alkynyl, aryl, heteroaryl, heteroarylium, cycloalkyl, heterocyclyl, C(A)R¹²⁹, NR¹³⁰R¹³¹ and SiR¹²²R¹²³R¹²⁴;

D is O or NR¹²⁵;

a is 0, 1 or 2;

when a is 1 or 2, R¹¹² is selected from hydrogen, alkyl, alkenyl, alkynyl, aryl, heteroaryl, heteroarylium, cycloalkyl, heterocyclyl, halo, pseudohalo, OR¹²⁵, SR¹²⁵ and NR¹³²R¹³³;

when a is 0, R¹¹² is selected from hydrogen, alkyl, alkenyl, alkynyl, aryl, heteroaryl, heteroarylium, cycloalkyl, heterocyclyl, SR¹²⁵ and C(A)R¹²⁹;

R¹¹⁵, R¹¹⁶ and R¹¹⁷ are each independently selected from (a) and (b) as follows:

(a) hydrogen, alkyl, alkenyl, alkynyl, aryl, heteroaryl, heteroarylium, cycloalkyl, heterocyclyl, C(A)R¹²⁹, OR¹²⁵ or NR¹³²R¹³³; or

(b) any two of R¹¹⁵, R¹¹⁶ and R¹¹⁷ together form alkylene, alkenylene, alkynylene, heteroalkylene, and the other is selected as in (a);

R¹²², R¹²³ and R¹²⁴ are selected as in (i) or (ii) as follows:

(i) R¹²², R¹²³ and R¹²⁴ are each independently hydrogen, alkyl, alkenyl, alkynyl, aryl, heteroaryl, heteroarylium, cycloalkyl, heterocyclyl, OR¹²⁵ or NR¹³²R¹³³; or

(ii) any two of R¹²², R¹²³ and R¹²⁴ together form alkylene, alkenylene, alkynylene, heteroalkylene; and the other is selected as in (i);

R¹²⁵ is hydrogen, alkyl, alkenyl, alkynyl, aryl, heteroaryl, heteroarylium, cycloalkyl or heterocyclyl; in some embodiments, where R¹²⁵ is alkyl, alkenyl, or alkynyl, R¹²⁵ is optionally substituted with aryl, heteroaryl, heteroarylium, cycloalkyl or heterocyclyl;

R¹²⁶ is hydrogen, alkyl, alkenyl, alkynyl, aryl, heteroaryl, heteroarylium, cycloalkyl, heterocyclyl, OR¹²⁵ or NR¹³⁴R¹³⁵; where R¹³⁴ and R¹³⁵ are each independently hydrogen, alkyl, alkenyl, alkynyl, aryl, heteroaryl, heteroarylium, cycloalkyl, heterocyclyl, OR¹³⁶ or NR¹³²R¹³³, or R¹³⁴ and R¹³⁵ together form alkylene, alkenylene, alkynylene, heteroalkylene, where R¹³⁶ is hydrogen, alkyl, alkenyl, alkynyl, aryl, heteroaryl, heteroarylium, cycloalkyl or heterocyclyl;

R¹²⁷ and R¹²⁸ are selected as in (i) or (ii) as follows:

(i) R¹²⁷ and R¹²⁸ are each independently hydrogen, alkyl, alkenyl, alkynyl, aryl, heteroaryl, heteroarylium, cycloalkyl, heterocyclyl, OR¹²⁵, NR¹³⁷R¹³⁸ or C(A)R¹³⁹, where R¹³⁷ and R¹³⁸ are each independently hydrogen, alkyl, alkenyl, alkynyl, aryl, heteroaryl, heteroarylium, cycloalkyl or heterocyclyl, or together form alkylene, alkenylene, alkynylene, heteroalkylene; and R¹³⁹ is hydrogen, alkyl, alkenyl, alkynyl, aryl, heteroaryl, heteroarylium, cycloalkyl, heterocyclyl, OR¹⁴⁰ or NR¹³²R¹³³, where R¹⁴⁰ is alkyl, alkenyl, alkynyl, aryl, heteroaryl, heteroarylium, cycloalkyl, heterocyclyl; or

(ii) R¹²⁷ and R¹²⁸ together form alkylene, alkenylene, alkynylene, heteroalkylene;

R¹²⁹ is hydrogen, alkyl, alkenyl, alkynyl, aryl, heteroaryl, heteroarylium, cycloalkyl, heterocyclyl, OR¹⁴⁰ or NR¹³²R¹³³;

R¹³⁰ and R¹³¹ are each independently hydrogen, alkyl, alkenyl, alkynyl, aryl, heteroaryl, heteroarylium, cycloalkyl, heterocyclyl or C(A)R¹⁴¹1, where R¹⁴¹ is alkyl, alkenyl, alkynyl, aryl, heteroaryl, heteroarylium, cycloalkyl, heterocyclyl, OR¹²⁵ or NR¹³²R¹³³; or R¹³⁰ and R¹³¹ together form alkylene, alkenylene, alkynylene, heteroalkylene;

R¹³² and R¹³³ are each independently hydrogen, alkyl, alkenyl, alkynyl, aryl, heteroaryl, heteroarylium, cycloalkyl, heterocyclyl, or R¹³² and R¹³³ together form alkylene, alkenylene, alkynylene, heteroalkylene; and

R¹⁰ is hydrogen, alkyl, alkenyl, alkynyl, cycloalkyl, heterocyclyl, aryl or heteroaryl;

where Ar¹, R⁷, R⁸, R⁹ and R¹⁰ are each independently unsubstituted or substituted with one or more, in one embodiment one, two or three substituents, each independently selected from Q¹, where Q¹ is halo, pseudohalo, hydroxy, oxo, thia, nitrite, nitro, formyl, mercapto, hydroxycarbonyl, hydroxycarbonylalkyl, hydroxycarbonylalkenyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, alkoxyxarbonylalkoxy, aryloxycarbonyl, aryloxycarbonylalkyl, aralkoxycarbonyl, aralkoxycarbonylalkyl, aralkoxycarbonylalkoxy, arylcarbonylalkyl, aminocarbonyl, aminocarbonylalkyl, aminocarbonylalkoxy, alkylaminocarbonyl, alkylaminocarbonylalkyl, alkylaminocarbonylalkoxy, dialkylaminocarbonyl, dialkylaminocarbonylalkyl, dialkylaminocarbonylalkoxy, arylaminocarbonyl, arylaminocarbonylalkyl, arylaminocarbonylalokoxy, diarylaminocarbonyl, diarylaminocarbonylalkyl, diarylaminocarbonyl alkoxy, arylalkylaminocarbonyl, arylalkylamninocarbonylalkyl, arylalkylaminocarbonylalkoxy, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R¹⁵²R¹⁵³, P(R¹⁵⁰)₂, P(═O)(R¹⁵⁰)₂, OP(═O)(R¹⁵⁰)₂, —NR¹⁶⁰C(═O)R¹⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; azido, tetrazolyl or two Q¹ groups, which substitute atoms in a 1,2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q¹ groups, which substitute the same atom, together form alkylene; and

each Q¹ is independently unsubstituted or substituted with one or more substituents, in one embodiment one, two or three substituents, each independently selected from Q²;

each Q² is independently halo, pseudohalo, hydroxy, oxo, thia, nitrile, nitro, formyl, mercapto, hydroxycarbonyl, hydroxycarbonylalkyl, hydroxycarbonylalkenyl alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonyl aminoalkyl, aryloxycarbonylaminoalkyl, aryloxyarylcarbonyl amino, aryloxycarbonylamino, alkylsulfonylamino, arylsulfonylamino, heteroarylsulfonylamino, heterocyclylsulfonylamino, heteroarylthio, azido, —N⁺R¹⁵¹R¹⁵²R¹⁵³, P(R¹⁵⁰)₂, P(═O)(R¹⁵⁰)₂, OP(═O)(R¹⁵⁰)₂, —NR¹⁶⁰C(═O)R¹⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q² groups, which substitute atoms in a 1,2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q² groups, which substitute the same atom, together form alkylene;

R¹⁵⁰ is hydroxy, alkoxy, aralkoxy, alkyl, heteroaryl, heterocyclyl, aryl or —NR¹⁷⁰R¹⁷¹, where R¹⁷⁰ and R¹⁷¹ are each independently hydrogen, alkyl, aralkyl, aryl, heteroaryl, heteroaralkyl or heterocyclyl, or R¹⁷⁰ and R¹⁷¹ together form alkylene, azaalkylene, oxaalkylene or thiaalkylene;

R¹⁵¹, R¹⁵² and R¹⁵³ are each independently hydrogen, alkyl, aryl, aralkyl, heteroaryl, heteroaralkyl, heterocyclyl or heterocyclylalkyl;

R¹⁶⁰ is hydrogen, alkyl, aryl, aralkyl, heteroaryl, heteroaralkyl, heterocyclyl or heterocyclylalkyl; and

R¹⁶³ is alkoxy, aralkoxy, alkyl, heteroaryl, heterocyclyl, aryl or —NR¹⁷⁰R¹⁷¹.

In some embodiments Ar¹ is aryl, heteroaryl, or cycloalkyl, and is unsubstituted or substituted with alkyl, alkenyl, alkynyl, heteroaryl, halo, pseudohalo, dialkylamino, aryloxy, aralkoxy, haloalkyl, alkoxy, haloalkoxy, cycloalkyl, or COOR, where R is hydrogen or alkyl;

R⁷ is hydrogen or NRR, where R is hydrogen or alkyl;

R⁸ and R⁹ are each independently selected from (i) and (ii) as follows:

(i) R⁸ and R⁹ together with the atoms to which they are attached form a fused phenyl ring, which is unsubstituted or substituted with halo, pseudohalo, alkyl, alkoxy, cycloalkyl, fused cycloalkyl, fused heterocyclyl, fused heteroaryl, or fused aryl, which is unsubstituted or substituted with halo, pseudohalo, alkyl, alkoxy, aryl, cycloalkyl, heterocyclyl, fused aryl, fused heterocyclyl, and fused cycloalkyl; and

(ii) R⁸ is CN or COOR²⁰⁰, where R²⁰⁰ is hydrogen, alkyl, alkenyl, alkynyl, cycloalkyl, heterocyclyl, aryl or heteroaryl; and R⁹ is hydrogen, alkyl or alkylthio; and

R¹⁰ is hydrogen;

where Ar¹, R⁷, R⁸, R⁹ and R¹⁰ are each independently unsubstituted or substituted with one or more, in one embodiment one, two or three substituents, each independently selected from Q¹.

In some embodiments Ar¹ is phenyl, naphthyl, pyridyl, furyl, or thienyl, and is unsubstituted or substituted with alkyl, alkenyl, halo, pseudohalo, dialkylamino, aryloxy, haloalkyl, alkoxy, aryloxy, cycloalkyl, heterocyclyl, fused heterocyclyl, aryl, fused aryl, heteroaryl, fused heteroaryl, or COOR, where R is hydrogen or alkyl.

In some embodiments Ar¹ is substituted with methyl, fluoro, bromo, chloro, iodo, dimethylamino, phenoxy, trifluoromethyl or methoxycarbonyl.

In some embodiments Ar¹ is phenyl, 2-thienyl, 3-thienyl, 2-furyl, 3-furyl, 5-chloro-2-thienyl, 5-bromo-2-thienyl, 3-methyl-2-thienyl, 5-methyl-2-thienyl, 5-ethyl-2-thienyl, 2-methylphenyl, 3-methylphenyl, 4-fluoro-3-bromophenyl, 2-fluorophenyl, 3,4-difluorophenyl, 2-chlorophenyl, 3-chlorophenyl, 3,4-dichlorophenyl, 3,4,5,-methoxyphenyl, 2,4-methoxyphenyl, 2-fluoro-5-bromophenyl, 4-dimethylaminophenyl, 3-trifluoromethyl, 3-bromophenyl, 2-trifluoromethyl-4-fluorophenyl, 3-trifluoromethyl-4-fluorophenyl, 2-fluoro-3-chlorophenyl, 3-bromo-4-fluorophenyl, perfluorophenyl, 3-pyridyl, 4-pyridyl, 4-bromophenyl, 4-chlorophenyl, 3-phenoxyphenyl, 2,4-dichlorophenyl, 2,3-difluorophenyl, 2-chlorophenyl, 2-fluoro-6-chlorophenyl, 1-naphthyl, 4-trifluoromethylphenyl, 2-trifluoromethylphenyl, 4-trifluoromethoxyphenyl, or 4-methoxycarbonylphenyl.

In some embodiments R⁷ is hydrogen or dialkylamino, or is hydrogen or diethylamino.

In some embodiments R⁸ and R⁹ are each independently selected from (i) and (ii) as follows:

(i) R⁸ and R⁹ together with the atoms to which they are attached form a fused phenyl ring, which is unsubstituted or substituted with methyl, chloro, methoxy, cyclopentyl, fused cyclopentyl, or another fused phenyl ring, which is unsubstituted or substituted with bromo; and

(ii) R⁸ is CN or COOR²⁰⁰, where R²⁰⁰ is methyl, benzyl, ethyl, 4-methoxybenzyl or 2-phenylethyl; and R⁹ is methyl, methylthio or phenylaminocarbonylmethylthio.

In various embodiments, the compound is represented by one of Formulas IIb-IIp:

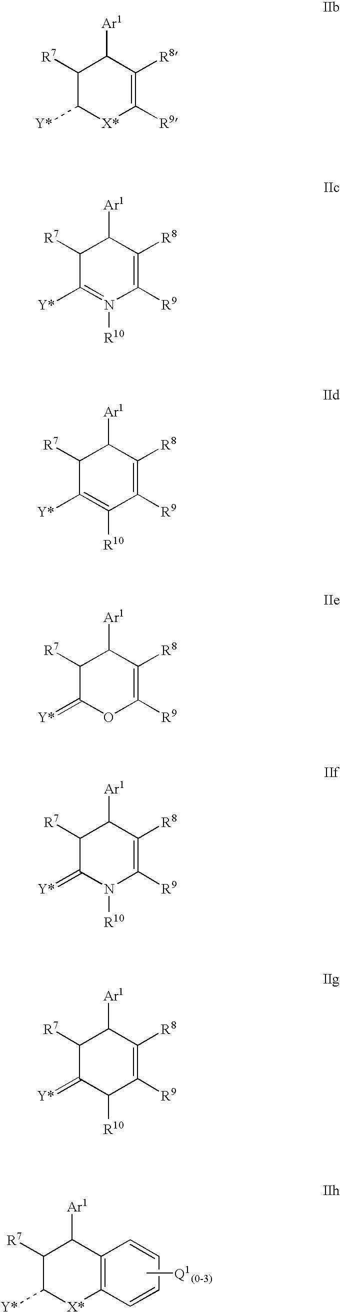

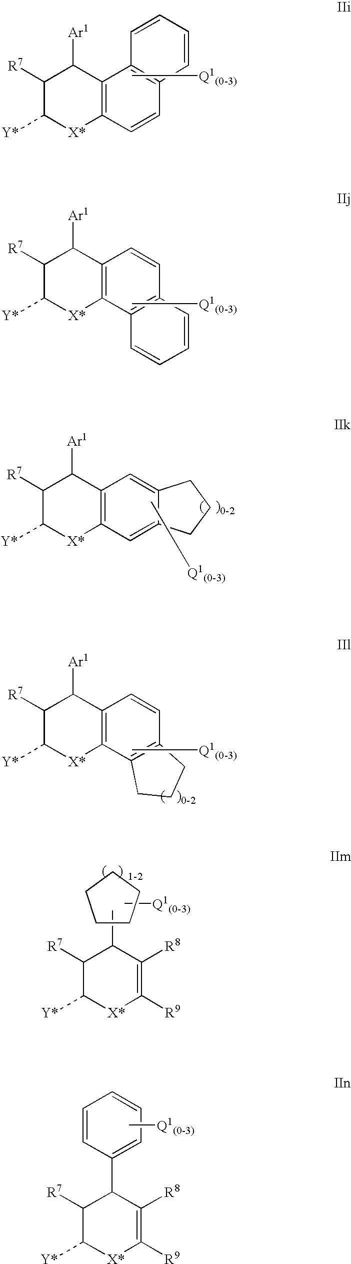

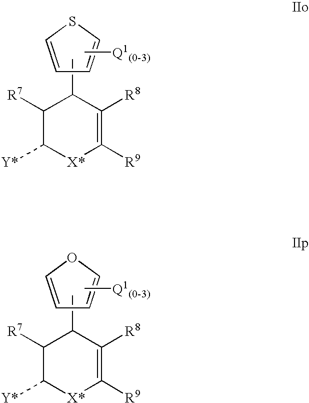

In Formulas IIb-IIp, the variables have the values described herein above for Formulas II and IIa, where X* and Y* are selected such that one of the dashed bonds (- - -) is a single bond and the other is a double bond. In various embodiments represented by Formula Ib, R⁸′ and R⁹′ are independently selected from hydrogen, alkyl, alkenyl, alkynyl, aryl, heteroaryl, heteroarylium, cycloalkyl, heterocyclyl, C(A)R¹¹⁰, halo, pseudohalo, OR¹¹¹, S(D)ₐR′¹², NR¹¹⁵R¹¹⁶ or N⁺R¹¹⁵R¹¹⁶R¹¹⁷; in some embodiments, R^{8′} is CN or COOR²⁰⁰, where R²⁰⁰ is hydrogen, alkyl, alkenyl, alkynyl, cycloalkyl, heterocyclyl, aryl or heteroaryl; and R^{9′} is hydrogen, alkyl or alkylthio; and in some embodiments, R^{8′} is CN or COOR²⁰⁰, where R²⁰⁰ is methyl, benzyl, ethyl, 4-methoxybenzyl or 2-phenylethyl; and R^{9′} is methyl, methylthio or phenylaminocarbonylmethylthio. In various embodiments of Formulas IIh-IIp, each Q¹ is independently selected from halogen, alkyl, alkoxy, nitro, CN, N₃, aryl, aryloxy, arylalkyloxy, alkynyl, amino, alkylamino, heterocyclyl, heteroaryl, substituted carboxyl (e.g., CO₂-alkyl, CO₂-benzyl), haloalkyl, and haloalkoxy, or two adjacent Q¹, on the same phenyl or adjacent fused phenyl rings, together form a cycloalkyl or heterocyclyl ring fused with the phenyl or adjacent fused phenyl rings. In Formulas IIh-IIp, the bond line from Q¹ indicates that each Q¹ may independently be bonded to any ring crossed by the bond line.

In some embodiments, the compound is represented by one of Formulas IIq, IIr, and IIs:

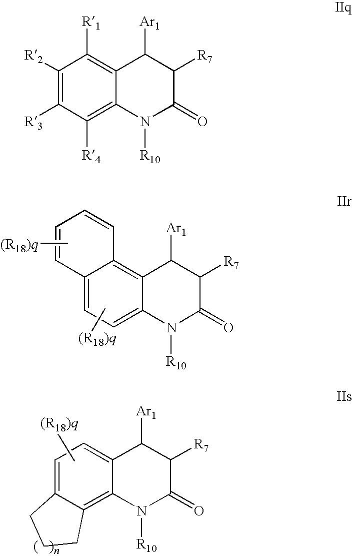

In Formulas IIq, IIr, and IIs, Ar¹, R⁷, and R¹⁰ can have the values recited herein; and each q is independently 0, 1, or 2;

n is 0, 1 or 2;

R′1, R′2, R′3, R′4, and each R18 are independently selected from hydrogen, alkyl, alkenyl, alkynyl, aryl, heteroaryl, heteroarylium, cycloalkyl, heterocyclyl, C(A)R¹¹⁰, halo, pseudohalo, OR¹¹¹, S(D)ₐR¹¹², NR¹¹⁵R¹¹⁶ or N⁺R¹¹⁵R¹¹⁷, wherein values for A, R¹¹⁰, R¹¹¹, D, a, R¹¹², R¹¹⁵, R¹¹⁶ and R¹¹⁷ are selected as described herein above.

In some embodiments the compound is selected from the compounds in Table II. In certain embodiments, the compound is selected from compounds II.1-II.95 in Table II; in some embodiments, the compound is selected from compounds II.1-II.69 in Table II. In some embodiments, the compound is selected from compounds II.1-II.3 and II.70-II.95 in Table II. In some embodiments, the compound is selected from compounds II.4-II.69 in Table II.

### C. Preparation of the Compounds

The compounds for use in the compositions and methods provided herein may be obtained from commercial sources (e.g., Aldrich Chemical Co., Milwaukee, Wis.), may be prepared by methods well known to those of skill in the art, or may be prepared by the methods shown herein. One of skill in the art would be able to prepare all of the compounds for use herein by routine modification of these methods using the appropriate starting materials.

Certain of the compounds provided herein may be made by the synthetic route shown below. Briefly, aryl amines or heteroaryl amines are converted to 1 using the corresponding nitrile. Compound 1 can also be synthesized in other ways including from aryl halides or heteroaryl halides using the corresponding guanidinium salt. Compound 1 is treated with acyl halides or anhydrides to make the corresponding acylated compound 2, which can be converted to the corresponding amide 3 by reaction with ammonia. Compound 1 is converted to a five membered heterocyclic compound 4 by reagent 10 and a suitable base such as pyridine or dimethyl amino pyridine in dichloromethane. Five membered heterocyclic compounds like 15f can also be generated by guanidation of the arylamine with compound 15a, followed by cyclization and acylation with compounds 15c and 15e, respectively. Compound 1 is converted to a six membered heterocyclic compound 5 by reagent 11 or reagent 12 and a suitable base. Compound 1 is converted to six membered heterocyclic compound 6 by reagent 13 and a base. Compound 1 is converted to six membered heterocyclic compound 7 by reagent 14 with a suitable base and solvent.

Aryl amine or a heteroaryl amine is converted to compound 8 by reagent 14 with a suitable base and solvent. Compound 8 can be further treated with ammonia to make the corresponding imine, which is acylated to yield compound 9.

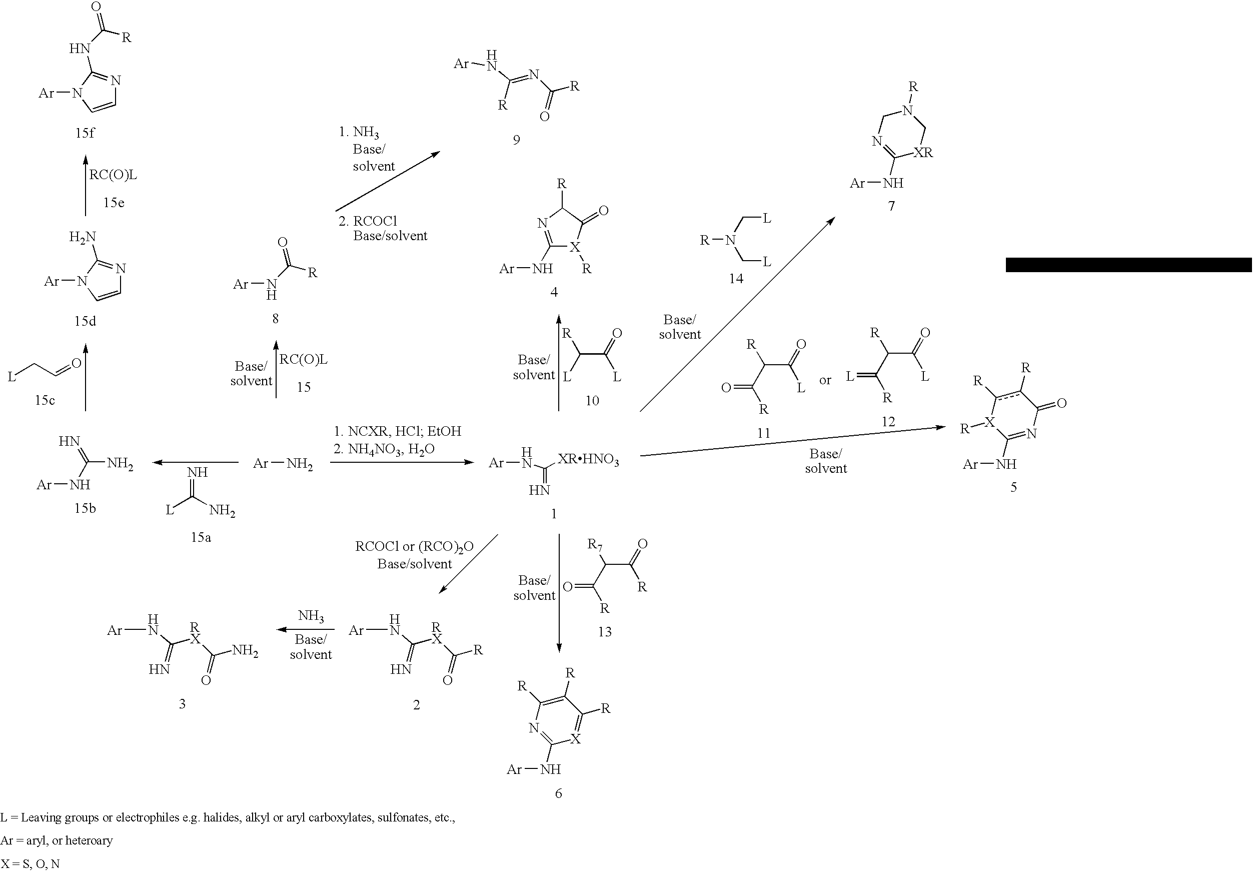

Further compounds provided herein may be prepared by the scheme shown below. Briefly, amine 19 is acylated by treatment with acetic anhydride and base. This acyl intermediate product is then treated with a suitable aldehyde and Lewis or protic acid to synthesize lactam 20. The nitrogen of the lactam 20 can be protected and the carbon adjacent to the carbonyl functionalized by standard substitution reactions.

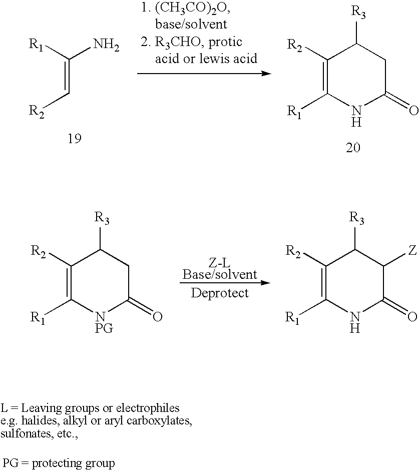

Other compounds provided herein may be synthesized according to the following scheme. Briefly, aldehyde 21 and methyl acetate undergo a condensation reaction to yield an unsaturated ester, which is hydrolyzed to the corresponding acid by a suitable base. The acid can then be converted directly to unsaturated carbonyl 23 by treatment with protic acid. The acid can also be converted to the corresponding acid chloride 22 by treatment with thionyl chloride, the acid chloride 22 can then undergo a Friedel Crafts acylation with 24 to form an unsaturated carbonyl 23.

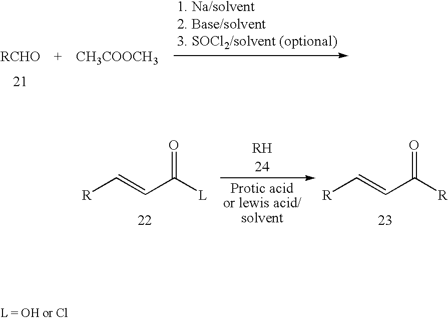

Further compounds provided herein may be synthesized according to the scheme shown below. Briefly, hydrazine 24 is converted to amine 25 by treating it with amide 28 and base. The amine 25 can be acylated with 29 to yield 26. Hydrazine 24 is converted to pyrrole 27 by treatment with a dicarbonyl compound 30.

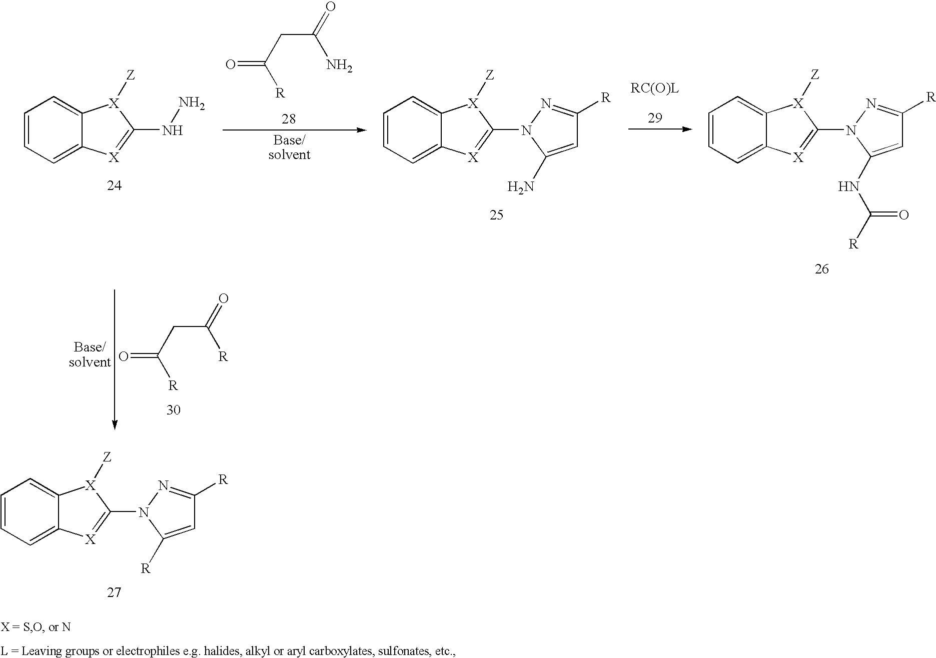

Further compounds provided herein may be synthesized according to schemes 1-5 shown below. Briefly, compound 19 is cyclyized by reaction with basic acetic anhydride followed by acid catalyzed reaction with an aldehyde to give ring compound 20. In another example, in Scheme 2, the amine corresponding to compound 19 can be reacted with an acid or acid halide using a base coupling agent, followed by cyclization with a lewis or protic acid. Scheme 4 shows a cyclization using two alkenes and a lewis acid such as AlCl₃. Schemes 6 and 7 show additional base coupling reactions to give the cyclized product. Scheme 3 shows a deprotection reaction on the ring nitrogen. Scheme shows a conversion of a cyclic amide to an amino imine.

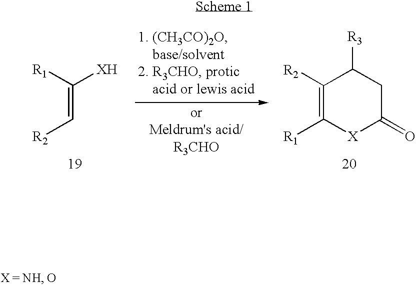

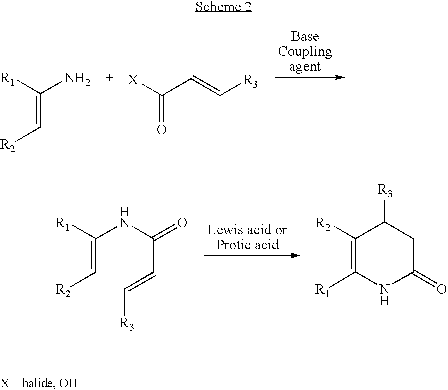

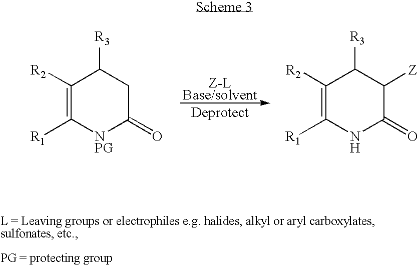

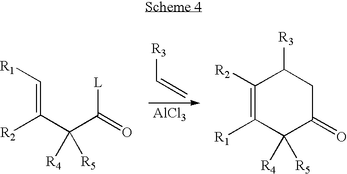

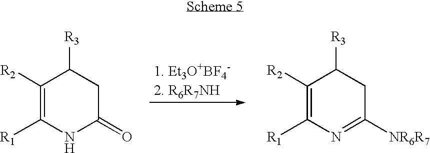

### D. Formulation of Pharmaceutical Compositions

The pharmaceutical compositions provided herein contain therapeutically effective amounts of one or more of the compounds provided herein that are useful in the treatment or amelioration of one or more of the symptoms of diseases or disorders characterized by impaired protein trafficking, and a pharmaceutically acceptable carrier. Pharmaceutical carriers suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients.

The compositions contain one or more compounds provided herein. The compounds are, in one embodiment, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives thereof is (are) mixed with a suitable pharmaceutical carrier. The compounds may be derivatized as the corresponding salts, esters, enol ethers or esters, acetals, ketals, orthoesters, hemiacetals, hemiketals, acids, bases, solvates, hydrates or prodrugs prior to formulation, as described above. The concentrations of the compounds in the compositions are effective for delivery of an amount, upon administration, that treats or ameliorates one or more of the symptoms of diseases or disorders characterized by impaired protein trafficking.

In one embodiment, the compositions are formulated for single dosage administration. To formulate a composition, the weight fraction of compound is dissolved, suspended, dispersed or otherwise mixed in a selected carrier at an effective concentration such that the treated condition is relieved or one or more symptoms are ameliorated.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in systems described herein (see, e.g., Examples 1 and 2), and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of diseases or disorders characterized by impaired protein trafficking, as described herein.

In one embodiment, a therapeutically effective dosage should produce a serum concentration of active ingredient of from about 0.1 ng/ml to about 50-100 μg/ml. The pharmaceutical compositions, in another embodiment, should provide a dosage of from about 0.001 mg to about 2000. mg of compound per kilogram of body weight per day. Pharmaceutical dosage unit forms are prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof The pharmaceutically therapeutically active compounds and derivatives thereof are, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975.

Dosage forms or compositions containing active ingredient in the range of 0.005% to 100% with the balance made up from non-toxic carrier may be prepared. Methods for preparation of these compositions are known to those skilled in the art. The contemplated compositions may contain 0.001%-100% active ingredient, in one embodiment 0.1-95%, in another embodiment 75-85%.

1. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

a. Solid Compositions for Oral Administration

In certain embodiments, the formulations are solid dosage forms, in one embodiment, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polyvinylpyrrolidone, povidone, crospovidones, sucrose and starch paste. Lubricants include talc, starch, magnesium or calcium stearate, lycopodium and stearic acid Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The compound, or pharmaceutically acceptable derivative thereof, could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

b. Liquid Compositions for Oral Administration

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is in one embodiment encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. No. RE28,819 and U.S. Pat. No. 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

2. Injectables, Solutions and Emulsions

Parenteral administration, in one embodiment characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles,.nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

3. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

4. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, in one embodiment, have diameters of less than 50 microns, in one embodiment less than 10 microns.

The compounds may be formulated for local or topical application, such as for topical application to the skin and mucous membranes, such as in the eye, in the form of gels, creams, and lotions and for application to the eye or for intracistemal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01% -10% isotonic solutions, pH about 5-7, with appropriate salts.

5. Compositions for Other Routes of Administration

Other routes of administration, such as transdermal patches, including iontophoretic and electrophoretic devices, and rectal administration, are also contemplated herein.

Transdermal patches, including iotophoretic and electrophoretic devices, are well known to those of skill in the art. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in one embodiment, is about 2 to 3 gm.

Tablets and capsules for rectal administration are manufactured using the same pharmaceutically acceptable substance and by the same methods as for formulations for oral administration.

6. Targeted Formulations

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate-buffered saline lacking divalent cations (PBS) is added and the flask shaken until the lipid film is dispersed. The resulting vesicles are washed to remove unencapsulated compound, pelleted by centrifugation, and then resuspended in PBS.

7. Articles of Manufacture

The compounds or pharmaceutically acceptable derivatives may be packaged as articles of manufacture containing packaging material, a compound or pharmaceutically acceptable derivative thereof provided herein, which is effective for treatment or amelioration of one or more symptoms of diseases or disorders characterized by impaired protein trafficking, within the packaging material, and a label that indicates that the compound or composition, or pharmaceutically acceptable derivative thereof, is used for treatment or amelioration of one or more symptoms of diseases or disorders characterized by impaired protein trafficking.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease or disorder in which impaired protein trafficking is implicated as a mediator or contributor to the symptoms or cause.

8. Sustained Release Formulations

Also provided are sustained release formulations to deliver the compounds to the desired target (i.e. brain or systemic organs) at high circulating levels (between 10⁻⁹ and 10⁻⁴ M). In a certain embodiment for the treatment of a disorder characterized by impaired protein trafficking, the circulating levels of the compounds are maintained up to 10⁻⁷ M.

It is understood that the compound levels are maintained over a certain period of time as is desired and can be easily determined by one skilled in the art. In one embodiment, the administration of a sustained release formulation is effected so that a constant level of therapeutic compound is maintained between 10⁻⁸ and 10⁻⁶M between 48 to 96 hours in the sera.

Such sustained and/or timed release formulations may be made by sustained release means of delivery devices that are well known to those of ordinary skill in the art, such as those described in U.S. Pat. Nos. 3,845,770; 3,916,899; 3,536,809; 3,598,123; 4,008,719; 4,710,384; 5,674,533; 5,059,595; 5,591,767; 5,120,548; 5,073,543; 5,639,476; 5,354,556 and 5,733,566, the disclosures of which are each incorporated herein by reference. These pharmaceutical compositions can be used to provide slow or sustained release of one or more of the active compounds using, for example, hydroxypropylmethyl cellulose, other polymer matrices, gels, permeable membranes, osmotic systems, multilayer coatings, microparticles, liposomes, microspheres, or the like. Suitable sustained release formulations known to those skilled in the art, including those described herein, may be readily selected for use with the pharmaceutical compositions provided herein. Thus, single unit dosage forms suitable for oral administration, such as, but not limited to, tablets, capsules, gelcaps, caplets, powders and the like, that are adapted for sustained release are contemplated herein.

In one embodiment, the sustained release formulation contains active compound such as, but not limited to, microcrystalline cellulose, maltodextrin, ethylcellulose, and magnesium stearate. As described above, all known methods for encapsulation which are compatible with properties of the disclosed compounds are contemplated herein. The sustained release formulation is encapsulated by coating particles or granules of the pharmaceutical compositions provided herein with varying thickness of slowly soluble polymers or by microencapsulation. In one embodiment, the sustained release formulation is encapsulated with a coating material of varying thickness (e.g. about 1 micron to 200 microns) that allow the dissolution of the pharmaceutical composition about 48 hours to about 72 hours after administration to a mammal. In another embodiment, the coating material is a food-approved additive.

In another embodiment, the sustained release formulation is a matrix dissolution device that is prepared by compressing the drug with a slowly soluble polymer carrier into a tablet. In one embodiment, the coated particles have a size range between about 0.1 to about 300 microns, as disclosed in U.S. Pat. Nos. 4,710,384 and 5,354,556, which are incorporated herein by reference in their entireties. Each of the particles is in the form of a micromatrix, with the active ingredient uniformly distributed throughout the polymer.

Sustained release formulations such as those described in U.S. Pat. No. 4,710,384, which is incorporated herein by reference in its entirety, having a relatively high percentage of plasticizer in the coating in order to permit sufficient flexibility to prevent substantial breakage during compression are disclosed. The specific amount of plasticizer varies depending on the nature of the coating and the particular plasticizer used. The amount may be readily determined empirically by testing the release characteristics of the tablets formed. If the medicament is released too quickly, then more plasticizer is used. Release characteristics are also a function of the thickness of the coating. When substantial amounts of plasticizer are used, the sustained release capacity of the coating diminishes. Thus, the thickness of the coating may be increased slightly to make up for an increase in the amount of plasticizer. Generally, the plasticizer in such an embodiment will be present in an amount of about 15 to 30% of the sustained release material in the coating, in one embodiment 20 to 25%, and the amount of coating will be from 10 to 25% of the weight of the active material, and in another embodiment, 15 to 20% of the weight of active material. Any conventional pharmaceutically acceptable plasticizer may be incorporated into the coating.

The compounds provided herein can be formulated as a sustained and/or timed release formulation. All sustained release pharmaceutical products have a common goal of improving drug therapy over that achieved by their non-sustained counterparts. Ideally, the use of an optimally designed sustained release preparation in medical treatment is characterized by a minimum of drug substance being employed to cure or control the condition. Advantages of sustained release formulations may include: 1) extended activity of the composition, 2) reduced dosage frequency, and 3) increased patient compliance. In addition, sustained release formulations can be used to affect the time of onset of action or other characteristics, such as blood levels of the composition, and thus can affect the occurrence of side effects.

The sustained release formulations provided herein are designed to initially release an amount of the therapeutic composition that promptly produces the desired therapeutic effect, and gradually and continually release of other amounts of compositions to maintain this level of therapeutic effect over an extended period of time. In order to maintain this constant level in the body, the therapeutic composition must be released from the dosage form at a rate that will replace the composition being metabolized and excreted from the body.

The sustained release of an active ingredient may be stimulated by various inducers, for example pH, temperature, enzymes, water, or other physiological conditions or compounds.

Preparations for oral administration may be suitably formulated to give controlled release of the active compound. In one embodiment, the compounds are formulated as controlled release powders of discrete microparticles that can be readily formulated in liquid form. The sustained release powder comprises particles containing an active ingredient and optionally, an excipient with at least one non-toxic polymer.

The powder can be dispersed or suspended in a liquid vehicle and will maintain its sustained release characteristics for a useful period of time. These dispersions or suspensions have both chemical stability and stability in terms of dissolution rate. The powder may contain an excipient comprising a polymer, which may be soluble, insoluble, permeable, impermeable, or biodegradable. The polymers may be polymers or copolymers. The polymer may be a natural or synthetic polymer. Natural polymers include polypeptides (e.g., zein), polysaccharides (e.g., cellulose), and alginic acid. Representative synthetic polymers include those described, but not limited to, those described in column 3, lines 33-45 of U.S. Pat. No. 5,354,556, which is incorporated by reference in its entirety. Particularly suitable polymers include those described, but not limited to those described in column 3, line 46-column 4, line 8 of U.S. Pat. No. 5,354,556 which is incorporated by reference in its entirety.

The sustained release compositions provided herein may be formulated for parenteral administration, e.g., by intramuscular injections or implants for subcutaneous tissues and various body cavities and transdermal devices. In one embodiment, intramuscular injections are formulated as aqueous-or oil suspensions. In an aqueous suspension, the sustained release effect is due to, in part, a reduction in solubility of the active compound upon complexation or a decrease in dissolution rate. A similar approach is taken with oil suspensions and solutions, wherein the release rate of an active compound is determined by partitioning of the active compound out of the oil into the surrounding aqueous medium. Only active compounds which are oil soluble and have the desired partition characteristics are suitable. Oils that may be used for intramuscular injection include, but are not limited to, sesame, olive, arachis, maize, almond, soybean, cottonseed and castor oil.

A highly developed form of drug delivery that imparts sustained release over periods of time ranging from days to years is to implant a drug-bearing polymeric device subcutaneously or in various body cavities. The polymer material used in an implant, which must be biocompatible and nontoxic, include but are not limited to hydrogels, silicones, polyethylenes, ethylene-vinyl acetate copolymers, or biodegradable polymers.

### E. Evaluation of the Activity of the Compounds

The activity of the compounds as modulators of protein trafficking may be measured in the assays described herein that evaluate the ability of a compound to rescue an impairment in protein trafficking. For example, the yeast mutant cell line ypt1^{ts} can be used to identify compounds that rescue cells from the lethal phenotype of a mutant YPT1 allele (see, e.g., Examples and Schmitt et al. (1988) Cell 53:635-47). The activity may be measured, for example, in a whole yeast cell assay using 384-well screening protocol and an optical density measurement.

Table III details human orthologs of the yeast genes YPT1 and SAR1. As detailed herein, a cell (e.g., a mammalian cell or a yeast cell) that exhibits reduced expression or activity of a protein required for protein trafficking (e.g., a protein of Table III) can be used to screen candidate agents for their ability to rescue the cell from a protein trafficking defect.

TABLE III
Human Counterparts of Yeast Genes YPT1 and SAR1

| | | DNA Accession | Protein Accession |
|---|---|---|---|
| Yeast Gene | Human Gene | Number | Number |
| Name | Name | (Human Gene) | (Human Gene) |
| YPT1 | Rab1a | NM_004161 | NP_004152.1 |
| | Rab1b | NM_030981 | NP_112243.1 |
| | Rab8b | NM_016530 | NP_057614.1 |
| | Rab8a | NM_005370 | NP_005361.2 |
| | Rab10 | NM_016131 | NP_057215.2 |
| | Rab13 | NM_002870 | NP_002861.1 |
| | Rab35 | NM_006861 | NP_006852.1 |
| | Rab11b | NM_004218 | NP_004209.1 |
| | Rab30 | NM_014488 | NP_055303.2 |
| | Rab11a | NM_004663 | NP_004654.1 |
| | Rab3a | NM_002866 | NP_002857.1 |
| | Rab3c | NM_138453 | NP_612462.1 |
| | Rab3d | NM_004283 | NP_004274.1 |
| | Rab3b | NM_002867 | NP_002858.2 |
| | Rab2 | NM_002865 | NP_002856.1 |
| | Rab43 | NM_198490 | NP_940892.1 |
| | Rab4a | NM_004578 | NP_004569.2 |
| | Rab2b | NM_032846 | NP_116235.2 |
| | Rab4b | NM_016154 | NP_057238.2 |
| | Rab25 | NM_020387 | NP_065120.1 |
| | Rab14 | NM_016322 | NP_057406.2 |
| | Rab37 | NM_001006638 | NP_001006639.1 |
| | Rab18 | NM_021252 | NP_067075.1 |
| | Rab5b | NM_002868 | NP_002859.1 |
| | Rab33a | NM_004794 | NP_004785.1 |
| | Rab26 | NM_014353 | NP_055168.2 |
| | Rab5a | NM_004162 | NP_004153.2 |
| | Rab19b | NM_001008749 | NP_001008749.1 |
| | Rab5c | NM_201434 | NP_958842.1 |
| | Rab33b | NM_031296 | NP_112586.1 |
| | Rab39b | NM_171998 | NP_741995.1 |
| | Rab39 | NM_017516 | NP_059986.1 |
| | Rab31 | NM_006868 | NP_006859.2 |
| | Rab15 | NM_198686 | NP_941959.1 |
| | Rab40c | NM_021168 | NP_066991.2 |
| | Rab27b | NM_004163 | NP_004154.2 |
| | Rab22a | NM_020673 | NP_065724.1 |
| | Rab6b | NM_016577 | NP_057661.2 |
| | Rab40b | NM_006822 | NP_006813.1 |
| | Rasef | NM_152573 | NP_689786.2 |
| | Rab21 | NM_014999 | NP_055814.1 |
| | Rab27a | NM_183236 | NP_899059.1 |
| | Loc286526 | NM_001031834 | NP_001027004.1 |
| | Rab40a | NM_080879 | NP_543155.2 |
| | Rab6a | NM_198896 | NP_942599.1 |
| | Rab17 | NM_022449 | NP_071894.1 |
| | Rab6c | NM_032144 | NP_115520.1 |
| | Rab7 | NM_004637 | NP_004628.4 |
| | Rab9a | NM_004251 | NP_004242.1 |
| | Rab711 | NM_003929 | NP_003920.1 |
| | Rab9b | NM_016370 | NP_057454.1 |
| | Rab34 | NM_031934 | NP_114140.2 |
| | Rab7b | NM_177403 | NP_796377.2 |
| | Rab41 | NM_001032726 | NP_001027898.1 |
| | Rab23 | NM_183227 | NP_899050.1 |
| | Rab32 | NM_006834 | NP_006825.1 |
| | Rab38 | NM_022337 | NP_071732 |
| | Rab36 | NM_004914 | NP_004905 |
| | Rab28 | NM_001017979 | NP_001017979 |
| | Rab20 | NM_017817 | NP_060287 |
| | Rab12 | NM_001025300 | NP_001020471 |
| SAR1 | Sar1a | NM_020150 | NP_064535 |
| | Sar1b | NM_001033503 | NP_001028675 |
| SEC23 | Sec23a | NM_006364.2 | NP_006355.2 |
| | Sec23b | NM_006363.4 | NP_006354 |

In addition, efficacy of a compound can be evaluated before (first in time), concomitantly or subsequently to the above-mentioned test modalities by monitoring, e.g., (i) modulation (e.g., an improvement) of the stability of a trafficking defective protein, (ii) modulation (e.g., an improvement) of proper, physiological trafficking of the trafficking defective protein, or (iii) modulation (e.g., a restoration) in one or more functions of a trafficking defective protein. For example, in some cases, proteins (e.g., protein mutants such as ΔF508 CFTR) are prematurely degraded. Thus, the efficacy of a given compound to modulate protein trafficking can be determined by monitoring the stability of a protein in the presence as compared to the absence of the compound. For example, cells expressing a trafficking defective protein (e.g., expressing endogenously or expressing an exogenous transgene encoding a trafficking defective protein such as ΔF508 CFTR) can be cultured in the presence or absence of a compound for at least 1 hour (e.g., at least 2 hours, at least 4 hours, at least 6 hours, at least 8 hours, at least 12 hours, at least 16 hours, at least 24 hours, at least 36 hours, or at least 48 hours). Cell lysates can be prepared from the different populations of cells, suspended in Laemmli buffer (with or without reducing agent) and subjected to sodium dodecyl sulfate-polyacrylamide gel electrophoresis (SDS-PAGE). Using antibodies that specifically recognize the trafficking defective protein (e.g., CFTR), the amount of the protein in the presence as compared to in the absence of a compound can be determined by western or dot-blotting techniques. An increase in the amount of a trafficking defective protein in the presence of a compound as compared to in the absence of the compound indicates that the compound modulates (e.g., stabilizes) a trafficking defective protein (Vij et al. (2006) J. Biol. Chem. 281(25):17369-17378). Where a modified state (e.g., glycosylation or phosphorylation) of a protein is indicative of increased stability, a change in the modified state of a protein can also be used to determine if a compound stabilizes the trafficking defective protein. For example, the amount of glycosylated CFTR (e.g., ΔF509 CFTR) can be assessed in the presence as compared to the absence of a compound. An increase in the glycosylated form of the protein is an indicated that the compound has stabilized CFTR (e.g., ΔF508 CFTR).

It is understood that routine adaptation of this assay can be used to monitor any trafficking defective protein. Furthermore, steady-state levels (e.g., protein turnover or the degradation rate) of a protein can also be monitored in the presence and absence of a compound (e.g., see Van Goor et al. (2006) Am. J. Physiol. Lung. Cell Mol. Physiol. 290:L1117-L1130).

Another method of determining modulation of a trafficking defective protein is an in situ staining method. For example, where a protein (e.g., ΔF508 CFTR or G601S-hERG) is prematurely degraded before reaching the cell surface, the efficacy of a compound to modulate the trafficking defective protein can be determined as a change (e.g., an increase) in the amount of surface expression of the protein. Thus, an increase in the amount protein expression at the cell surface in the presence of a compound as compared to the surface expression in the absence of a compound indicates that compound modulates (e.g., stabilizes) the trafficking defective protein. Immunostaining methods are well known to those of skill in the art and include embodiments where the cells are still viable (e.g., confocal microscopy of live cells such as mammalian cells) or staining of fixed cells (e.g., immunohistochemistry). The cells can be attached to a solid support (e.g., a tissue culture plate or poly-lysine coated glass slide) or can be in solution (e.g., for fluorescence assisted cell sorting (FACS) analysis). A primary antibody specific for a trafficking defective protein are applied (e.g., administered, delivered, contacted) to cells. The primary antibody itself can be labeled with a detectable label (e.g., a different colored fluorophore (e.g., rhodamine, texas red, FITC, Green fluorescent protein, Cy3, Cy5). Alternatively, a secondary agent, such as a secondary antibody, can be detectably labeled and the primary antibody unlabeled. The primary antibody can also be conjugated to a first member of a binding pair (e.g., biotin or streptavidin) and the second member of the binding pair detectably labeled. Use of an appropriate microscope (e.g., a confocal microscope) with the appropriate optical filters can identify the position of the labeled antibodies in a given cell. An increase in signal from the detectable label from the cell surface indicates that more protein is expressed on the cell surface. Of course, it is understood that this method can be applied to trafficking defective proteins that localize to other compartments (e.g., organelles such as nucleus, lysosome, ER, Golgi, or mitochondria) of the cell. It can be useful to use another antibody or dye to identify another control protein known to localize to the given compartment of interest. Typically, the second protein is labeled with a different detectable label than the trafficking defective protein of interest. The position of both labels is then determined by the preceding methods. When each of the positions of the two proteins are determined (i.e., the location of their respective detectable label within the cell as determined by antibody binding), if they are found to occupy the same space, the two proteins are said to co-localize and thus, the trafficking defective protein has localized to the proper cellular position (i.e., when two proteins co-localize in the absence of a compound but do not co-localize in the presence of a compound, this can indicate that the compound has inhibited the interaction between the two proteins). Examples of this method are described in, for example, Morello et al. (2000) J. Clin. Invest. 105(7):887-895 and Liu et al. (2003) Proc. Natl. Acad. Sci. USA 100(26):15824-15829. Optionally the cells can be fixed, for example, using paraformaldehyde or formaldehyde, and permeabilized using a detergent (e.g., Triton-X100).

The efficacy of a compound to modulate a trafficking defective protein can also be assessed by monitoring an increase in the activity of the trafficking defective protein. For example, the ΔF508 CFTR is a PKA-regulated chloride channel, and thus an increase in the stability of the CFTR protein can be determined by an increase in, e.g., membrane potential response to forskolin or induction of cAMP-mediated chloride efflux (see, e.g., Vij et al. (2006) J. Biol. Chem. 281(25):17369-17378 and Van Goor et al. (2006) Am. J. Physiol. Lung. Cell Mol. Physiol. 290:L1117-L1130). Alpha-galactosidase-A, the trafficking defective protein in Fabry's disease, is an enzyme that metabolizes certain lipids. Therefore, the efficacy of a compound to modulate alpha-galactosidase-A can be determined by assessing the cellular activity of alpha-galactosidase in the presence as compared to in the absence of a compound. An increase in activity in the presence of the compound as compared to in the absence of the compound indicates that compound modulates (e.g., stabilizes) the alpha-galactosidase-A protein. Methods of monitoring for alpha-galactosidase activities in cells can be found in, e.g., Ioannou et al. (1998) Biochem. J. 332:789-797. Methods for monitoring the in vitro and in vivo enzymatic activities of trafficking defective proteins causative of their respective disorder characterized by impaired protein traffickings, other than CFTR and alpha-galactosidase-A, are known in the art.

Protein trafficking (e.g., endoplasmic reticulum-mediated protein trafficking) can also be detected and measured using in vitro (cell-free) methods. Thus, the efficacy of a compound to modulate, e.g., a trafficking defective protein or various steps of protein trafficking (e.g., formation or docking of COPII vesicles) can be determined using such in vitro methods. Suitable in vitro methods for detecting or measuring endoplasmic-reticulum mediated protein trafficking are described in, e.g., Rexach et al. (1991) J Cell Biol. 114(2):219-229; Segev (1991) Science 252(5012):1553-1556; Balch et al. (1984) Cell 39(2 Pt 1):405-416; Wattenberg (1991) J Electron Microsc Tech 17(2):150-164; Beckers et al. (1989) J. Cell Biol. 108(4):1245-1256; and Moreau et al. (1991) J Biol. Chem 266(7):4322-4328, the contents of each of which are incorporated herein by reference in their entirety. For example, transfer of a protein of interest from endoplasmic reticulum to Golgi can be detected or measured. First, a reporter protein is labeled in a cell, e.g., by metabolically labeling the protein using ³⁵S-methionine or by expressing a detectably-labeled form of the protein in a cell (a fusion protein comprising the protein of interest and green fluorescent protein). “Donor” membrane fractions containing endoplasmic reticulum can be obtained from the cells containing labeled protein. “Acceptor” membrane fractions containing Golgi apparatus can be prepared from cells not containing labeled protein. Transport of the labeled protein is accompanied by post-translational modification. Often the reporter protein is a glycoprotein whose carbohydrate chains are modified during ER to Golgi transport. Acceptor and donor fractions are mixed and incubated with required cofactors. Transport is monitored by the increase in the post-translationally modified form of the labeled protein. Methods for detecting the post-translationally modified labeled protein are described herein and can include western,dot blotting, lectin binding, and suspectability to glycosidases. When the detectable label is a fluorescent or luminescent label, a fluorimeter or luminometer can be utilized. When the detectable label is a radioactive label (see below), scintillation counter, X-ray film, or radiometer. It is understood that a protein need not be detectably labeled. A protein initially present in the Donor fraction (e.g., a protein specifically expressed in the Donor cell population), but not present in the Acceptor fraction can be distinguished using, e.g., western blotting techniques.

In vitro methods of detecting protein trafficking (e.g., endoplasmic reticulum-mediated protein trafficking) can also involve measuring vesicle budding, uncoating, tethering, or docking or fusion with the Golgi apparatus (see, e.g.,. Rexach et al., supra, and Bonifacino et al. (2004) Cell 116:153-166).

To determine if a compound modulates the in vitro transfer of a protein from endoplasmic reticulum to Golgi (e.g., any step of the transfer of a protein from endoplasmic reticulum to Golgi), a compound can be contacted to the Acceptor fraction, Donor fraction, or both before or during the incubation. The compound could be added to either Donor or Acceptor cell populations prior to preparing the membrane fractions. As described herein (see, e.g., Examples), compounds that inhibit the proteasome (e.g., proteosome expression or activity) can also be screened through the assays described herein (e.g., ypt1^{ts} mutant assay) to determine if they rescue endoplasmic reticulum-mediated transport. In vitro and in vivo (cell-based) methods of detecting and/or measuring proteasome activity are known in the art and are described, for example, in Chuhan et al. (2006) Br. J. Cancer 95(8):961-965; Rubin et al. (1998) EMBO J. 17(17):4909-4919; Glickman et al. (1999) Mol. Biol. Rep. 26(1-2):21-8; and Grimes al. (2005) Int. J. Oncol. 27(4):1047-1052. In vitro methods of determining whether a candidate compound inhibits the proteasome, e.g., proteasome activity, can include contacting isolated proteasome complexes with a candidate compound and measuring the activity of the isolated proteasomes contacted with the candidate compound. A decrease in the activity of a proteasome contacted with a compound as compared to proteasome activity in the absence of the compound indicates that the candidate compound inhibits proteasome activity in vitro. In vivo methods of determining whether a candidate compound inhibits the proteasome can include, e.g., contacting a cell with a candidate compound and measuring the activity of proteasomes in the cell. For example, measuring the turnover of proteins known to be degraded by the proteasome. A decrease in the activity of proteasomes in a cell contacted with a compound as compared to proteasome activity in a cell in the absence of the compound indicates that the candidate compound inhibits proteasome activity in vivo. Examples of proteosome inhibitors include, e.g., MG132, MG15, LLnL, ALLnL, bortezomib/PS-341/Velcade®, NPI-0052, epoxomicin, and lactacystin (Myung et al. (2001) Med. Res. Reviews 21(4):245-273; Montagut et al. (2006) Clin Transl Oncol. 8(5):313-317; and Chuhan et al. (2006) Br. J. Cancer 95(8):961-965).

Compounds that inhibit transcription (e.g., synthesis of mRNA) can also be screened through the assays described herein (e.g., ypt1^{ts} mutant assay) to determine if they rescue endoplasmic reticulum-mediated transport. In vitro and in vivo (cell-based) methods of detecting and/or measuring mRNA transcription are known in the art and include, e.g., measuring the amount of mRNA by RT-PCR, northern blotting, gene chip analysis, and in situ hybridization techniques. Methods of determining whether a candidate compound inhibits transcription can include, e.g., contacting a cell with a candidate compound and measuring the transcription of a gene of interest in the cell. A decrease in the amount of transcription of a gene in a cell contacted with a compound as compared to the amount in a cell in the absence of the compound indicates that the candidate compound inhibits transcription. Examples of transcription inhibitors-include, e.g., rapamycin, cyclosporine, doxorubicin, and actinomycin D.

Compounds that inhibit translation (e.g., translation of mRNA into protein) can also be screened through the assays described herein (e.g., ypt1^{ts} mutant assay) to determine if they rescue endoplasmic reticulum-mediated transport. In vitro and in vivo (cell-based) methods of detecting and/or measuring translation are known in the art and include, e.g., detecting protein expression using western blotting, dot-blotting, and enzyme-linked immunosorbent assay (ELISA) techniques. Methods of determining whether a candidate compound inhibits translation can include, e.g., contacting a cell with a candidate compound and measuring the amount of a polypeptide of interest in the cell. A decrease in the amount of the polypeptide in a cell contacted with a compound as compared to the amount in a cell in the absence of the compound can indicate that the candidate compound inhibits translation. Examples of translation inhibitors include, e.g., cycloheximide, doxorubicin, anisomycin, cycloheximide, emetine, harringtonine, chloramphenicol, and puromycin (see, e.g., Sah et al. (2003) J. Biol. Chem. 278(23):20593-20602). It is understood that compounds can inhibit translation directly or indirectly, e.g., a compound that inhibits transcription of a gene can also indirectly result in a decrease in translation.

Compounds that inhibit heat shock proteins (e.g., inhibit the activity of heat shock proteins) can also be screened through the assays described herein (e.g., ypt1^{ts} mutant assay) to determine if they rescue endoplasmic reticulum-mediated transport. Heat shock proteins include, e.g., Hsp90, Hsp70, Hsp60, Hsp40, and Hsp27, and are described in, e.g., Lindquist et al. (1988) 22:631-677. Methods of detecting and/or measuring the activity of heat shock proteins are known in the art and include, e.g., detecting or measuring stability or activity of target proteins known to regulated by heat shock proteins such as pp60v-src kinase (see, e.g., Xu et al. (1993) Proc. Natl. Acad. Sci. USA 90(15):7074-7078). Methods of determining whether a candidate compound inhibits a heat shock protein can include, e.g., contacting a cell with a candidate compound and measuring the stability or activity of a protein of interest in the cell. A decrease in the activity (or amount) of a protein in a cell contacted with a compound as compared to the activity (or amount) in a cell in the absence of the compound can indicate that the candidate compound inhibits a heat shock protein. Heat shock proteins also regulate the viability of cells following exposure to certain types of stress, e.g., elevated temperatures. Thus, inhibition of heat shock proteins can also be determined as a increase in heat-shock-induced cellular toxicity in cells treated with a candidate compound as compared to non-treated cells. It is understood that compounds that reduce expression of heat shock protein or heat shock protein mRNA are also considered inhibitors of heat shock proteins. Examples of heat shock protein inhibitors include, e.g., novobiocin, anasamysin, geldanamycin, radicicol, and shepherdins (Cox et al. (2003) Mol. Pharmacol. 64(6):1549-1556 and Xiao et al. (2006) Mini Rev. Med Chem. 6(10):1137-1143).

Compounds that inhibit sphingolipid biosynthesis (e.g., compounds that inhibit the activity or expression of inositol phosphorylceramide synthase) can also be screened through the assays described herein (e.g., ypt1^{ts} mutant assay) to determine if they rescue endoplasmic reticulum-mediated transport. Sphingolipids include, e.g., ceremide, sphingomyelin, and glycosphingolipids. Methods of detecting and/or measuring sphingolipid biosynthesis (e.g.,the production or amount of a sphingolipid) are described in, e.g., Andreani et al. (2006) Anal Biochem. 358(2):239-46. Methods of determining whether a candidate compound inhibits sphingolipid biosynthesis can include, e.g., contacting a cell with a candidate compound and measuring the amount of a sphingolipid of interest in the cell. A decrease in the amount of a sphingolipid in a cell contacted with a compound as compared to the amount in a cell in the absence of the compound can indicate that the candidate compound inhibits sphingolipid biosynthesis. The activity of specific enzymes involved in the biosynthesis of sphingolipids can also be measured in the presence and absence of a compound. A decrease in the activity of an enzyme in the presence of a candidate compound as compared to the activity in the absence of a compound is an indication that the compound inhibits the enzyme. Enzymes involved in sphingolipid metabolism include, but are not limited to, inositol phosphorylceramide synthase.

Compounds that inhibit glycosylation (e.g., compounds that inhibit the activity or expression of a protein glycosylase) can also be screened through the assays described herein (e.g., ypt1^{ts} mutant assay) to determine if they rescue endoplasmic reticulum-mediated transport. Glycosylases, whose activity can be inhibited by such compounds, include GlcNAc transferase, glucosidase I and II, and alpha-mannosidase I and II. Methods of detecting and/or measuring the protein glycosylation are known in the art and described in, e.g., Paulik et al. (1999) Archives of Biochem. Biophys. 367(2):265-273. Inhibitors of protein glycosylation include, e.g., tunicamycin, glucosamine, and swainsonine, deoxymannojirimycin, and casanospermine (see, e.g., Mori et al. (1992) EMBO J 11(7):2583-93).

Suitable, but not an exhaustive list of, methods of screening for compounds that inhibit, e.g., translation, transcription, glycosylation, sphingolipid biosynthesis, or the proteasome, are provided below.

F. Suppression of sec23^{st} and sar1^{ts} Mutant Phenotypes

Tables 2 and 3 list GenBank™ Accession Numbers corresponding to the nucleotide and protein sequences for each of the human genes identified herein. As detailed in the following sections, these nucleotide and protein sequences can be used to generate compounds (including but not limited to nucleic acids, peptides, antibodies) that modulate expression of genes or activity of encoded gene products. The genes described herein as modulators of Sec23^{ts} or Sar1^{ts} mutant phenotype (e.g., an impairment of endoplasmic-reticulum-mediated protein trafficking) are referred to in subsequent sections (e.g., regarding screening assays) as “target genes” and the encoded proteins are referred to as “target proteins.”

TABLE IV
Overexpression Suppressors of Sec23 and Sar1

| | | DNA Accession | Protein Accession |
|---|---|---|---|
| Yeast Gene | Human Gene | Number | Number |
| Name | Name | (Human Gene) | (Human Gene) |
| SEC12 | Sec12 | NM_013388 | Q9HCU5 |
| SED4 | unknown | | |
| SEC16 | unknown | | |
| HRD3 | SEL1L | NM_005065 | NP_005056 |
| | C20orf50 | AL109657 | CAI22078 |
| IRE1 | Ire1 | NM_001433 | NP_001424 |
| STS1 | unknown | | |
| SEC24 | Sec24A | AJ131244 | CAA10334 |
| | Sec24B | NM_006323 | NP_006314 |
| | Sec24C | NM_198597 | NP_940999 |
| | Sec24D | NM_014822 | NP_055637 |

TABLE V
Loss of Function Suppressors of sec23^{ts}

| | | DNA Accession | Protein Accession |
|---|---|---|---|
| Yeast Gene | Human Gene | Number | Number |
| Name | Name | (Human Gene) | (Human Gene) |
| Bst1 | PGAP1 | NM_024989 | NP_079265 |
| Emp24 | TMED2 | NM_006815 | NP_006806 |
| | TMED10 | NM_006827 | NP_006818 |
| | TMED7 | NM_181836 | NP_861974 |

Compounds that inhibit the expression or activity of Bst1 (or human PGAP1) or Emp24 (or human TMED2, TMED10, and TMED7) are expected to rescue impaired endoplasmic-reticulum-mediated protein trafficking. Compounds that that enhance the expression or activity of SEC12 (or human Sec12), SED4, SEC16, HRD3 (or human SEL1L or C20Orf50), IRE1 (or human Ire1), STS1, or SEC24 (or human Sec24A, Sec24B, Sec24C, or Sec24D) are expected to rescue impaired endoplasmic-reticulum-mediated protein trafficking.

As detailed herein, the sar1^{ts} and sec23^{ts} yeast mutants exhibit impaired impaired endoplasmic-reticulum-mediated protein trafficking. Several genes are known to be suppressors of loss of function mutations of SAR1 and SEC23. As a result, compounds that enhance the expression or activity of these sar1^{ts} or sec23^{ts} suppressor genes are also expected to rescue impaired endoplasmic-reticulum-mediated protein trafficking.

### Screening Assays

The methods described herein include methods (also referred to herein as “screening assays”) for identifying compounds that modulate (i.e., increase or decrease) expression or activity of selected target genes or their protein products. Such compounds include, e.g., polypeptides, peptides, antibodies, peptidomimetics, peptoids, small inorganic molecules, small non-nucleic acid organic molecules, nucleic acids (e.g., anti-sense nucleic acids, siRNA, oligonucleotides, synthetic oligonucleotides), carbohydrates, or other agents that bind to the target proteins, have a stimulatory or inhibitory effect on, for example, expression of a target gene or activity of a target protein. Compounds thus identified can be used to modulate the expression or activity of target genes or target proteins in a therapeutic protocol.

In general, screening assays involve assaying the effect of a test agent on expression or activity of a target nucleic acid or target protein in a test sample (i.e., a sample containing the target nucleic acid or target protein). Expression or activity in the presence of the test compound or agent can be compared to expression or activity in a control sample (i.e., a sample containing the target protein that is incubated under the same conditions, but without the test compound). A change in the expression or activity of the target nucleic acid or target protein in the test sample compared to the control indicates that the test agent or compound modulates expression or activity of the target nucleic acid or target protein and is a candidate agent.

Compounds can be tested for their ability to modulate one or more activities mediated by a target protein described herein. For example, compounds that modulate expression of a gene or activity of a protein listed in Table IV or V can be tested for their ability to modulate toxicity in cells exhibiting impaired endoplasmic-reticulum-mediated protein trafficking. Methods of assaying a compound for such activities are known in the art (and described herein). In some cases, a compound is tested for it's ability to directly affect target gene expression or binding to a target protein (e.g., by decreasing the amount of target RNA in a cell or decreasing the amount of target protein in a cell) and tested for its ability to modulate a metabolic effect associated with the target protein.

In one embodiment, assays are provided for screening candidate or test molecules that are substrates of a target protein or a biologically active portion thereof in a cell. In another embodiment, the assays are for screening candidate or test compounds that bind to a target protein or modulate the activity of a target protein, or a biologically active portion thereof. Such compounds include those that disrupt the interaction between a target protein and its ligand.

The test compounds used in the methods can be obtained using any of the numerous approaches in the art including combinatorial library methods, including: biological libraries; peptoid libraries (libraries of molecules having the functionalities of peptides, but with a novel, non-peptide backbone which are resistant to enzymatic degradation but which nevertheless remain bioactive; e.g., Zuckermann et al. (1994) J. Med. Chem. 37:2678); spatially addressable parallel solid phase or solution phase libraries; synthetic library methods requiring deconvolution; the “one-bead one-compound” library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the literature, for example in: DeWitt et al., Proc. Natl. Acad. Sci. USA, 90:6909, 1993; Erb et al., Proc. Natl. Acad. Sci. USA, 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl., 33:2061, 1994; and Gallop et al., J. Med. Chem., 37:1233, 1994.

Libraries of compounds may be presented in solution (e.g., Houghten, Bio/Tecbniques, 13:412421, 1992), or on beads (Lam, Nature, 354:82-84, 1991), chips (Fodor, Nature 364:555-556, 1993), bacteria (U.S. Pat. No. 5,223,409), spores (U.S. Pat. Nos. 5,571,698; 5,403,484; and 5,223,409), plasmids (Cull et al., Proc. Natl. Acad. Sci. USA, 89:1865-1869, 1992) orphage (Scott and Smith, Science, 249:386-390, 1990; Devlin, Science, 249:404-406, 1990; Cwirla et al., Proc. Natl. Acad. Sci. USA, 87:6378-6382, 1990; and Felici, J. Mol. Biol., 222:301-310, 1991).

In one embodiment, a cell-based assay is employed in which a cell that expresses a target protein or biologically active portion thereof is contacted with a test compound. The ability of the test compound to modulate expression or activity of the target protein is then determined. The cell, for example, can be a yeast cell or a cell of mammalian origin, e.g., rat, mouse, or human.

The ability of the test compound to bind to a target protein or modulate target protein binding to a compound, e.g., a target protein substrate, can also be evaluated. This can be accomplished, for example, by coupling the compound, e.g., the substrate, with a radioisotope or enzymatic label such that binding of the compound, e.g., the substrate, to the target protein can be determined by detecting the labeled compound, e.g., substrate, in a complex. Alternatively, the target protein can be coupled with a radioisotope or enzymatic label to monitor the ability of a test compound to modulate target protein binding to a target protein substrate in a complex. For example, compounds (e.g., target protein substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a target protein substrate) to interact with target protein with or without the labeling of any of the interactants can be evaluated. For example, a microphysiometer can be used to detect the interaction of a compound with a target protein without the labeling of either the compound or the target protein (McConnell et al., Science 257:1906-1912, 1992). As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between a compound and a target protein.

In yet another embodiment, a cell-free assay is provided in which a target protein or biologically active portion thereof is contacted with a test compound and the ability of the test compound to bind to the target protein or biologically active portion thereof is evaluated. In general, biologically active portions of target proteins to be used in assays described herein include fragments that participate in interactions with other molecules, e.g., fragments with high surface probability scores.

Cell-free assays involve preparing a reaction mixture of the target protein and the test compound under conditions and for a time sufficient to allow the two components to interact and bind, thus forming a complex that can be removed and/or detected.

The interaction between two molecules can also be detected using fluorescence energy transfer (FET) (see, for example, Lakowicz et al., U.S. Pat. No. 5,631,169; Stavrianopoulos et al., U.S. Pat. No. 4,868,103). A fluorophore label on the first, “donor” molecule is selected such that its emitted fluorescent energy will be absorbed by a fluorescent label on a second, “acceptor” molecule, which in turn is able to fluoresce due to the absorbed energy. Alternately, the “donor” protein molecule may use the natural fluorescent energy of tryptophan residues. Labels are chosen that emit different wavelengths of light, such that the “acceptor” molecule label may be differentiated from that of the “donor.” Since the efficiency of energy transfer between the labels is related to the distance separating the molecules, the spatial relationship between the molecules can be assessed. In a situation in which binding occurs between the molecules, the fluorescent emission of the “acceptor” molecule label in the assay should be maximal. A FET binding event can be conveniently measured through standard fluorometric detection means well known in the art (e.g., using a fluorimeter).

In another embodiment, the ability of a target protein to bind to a target molecule can be determined using real-time Biomolecular Interaction Analysis (BIA) (e.g., Sjolander et al., Anal. Chem., 63:2338-2345, 1991, and Szabo et al., Curr. Opin. Struct. Biol., 5:699-705, 1995). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In various of these assays, the target protein or the test substance is anchored onto a solid phase. The target protein/test compound complexes anchored on the solid phase can be detected at the end of the reaction. Generally, the target protein is anchored onto a solid surface, and the test compound (which is not anchored) can be labeled, either directly or indirectly, with detectable labels discussed herein.

It may be desirable to immobilize either the target protein, an anti-target protein antibody, or its target molecule to facilitate separation of complexed from uncomplexed forms of one or both of the proteins, as well as to accommodate automation of the assay. Binding of a test compound to a target protein, or interaction of a target protein with a target molecule in the presence and absence of a test compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, glutathione-S-transferase/target protein fusion proteins or glutathione-S-transferase/target fusion proteins can be adsorbed onto glutathione Sepharose™ beads (Sigma Chemical, St. Louis, Mo.) or glutathione derivatized microtiter plates, which are then combined with the test compound or the test compound and either the non-adsorbed target protein. The mixture is then incubated under conditions conducive to complex formation (e.g., at physiological conditions for salt and pH). Following incubation, the beads or microtiter plate wells are washed to remove any unbound components, the matrix immobilized in the case of beads, and the complex determined either directly or indirectly, for example, as described above. Alternatively, the complexes can be dissociated from the matrix, and the level of target protein binding or activity determined using standard techniques.

Other techniques for immobilizing a target protein on matrices include using conjugation of biotin and streptavidin. Biotinylated target protein can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

To conduct the assay, the non-immobilized component is added to the coated surface containing the anchored component. After the reaction is complete, unreacted components are removed (e.g., by washing) under conditions such that any complexes formed will remain immobilized on the solid surface. The complexes anchored on the solid surface can be detected in a number of ways. Where the previously non-immobilized component is pre-labeled, the presence of a label immobilized on the surface indicates that complexes were formed. Where the previously non-immobilized component is not pre-labeled, an indirect label can be used to detect complexes anchored on the surface; e.g., using-a-labeled antibody specific for the immobilized component (the antibody, in turn, can be directly labeled or indirectly labeled with, e.g., a labeled anti-Ig antibody).

In some cases, the assay is performed utilizing antibodies reactive with target protein, but which do not interfere with binding of the target protein to its target molecule. Such antibodies can be derivatized to the wells of the plate, and unbound target protein trapped in the wells by antibody conjugation. Methods for detecting such complexes, in addition to those described above for the GST-immobilized complexes, include immunodetection of complexes using antibodies reactive with the target protein or target molecule, as well as enzyme-linked assays which rely on detecting an enzymatic activity associated with the target protein.

Alternatively, cell-free assays can be conducted in a liquid phase. In such an assay, the reaction products are separated from unreacted components, by any of a number of standard techniques, including but not limited to: differential centrifugation (see, for example, Rivas and Minton, Trends Biochem. Sci., 18:284-7, 1993); chromatography (gel filtration chromatography, ion-exchange chromatography); electrophoresis (e.g., Ausubel et al., eds. Current Protocols in Molecular Biology 1999, J. Wiley: N.Y.); and immunoprecipitation (see, for example, Ausubel et al., eds., 1999, Current Protocols in Molecular Biology, J. Wiley: N.Y.). Such resins and chromatographic techniques are known to one skilled in the art (e.g., Heegaard, J. Mol. Recognit., 11: 141-148, 1998; Hage et al., J. Chromatogr. B. Biomed. Sci. Appl., 699:499-525, 1997). Further, fluorescence energy transfer may also be conveniently utilized, as described herein, to detect binding without further purification of the complex from solution.

The assay can include contacting the target protein or a biologically active portion thereof with a known compound that binds to the target protein to form an assay mixture, contacting the assay mixture with a test compound, and determining the ability of the test compound to interact with the target protein, wherein determining the ability of the test compound to interact with the target protein includes determining the ability of the test compound to preferentially bind to the target protein or biologically active portion thereof, or to modulate the activity of a target molecule, as compared to the known compound.

A target protein can, in vivo, interact with one or more cellular or extracellular macromolecules, such as proteins. For the purposes of this discussion, such cellular and extracellular macromolecules are referred to herein as “binding partners.” Compounds that disrupt such interactions are useful for regulating the activity of the target protein. Such compounds can include, but are not limited, to molecules such as antibodies, peptides, and small molecules. In general, target proteins for use in identifying agents that disrupt interactions are the target proteins identified herein. In alternative embodiments, the invention provides methods for determining the ability of the test compound to modulate the activity of a target protein through modulation of the activity of a downstream effector of a target protein. For example, the activity of the effector molecule on an appropriate target can be determined, or the binding of the effector to an appropriate target can be determined, as described herein.

To identify compounds that interfere with the interaction between the target protein and its binding partner(s), a reaction mixture containing the target protein and the binding partner is prepared, under conditions and for a time sufficient, to allow the two products to form a complex. To test an inhibitory agent, the reaction mixture is provided in the presence (test sample) and absence (control sample) of the test compound. The test compound can be initially included in the reaction mixture, or can be added at a time subsequent to the addition of the target gene and its cellular or extracellular binding partner. Control reaction mixtures are incubated without the test compound or with a control compound. The formation of complexes between the target protein and the cellular or extracellular binding partner is then detected. The formation of a complex in the control reaction, and less formation of complex in the reaction mixture containing the test compound, indicates that the compound interferes with the interaction of the target protein and the interactive binding partner. Such compounds are candidate compounds for inhibiting the expression or activity or a target protein. Additionally, complex formation within reaction mixtures containing the test compound and normal target protein can also be compared to complex formation within reaction mixtures containing the test compound and mutant target gene product. This comparison can be important in those cases wherein it is desirable to identify compounds that disrupt interactions of mutant but not normal target protein.

Binding assays can be carried out in a liquid phase or in heterogenous formats. In one type of heterogeneous assay system, either the target protein or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

To conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

In another embodiment, modulators of target expression (RNA or protein) are identified. For example, a cell or cell-free mixture is contacted with a test compound and the expression of target mRNA or protein evaluated relative to the level of expression of target mRNA or protein in the absence of the test compound. When expression of target mRNA or protein is greater in the presence of the test compound than in its absence, the test compound is identified as a stimulator (candidate compound) of target mRNA or protein expression. Alternatively, when expression of target mRNA or protein is less (statistically significantly less) in the presence of the test compound than in its absence, the test compound is identified as an inhibitor (candidate compound) of target mRNA or protein expression. The level of target mRNA or protein expression can be determined by methods described herein and methods known in the art such as Northern blot or Western blot for detecting target mRNA or protein.

In another aspect, the methods described herein pertain to a combination of two or more of the assays described herein. For example, a modulating agent can be identified using a cell-based or a cell-free assay, and the ability of the agent to modulate the activity of a target protein can be confirmed in vivo, e.g., in an animal such as an animal model for a disorder characterized by impaired protein trafficking such as Cystic fibrosis or any others described herein.

This invention further pertains to novel agents identified by the above-described screening assays. Accordingly, it is within the scope of this invention to further use an agent (compound) identified as described herein (e.g., a target protein modulating agent, an anti sense nucleic acid molecule, an siRNA, a target protein-specific antibody, or a target protein-binding partner) in an appropriate animal model to determine the efficacy, toxicity, side effects, or mechanism of action, of treatment with such an agent. Furthermore, novel agents identified by the above-described screening assays can be used for treatments as described herein.

Compounds that modulate target protein expression or activity (target protein modulators) can be tested for their ability to affect metabolic effects associated with the target protein, e.g., with decreased expression or activity of target protein using methods known in the art and methods described herein. For example, the ability of a compound to modulate alpha-synuclein mediated toxicity can be tested using an in vitro or in vivo model for a disorder characterized by impaired protein trafficking such as Cystic fibrosis or any others described herein.

### Target Protein Modulators

Methods of modulating target protein expression or activity can be accomplished using a variety of compounds including nucleic acid molecules that are targeted to a target nucleic acid sequence or fragment thereof, or to a target protein. Compounds that may be useful for inhibiting target protein expression or activity include polynucleotides, polypeptides, small non-nucleic acid organic molecules, small inorganic molecules, antibodies or fragments thereof, antisense oligonucleotides, siRNAs, and ribozymes. Methods of identifying such compounds are described herein.

### RNA Inhibition (RNAi)

Molecules that are targeted to a target RNA are useful for the methods described herein, e.g., inhibition of target protein expression, e.g., for treating synucleinopathies such as Parkinson's disease. Examples of nucleic acids include siRNAs. Other such molecules that function using the mechanisms associated with RNAi can also be used including chemically modified siRNAs and vector driven expression of hairpin RNA that are then cleaved to siRNA. The nucleic acid molecules or constructs that are useful as described herein include dsRNA (e.g., siRNA) molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is complementary to the first strand. The dsRNA molecules can be chemically synthesized, can transcribed be in vitro from a DNA template, or can be transcribed in vivo from, e.g., shRNA. The dsRNA molecules can be designed using methods known in the art, e.g., Dharmacon.com (see, siDESIGN CENTER) or “The siRNA User Guide,” available on the Internet at mpibpc.gwdg.de/abteilunge-n/100/105/sirna.html.

Negative control siRNAs (“scrambled”) generally have the same nucleotide composition as the selected siRNA, but without significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. Controls can also be designed by introducing an appropriate number of base mismatches into the selected siRNA sequence.

The nucleic acid compositions that are useful for the methods described herein include both siRNA and crosslinked siRNA derivatives. Crosslinking can be used to alter the pharmacokinetics of the composition, for example, to increase half-life in the body. Thus, the invention includes siRNA derivatives that include siRNA having two complementary strands of nucleic acid, such that the two strands are crosslinked. For example, a 3′ OH terminus of one of the strands can be modified, or the two strands can be crosslinked and modified at the 3′OH terminus. The siRNA derivative can contain a single crosslink (e.g., a psoralen crosslink). In some cases, the siRNA derivative has at its 3′ terminus a biotin molecule (e.g., a photocleavable biotin), a peptide (e.g., a Tat peptide), a nanoparticle, a peptidomimetic, organic compounds (e.g., a dye such as a fluorescent dye), or dendrimer. Modifying SiRNA derivatives in this way can improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The nucleic acid compositions described herein can be unconjugated or can be conjugated to another moiety, such as a nanoparticle, to enhance a property of the compositions, e.g., a pharmacokinetic parameter such as absorption, efficacy, bioavailability, and/or half-life. The conjugation can be accomplished using methods known in the art, e.g., using the methods of Lambert et al., Drug Deliv. Rev., 47, 99-112, 2001 (describes nucleic acids loaded to polyalkylcyanoacrylate (PACA) nanoparticles); Fattal et al., J. Control Release, 53:137-143, 1998 (describes nucleic acids bound to nanoparticles); Schwab et al., Ann. Oncol., 5 Suppl. 4:55-8, 1994 (describes nucleic acids linked to intercalating agents, hydrophobic groups, polycations or PACA nanoparticles); and Godard et al., Eur. J. Biochem., 232:404-410, 1995 (describes nucleic acids linked to nanoparticles).

The nucleic acid molecules can also be labeled using any method known in the art; for instance, the nucleic acid compositions can be labeled with a fluorophore, e.g., Cy3, fluorescein, or rhodamine. The labeling can be carried out using a kit, e.g., the SILENCER.™. siRNA labeling kit (Ambion). Additionally, the molecule can be radiolabeled, e.g., using ³H, ³²P, or other appropriate isotope.

Synthetic siRNAs can be delivered into cells by cationic liposome transfection and electroporation. Sequences that are modified to improve their stability can be used. Such modifications can be made using methods known in the art (e.g., siSTABLE™, Dharmacon). Such stabilized molecules are particularly useful for in vivo methods such as for administration to a subject to decrease target protein expression. Longer term expression can also be achieved by delivering a vector that expresses the siRNA molecule (or other nucleic acid) to a cell, e.g., a fat, liver, or muscle cell. Several methods for expressing siRNA duplexes within cells from recombinant DNA constructs allow longer-term target gene suppression in cells, including mammalian Pol III promoter systems (e.g., HI or U6/snRNA promoter systems (Tuschl, Nature Biotechnol., 20:440-448, 2002) capable of expressing functional double-stranded siRNAs; (Bagella et al., J. Cell. Physiol., 177:206-1998; Lee et al., Nature Biotechnol., 20:500-505, 2002; Paul et al., Nature Biotechnol., 20:505-508, 2002; Yu et al., Proc. Natl. Acad. Sci. USA, 99(9):6047-6052, 2002; Sui et al., Proc. Natl. Acad. Sci. USA, 99(6):5515-5520, 2002). Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella et al., 1998, supra; Lee et al., 2002, supra; Paul et al., 2002, supra; Yu et al., 2002, supra; Sui et al., 2002, supra). Constructs containing siRNA sequence under the control of T7 promoter also make functional siRNAs when cotransfected into the cells with a vector expression T7 RNA polymerase (Jacque, Nature, 418:435-438, 2002).

Animal cells express a range of noncoding RNAs of approximately 22 nucleotides termed micro RNA (miRNAs) and can regulate gene expression at the post transcriptional or translational level during animal development. miRNAs are excised from an approximately 70 nucleotide precursor RNA stem-loop. By substituting the stem sequences of the miRNA precursor with miRNA sequence complementary to the target mRNA, a vector construct that expresses the novel miRNA can be used to produce siRNAs to initiate RNAi against specific mRNA targets in mammalian cells (Zeng, Mol. Cell, 9:1327-1333, 2002). When expressed by DNA vectors containing polymerase III promoters, micro-RNA designed hairpins can silence gene expression (McManus, RNA 8:842-850, 2002). Viral-mediated delivery mechanisms can also be used to induce specific silencing of targeted genes through expression of siRNA, for example, by generating recombinant adenoviruses harboring siRNA under RNA Pol II promoter transcription control (Xia et al., Nat Biotechnol., 20(10): 1006-10, 2002).

Injection of the recombinant adenovirus vectors into transgenic mice expressing the target genes of the siRNA results in in vivo reduction of target gene expression. In an animal model, whole-embryo electroporation can efficiently deliver synthetic siRNA into post-implantation mouse embryos (Calegari et al., Proc. Natl. Acad. Sci. USA, 99:14236-14240, 2002). In adult mice, efficient delivery of siRNA can be accomplished by “high-pressure” delivery technique, a rapid injection (within 5 seconds) of a large volume of siRNA containing solution into animal via the tail vein (Liu, Gene Ther., 6:1258-1266, 1999; McCaffrey, Nature, 418:38-39, 2002; Lewis, Nature Genetics, 32:107-108, 2002). Nanoparticles and liposomes can also be used to deliver siRNA into animals. Likewise, in some embodiments, viral gene delivery, direct injection, nanoparticle particle-mediated injection, or liposome injection may be used to express siRNA in humans.

In some cases, a pool of siRNAs is used to modulate the expression of a target gene. The pool is composed of at least 2, 3, 4, 5, 8, or 10 different sequences targeted to the target gene.

SiRNAs or other compositions that inhibit target protein expression or activity are effective for ameliorating undesirable effects of a disorder related to alpha synuclein toxicity when target RNA levels are reduced by at least 25%, 50%, 75%, 90%, or 95%. In some cases, it is desired that target RNA levels be reduced by not more than 10%, 25%, 50%, or 75%. Methods of determining the level of target gene expression can be determined using methods known in the art. For example, the level of target RNA can be determined using Northern blot detection on a sample from a cell line or a subject. Levels of target protein can also be measured using, e.g., an immunoassay method.

### Antisense Nucleic Acids

Antisense nucleic acids are useful for inhibiting a target protein. Such antisense nucleic acid molecules, i.e., nucleic acid molecules whose nucleotide sequence is complementary to all or part of an mRNA encoding a target protein. An antisense nucleic acid molecule can be antisense to all or part of a non-coding region of the coding strand of a nucleotide sequence encoding a target protein. The non-coding regions (“5′ and 3′ untranslated regions”) are the 5′ and 3′ sequences that flank the coding region and are not translated into amino acids.

Based upon the nucleotide sequences disclosed herein, one of skill in the art can easily choose and synthesize any of a number of appropriate antisense molecules to target a gene described herein. For example, a “gene walk” comprising a series of oligonucleotides of 15-30 nucleotides spanning the length of a nucleic acid (e.g., a target nucleic acid) can be prepared, followed by testing for inhibition of expression of the gene. Optionally, gaps of 5-10 nucleotides can be left between the oligonucleotides to reduce the number of oligonucleotides synthesized and tested.

An antisense oligonucleotide can be, for example, about 5, 10, 15, 20, 25, 30, 35, 40, 45, or 50 nucleotides or more in length. An antisense nucleic acid described herein can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The new antisense nucleic acid molecules can be administered to a mammal, e.g., a human patient. Alternatively, they can be generated in situ such that they hybridize with or bind to cellular mRNA and/or genomic DNA encoding a selected polypeptide to thereby inhibit expression, e.g., by inhibiting transcription and/or translation. The hybridization can be by conventional nucleotide complementarities to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. For example, to achieve sufficient intracellular concentrations of the antisense molecules, vector constructs can be used in which the antisense nucleic acid molecule is placed under the control of a strong pol II or pol III promoter.

An antisense nucleic acid molecule can be an alpha-anomeric nucleic acid molecule. An alpha-anomeric nucleic acid molecule forms specific double-stranded hybrids with complementary RNA in which, contrary to the usual, beta-units, the strands run parallel to each other (Gaultier et al., Nucleic Acids Res., 15:6625-6641, 1987). The antisense nucleic acid molecule can also comprise a 2′-o-methylribonucleotide (Inoue et al., Nucleic Acids Res., 15:6131-6148, 1987) or a chimeric RNA-DNA analog (Inoue et al., FEBS Lett., 215:327-330, 1987).

Antisense molecules that are complementary to all or part of a target gene described herein are also useful for assaying expression of such genes using hybridization methods known in the art. For example, the antisense molecule can be labeled (e.g., with a radioactive molecule) and an excess amount of the labeled antisense molecule is hybridized to an RNA sample. Unhybridized labeled antisense molecule is removed (e.g., by washing) and the amount of hybridized antisense molecule measured. The amount of hybridized molecule is measured and used to calculate the amount of expression of the target gene. In general, antisense molecules used for this purpose can hybridize to a sequence from a target gene under high stringency conditions such as those described herein. When the RNA sample is first used to synthesize cDNA, a sense molecule can be used. It is also possible to use a double-stranded molecule in such assays as long as the double-stranded molecule is adequately denatured prior to hybridization.

### Ribozymes

Ribozymes that have specificity for a target nucleic acid sequence can also be used to inhibit target gene expression. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach, Nature, 334:585-591, 1988)) can be used to catalytically cleave mRNA transcripts to thereby inhibit translation of the protein encoded by the mRNA. Methods of designing and producing ribozymes are known in the art (see, e.g., Scanlon, 1999, Therapeutic Applications of Ribozymes, Humana Press). A ribozyme having specificity for a target nucleic acid molecule or fragment thereof can be designed based upon the nucleotide sequence of a target cDNA. For example, a derivative of a Tetrahymena L-19 IVS RNA can be constructed in which the nucleotide sequence of the active site is complementary to the nucleotide sequence to be cleaved in a target RNA (Cech et al. U.S. Pat. No. 4,987,071; and Cech et al., U.S. Pat. No. 5,116,742). Alternatively, an mRNA encoding a target protein or fragment thereof can be used to select a catalytic RNA having a specific ribonuclease activity from a pool of RNA molecules (See, e.g., Bartel and Szostak, Science, 261:1411-1418, 1993).

Nucleic acid molecules that form triple helical structures can also be used to modulate target protein expression. For example, expression of a target protein can be inhibited by targeting nucleotide sequences complementary to the regulatory region of the gene encoding the polypeptide (e.g., the promoter and/or enhancer) to form triple helical structures that prevent transcription of the gene in target cells. See generally Helene, Anticancer Drug Des., 6(6):569-84, 1991; Helene, Ann. N. Y Acad. Sci., 660:27-36, 1992; and Maher, Bioassays, 14(12):807-15, 1992.

A nucleic acid molecule for use as described herein can be modified at the base moiety, sugar moiety or phosphate backbone to improve, e.g., the stability, hybridization, or solubility of the molecule. For example, the deoxyribose phosphate backbone of a nucleic acid can be modified to generate peptide nucleic acids (see Hyrup et al., Bioorganic & Medicinal Chem., 4(1): 5-23, 1996). Peptide nucleic acids (PNAs) are nucleic acid mimics, e.g., DNA mimics, in which the deoxyribose phosphate backbone is replaced by a pseudopeptide backbone and only the four natural nucleobases are retained. The neutral backbone of PNAs allows for specific hybridization to DNA and RNA under conditions of low ionic strength. The synthesis of PNA oligomers can be performed using standard solid phase peptide synthesis protocols, e.g., as described in Hyrup et al., 1996, supra; Perry-O'Keefe et al., Proc. Natl. Acad. Sci. USA, 93: 14670-675, 1996.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup, 1996, supra; or as probes or primers for DNA sequence and hybridization (Hyrup, 1996, supra; Perry-O'Keefe et al., Proc. Natl. Acad. Sci. USA, 93: 14670-675, 1996).

PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which may combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup, 1996, supra, and Finn et al., Nucleic Acids Res., 24:3357-63, 1996. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., Nucleic Acids Res., 17:5973-88, 1989). PNA monomers are then coupled in a stepwise manner to produce a chimeric molecule with a 5′ PNA segment and a 3′ DNA segment (Finn et al., Nucleic Acids Res., 24:3357-63, 1996). Alternatively, chimeric molecules can be synthesized with a 5′ DNA segment and a 3′ PNA segment (Peterser et al., Bioorganic Med. Chem. Lett., 5:1119-11124, 1975).

A nucleic acid targeting a target nucleic acid sequence can include appended groups such as peptides (e.g., for targeting host cell receptors in vivo), or agents facilitating transport across the cell membrane (see, e.g., Letsinger et al., Proc. Natl. Acad. Sci. USA, 86:6553-6556, 1989; Lemaitre et al., Proc. Natl. Acad. Sci. USA, 84:648-652, 1989; WO 88/09810) or the blood-brain barrier (see, e.g., WO 89/10134). In addition, oligonucleotides can be modified with hybridization-triggered cleavage agents (see, e.g., Krol et al., Bio/Techniques, 6:958-976, 1988) or intercalating agents (see, e.g., Zon, Pharm. Res., 5:539-549, 1988). To this end, the oligonucleotide may be conjugated to another molecule, e.g., a peptide, hybridization triggered cross-linking agent, transport agent, or a hybridization-triggered cleavage agent.

### Polypeptides

Isolated target proteins, fragments thereof, and variants thereof are provided herein. These polypeptides can be used, e.g., as immunogens to raise antibodies, in screening methods, or in methods of treating subjects, e.g., by administration of the target proteins. An “isolated” or “purified” polypeptide or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of polypeptides in which the polypeptide of interest is separated from cellular components of the cells from which it is isolated or recombinantly produced. Thus, a polypeptide that is substantially free of cellular material includes preparations of polypeptides having less than about 30%, 20%, 10%, or 5% (by dry weight) of heterologous protein (also referred to herein as “contaminating protein”). In general, when the polypeptide or biologically active portion thereof is recombinantly produced, it is also substantially free of culture medium, i.e., culture medium represents less than about 20%, 10%, or 5% of the volume of the protein preparation. In general, when the polypeptide is produced by chemical synthesis, it is substantially free of chemical precursors or other chemicals, i.e., it is separated from chemical precursors or other chemicals that are involved in the synthesis of the polypeptide. Accordingly such preparations of the polypeptide have less than about 30%, 20%, 10%, or 5% (by dry weight) of chemical precursors or compounds other than the polypeptide of interest.

Expression of target proteins can be assayed to determine the amount of expression. Methods for assaying protein expression are known in the art and include Western blot, immunoprecipitation, and radioimmunoassay.

As used herein, a “biologically active portion” of a target protein includes a fragment of a target protein that participates in an interaction between a target proteins and a non-target protein. Biologically active portions of a target protein include peptides including amino acid sequences sufficiently homologous to the amino acid sequence of a target protein that includes fewer amino acids than a full-length target protein, and exhibits at least one activity of a target protein. Typically, biologically active portions include a domain or motif with at least one activity of the target protein. A biologically active portion of a target protein can be a polypeptide that is, for example, 10, 25, 50, 100, 200 or more amino acids in length. Biologically active portions of a target protein can be used as targets for developing agents that modulate a target protein mediated activity, e.g., compounds that inhibit target protein activity.

In some embodiments, the target protein has a sequence identical to a sequence disclosed herein (e.g., an amino acid sequence found under a GenBank™ Accession Number listed in Table III). Other useful polypeptides are substantially identical (e.g., at least about 45%, 55%, 65%, 75%, 85%, 95%, or 99% identical) to a sequence disclosed herein (e.g., an amino acid sequence found under a GenBank™ Accession Number listed in Table III) and (a) retains the. functional activity of the target protein yet differs in amino acid sequence due to natural allelic variation or mutagenesis, or (b) exhibits an altered functional activity (e.g., as a dominant negative) where desired. Provided herein are variants that have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the polypeptide. -An antagonist of a polypeptide can inhibit one or more of the activities of the naturally occurring form of the polypeptide by, for example, competitively binding to a downstream or upstream member of a cellular signaling cascade that includes the polypeptide. Thus, specific biological effects can be elicited by treatment with a variant of limited function. Treatment of a subject with a variant having a subset of the biological activities of the naturally occurring form of the polypeptide can have fewer side effects in a subject relative to treatment with the naturally occurring form of the polypeptide. In some embodiments, the variant target protein is a dominant negative form of the target protein. Dominant negatives are desired, e.g., in methods in which inhibition of target protein action is desired.

Also provided herein are chimeric or fusion proteins.

The comparison of sequences and determination of percent identity between two sequences is accomplished using a mathematical algorithm. The percent identity between two amino acid sequences is determined using the Needleman and Wunsch, J. Mol. Biol., 48:444-453, 1970) algorithm, which has been incorporated into the GAP program in the GCG software package (available on the Internet at gcg.com), using either a Blossum 62 matrix or a PAM250 matrix, and a gap weight of 16 and a length weight of 1. The percent identity between two nucleotide sequences is determined using the GAP program in the GCG software package (also available on the Internet at gcg.com), using a NWSgapdna.CMP matrix, a gap weight of 40, and a length weight of 1.

In general, percent identity between amino acid sequences referred to herein is determined using the BLAST 2.0 program, which is available to the public on the Internet at ncbi.nlm.nih.gov/BLAST. Sequence comparison is performed using an ungapped alignment and using the default parameters (Blossum 62 matrix, gap existence cost of 11, per residue gap cost of 1, and a lambda ratio of.0.85). The mathematical algorithm used in BLAST programs is described in Altschul et al., Nucleic Acids Research 25:3389-3402, 1997.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a target protein is generally replaced with another amino acid residue from the same side chain family. Alternatively, mutations can be introduced randomly along all or part of a target protein coding sequence, such as by saturation mutagenesis, and the resultant mutants can be screened for target protein biological activity to identify mutants that retain activity. The encoded protein can be expressed recombinantly and the activity of the protein can be determined.

### Antibodies

A target protein, or a fragment thereof, can be used as an immunogen to generate antibodies using standard techniques for polyclonal and monoclonal antibody preparation. The full-length polypeptide or protein can be used or, alternatively, antigenic peptide fragments can be used as immunogens. The antigenic peptide of a protein comprises at least 8 (e.g., at least 10, 15, 20, or 30) amino acid residues of the amino acid sequence of a target protein, and encompasses an epitope of a target protein such that an antibody raised against the peptide forms a specific immune complex with the polypeptide.

An immunogen typically is used to prepare antibodies by immunizing a suitable subject (e.g., rabbit, goat, mouse or other mammal). An appropriate immunogenic preparation can contain, for example, a recombinantly expressed or a chemically synthesized polypeptide. The preparation can further include an adjuvant, such as Freund's complete or incomplete adjuvant, or similar immunostimulatory agent.

Polyclonal antibodies can be prepared as described above by immunizing a suitable subject with a target protein as an immunogen. The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein, Nature, 256:495-497, 1975, the human B cell hybridoma technique (Kozbor et al., Immunol. Today, 4:72, 1983), the EBV-hybridoma technique (Cole et al., Monoclonal Antibodies and Cancer Therapy, Alan R. Liss, Inc., pp. 77-96, 1985) or trioma techniques. The technology for producing hybridomas is well known (see generally Current Protocols in Immunology, 30 1994, Coligan et al. (eds.) John Wiley & Sons, Inc., New York, N.Y.). Hybridoma cells producing a monoclonal antibody are detected by screening the hybridoma culture supernatants for antibodies that bind the polypeptide of interest, e.g., using a standard ELISA assay.

As an alternative to preparing monoclonal antibody-secreting hybridomas, a monoclonal antibody directed against a polypeptide can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No.5,223,409; WO 92118619; WO 91/17271; WO 92/20791; WO 92/15679; WO 93/01288; WO 92/01047; WO 92/09690; WO 90/02809; Fuchs et al., Bio/Technology, 9:1370-1372, 1991; Hay et al., Hum. Antibod. Hybridomas, 3:81-85, 1992; Huse et al., Science, 246:1275-1281, 1989; Griffiths et al., EMBO J., 12:725-734, 1993.

Additionally, recombinant antibodies, such as chimeric and humanized monoclonal antibodies, including both human and non-human portions, which can be made using standard recombinant DNA techniques, are provided herein. Such chimeric and humanized monoclonal antibodies can be produced by recombinant DNA techniques known in the art, for example using methods described in WO 87/02671; European Patent Application 184,187; European Patent Application 171,496; European Patent Application 173,494; WO 86/01533; U.S. Pat. No. 4,816,567; European Patent Application 125,023; Better et al., Science, 240:1041-1043, 1988; Liu et al., Proc. Natl. Acad. Sci. USA 84:3439-3443, 1987; Liu et al., J. Immunol., 139:3521-3526, 1987; Sun et al., Proc. Natl. Acad. Sci. USA, 84:214-218, 1987; Nishimura et al., Canc. Res., 47:999-1005, 1987; Wood et al., Nature, 314:446-449, 1985; and Shaw et al., J. Natl. Cancer Inst., 80: 1553-1559, 1988); Morrison, Science, 229:1202-1207, 1985; Oi et al., Bio/Techniques, 4:214, 1986; U.S. Pat. No. 5,225,539; Jones et al., Nature, 321:552-525, 1986; Verhoeyan et al., Science, 239:1534, 1988; and Beidler et al., J. Immunol., 141:4053-4060, 1988.

Completely human antibodies are particularly desirable for therapeutic treatment of human patients. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a target protein. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, and IgE antibodies. For an overview of this technology for producing human antibodies, see Lonberg and Huszar (Int. Rev. Immunol., 13:65-93, 1995). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806.

Completely human antibodies that recognize a selected epitope can be generated using a technique referred to as “guided selection.” In this approach a selected non-human monoclonal antibody, e.g., a murine antibody, is used to guide the selection of a completely human antibody recognizing the same epitope. (Jespers et al., Biotechnology, 12:899-903, 1994).

An antibody directed against a target protein can be used to detect the polypeptide (e.g., in a cellular lysate or cell supernatant) to evaluate its abundance and pattern of expression. The antibodies can also be used diagnostically to monitor protein levels in tissue as part of a clinical testing procedure, e.g., for example, to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

### G. Methods of Treating a Disorder Characterized by Impaired Protein Trafficking

GTP-bound Rab proteins such as Rab1, the homolog of yeast ypt1, are involved in the global regulation of vesicle transport. As detailed throughout the specification and in the Examples, compounds identified in the ypt^{ts} mutant rescue screening assay can be useful to stabilize trafficking defective proteins, e.g., by modulating the Rab-ypt1 pathway. Thus, the compounds disclosed herein (and pharmaceutical compositions comprising same) can be useful in methods to treat one or more symptoms of a variety of disorders characterized by impaired protein trafficking. As described in Example 4, compounds identified using the ypt1^{ts} mutant rescue screen are also capable of stabilizing ΔF508 CFTR. Thus the compounds described herein can be particularly useful in treating or preventing one or more symptoms of cystic fibrosis.

Types of disorders characterized by impaired protein trafficking that could be treated through the administration of one or more compounds (or pharmaceutical compositions of the same) described herein can include, e.g., hereditary emphysema, hereditary hemochromatosis, oculocutaneous albinism, protein C deficiency, type I hereditary angioedema, congenital sucrase-isomaltase deficiency, Crigler-Najjar type II, Laron syndrome, hereditary Myeloperoxidase, primary hypothyroidism, congenital long QT syndrome, tyroxine binding globulin deficiency, familial hypercholesterolemia, familial chylomicronemia, abeta-lipoproteinema, low plasma lipoprotein a levels, hereditary emphysema with liver injury, congenital hypothyroidism, osteogenesis imperfecta, hereditary hypofibrinogenemia, alpha-1 antichymotrypsin deficiency, nephrogenic diabetes insipidus, neurohypophyseal diabetes, insipidus, Charcot-Marie-Tooth syndrome, Pelizaeus Merzbacher disease, von Willebrand disease type IIA, combined factors V and VIII deficiency, spondylo-epiphyseal dysplasia tarda, choroideremia, I cell disease, Batten disease, ataxia telangiectasias, acute lymphoblastic leukemia, acute myeloid leukemia, myeloid leukemia, ADPKD-autosomal dominant polycystic kidney disease, microvillus inclusion disease, tuberous sclerosis, oculocerebro-renal syndrome of Lowe, amyotrophic lateral sclerosis, myelodysplastic syndrome, Bare lymphocyte syndrome, Tangier disease, familial intrahepatic cholestasis, X-linked adreno-leukodystrophy, Scott syndrome, Hermansky-Pudlak syndrome types 1 and 2, Zellweger syndrome, rhizomelic chondrodysplasia puncta, autosomal recessive primary hyperoxaluria, Mohr Tranebjaerg syndrome, spinal and bullar muscular atrophy, primary ciliary diskenesia (Kartagener's syndrome), Miller Dieker syndrome, lissencephaly, motor neuron disease, Usher's syndrome, Wiskott-Aldrich syndrome, Optiz syndrome, Huntington's disease, hereditary pancreatitis, anti-phospholipid syndrome, overlap connective tissue disease, Sjögren's syndrome, stiff-man syndrome, Brugada syndrome, congenital nephritic syndrome of the Finnish type, Dubin-Johnson syndrome, X-linked hypophosphosphatemia, Pendred syndrome, persistent hyperinsulinemic hypoglycemia of infancy, hereditary spherocytosis, aceruloplasminemia, infantile neuronal ceroid lipofuscinosis, pseudoachondroplasia and multiple epiphyseal, Stargardt-like macular dystrophy, X-linked. Charcot-Marie-Tooth disease, autosomal dominant retinitis pigmentosa, Wolcott-Rallison syndrome, Cushing's disease, limb-girdle muscular dystrophy, mucoploy-saccharidosis type IV, hereditary familial amyloidosis of Finish, Anderson disease, sarcoma, chronic myelomonocytic leukemia, cardiomyopathy, faciogenital dysplasia, Torsion disease, Huntington and spinocerebellar ataxias, hereditary hyperhomosyteinemia, polyneuropathy, lower motor neuron disease, pigmented retinitis, seronegative polyarthritis, interstitial pulmonary fibrosis, Raynaud's phenomenon, Wegner's granulomatosis, preoteinuria, CDG-Ia, CDG-Ib, CDG-Ic, CDG-Id, CDG-Ie, CDG-If, CDG-IIa, CDG-IIb, CDG-IIc, CDG-IId, Ehlers-Danlos syndrome, multiple exostoses, Griscelli syndrome (type 1 or type 2), or X-linked non-specific mental retardation. In addition, disorders characterized by impaired protein trafficking can also include lysosomal storage disorders such as, but not limited to, Fabry disease, Farber disease, Gaucher disease, GM₁-gangliosidosis, Tay-Sachs disease, Sandhoff disease, GM₂ activator disease, Krabbe disease, metachromatic leukodystrophy, Niemann-Pick disease (types A, B, and C), Hurler disease, Scheie disease, Hunter disease, Sanfilippo disease, Morquio disease, Maroteaux-Lamy disease, hyaluronidase deficiency, aspartylglucosaminuria, filcosidosis, mannosidosis, Schindler disease, sialidosis type 1, Pompe disease, Pycnodysostosis, ceroid lipofuscinosis, cholesterol ester storage disease, Wolman disease, Multiple sulfatase, galactosialidosis, mucolipidosis (types II, III, and IV), cystinosis, sialic acid storage disorder, chylomicron retention disease with Marinesco-Sjögren syndrome, Hermansky-Pudlak syndrome, Chediak-Higashi syndrome, Danon disease, or Geleophysic dysplasia.

Symptoms of a disorder characterized by impaired protein trafficking are numerous and diverse and can include one or more of, e.g., anemia, fatigue, bruising easily, low blood platelets, liver enlargement, spleen enlargement, skeletal weakening, lung impairment, infections (e.g., chest infections or pneumonias), kidney impairment, progressive brain damage, seizures, extra thick meconium, coughing, wheezing, excess saliva or mucous production, shortness of breath, abdominal pain, occluded bowel or gut, fertility problems, polyps in the nose, clubbing of the finger/toe nails and skin, pain in the hands or feet, angiokeratoma, decreased perspiration, corneal and lenticular opacities, cataracts, mitral valve prolapse and/or regurgitation, cardiomegaly, temperature intolerance, difficulty walking, difficulty swallowing, progressive vision loss, progressive hearing loss, hypotonia, macroglossia, areflexia, lower back pain, sleap apnea, orthopnea, somnolence, lordosis, or scoliosis. It is understood that due to the diverse nature of the trafficking defective proteins and the resulting disease phenotypes (e.g., a disorder characterized by impaired protein trafficking), a given disorders will generally present only symptoms characteristic to that particular disorder. For example, a patient with cystic fibrosis can present a particular subset of the above-mentioned symptoms such as, but not limited to, persistent coughing, excess saliva and mucus production, wheezing, coughing, shortness of breath, enlarged liver and/or spleen, polyps of the nose, diabetes, fertility problems, increased infections (e.g., respiratory infections such as pneumonias), or occluded gut or bowel.

Depending on the specific nature of the disorder, a patient can present these symptoms at any age. In many cases, symptoms can present in childhood or in early adulthood. For example, symptoms of cystic fibrosis often present at birth when a baby's gut becomes blocked by extra-thick muconium.

Following administration of one or more of the disclosed compounds (or pharmaceutical compositions) to a subject (e.g., a human patient), the efficacy of the treatment in ameliorating one or more symptoms of a disorder characterized by impaired protein trafficking can be assessed by comparing the number and/or severity of one or more symptoms presented by a patient before and after treatment. Alternatively, where administration of the compounds is used to prevent the occurrence of a disorder characterized by impaired protein trafficking, treatment efficacy can be assessed as a delay in presentation of, or a failure to present, one or more symptoms of a disorder characterized by impaired protein trafficking. The efficacy of a treatment (e.g., a compound or composition described herein) over time. (e.g., a progressive improvement) in ameliorating one or more symptoms of a disorder characterized by impaired protein trafficking can be determined by assessing, e.g., the number or severity of one or more symptoms at multiple time points following treatment. For example, a subject (e.g., a patient) can have an initial assessment of the severity of his or her disorder (e.g., the number or severity of one or more symptoms of a disorder characterized by impaired protein trafficking), administered treatment, and then assessed subsequently to the treatment two or more times (e.g., at one week and one month; at one month at two months; at two weeks, one month, and six months; or six weeks, six months, and a year). Where one or more compounds or compositions are administered to a subject for a limited period of time (e.g., a predetermined duration) or number of administrations, the effect of treatment on ameliorating one or more symptoms of a disorder characterized by impaired protein trafficking can be assessed at various time points after the final treatment. For example, following the last administration of a dose of one or more compounds, the number or severity of a patient's symptoms can be assessed at 1 month (e.g., at 2 months, at 6 months, at one year, at two years, at 5 years or more) subsequent to the final treatment.

The efficacy of a treatment with one or more compounds (or compositions) described herein on one or more symptoms of a disorder characterized by impaired protein trafficking can be assessed as a monotherapy or as part of a multi-therapeutic regimen. For example, the compound(s) can be administered in conjunction with other clinically relevant treatments for disorder characterized by impaired protein traffickings including, but not limited to, physical or respiratory therapy, antibiotics, anti-asthma therapies, cortisteroids, vitamin supplements, pulmozyme treatments, Cerezyme®, Ceredase®, Myozyme®, insulin, Fabryzyme®, dialysis, transplants (e.g., liver or kidney), stool softeners or laxatives, anti-blot clotting agents (anti-coagulants), pain medications, and/or angioplasty. It is understood that due to the diverse activities of trafficking defective proteins and the diverse clinical manifestations of the associated disorders (e.g., Fabry's disease, cystic fibrosis, Gaucher's disease, Pompe disease, and the like) the “other clinically relevant treatments” can also include treatments beyond those above. For example, other or additional clinically relevant treatments for cystic fibrosis include, e.g., antibiotics, pulmozyme treatments, vitamin supplements, stool softeners or laxatives, insulin for cystic-fibrosis related diabetes, anti-asthma therapies, or corticosteroids.

A compound or pharmaceutical composition thereof described herein can be administered to a subject as a combination therapy with another treatment (another active ingredients), e.g., a treatment for a disorder characterized by impaired protein trafficking such as cystic fibrosis or a lysosomal storage disease. For example, the combination therapy can include administering to the subject (e.g., a human patient) one or more additional agents that provide a therapeutic benefit to the subject who has, or is at risk of developing, (or suspected of having) a disorder characterized by impaired protein trafficking such as cystic fibrosis. Thus, the compound or pharmaceutical composition and the one or more additional agents are administered at the same time. Alternatively, the compound can be administered first in time and the one or more additional agents administered second in time. The one or more additional agents can be administered first in time and the compound administered second in time. The compound can replace or augment a previously or currently administered therapy (also, see below). For example, upon treating with a compound of the invention, administration of the one or more additional agents can cease or diminish, e.g., be administered at lower levels. Administration of the previous therapy can also be maintained. In some instances, a previous therapy can be maintained until the level of the compound (e.g., the dosage or schedule) reaches a level sufficient to provide a therapeutic effect. The two therapies can be administered in combination.

It will be appreciated that in instances where a previous therapy is particularly toxic (e.g., a treatment for disorder characterized by impaired protein trafficking carrying significant side-effect profiles) or poorly tolerated by a subject (e.g., a patient), administration of the compound can be used to offset and/or lessen the amount of the previous therapy to a level sufficient to give the same or improved therapeutic benefit, but without the toxicity.

In some instances, when the subject is administered a compound or pharmaceutical composition of the invention, the first therapy is halted. The subject can be monitored for a first pre-selected result, e.g., an improvement in one or more symptoms of a disorder characterized by impaired protein trafficking such as any of those described herein (e.g., see above). In some cases, where the first pre-selected result is observed, treatment with the compound is decreased or halted. The subject can then be monitored for a second pre-selected result after treatment with the compound is halted, e.g., a worsening of a symptom of disorder characterized by impaired protein trafficking. When the second pre-selected result is observed, administration of the compound to the subject can be reinstated or increased, or administration of the first therapy reinstated, or the subject is administered both a compound and first therapy, or an increased amount of the compound and the first therapeutic regimen.

Methods of assessing the effect of a therapy (e.g., a compound or composition of the invention) are known in the art of medicine and include assessing the change (e.g., the improvement) in one or more symptoms of a disorder characterized by impaired protein trafficking such as any of those described herein (see above). In addition, while the invention is not limited by any particular theory or mechanism of action, because the compounds identified herein can function at the molecular level to correct the disorder characterized by impaired protein trafficking, assessing the effect of a therapy on patient having a disorder characterized by impaired protein trafficking can be done by assessing, e.g., (i) an improvement of the stability of a trafficking defective protein, (ii) improvement of proper, physiological trafficking of the trafficking defective protein, or (iii) a restoration in one or more functions of a trafficking defective protein (see above under “E. Evaluation of the Activity of the Compounds”).

In particular, efficacy of treatment (e.g., administration of one or more compounds or pharmaceutical compositions described herein) of cystic fibrosis can be monitored, e.g., by performing a “sweat test” before an after treatment. The sweat test is generally conducted by a physician or medical practitioner. A colorless, odorless chemical is placed on the skin, which causes it to sweat, and a device collects the sweat. A sweat test can take 30 minutes to 1 hour, depending on how long it takes to collect the subject's perspiration. Chloride levels in the subject's perspiration are measured (e.g., using a Sweat-Chem™ Sweat Conductivity Analyzer, Discovery Diagnostics, Ontario, Canada) and, for example, a relative score of <40 indicates normality, a score of 40-59 is an intermediate range, and a score of >60 indicates that the subject still has profound disease. Efficacy of a treatment of cystic fibrosis can also be determined using a nasal potential difference (NPD) test. The test is especially useful for subjects (e.g., patients) who have normal chloride levels as determined by sweat tests. The NPD test requires 2 electrodes, connected to a voltmeter such as the Tholy-Medicap® device), one placed on the nasal mucosa of the inferior turbinate and the other placed subcutaneously on the forearm. Generally, a reading less than −40 mV is considered abnormal. Thus, a patient who's NPD test readings improve to over −40 mV can be one considered to improve (see, for example, Domingo-Ribas et al. (2006) Arch Bronconeumol. 42:33-38).

### H. Methods of Producing a Protein

The compounds described herein enhance endoplasmic reticulum-mediated transport and thus can be used in methods to enhance protein production in a cell. The protein produced by the methods can be a naturally occurring or a non-naturally occurring protein. The protein can be produced naturally by a cell (e.g., without any genetic manipulation of the cell), can be encoded by a heterologous nucleic acid introduced into a cell, or can be produced by a cell following the insertion or activation of sequences that regulate expression of a gene encoding the protein.

A “heterologous nucleic acid” refers to a nucleotide sequence that has been introduced into a cell by the use of recombinant techniques. Accordingly, a “heterologous nucleic acid” present in a given cell does not naturally occur in the cell (e.g., has no corresponding identical sequence in the genome of the cell) and/or is present in the cell at a location different than that where a corresponding identical sequence naturally exists (e.g., the nucleotide sequence is present in a different location in the genome of the cell or is present in the cell as a construct not integrated in the genome).

Any protein that is produced by a cell can be used in the methods described herein. For example, proteins such as cytokines, lymphokines, and/or growth factors can be produced. Examples of such proteins include, but are not limited to, Erythropoietin, Interleukin 1-Alpha, Interleukin 1-Beta, Interleukin-2, Interleukin-3, Interleukin-4, Interleukin-5, Interleukin-6, Interleukin-7, Interleukin-8, Interleukin-9, Interleukin-10, Interleukin-11, Interleukin-12, Interleukin-13, Interleukin-14, Interleukin-15, Lymphotactin, Lymphotoxin Alpha, Monocyte Chemoattractant Protein-1, Monocyte Chemoattractant Protein-2, Monocyte Chemoattractant Protein-3, Megapoietin, Oncostatin M, Steel Factor, Thrombopoietin, Vascular Endothelial Cell Growth Factor, Bone Morphogenetic Proteins, Interleukin-1 Receptor Antagonist, Granulocyte-Colony Stimulating Factor, Leukemia Inhibitory Factor, Granulocyte-Macrophage Colony-Stimulating Factor, Macrophage Colony-Stimulating Factor, Interferon Gamma, Interferon Beta, Fibroblast Growth Factor, Tumor Necrosis Factor Alpha, Tumor Necrosis Factor Beta, Transforming Growth Factor Alpha, Gonadotropin, Nerve Growth Factor, Platelet-Derived Growth Factor, Macrophage Inflammatory Protein 1 Alpha, Macrophage Inflammatory Protein 1 Beta, and Fas Ligand. Cells producing a non-naturally occurring, variant of any the above polypeptides can also be used in the methods described herein.

In addition to the proteins described above, the methods described herein can also be used to produce a fusion protein that contains all or a portion of a given protein fused to a sequence of amino acids that direct secretion of the fusion protein from a cell. In some cases, such fusion proteins can allow for the secretion of a polypeptide sequence that is not typically secreted from a cell. For example, all or a portion of a protein (e.g., a membrane associated protein such as a receptor or an intracellular protein) can be fused to a portion of an immunoglobulin molecule (e.g., to the hinge region and constant region CH2 and CH3 domains of a human IgG1 heavy chain).

The protein produced by the methods described herein can be an antibody or an antigen-binding fragment of an antibody. The antibody can be directed against an antigen, e.g., a protein antigen such as a soluble polypeptide or a cell surface receptor. For example, the antibody can be directed against a cell surface receptor involved in immune cell activation, a disease-associated antigen, or an antigen produced by a pathogen. The term “antibody” refers to an immunoglobulin molecule or an antigen-binding portion thereof. As used herein, the term “antibody” refers to a protein containing at least one, for example two, heavy chain variable regions (“VH”), and at least one, for example two, light chain variable regions (“VL”). The VH and VL regions can be further subdivided into regions of hypervariability, termed “complementarity determining regions” (“CDR”), interspersed with regions that are more conserved, termed “framework regions” (FR). The antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region contains three domains, CH1, CH2, and CH3. The light chain constant region contains one domain, CL. The variable region of the heavy and light chains contains a binding domain that interacts with an antigen.

The protein can be a fully human antibody (e.g., an antibody made in a mouse genetically engineered to produce an antibody from a human immunoglobulin sequence), a humanized antibody, or a non-human antibody, e.g., a rodent (mouse or rat), goat, or primate (e.g., monkey) antibody.

The following are examples of the practice of the invention. They are not to be construed as limiting the scope of the invention in any way.

### Examples

### Example 1

### Compounds that Restore Growth of a ypt1^{ts} Mutant

The yeast mutant cell line ypt1^{ts} suppresses, in a temperature dependent fashion, the dominant-lethal phenotype of a mutant YPT1 allele (Schmitt et al. (1988) Cell 53:635-47). The yeast mutant cell line ypt1^{ts} contains an allele of YPT1 that has two point mutations: one that changes an asparagine at position 121 to a isoleucine (N121I) and another that changes an alanine at position 161 to a valine (A161V). The N121I mutation causes dominant lethality by itself, but lethality is suppressed by the second mutation, resulting in a recessive loss of function phenotype at the restrictive temperatures. ypt1^{ts} cells grow normally at temperatures up to 25° C., but are growth arrested at 37° C. (Id.). At the non-permissive temperature of 37° C., ypt1^{ts} mutants accumulate ER membranes, small vesicles, and unprocessed invertase and exhibit cytoskeletal defects and enhanced calcium uptake (Id.). ypt1^{ts} mutant cells can be rescued from growth arrest by the provision of extracellular calcium (Id.).

Compounds that rescue cells from alpha-synuclein toxicity were screened to assess their ability to restore growth of ypt1^{ts} cells. The effect of the compounds was measured on ypt1^{ts} cells cultured at room temperature (permissive temperature), 37° C. (non-permissive temperature), and 35° C. (semi-permissive temperature). Certain compounds (and analogs thereof) that rescue alpha-synuclein toxicity were found to also rescue ypt1^{ts} toxicity.

To determine if the test compounds could rescue the ypt1^{ts} mutant phenotype, ypt1^{ts} cells were grown overnight in synthetic complete (SC) media supplementd with 2% glucose at room temperature. Log phase cells were diluted into SC 2% glucose media to an OD600 of 0.003. 100 μL of this culture was then aliquoted into each well of 96-well flat bottom microtiter plates. 1 μL of the test compounds dissolved in DMSO (at a concentration range from 5 mM-0.005 mM) or of DMSO alone was added to each well (50 uM-0.05 uM final concentration in 1% DMSO). Plates were mixed by vortexing and incubated at 35° C. and 37° C. Compound rescue of the ypt1^{ts} temperature sensitive defect was assessed by measuring the OD₆₀₀ (optical density at 600 nm; cell growth) of the cultures. Plates incubated at 35° C. were measured at 24 and 40 hours incubation time while plates grown at 37° C. were measured after 40 hours of incubation.

Assays monitoring the rescue of ypt1^{ts} mutants were performed using a vehicle, a positive control (calcium), active compounds obtained from the alpha-synuclein screen (Cpd. I.1 and Cpd. II.1), alpha-synuclein-active analogs of Cpd. I.1 (Cpd. I.2 and Cpd. I.3), and alpha-synuclein-inactive analogs of Cpd. I.1 (Cpd. I.4 and Cpd. I.5).

As expected, calcium (the positive control) rescued ypt1^{ts} at both 35° C. and 37° C.. In addition, compounds Cpd. I.1 and Cpd. II.1 were both found to rescue ypt1^{ts} at 35° C. and 37° C.. Active analogs of Cpd. I.1 (Cpd. I.2 and Cpd. I.3) also rescued ypt1^{ts} loss of function, whereas inactive Cpd. I.1 analogs (Cpd. I.4 and Cpd. I.5) did not.

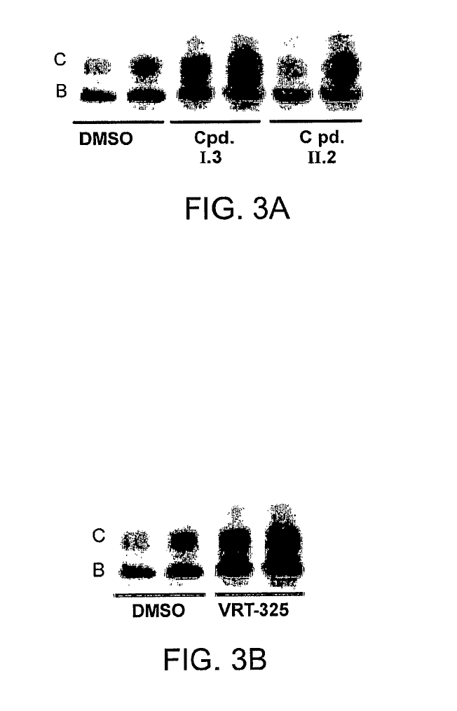

Furthermore, Cpd. II.3 was also tested for its ability to rescue the ypt1^{ts} mutant phenotype. ypt1^{ts} cells were cultured at 37° C. for 40 hours in the presence of 5.0 μM Cpd. II.3, 2.0 μM Cpd. I.3, or DMSO as a control. Cpd. II.3, as well as Cpd. I.3, rescued the ypt1^{ts} phenotype (FIG. 2).

Cpds. I.7-I.35, I.58-I.75, II.4-II.69, and II.96-II.134 were also tested in the ypt1^{ts} rescue assay described above. Cpds. I.7-I.35 and II.4-II.69 rescued the ypt1^{ts} phenotype Cpds. I.58-I.75 and II.96-II.134 showed activity in the ypt1^{ts} assay at higher concentrations.

The finding that the above compounds can rescue the ypt1^{ts} protein trafficking defect indicates that the compounds can be used to treat or prevent a variety of disorders characterized by impaired protein trafficking.

### Example 2

### Doxorubicin, Cycloheximide, Hygromycin, Novobiocin, Aureobasidin and Tunicamycin Restore Growth of a ypt^{ts} Mutant

In addition to the compounds described in Example 1, several additional compounds were also tested in the ypt1^{ts} growth screen. These screening assays identified doxorubicin, cycloheximide, hygromycin, novobiocin, aureobasidin, and tunicamycin as effective at rescuing ypt1 loss of function and restoring growth of ypt1^{ts} at 35° C. and 37° C.. The finding that these compounds can rescue the ypt1^{ts} protein trafficking defect indicates that the compounds can be used to treat or prevent a variety of disorders characterized by impaired protein trafficking.

### Example 3

### Proteosome Inhibitors Rescue ypt1^{ts} Mutant Phenotype

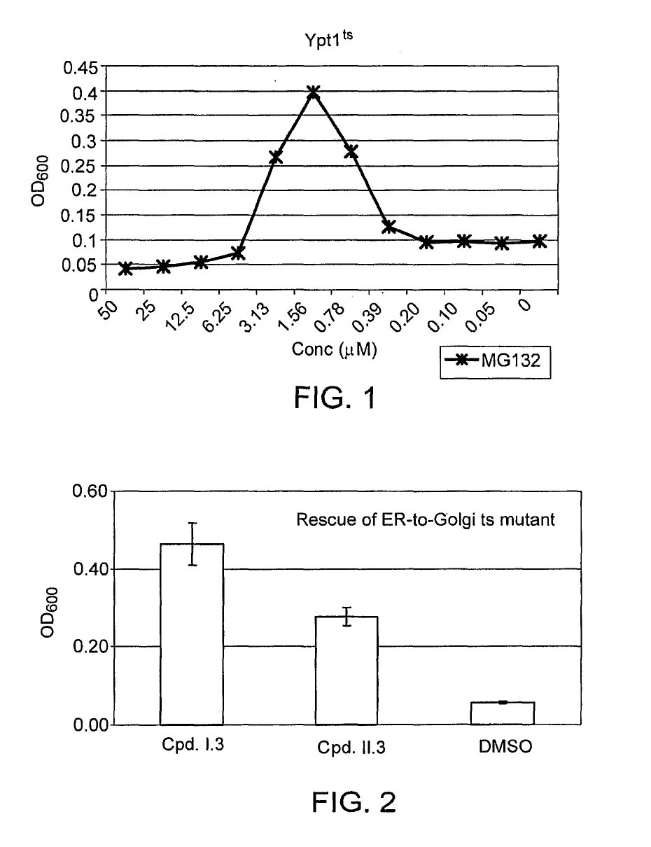

Proteasome inhibitors such as bortezomib (PS-341/Velcade) have been shown using cell-based studies to stabilize the ΔF508 CFTR mutant, preventing its premature degradation and restoring cellular chloride efflux (Vij et al. (2006) J. Biol. Chem. 281:17369-17378). The proteasome inhibitor MG132 (Sigma-Aldrich, St. Louis, Mo.) was tested for its ability to rescue the ypt1ts mutant phenotype. ypt1^{ts} mutant cells were plated in 96 well-tissue culture plates and cultured at 37° C. (non-permissive temperature, see above) for 40 hours in the presence of various concentrations of MG132 (range from 0.05-50 μM) (FIG. 1). While cells cultured at the non-permissive temperature exhibited severe growth inhibition in the absence of MG132, intermediate concentrations of the compound rescued ypt1^{ts} loss of function.

These data indicate that the ypt1^{ts} mutant screening assay can be useful in identifying compounds that can treat cystic fibrosis. In addition, these results indicate that compounds useful in treating ΔF508 CFTR (i.e., in treating one specific type of trafficking disorder), have broader activity in treating a wide-range of disorders characterized by impaired protein trafficking such as any of those described herein.

### Example 4

### ypt1^{ts} Mutant Active Compounds Stabilize ΔF50 CTFR

Selected compounds identified in the ypt1^{ts} screen were further tested for their ability to stabilize ΔF508 CTFR. CFBE cells, a cell line generated by transformation of cystic fibrosis tracheo-bronchial cells (ΔF508 CTFR homozygous) with SV40 (Bruscia et al. (2002) Gene Ther. 9(11):683-685), were cultured with 10 μM Cpd. I.3, 10 μM Cpd. II.2, or 10 μM VRT-325 for 16 hours at 37° C. (VRT-325 is described in, e.g., Van Goor et al. (2006) Am. J. Physiol. Lung Cell Mol. Physiol. 290:L1117-L1130). A population of cells was also cultured with the dimethyl sulfoxide (DMSO) solvent as a control.

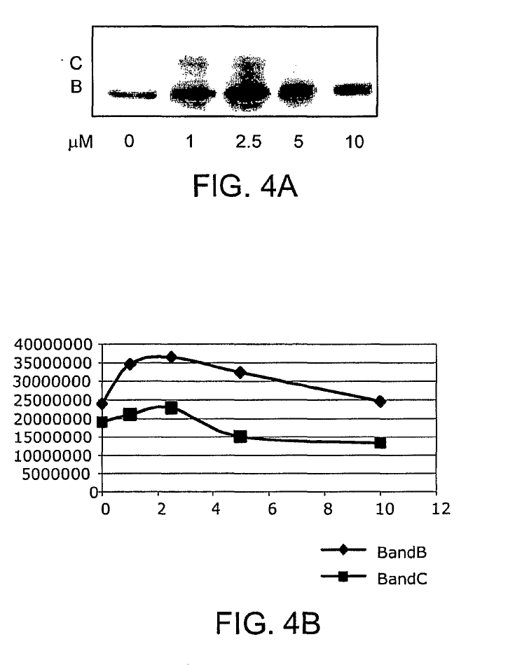

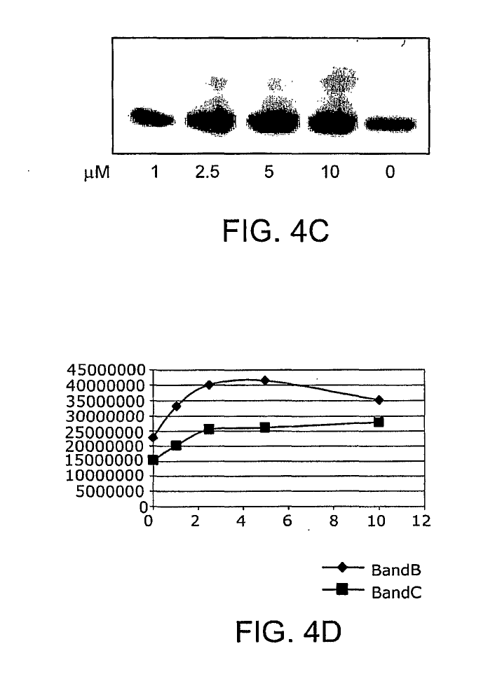

Following incubation, cells were lysed, solubilized in Laemmli buffer, and subjected to SDS-PAGE. CFTR protein was visualized by western blotting using an antibody specific for CFTR. Culturing CFBE cells with Cpd. I.3 or Cpd. II.2 increased the amount of cellular ΔF508 CFTR protein (see band “B,” FIG. 3A). Cpd. I.3 and Cpd. II.2 also increased the amount of the glycosylated form of ΔF508 CFTR (see band “C,” FIG. 4A), indicating that there was increased trafficking of this protein through the Golgi apparatus. The effects of Cpd. I.3 or Cpd. II.2 on stabilizing ΔF508 CFTR were comparable or better than the effects of the known CFTR stabilizer VRT-325 (FIG. 3B).

Next, to test the effect of different concentrations of Cpd. I.3 and Cpd. II.2 on ΔF508 CFTR, a dose response experiment was performed. CFBE cells were grown at 37° C. for 16 hours in the presence of 1, 2.5, 5, or 10 μM Cpd. I.3 or Cpd. II.2. Following incubation, lysates were prepared from the various treated cell populations, the lysates solubilized in Laemmli buffer, and subjected to SDS-PAGE. The relative amounts of glysosylated (band “C”) and unglycosylated (band “B”) ΔF508 CFTR protein were visualized by western blotting as above (FIGS. 4A and 4C). The band intensities were quantitated by scanning and densitometry. As compared to the amount of protein in the absence of compound, all concentrations tested (1-10 μM) showed an increase in the amount of glycosylated and unglycosylated ΔF508 CFTR proteins with both compounds (FIGS. 4A and 4C). Dose response curves generated from the western blot data showed that, in this assay, efficacy reached a maximum at 1-2.5 mM and 2.5-5 mM for Cpd. I.3 (FIG. 4B) and Cpd. II.2 (FIG. 4D), respectively.

Taken together, these data indicate that compounds identified in the ypt1^{ts} mutant rescue screening assay can stabilize ΔF508 CFTR protein and thus are useful in treating cystic fibrosis.

### Example 5

### Compounds that Restore Growth of a sar1^{st} Mutant

The sar1^{st} mutant yeast strain (ATCC, Manassas, Va.) carries a temperature sensitive mutant allele of the SAR1 gene, which permits the strain to grow at 25° C., but undergo growth arrest at 35° C. or higher. Inactivation of the mutant Sar1^{ts} protein at 35° C. prevents the formation of transport vesicles at the ER, causing a block in ER to golgi trafficking (Saito et al. (1998) J. Biochem. (Tokyo) 124(4):816-823).

To identify compounds that rescue the sar1^{ts} mutant phenotype, the mutant strain was first grown at 25° C. in rich media overnight. The strain was then diluted to an OD₆₀₀ of 0.004 in SC media with 2% glucose, and mixed with various dilutions of test compounds (0.05 to 50 μM) in SC media with 2% glucose. The cells were then incubated at 25° C. or 35° C. for 72 hours. Rescue of the sar1^{ts} mutant phenotype was scored as an increase in the OD₆₀₀ (concentration of the yeast cells) cultured in the presence of a test compound as compared to cells cultured cultured in the absence of the test compound.

In addition to control compounds cycloheximide and hygromycin, the following test compounds were determined using the above assay to rescue the sar1^{ts} mutant phenotype: Cpd. I.1, Cpd. I.3, Cpd. I.5, and Cpd. I.6. Activity was not detected in this assay for Cpds II.2, II.59, II.57, II.27, II.1, II.12, doxorubicin, and aureobasidin.

### Example 6

### Compounds that Restore Growth of a sec23^{ts} Mutant

The sec23-2^{ts} mutant yeast strain carries a temperature sensitive mutant allele of the SEC23 gene, which permits the strain to grow normally at 25° C., but undergo growth arrest at 30° C. or higher. Inactivation of the Sec23 temperature-sensitive mutant protein at the restrictive temperature prevents the formation of transport vesicles at the ER resulting in a block in ER to golgi trafficking (see, e.g., Hicke et al. (1989) EMBO J. 8(6):1677-1684 and Castillo-Flores et al. (2005) J. Biol. Chem. 280(40):34033-34041).

To identify compounds that rescue the sec23^{ts} mutant phenotype, the mutant strain was first grown at 25° C. in rich media overnight. The strain was then diluted to an OD₆₀₀ of 0.004 in SC media with 2% glucose, and mixed with various dilutions of test compounds compounds compounds (0.05 to 50 μM) in SC media with 2% glucose. The cells were then incubated at 25° C. or 30° C. for 24 hours. Rescue of the sec23^{ts} mutant phenotype was scored as an increase in the OD₆₀₀ of cells cultured in the presence of the a compound as compared to cells cultured in the absence of the test compound.

Cpd. I.1 and Cpd. I.3 were determined to rescue the ses23^{ts} mutant phenotype. Activity was not detected in this assy for Cpds I.5, II.2, I.6, II.59, II.57, II.27, II.1, II.12, cycloheximide, doxorubicin, and aureobasidin.

### Other Embodiments

It is to be understood that, while the invention has been described in conjunction with the detailed description thereof, the foregoing description is intended to illustrate and not limit the scope of the invention. Other aspects, advantages, and modifications of the invention are within the scope of the claims set forth below.

TABLE I

| | |
|---|---|
| | I.1 |
| 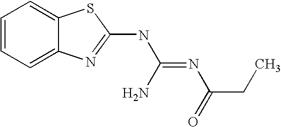 | |
| 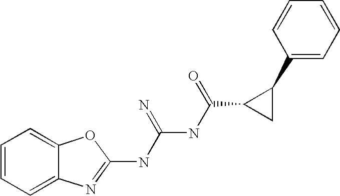 | I.2 |
| 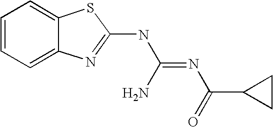 | I.3 |
| 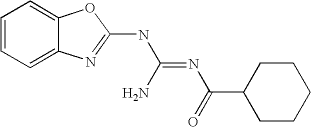 | I.4 |
| 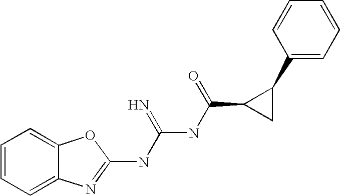 | I.5 |
| 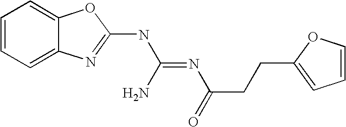 | I.6 |
| 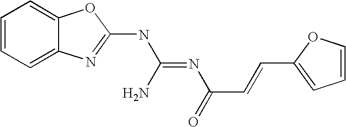 | I.7 |
| 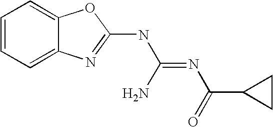 | I.8 |
| 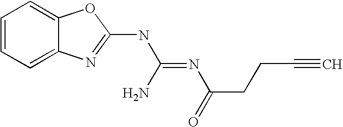 | I.9 |

| | |
|---|---|
| | I.10 |
| 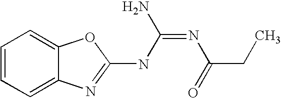 | |
| 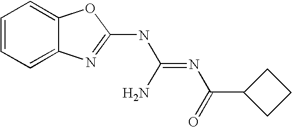 | I.11 |
| 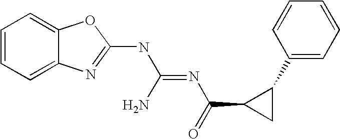 | I.12 |
| 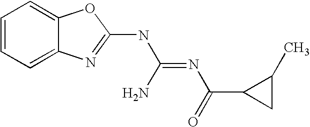 | I.13 |
| 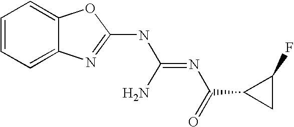 | I.14 |
| 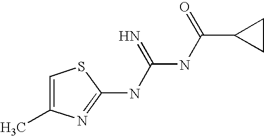 | I.15 |
| 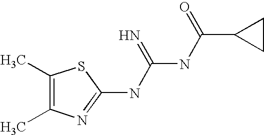 | I.16 |
| 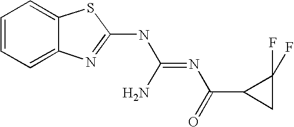 | I.17 |
| 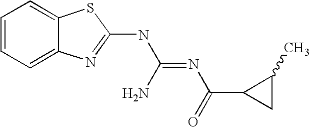 | I.18 |
| 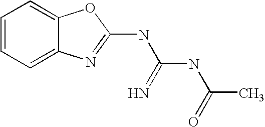 | I.19 |
| 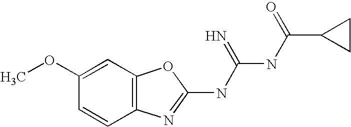 | I.20 |
| 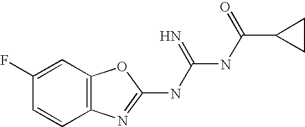 | I.21 |
| 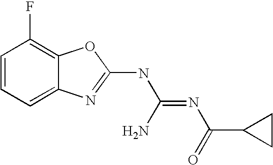 | I.22 |
| 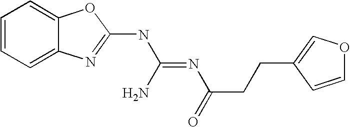 | I.23 |
| 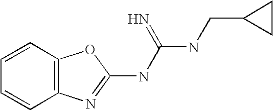 | I.24 |
| 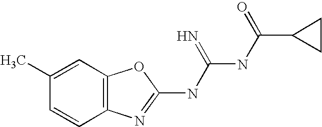 | I.25 |
| 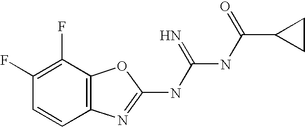 | I.26 |
| 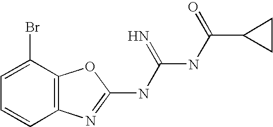 | I.27 |
| 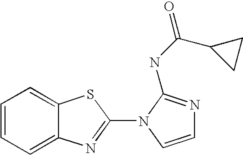 | I.28 |
| 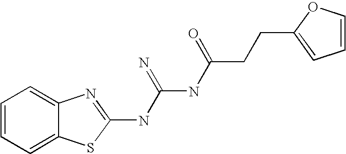 | I.29 |
| 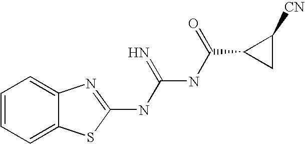 | I.30 |
| 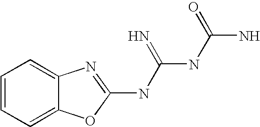 | I.31 |
| 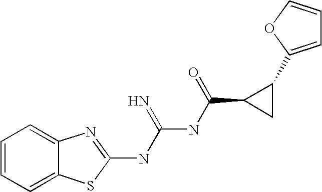 | I.32 |
| 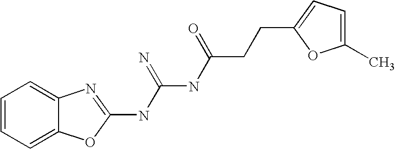 | I.33 |
| 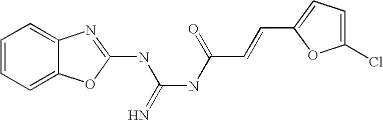 | I.34 |
| 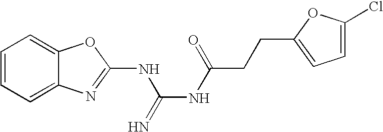 | I.35 |
| 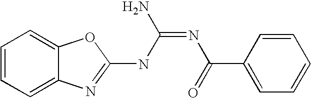 | I.36 |
| 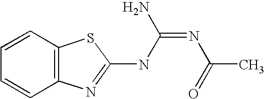 | I.37 |
| 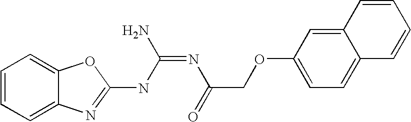 | I.38 |
| 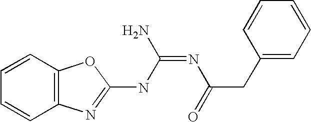 | I.39 |
| 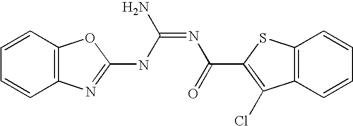 | I.40 |
| 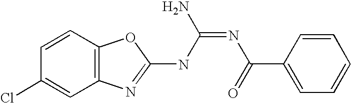 | I.41 |
| 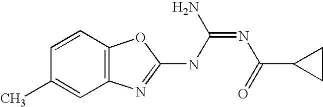 | I.42 |
| 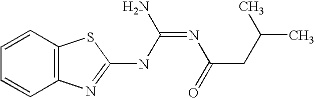 | I.43 |
| 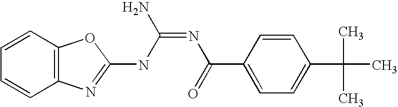 | I.44 |
| 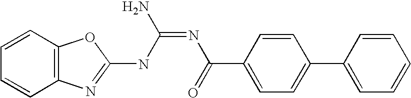 | I.45 |
| 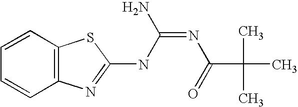 | I.46 |
| 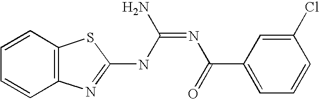 | I.47 |
| 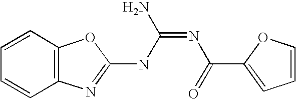 | I.48 |
| 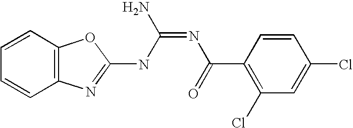 | I.49 |
| 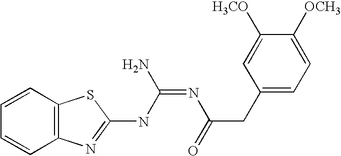 | I.50 |
| 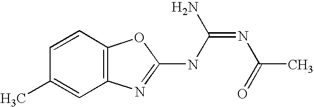 | I.51 |
| 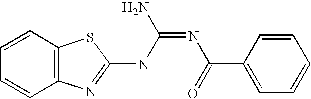 | I.52 |
| 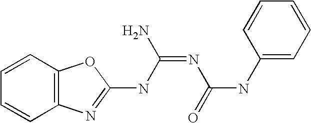 | I.53 |
| 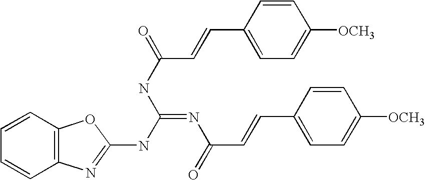 | I.54 |
| 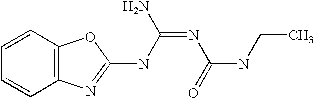 | I.55 |
| 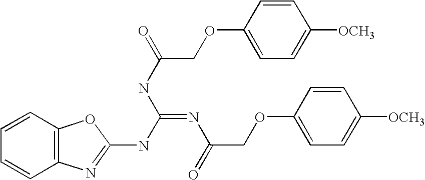 | I.56 |
| 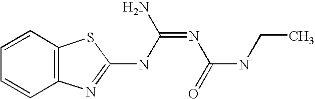 | I.57 |
| 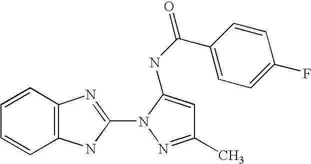 | I.58 |
| 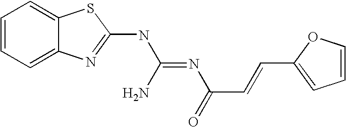 | I.59 |
| 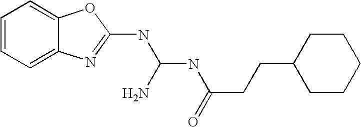 | I.60 |
| 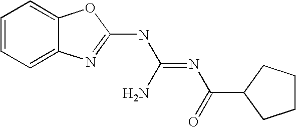 | I.61 |
| 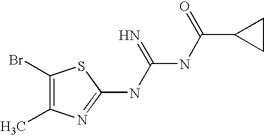 | I.62 |
| 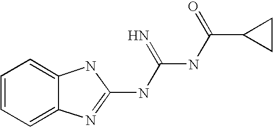 | I.63 |
| 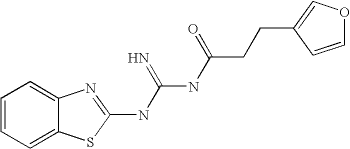 | I.64 |
| 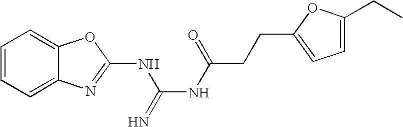 | I.65 |
| 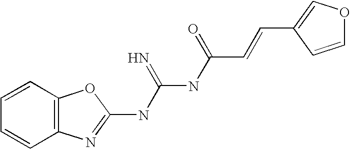 | I.66 |
| 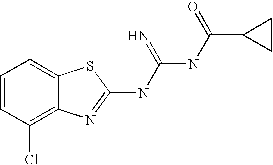 | I.67 |
| 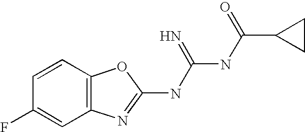 | I.68 |
| 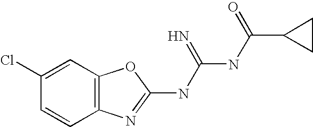 | I.69 |
| 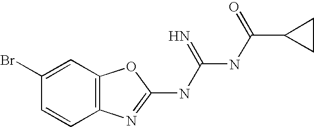 | I.70 |
| 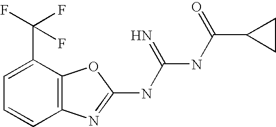 | I.71 |
| 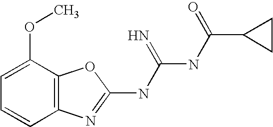 | I.72 |
| 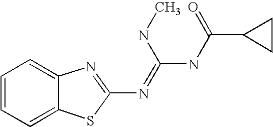 | I.73 |
| 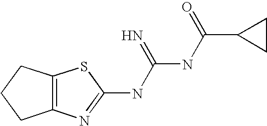 | I.74 |
| 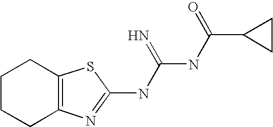 | I.75 |

TABLE II

| | |
|---|---|
| | II.1 |
| 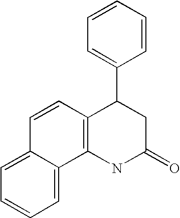 | |
| 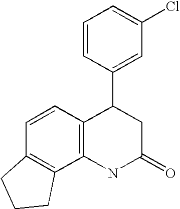 | II.2 |
| 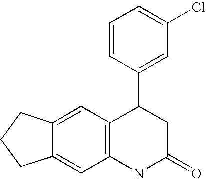 | II.3 |
| 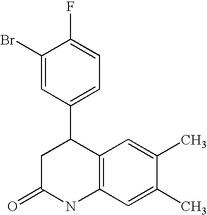 | II.4 |
| 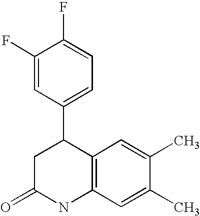 | II.5 |
| 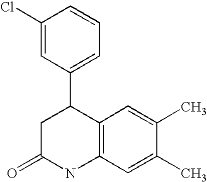 | II.6 |
| 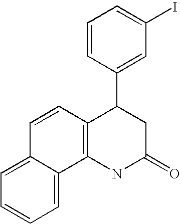 | II.7 |
| 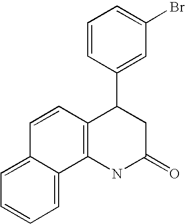 | II.8 |
| 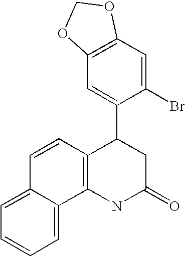 | II.9 |
| 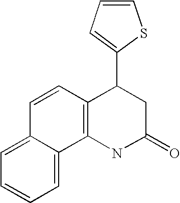 | II.10 |
| 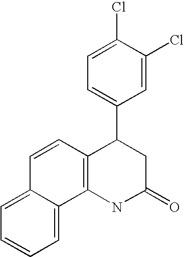 | II.11 |
| 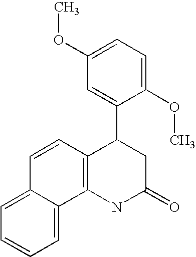 | II.12 |
| 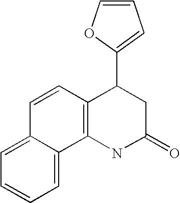 | II.13 |
| 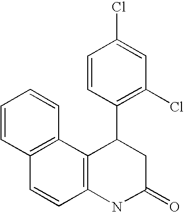 | II.14 |
| 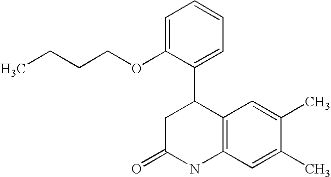 | II.15 |
| 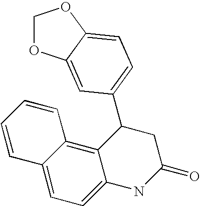 | II.16 |
| 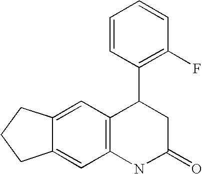 | II.17 |
| 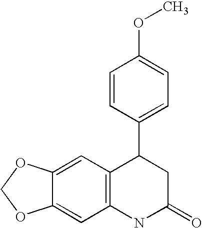 | II.18 |
| 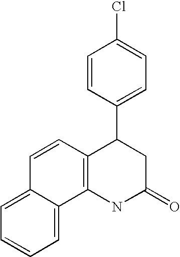 | II.19 |
| 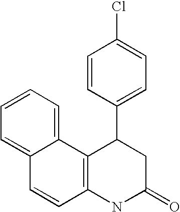 | II.20 |
| 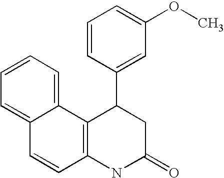 | II.21 |
| 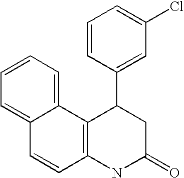 | II.22 |
| 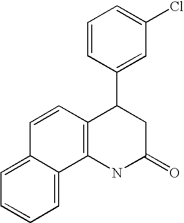 | II.23 |
| 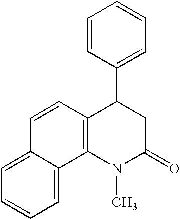 | II.24 |
| 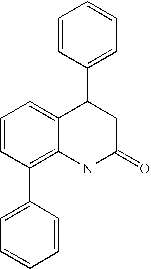 | II.25 |
| 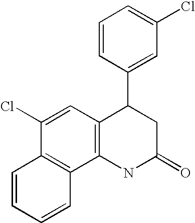 | II.26 |
| 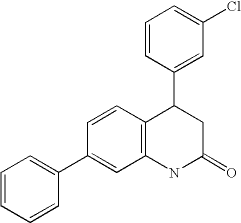 | II.27 |
| 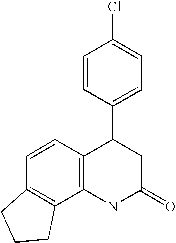 | II.28 |
| 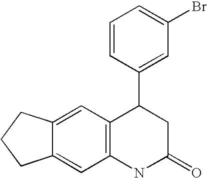 | II.29 |
| 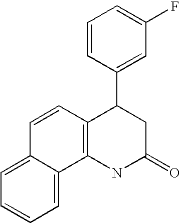 | II.30 |
| 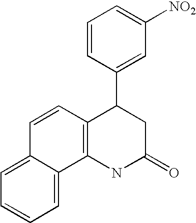 | II.31 |
| 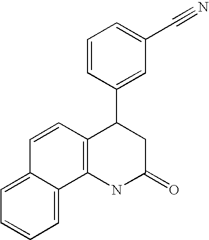 | II.32 |
| 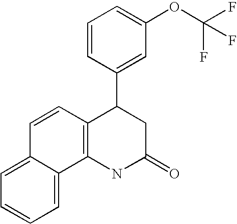 | II.33 |
| 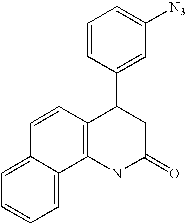 | II.34 |
| 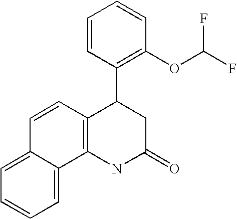 | II.35 |
| 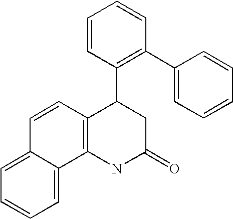 | II.36 |
| 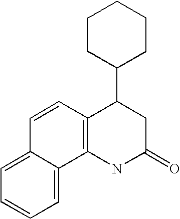 | II.37 |
| 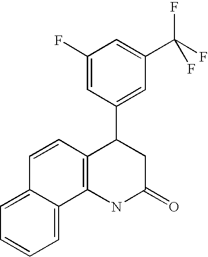 | II.38 |
| 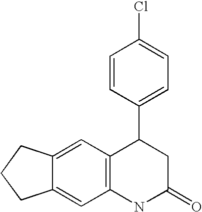 | II.39 |
| 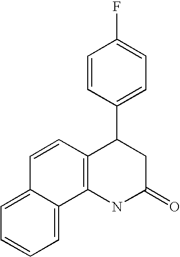 | II.40 |
| 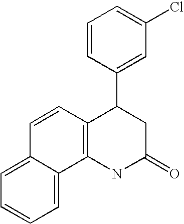 | II.41 |
| 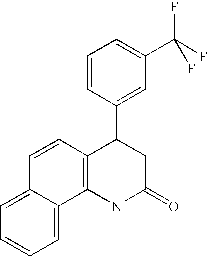 | II.42 |
| 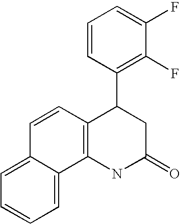 | II.43 |
| 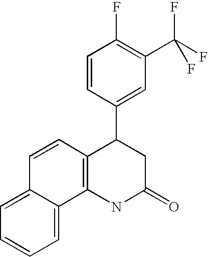 | II.44 |
| 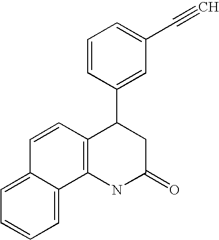 | II.45 |
| 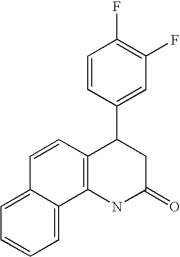 | II.46 |
| 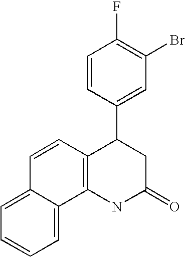 | II.47 |
| 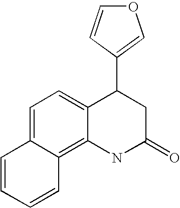 | II.48 |
| 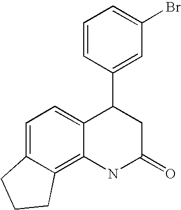 | II.49 |
| 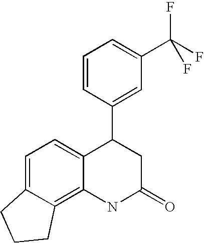 | II.50 |
| 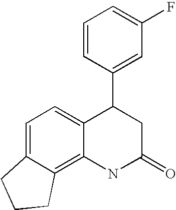 | II.51 |
| 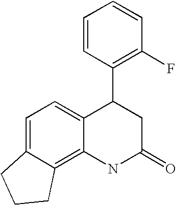 | II.52 |
| 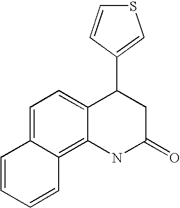 | II.53 |
| 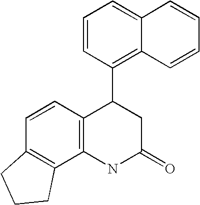 | II.54 |
| 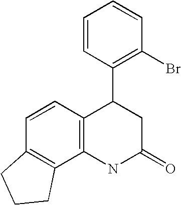 | II.55 |
| 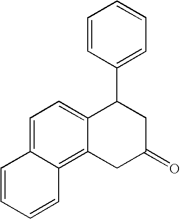 | II.56 |
| 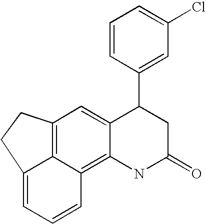 | II.57 |
| 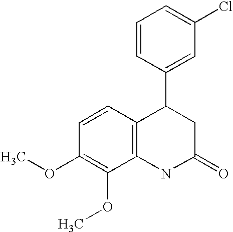 | II.58 |
| 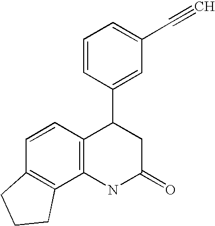 | II.59 |
| 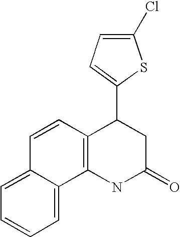 | II.60 |
| 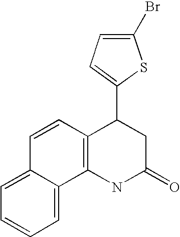 | II.61 |
| 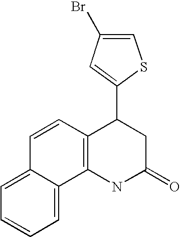 | II.62 |
| 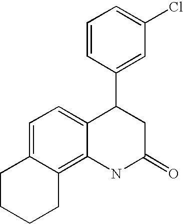 | II.63 |
| 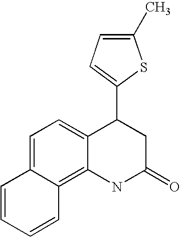 | II.64 |
| 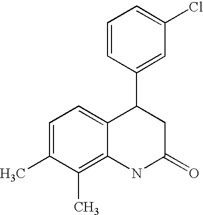 | II.65 |
| 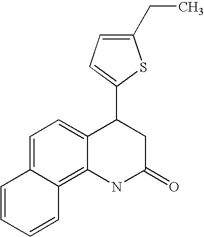 | II.66 |
| 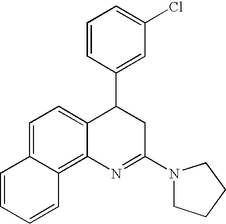 | II.67 |
| 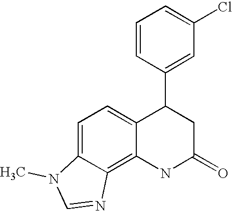 | II.68 |
| 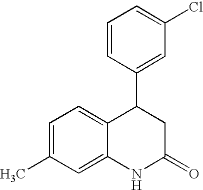 | II.69 |
| 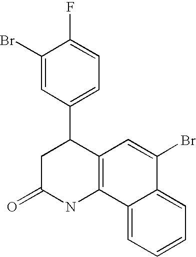 | II.70 |
| 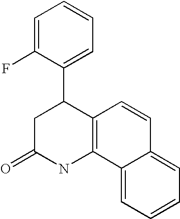 | II.71 |
| 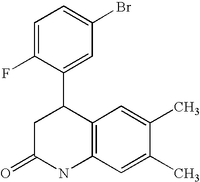 | II.72 |
| 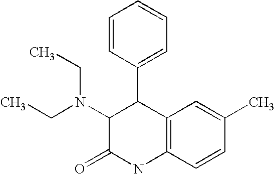 | II.73 |
| 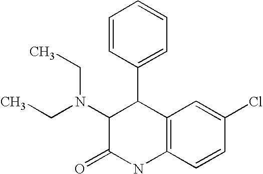 | II.74 |
| 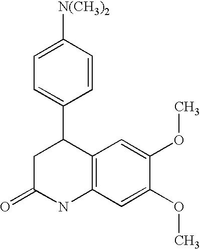 | II.75 |
| 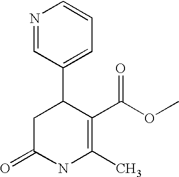 | II.76 |
| 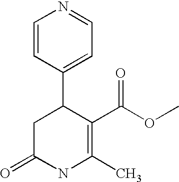 | II.77 |
| 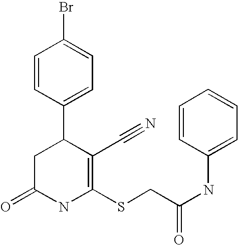 | II.78 |
| 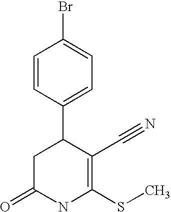 | II.79 |
| 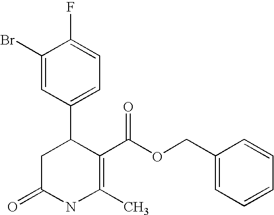 | II.80 |
| 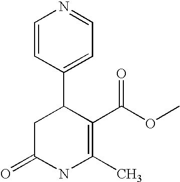 | II.81 |
| 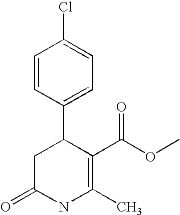 | II.82 |
| 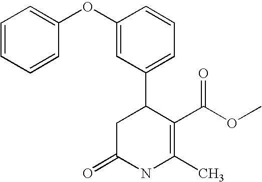 | II.83 |
| 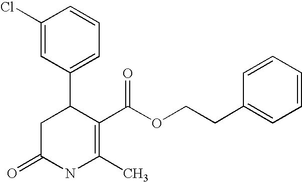 | II.84 |
| 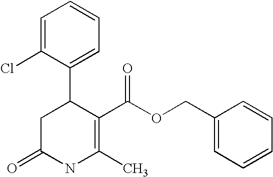 | II.85 |
| 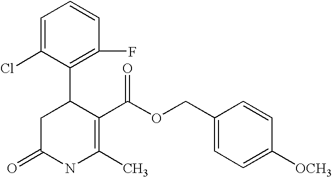 | II.86 |
| 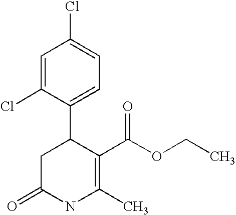 | II.87 |
| 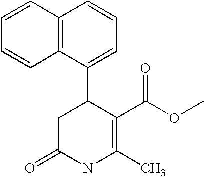 | II.88 |
| 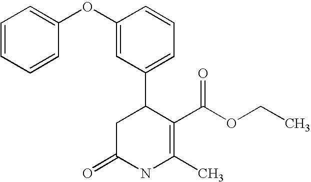 | II.89 |
| 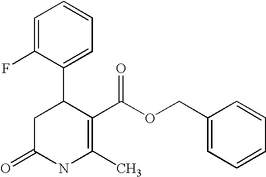 | II.90 |
| 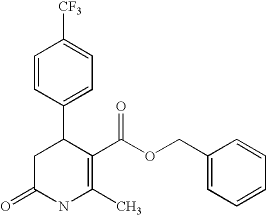 | II.91 |
| 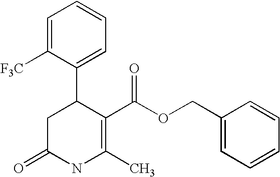 | II.92 |
| 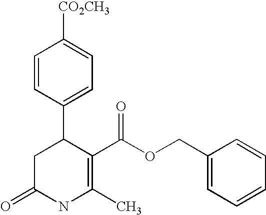 | II.93 |
| 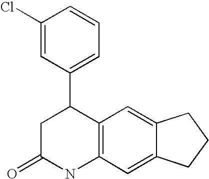 | II.94 |
| 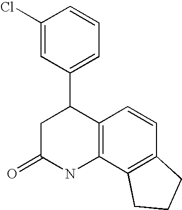 | II.95 |
| 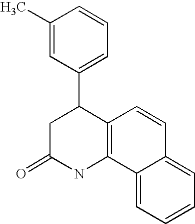 | II.96 |
| 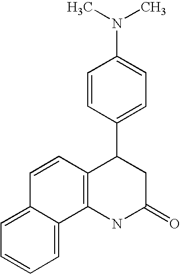 | II.97 |
| 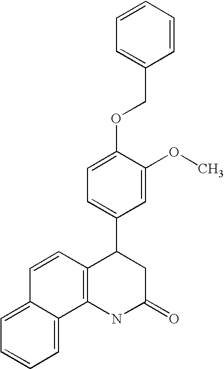 | II.98 |
| 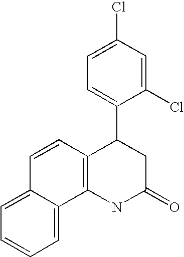 | II.99 |

| | |
|---|---|
| | II.100 |
| 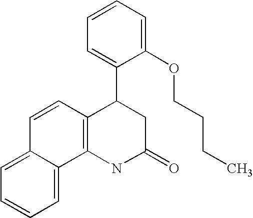 | |
| 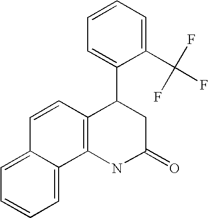 | II.101 |
| 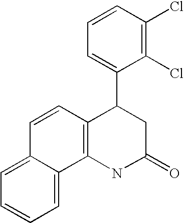 | II.102 |
| 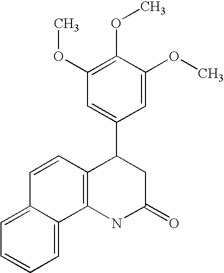 | II.103 |
| 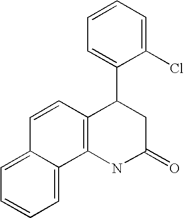 | II.104 |
| 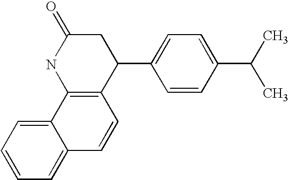 | II.105 |
| 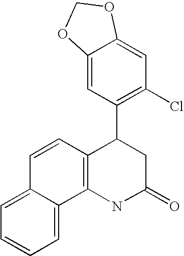 | II.106 |
| 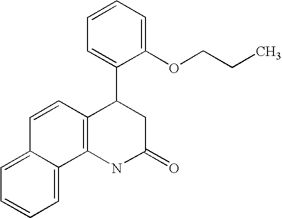 | II.107 |
| 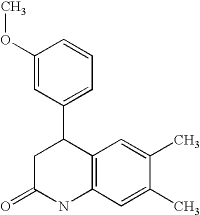 | II.108 |
| 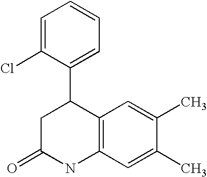 | II.109 |
| 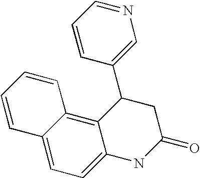 | II.110 |
| 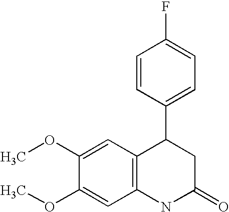 | II.111 |
| 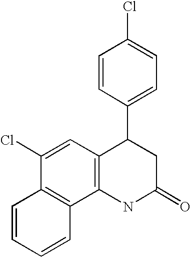 | II.112 |
| 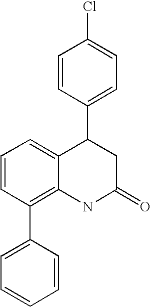 | II.113 |
| 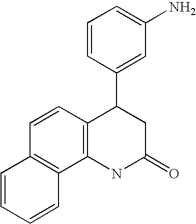 | II.114 |
| 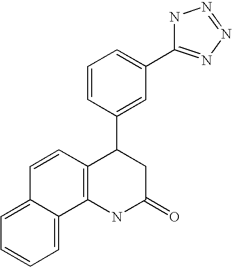 | II.115 |
| 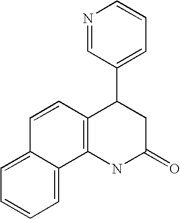 | II.116 |
| 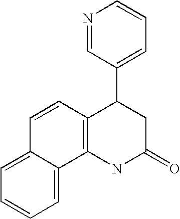 | II.116 |
| 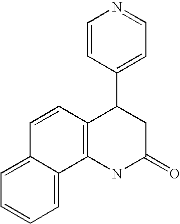 | II.117 |
| 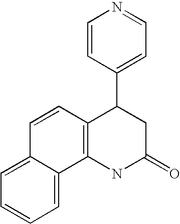 | II.117 |
| 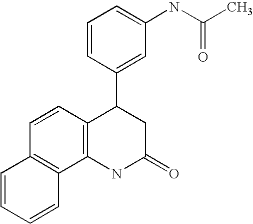 | II.118 |
| 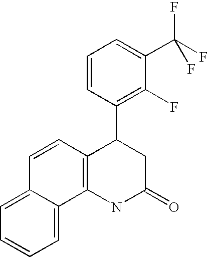 | II.119 |
| 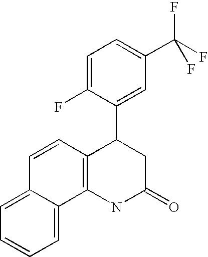 | II.120 |
| 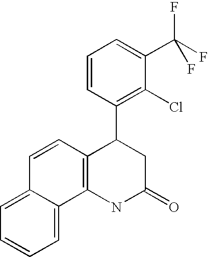 | II.121 |
| 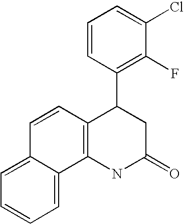 | II.122 |
| 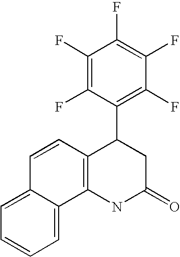 | II.123 |
| 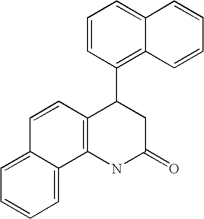 | II.124 |
| 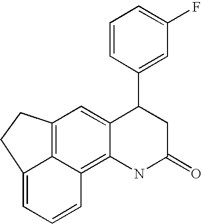 | II.125 |
| 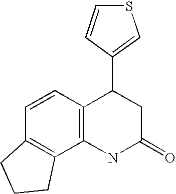 | II.126 |
| 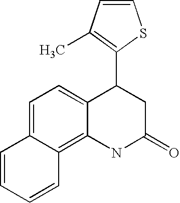 | II.127 |
| 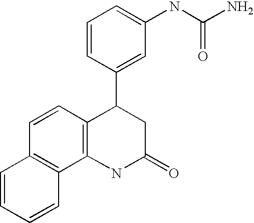 | II.128 |
| 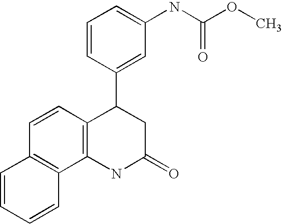 | II.129 |
| 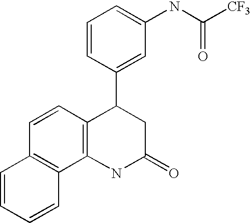 | II.130 |
| 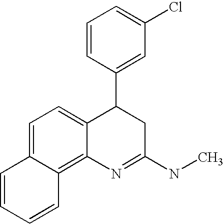 | II.131 |
| 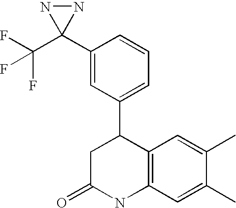 | II.132 |
| 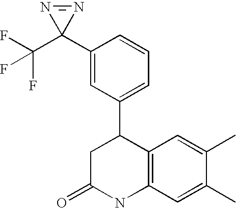 | II.133 |
| 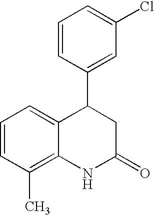 | II.134 |

1. A method of treating or preventing a disorder characterized by impaired protein trafficking, the method comprising administering to a subject a compound of Formula Ia: 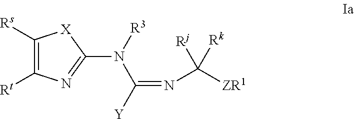
or a pharmaceutically acceptable derivative thereof, wherein:
R^{j} and R^{k} are independently selected from hydrogen, alkyl, alkenyl, alkynyl, cycloalkyl, heterocyclyl, aryl, heteroaryl or aralkyl; or, R^{j} and R^{k}, together with the carbon to which they are both bonded, are —C(═O)—, —CH(OR*)—, —C(═S)—, —CH(SR*)—, —CH(NR*R*′)— or —C(═NR*)—;
R* and R*′ are independently hydrogen, alkyl, alkenyl, alkynyl, cycloalkyl, heterocyclyl, aryl, heteroaryl or aralkyl;
R^{s} and R^{t} are independently selected from hydrogen, alkyl, halo, pseudohalo, alkenyl, alkynyl, cycloalkyl, heterocyclyl, aryl, heteroaryl or aralkyl; or, R^{s} and R^{t}, together with the carbon-carbon double bond between them, form a 4-6 membered cycloalkenyl, aryl, heterocyclyl, or heteroaryl ring, wherein the ring formed by R^{s} and R^{t} is optionally substituted with 0-4 substituents R²;
X is O, S or NR, where R is hydrogen, alkyl, alkenyl, alkynyl, cycloalkyl, heterocyclyl, aryl, heteroaryl or aralkyl;
Y is NRR″, OR′, SR′, or CRR″; where R″ is hydrogen, alkyl, alkenyl, alkynyl, cycloalkyl, heterocyclyl, aryl, heteroaryl or aralkyl, or R″, together with R³ and the atoms therebetween, is a 4-6 membered heterocyclyl or heteroaryl ring;
Z is a direct bond or NR;
R¹ is hydrogen, alkyl, alkenyl, alkynyl, cycloalkyl, heterocyclyl, aryl, heteroaryl, aralkyl, aralkenyl, heteroaralkyl or heteroaralkenyl;
n is 0 to 4;
R² is selected from (i) or (ii) as follows:
(i) hydrogen, alkyl, alkenyl, alkynyl, aryl, heteroaryl, heteroarylium, cycloalkyl, heterocyclyl, C(A)R¹¹⁰, halo, pseudohalo, OR¹¹¹, S(D)ₐR¹¹², NR¹¹⁵R¹¹⁶ or N⁺R¹¹⁵R¹¹⁶R¹¹⁷; or
(ii) any two R² groups, which substitute adjacent atoms on the ring, together form alkylene, alkenylene, alkynylene or heteroalkylene;
A is O, S or NR¹²⁵;
R¹¹⁰ is hydrogen, alkyl, alkenyl, alkynyl, aryl, heteroaryl, heteroarylium, cycloalkyl, heterocyclyl, C(A)R²⁶, halo pseudohalo, OR¹²⁵, SR¹²⁵, NR¹²⁷R¹²⁸ and SiR¹²²R¹²³R¹²⁴;
R¹¹¹ is hydrogen, alkyl, alkenyl, alkynyl, aryl, heteroaryl, heteroarylium, cycloalkyl, heterocyclyl, C(A)R¹²⁹, NR¹³⁰R¹³¹ and SiR¹²²R¹²³R¹²⁴;
D is O or NR¹²⁵;
a is 0, 1 or 2;
when a is 1 or 2, R¹¹² is selected from hydrogen, alkyl, alkenyl, alkynyl, aryl, heteroaryl, heteroarylium, cycloalkyl, heterocyclyl, halo, pseudohalo, OR¹²⁵, SR¹²⁵ and NR¹³²R¹³³;
when a is 0, R¹¹² is selected from hydrogen, alkyl, alkenyl, alkynyl, aryl, heteroaryl, heteroarylium, cycloalkyl, heterocyclyl, SR¹²⁵ and C(A)R¹²⁹;
R¹¹⁵, R¹¹⁶ and R¹¹⁷ are each independently selected from (a) and (b) as follows:
(a) hydrogen, alkyl, alkenyl, alkynyl, aryl, heteroaryl, heteroarylium, cycloalkyl, heterocyclyl, C(A)R¹²⁹, OR¹²⁵ or NR¹³²R¹³³; or
(b) any two of R¹¹⁵, R¹¹⁶ and R¹¹⁷ together form alkylene, alkenylene, alkynylene, heteroalkylene, and the other is selected as in (a);
R¹²², R¹²³ and R¹²⁴ are selected as in (i) or (ii) as follows:
(i) R¹²², R¹²³ and R¹²⁴ are each independently hydrogen, alkyl, alkenyl, alkynyl, aryl, heteroaryl, heteroarylium, cycloalkyl, heterocyclyl, OR¹²⁵ or NR¹³²R¹³³; or
(ii) any two of R¹²², R¹²³ and R¹²⁴ together form alkylene, alkenylene, alkynylene, heteroalkylene; and the other is selected as in (i);
R¹²⁵ is hydrogen, alkyl, alkenyl, alkynyl, aryl, heteroaryl, heteroarylium, cycloalkyl or heterocyclyl;
R¹²⁶ is hydrogen, alkyl, alkenyl, alkynyl, aryl, heteroaryl, heteroarylium, cycloalkyl, heterocyclyl, OR¹²⁵ or NR¹³⁴R¹³⁵; where R¹³⁴ and R¹³⁵ are each independently hydrogen, alkyl, alkenyl, alkynyl, aryl, heteroaryl, heteroarylium, cycloalkyl, heterocyclyl, OR¹³⁶ or NR¹³²R¹³³, or R¹³⁴ and R¹³⁵ together form alkylene, alkenylene, alkynylene, heteroalkylene, where R¹³⁶ is hydrogen, alkyl, alkenyl, alkynyl, aryl, heteroaryl, heteroarylium, cycloalkyl or heterocyclyl;
R¹²⁷ and R¹²⁸ are selected as in (i) or (ii) as follows:
(i) R¹²⁷ and R¹²⁸ are each independently hydrogen, alkyl, alkenyl, alkynyl, aryl, heteroaryl, heteroarylium, cycloalkyl, heterocyclyl, OR¹²⁵, NR¹³⁷R¹³⁸ or C(A)R¹³⁹, where R¹³⁷ and R¹³⁸ are each independently hydrogen, alkyl, alkenyl, alkynyl, aryl, heteroaryl, heteroarylium, cycloalkyl or heterocyclyl, or together form alkylene, alkenylene, alkynylene, heteroalkylene; and R¹³⁹ is hydrogen, alkyl, alkenyl, alkynyl, aryl, heteroaryl, heteroarylium, cycloalkyl, heterocyclyl, OR¹⁴⁰ or NR¹³²R¹³³, where R¹⁴⁰ is alkyl, alkenyl, alkynyl, aryl, heteroaryl, heteroarylium, cycloalkyl, heterocyclyl; or
(ii) R¹²⁷ and R¹²⁸ together form alkylene, alkenylene, alkynylene, heteroalkylene;
R¹²⁹ is hydrogen, alkyl, alkenyl, alkynyl, aryl, heteroaryl, heteroarylium, cycloalkyl, heterocyclyl, OR¹⁴⁰ or NR¹³²R¹³³;
R¹³⁰ and R¹³¹ are each independently hydrogen, alkyl, alkenyl, alkynyl, aryl, heteroaryl, heteroarylium, cycloalkyl, heterocyclyl or C(A)R¹⁴¹, where R¹⁴¹ is alkyl, alkenyl, alkynyl, aryl, heteroaryl, heteroarylium, cycloalkyl, heterocyclyl, OR¹²⁵ or NR¹³²R¹³³; or R¹³⁰ and R¹³¹ together form alkylene, alkenylene, alkynylene, heteroalkylene;
R¹³² and R¹³³ are each independently hydrogen, alkyl, alkenyl, alkynyl, aryl, heteroaryl, heteroarylium, cycloalkyl, heterocyclyl, or R¹³² and R¹³³ together form alkylene, alkenylene, alkynylene, heteroalkylene; and
R³ is hydrogen, alkyl, alkenyl, alkynyl, cycloalkyl, heterocyclyl, aryl or heteroaryl;
wherein X, Y, Z, R¹, R² and R³ are each independently unsubstituted or substituted with one or more substituents, in one embodiment one, two or three substituents, each independently selected from Q¹, where Q¹ is halo, pseudohalo, hydroxy, oxo, thia, nitrile, nitro, formyl, mercapto, hydroxycarbonyl, hydroxycarbonylalkyl, hydroxycarbonylalkenyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, alkoxyxarbonylalkoxy, aryloxycarbonyl, aryloxycarbonylalkyl, aralkoxycarbonyl, aralkoxycarbonylalkyl, aralkoxycarbonylalkoxy, arylcarbonylalkyl, aminocarbonyl, aminocarbonylalkyl, aminocarbonylalkoxy, alkylaninocarbonyl, alkylaminocarbonylalkyl, alkylaminocarbonylalkoxy, dialkylaminocarbonyl, dialkylaminocarbonylalkyl, dialkylaminocarbonylalkoxy, arylaminocarbonyl, arylaminocarbonylalkyl, arylaminocarbonylalokoxy, diarylaminocarbonyl, diarylaminocarbonylalkyl, diarylaminocarbonyl alkoxy, arylalkylaminocarbonyl, arylalkylaminocarbonylalkyl, arylalkylaminocarbonylalkoxy, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R¹⁵¹R¹⁵²R¹⁵³, P(R¹⁵⁰)₂, P(═O)(R¹⁵⁰)₂, OP(═O)(R¹⁵⁰)₂, —NR¹⁶⁰C(═O)R¹⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; azido, tetrazolyl or two Q¹ groups, which substitute atoms in a 1,2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q¹ groups, which substitute the same atom, together form alkylene; and
each Q¹ is independently unsubstituted or substituted with one or more substituents, in one embodiment one, two or three substituents, each independently selected from Q²;
each Q² is independently halo, pseudohalo, hydroxy, oxo, thia, nitrile, nitro, formyl, mercapto, hydroxycarbonyl, hydroxycarbonylalkyl, hydroxycarbonylalkenyl alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R¹⁵¹R¹⁵²R¹⁵³, P(R¹⁵⁰)₂, P(═O)(R¹⁵⁰)₂, OP(═O)(R¹⁵⁰)₂, —NR¹⁶⁰C(═O)R¹⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q² groups, which substitute atoms in a 1,2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q² groups, which substitute the same atom, together form alkylene;
R¹⁵⁰ is hydroxy, alkoxy, aralkoxy, alkyl, heteroaryl, heterocyclyl, aryl or —NR¹⁷⁰R¹⁷¹, where R¹⁷⁰ and R¹⁷¹ are each independently hydrogen, alkyl, aralkyl, aryl, heteroaryl, heteroaralkyl or heterocyclyl, or R¹⁷⁰ and R¹⁷¹ together form alkylene, azaalkylene, oxaalkylene or thiaalkylene;
R¹⁵¹, R¹⁵² and R¹⁵³ are each independently hydrogen, alkyl, aryl, aralkyl, heteroaryl, heteroaralkyl, heterocyclyl or heterocyclylalkyl;
R¹⁶⁰ is hydrogen, alkyl, aryl, aralkyl, heteroaryl, heteroaralkyl, heterocyclyl or heterocyclylalkyl; and
R¹⁶³ is alkoxy, aralkoxy, alkyl, heteroaryl, heterocyclyl, aryl or —NR¹⁷⁰R¹⁷¹,
wherein the disorder is not a synucleinopathy.

2. The method of claim 1, wherein the compound is represented by Formula I: 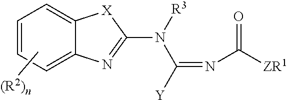
or a pharmaceutically acceptable derivative thereof, wherein:
where X is O, S or NR, where R is hydrogen, alkyl, alkenyl, alkynyl, cycloalkyl, heterocyclyl, aryl, heteroaryl or aralkyl;
Y is NRR′ or OH; where R′ is hydrogen, alkyl, alkenyl, alkynyl, cycloalkyl, heterocyclyl, aryl, heteroaryl or aralkyl;
Z is a direct bond or NR;
R¹ is hydrogen, alkyl, alkenyl, alkynyl, cycloalkyl, heterocyclyl, aryl, heteroaryl, aralkyl, aralkenyl, heteroaralkyl or heteroaralkenyl;
n is 0 to 4;
R² is selected from (i) or (ii) as follows:
(i) hydrogen, alkyl, alkenyl, alkynyl, aryl, heteroaryl, heteroarylium, cycloalkyl, heterocyclyl, C(A)R¹¹⁰, halo, pseudohalo, OR¹¹¹, S(D)ₐR¹¹², NR¹¹⁵R¹¹⁶ or N⁺R¹¹⁵R¹¹⁶R¹¹⁷; or
(ii) any two R² groups, which substitute adjacent atoms on the ring, together form alkylene, alkenylene, alkynylene or heteroalkylene;
A is O, S or NR¹²⁵;
R¹¹⁰ is hydrogen, alkyl, alkenyl, alkynyl, aryl, heteroaryl, heteroarylium, cycloalkyl, heterocyclyl, C(A)R¹²⁶, halo pseudohalo, OR¹²⁵, SR¹²⁵, NR¹²⁷R¹²⁸ and SiR¹²²R¹²³R¹²⁴;
R¹¹¹ is hydrogen, alkyl, alkenyl, alkynyl, aryl, heteroaryl, heteroarylium, cycloalkyl, heterocyclyl, C(A)R¹²⁹, NR¹³⁰R¹³¹ and SiR¹²²R¹²³R¹²⁴;
D is O or NR¹²⁵;
a is 0, 1 or 2;
when a is 1 or 2, R¹¹² is selected from hydrogen, alkyl, alkenyl, alkynyl, aryl, heteroaryl, heteroarylium, cycloalkyl, heterocyclyl, halo, pseudohalo, OR¹²⁵, SR¹²⁵ and NR¹³²R¹³³;
when a is 0, R¹¹² is selected from hydrogen, alkyl, alkenyl, alkynyl, aryl, heteroaryl, heteroarylium, cycloalkyl, heterocyclyl, SR¹²⁵ and C(A)R¹²⁹;
R¹¹⁵, R¹¹⁶ and R¹¹⁷ are each independently selected from (a) and (b) as follows:
(a) hydrogen, alkyl, alkenyl, alkynyl, aryl, heteroaryl, heteroarylium, cycloalkyl, heterocyclyl, C(A)R¹²⁹, OR¹²⁵ or NR¹³²R¹³³; or
(b) any two of R¹¹⁵, R¹¹⁶ and R¹¹⁷ together form alkylene, alkenylene, alkynylene, heteroalkylene, and the other is selected as in (a);
R¹²², R¹²³ and R¹²⁴ are selected as in (i) or (ii) as follows:
(i) R¹²², R¹²³ and R¹²⁴ are each independently hydrogen, alkyl, alkenyl, alkynyl, aryl, heteroaryl, heteroarylium, cycloalkyl, heterocyclyl, OR¹²⁵ or NR¹³²R¹³³; or
(ii) any two of R¹²², R¹²³ and R¹²⁴ together form alkylene, alkenylene, alkynylene, heteroalkylene; and the other is selected as in (i);
R¹²⁵ is hydrogen, alkyl, alkenyl, alkynyl, aryl, heteroaryl, heteroarylium, cycloalkyl or heterocyclyl;
R¹²⁶ is hydrogen, alkyl, alkenyl, alkynyl, aryl, heteroaryl, heteroarylium, cycloalkyl, heterocyclyl, OR¹²⁵ or NR¹³⁴R¹³⁵; where R¹³⁴ and R¹³⁵ are each independently hydrogen, alkyl, alkenyl, alkynyl, aryl, heteroaryl, heteroarylium, cycloalkyl, heterocyclyl, OR¹³⁶ or NR¹³²R¹³³, or R¹³⁴ and R¹³⁵ together form alkylene, alkenylene, alkynylene, heteroalkylene, where R¹³⁶ is hydrogen, alkyl, alkenyl, alkynyl, aryl, heteroaryl, heteroarylium, cycloalkyl or heterocyclyl;
R¹²⁷ and R¹²⁸ are selected as in (i) or (ii) as follows:
(i) R¹²⁷ and R¹²⁸ are each independently hydrogen, alkyl, alkenyl, alkynyl, aryl, heteroaryl, heteroarylium, cycloalkyl, heterocyclyl, OR¹²⁵, NR¹³⁷R¹³⁸ or C(A)R¹³⁹, where R¹³⁷ and R¹³⁸ are each independently hydrogen, alkyl, alkenyl, alkynyl, aryl, heteroaryl, heteroarylium, cycloalkyl or heterocyclyl, or together form alkylene, alkenylene, alkynylene, heteroalkylene; and R¹³⁹ is hydrogen, alkyl, alkenyl, alkynyl, aryl, heteroaryl, heteroarylium, cycloalkyl, heterocyclyl, OR¹⁴⁰ or NR¹³²R¹³³, where R¹⁴⁰ is alkyl, alkenyl, alkynyl, aryl, heteroaryl, heteroarylium, cycloalkyl, heterocyclyl; or
(ii) R¹²⁷ and R¹²⁸ together form alkylene, alkenylene, alkynylene, heteroalkylene;
R¹²⁹ is hydrogen, alkyl, alkenyl, alkynyl, aryl, heteroaryl, heteroarylium, cycloalkyl, heterocyclyl, OR¹⁴⁰ or NR¹³²R¹³³;
R¹³⁰ and R¹³¹ are each independently hydrogen, alkyl, alkenyl, alkynyl, aryl, heteroaryl, heteroarylium, cycloalkyl, heterocyclyl or C(A)R¹⁴¹, where R¹⁴¹ is alkyl, alkenyl, alkynyl, aryl, heteroaryl, heteroarylium, cycloalkyl, heterocyclyl, OR¹²⁵ or NR¹³²R¹³³; or R¹³⁰ and R¹³¹ together form alkylene, alkenylene, alkynylene, heteroalkylene;
R¹³² and R¹³³ are each independently hydrogen, alkyl, alkenyl, alkynyl, aryl, heteroaryl, heteroarylium, cycloalkyl, heterocyclyl, or R¹³² and R¹³³ together form alkylene, alkenylene, alkynylene, heteroalkylene; and
R³ is hydrogen, alkyl, alkenyl, alkynyl, cycloalkyl, heterocyclyl, aryl or heteroaryl;
wherein X, Y, Z, R¹, R² and R³ are each independently unsubstituted or substituted with one or more substituents, in one embodiment one, two or three substituents, each independently selected from Q¹, where Q¹ is halo, pseudohalo, hydroxy, oxo, thia, nitrile, nitro, formyl, mercapto, hydroxycarbonyl, hydroxycarbonylalkyl, hydroxycarbonylalkenyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, alkoxyxarbonylalkoxy, aryloxycarbonyl, aryloxycarbonylalkyl, aralkoxycarbonyl, aralkoxycarbonylalkyl, aralkoxycarbonylalkoxy, arylcarbonylalkyl, aminocarbonyl, aminocarbonylalkyl, aminocarbonylalkoxy, alkylaminocarbonyl, alkylaminocarbonylalkyl, alkylaminocarbonylalkoxy, dialkylaminocarbonyl, dialkylaminocarbonylalkyl, dialkylaminocarbonylalkoxy, arylaminocarbonyl, arylaminocarbonylalkyl, arylaminocarbonylalokoxy, diarylaminocarbonyl, diarylaminocarbonylalkyl, diarylaminocarbonyl alkoxy, arylalkylaminocarbonyl, arylalkylaminocarbonylalkyl, arylalkylaminocarbonylalkoxy, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R¹⁵¹R¹⁵²R¹⁵³, P(R¹⁵⁰)₂, P(═O)(R¹⁵⁰)₂, OP(═O)(R¹⁵⁰)₂, —NR¹⁶⁰C(═O)R¹⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; azido, tetrazolyl or two Q¹ groups, which substitute atoms in a 1,2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q¹ groups, which substitute the same atom, together form alkylene; and
each Q¹ is independently unsubstituted or substituted with one or more substituents, in one embodiment one, two or three substituents, each independently selected from Q²;
each Q² is independently halo, pseudohalo, hydroxy, oxo, thia, nitrile, nitro, formyl, mercapto, hydroxycarbonyl, hydroxycarbonylalkyl, hydroxycarbonylalkenyl alkyl, haloalkyl, polyhaloalkyl, aminoalkyl, diarninoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R¹⁵¹R¹⁵²R¹⁵³, P(R¹⁵⁰)₂, P(═O)(R¹⁵⁰)₂, OP(═O)(R¹⁵⁰)₂, —NR¹⁶⁰C(═O)R¹⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q² groups, which substitute atoms in a 1,2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q² groups, which substitute the same atom, together form alkylene;
R¹⁵⁰ is hydroxy, alkoxy, aralkoxy, alkyl, heteroaryl, heterocyclyl, aryl or —NR¹⁷⁰R¹⁷¹, where R¹⁷⁰ and R¹⁷¹ are each independently hydrogen, alkyl, aralkyl, aryl, heteroaryl, heteroaralkyl or heterocyclyl, or R¹⁷⁰ and R¹⁷¹ together forrn alkylene, azaalkylene, oxaalkylene or thiaalkylene;
R¹⁵¹, R¹⁵² and R¹⁵³ are each independently hydrogen, alkyl, aryl, aralkyl, heteroaryl, heteroaralkyl, heterocyclyl or heterocyclylalkyl;
R¹⁶⁰ is hydrogen, alkyl, aryl, aralkyl, heteroaryl, heteroaralkyl, heterocyclyl or heterocyclylalkyl; and
R¹⁶³ is alkoxy, aralkoxy, alkyl, heteroaryl, heterocyclyl, aryl or —NR¹⁷⁰R¹⁷¹,
wherein the disorder is not a synucleinopathy.

3. The method of claim 1, wherein:
X is O, S or NR, where R is hydrogen or alkyl;
Y is NRR′ or OH, where R is hydrogen or alkyl;
Z is a direct bond or NR;
R¹ is alkyl, alkenyl, alkynyl, cycloalkyl, aryl, heterocyclyl, heteroaryl, aralkyl, aralkenyl, heteroaralkyl, or heteroaralkenyl;
R² is halo, pseudohalo, alkoxy or alkyl;
n is 0 or 1;
R³ is hydrogen or alkyl;
wherein X, Y, Z, R¹, R² and R³ are each independently unsubstituted or substituted with one or more substituents, in one embodiment one, two or three substituents, each independently selected from Q¹.

4-13. (canceled)

14. The method of claim 1, wherein the compound is:

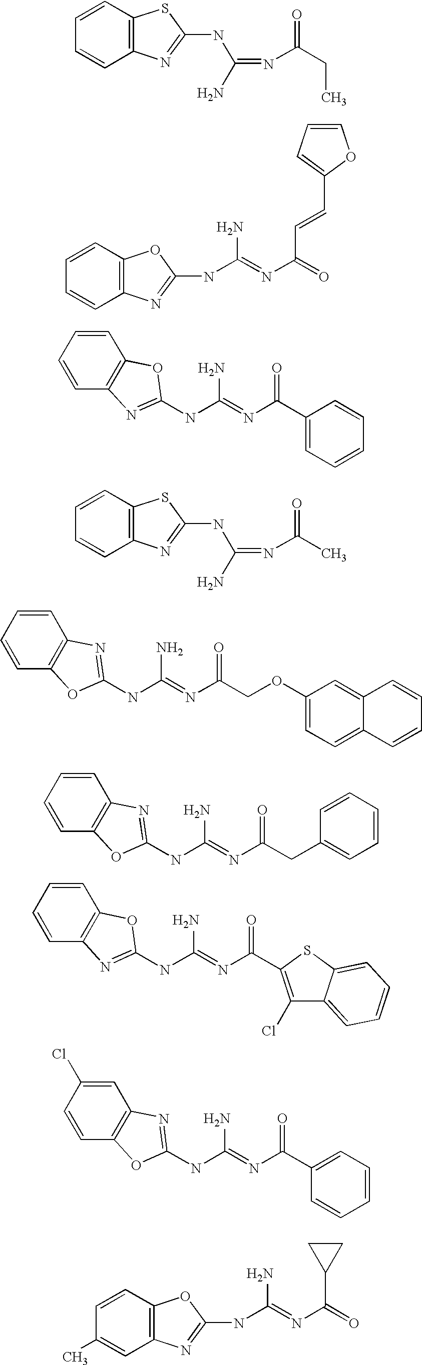

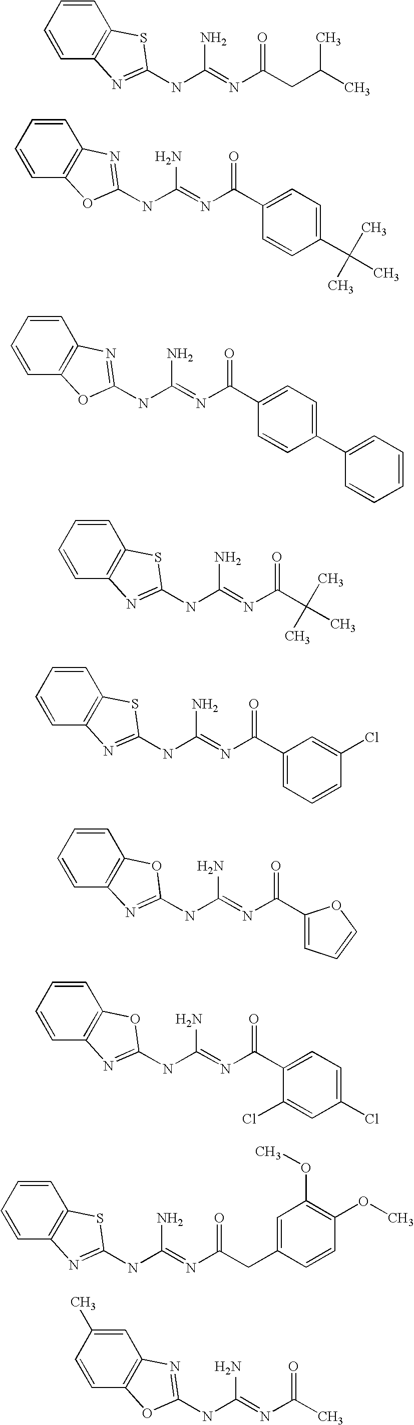

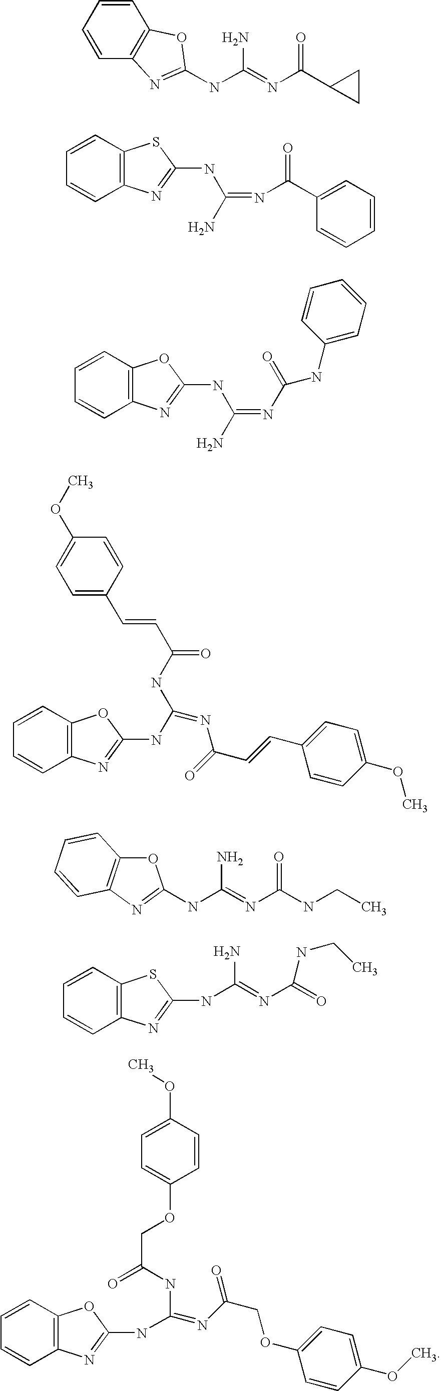

15. The method of claim 1, wherein the compound is:

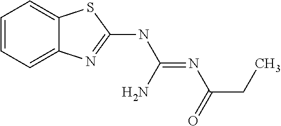

16. The method of claim 1, wherein the compound is:

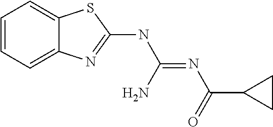

17. The method of claim 1, wherein the compound is selected from the compounds in Table I.

18. The method of claim 1, wherein the compound is represented by one of Formulas Ib-Im: 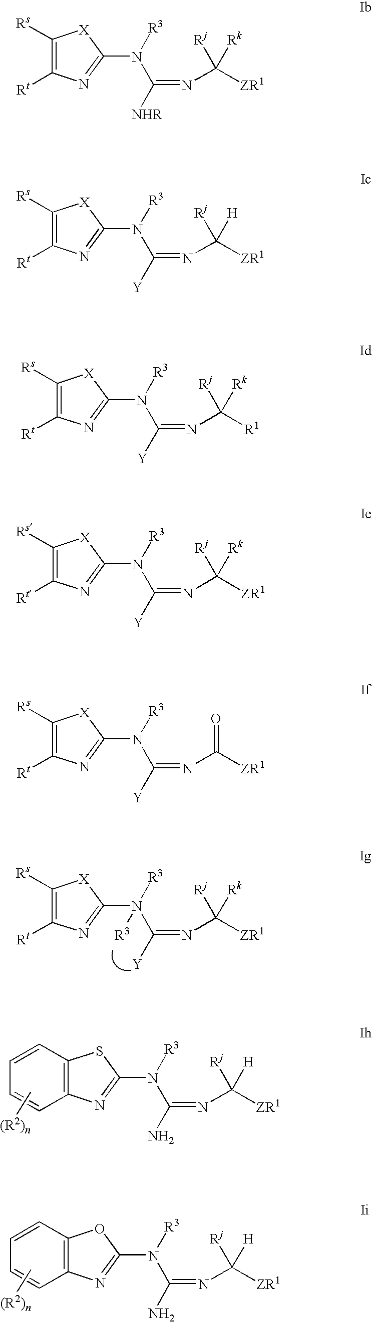 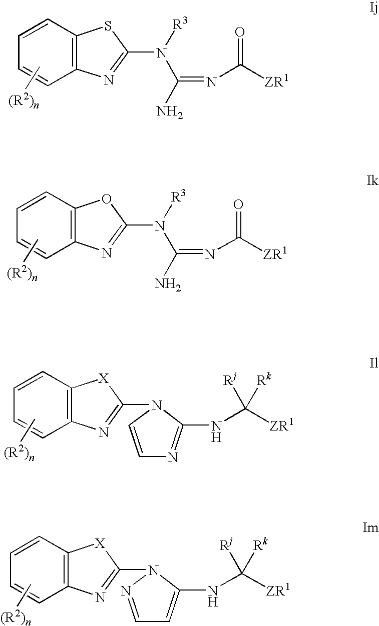
wherein
R¹ is hydrogen, alkyl, aryl, aralkyl, aralkenyl, alkynyl, heteroaryl, heteroaralkyl, heteroarylalkenyl, or cycloalkyl, each of which is substituted with 0, 1 or 2 groups selected from phenyl, alkyl, cycloalkyl, alkoxy, halo, pseudohalo, amino, alkylamino, or dialkylamino; and
R^{s}′ and R^{t}′ are independently selected from hydrogen, alkyl, halo, pseudohalo, alkenyl, alkynyl, cycloalkyl, heterocyclyl, aryl, heteroaryl and aralkyl.

19-22. (canceled)

23. The method of claim 18, wherein the compound is represented by Formula Ie: 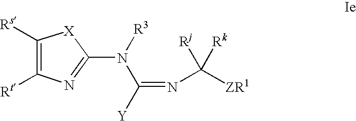
wherein R^{s}′ and R^{t}′ are independently selected from hydrogen, alkyl, and halo.

24. (canceled)

25. The method of claim 18, wherein the compound is represented by one of Formulas Ih-Im: 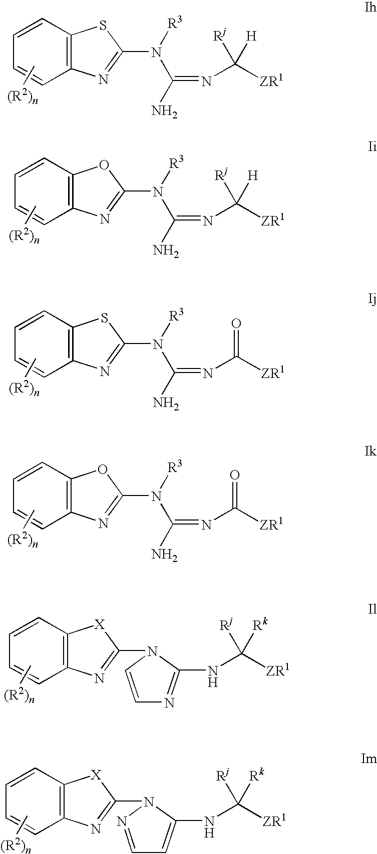
wherein
n is 0, 1 or 2; and
each R² is independently selected from halogen, alkyl, alkoxy, haloalkyl, and haloalkoxy.

26. (canceled)

27. A method of treating or preventing a disorder characterized by impaired protein trafficking, the method comprising administering to a subject a compound of Formula Ia: 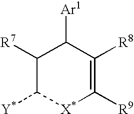
or a pharmaceutically acceptable derivative thereof, wherein:
X* is selected from the group consisting of —O—, ═N—, —N(R^{o})—, ═C(R^{o})— and —C(R^{o}R^{o}′)—;
Y* is selected from thr group consisting of ═O, —OR^{o}, ═NR^{o}′, —NR^{o}R^{o}′, ═CR^{o}R^{o}′ and —CHR^{o}R^{o}′; where X* and Y* are selected such that one of the dashed bonds (— — —) is a single bond and the other is a double bond, or both dashed bonds are single bonds;
each R^{o}′ is independently selected from the group consisting of hydrogen, halogen, pseudohalo, amino, amido, carboxamido, sulfonamide, carboxyl, alkyl, alkenyl, alkynyl, cycloalkyl, heterocyclyl, aryl, heteroaryl, aralkyl, alkoxy, cycloalkoxy, heterocycloxy, aryloxy, heteroaryloxy, and aralkyloxy;
each R^{o} is selected from the group consisting of hydrogen, alkyl, alkenyl, alkynyl, cycloalkyl, heterocyclyl, aryl, heteroaryl and aralkyl;
Ar¹ is aryl, heteroaryl, or cycloalkyl;
R⁷ is hydrogen, alkyl, alkenyl, alkynyl, cycloalkyl, heterocyclyl, aryl, heteroaryl or NRR, where R is hydrogen or alkyl;
R¹⁰ is hydrogen, alkyl, alkenyl, alkynyl, cycloalkyl, heterocyclyl, aryl or heteroaryl;
R⁸ and R⁹ are each independently selected from (i) or (ii) as follows:
(i) hydrogen, alkyl, alkenyl, alkynyl, aryl, heteroaryl, heteroarylium, cycloalkyl, heterocyclyl, C(A)R¹¹⁰, halo, pseudohalo, OR¹¹¹, S(D)ₐR¹¹², NR¹¹⁵R¹¹⁶ or N⁺R¹¹⁵R¹¹⁶R¹¹⁷; or
(ii) any two R² groups, which substitute adjacent atoms on the ring, together form alkylene, alkenylene, alkynylene or heteroalkylene;
A is O, S or NR¹²⁵;
R¹¹⁰ is hydrogen, alkyl, alkenyl, alkynyl, aryl, heteroaryl, heteroarylium, cycloalkyl, heterocyclyl, C(A)R¹²⁶, halo pseudohalo, OR¹²⁵, SR¹²⁵, NR¹²⁷R¹²⁸ and SiR¹²²R¹²³R¹²⁴;
R¹¹¹ is hydrogen, alkyl, alkenyl, alkynyl, aryl, heteroaryl, heteroarylium, cycloalkyl, heterocyclyl, C(A)R¹²⁶, NR¹³⁰R¹³¹ and SiR¹²²R¹²³R¹²⁴;
D is O or NR¹²⁵;
a is 0, 1 or 2;
when a is 1 or 2, R¹¹² is selected from hydrogen, alkyl, alkenyl, alkynyl, aryl, heteroaryl, heteroarylium, cycloalkyl, heterocyclyl, halo, pseudohalo, OR¹²⁵, SR¹²⁵ and NR¹³²R¹³³;
when a is 0, R¹¹² is selected from hydrogen, alkyl, alkenyl, alkynyl, aryl, heteroaryl, heteroarylium, cycloalkyl, heterocyclyl, SR¹²⁵ and C(A)R¹²⁹;
R¹¹⁵, R¹¹⁶ and R¹¹⁷ are each independently selected from (a) and (b) as follows:
(a) hydrogen, alkyl, alkenyl, alkynyl, aryl, heteroaryl, heteroarylium, cycloalkyl, heterocyclyl, C(A)R¹²⁹, OR¹²⁵ or NR¹³²R¹³³; or
(b) any two of R¹¹⁵, R¹¹⁶ and R¹¹⁷ together form alkylene, alkenylene, alkynylene, heteroalkylene, and the other is selected as in (a);
R¹²², R¹²³ and R¹²⁴ are selected as in (i) or (ii) as follows:
(i) R¹²², R¹²³ and R¹²⁴ are each independently hydrogen, alkyl, alkenyl, alkynyl, aryl, heteroaryl, heteroarylium, cycloalkyl, heterocyclyl, OR¹²⁵ or NR¹³²R¹³³; or
(ii) any two of R¹²², R¹²³ and R¹²⁴ together form alkylene, alkenylene, alkynylene, heteroalkylene; and the other is selected as in (i);
R¹²⁵ is hydrogen, alkyl, alkenyl, alkynyl, aryl, heteroaryl, heteroarylium, cycloalkyl or heterocyclyl;
R¹²⁶ is hydrogen, alkyl, alkenyl, alkynyl, aryl, heteroaryl, heteroarylium, cycloalkyl, heterocyclyl, OR¹²⁵ or NR¹³⁴R¹³⁵; where R¹³⁴ and R¹³⁵ are each independently hydrogen, alkyl, alkenyl, alkynyl, aryl, heteroaryl, heteroarylium, cycloalkyl, heterocyclyl, OR¹³⁶ or NR¹³²R¹³³, or R¹³⁴ and R¹³⁵ together form alkylene, alkenylene, alkynylene, heteroalkylene, where R¹³⁶ is hydrogen, alkyl, alkenyl, alkynyl, aryl, heteroaryl, heteroarylium, cycloalkyl or heterocyclyl;
R¹²⁷ and R¹²⁸ are selected as in (i) or (ii) as follows:
(i) R¹²⁷ and R¹²⁸ are each independently hydrogen, alkyl, alkenyl, alkynyl, aryl, heteroaryl, heteroarylium, cycloalkyl, heterocyclyl, OR¹²⁵, NR¹³⁷R¹³⁸ or C(A)R¹³⁹, where R¹³⁷ and R¹³⁸ are each independently hydrogen, alkyl, alkenyl, alkynyl, aryl, heteroaryl, heteroarylium, cycloalkyl or heterocyclyl, or together form alkylene, alkenylene, alkynylene, heteroalkylene; and R¹³⁹ is hydrogen, alkyl, alkenyl, alkynyl, aryl, heteroaryl, heteroarylium, cycloalkyl, heterocyclyl, OR¹⁴⁰ or NR¹³²R¹³³, where R¹⁴⁰ is alkyl, alkenyl, alkynyl, aryl, heteroaryl, heteroarylium, cycloalkyl, heterocyclyl; or
(ii) R¹²⁷ and R¹²⁸ together form alkylene, alkenylene, alkynylene, heteroalkylene;
R¹²⁹ is hydrogen, alkyl, alkenyl, alkynyl, aryl, heteroaryl, heteroarylium, cycloalkyl, heterocyclyl, OR¹⁴⁰ or NR¹³²R¹³³;
R¹³⁰ and R¹³¹ are each independently hydrogen, alkyl, alkenyl, alkynyl, aryl, heteroaryl, heteroarylium, cycloalkyl, heterocyclyl or C(A)R¹⁴¹, where R¹⁴¹ is alkyl, alkenyl, alkynyl, aryl, heteroaryl, heteroarylium, cycloalkyl, heterocyclyl, OR¹²⁵ or NR¹³²R¹³³; or R¹³⁰ and R¹³¹ together form alkylene, alkenylene, alkynylene, heteroalkylene;
R¹³² and R¹³³ are each independently hydrogen, alkyl, alkenyl, alkynyl, aryl, heteroaryl, heteroarylium, cycloalkyl, heterocyclyl, or R¹³² and R¹³³ together form alkylene, alkenylene, alkynylene, heteroalkylene; and
R¹⁰ is hydrogen, alkyl, alkenyl, alkynyl, cycloalkyl, heterocyclyl, aryl or heteroaryl;
where Ar¹, R⁷, R⁸, R⁹ and R¹⁰ are each independently unsubstituted or substituted with one or more, in one embodiment one, two or three substituents, each independently selected from Q¹, where Q¹ is halo, pseudohalo, hydroxy, oxo, thia, nitrile, nitro, formyl, mercapto, hydroxycarbonyl, hydroxycarbonylalkyl, hydroxycarbonylalkenyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, alkoxyxarbonylalkoxy, aryloxycarbonyl, aryloxycarbonylalkyl, aralkoxycarbonyl, aralkoxycarbonylalkyl, aralkoxycarbonylalkoxy, arylcarbonylalkyl, aminocarbonyl, aminocarbonylalkyl, aminocarbonylalkoxy, alkylaminocarbonyl, alkylaminocarbonylalkyl, alkylaminocarbonylalkoxy, dialkylaminocarbonyl, dialkylaminocarbonylalkyl, dialkylaminocarbonylalkoxy, arylaminocarbonyl, arylaminocarbonylalkyl, arylaminocarbonylalokoxy, diarylaminocarbonyl, diarylaminocarbonylalkyl, diarylaminocarbonyl alkoxy, arylalkylaminocarbonyl, arylalkylaminocarbonylalkyl, arylalkylaminocarbonylalkoxy, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylarninoalkyl, aryloxycarbonylaminoalkyl, aryloxyarylcarbonylamino, aryloxycarbonylamino, alkylsulfonylamino, arylsulfonylarnino, heteroarylsulfonylamino, heterocyclylsulfonylamino, heteroarylthio, azido, —N⁺R¹⁵¹R¹⁵²R¹⁵³, P(R¹⁵⁰)₂, P(═O)(R¹⁵⁰)₂, OP(═O)(R¹⁵⁰)₂, —NR¹⁶⁰C(═O)R¹⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; azido, tetrazolyl or two Q¹ groups, which substitute atoms in a 1,2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q¹ groups, which substitute the same atom, together form alkylene; and
each Q¹ is independently unsubstituted or substituted with one or more substituents, in one embodiment one, two or three substituents, each independently selected from Q²;
each Q² is independently halo, pseudohalo, hydroxy, oxo, thia, nitrile, nitro, foirmyl, mercapto, hydroxycarbonyl, hydroxycarbonylalkyl, hydroxycarbonylalkenyl alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R¹⁵¹R¹⁵²R¹⁵³, P(R¹⁵⁰)₂, P(═O)(R¹⁵⁰)₂, OP(═O)(R¹⁵⁰)₂, —NR¹⁶⁰C(═O)R¹⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q² groups, which substitute atoms in a 1,2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q² groups, which substitute the same atom, together form alkylene;
R¹⁵⁰ is hydroxy, alkoxy, aralkoxy, alkyl, heteroaryl, heterocyclyl, aryl or —NR¹⁷⁰R¹⁷¹, where R¹⁷⁰ and R¹⁷¹ are each independently hydrogen, alkyl, aralkyl, aryl, heteroaryl, heteroaralkyl or heterocyclyl, or R¹⁷⁰ and R¹⁷¹ together form alkylene, azaalkylene, oxaalkylene or thiaalkylene;
R¹⁵¹, R¹⁵² and R¹⁵³ are each independently hydrogen, alkyl, aryl, aralkyl, heteroaryl, heteroaralkyl, heterocyclyl or heterocyclylalkyl;
R¹⁶⁰ is hydrogen, alkyl, aryl, aralkyl, heteroaryl, heteroaralkyl, heterocyclyl or heterocyclylalkyl; and
R¹⁶³ is alkoxy, aralkoxy, alkyl, heteroaryl, heterocyclyl, aryl or —NR¹⁷⁰R¹⁷¹,
wherein the disorder is not a synucleinopathy.

28. The method of claim 27, wherein the compound is represented by Formula II: 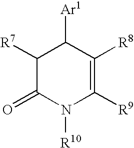
or a pharmaceutically acceptable derivative thereof, wherein:
Ar¹ is aryl, heteroaryl, or cycloalkyl;
R⁷ is hydrogen, alkyl, alkenyl, alkynyl, cycloalkyl, heterocyclyl, aryl, heteroaryl or NRR, where R is hydrogen or alkyl;
R¹⁰ is hydrogen, alkyl, alkenyl, alkynyl, cycloalkyl, heterocyclyl, aryl or heteroaryl;
R⁸ and R⁹ are each independently selected from (i) or (ii) as follows:
(i) hydrogen, alkyl, alkenyl, alkynyl, aryl, heteroaryl, heteroarylium, cycloalkyl, heterocyclyl, C(A)R¹¹⁰, halo, pseudohalo, OR¹¹¹, S(D)ₐR¹¹², NR¹¹⁵R¹¹⁶ or N⁺R¹¹⁵R¹¹⁶R¹¹⁷; or
(ii) any two R² groups, which substitute adjacent atoms on the ring, together form alkylene, alkenylene, alkynylene or heteroalkylene;
A is O, S or NR¹²⁵;
R¹¹⁰ is hydrogen, alkyl, alkenyl, alkynyl, aryl, heteroaryl, heteroarylium, cycloalkyl, heterocyclyl, C(A)R¹²⁶, halo pseudohalo, OR¹²⁵, SR¹²⁵, NR¹²⁷R¹²⁸nd SiR¹²²R¹²³R¹²⁴;
R¹¹¹ is hydrogen, alkyl, alkenyl, alkynyl, aryl, heteroaryl, heteroarylium, cycloalkyl, heterocyclyl, C(A)R¹²⁹, NR¹³⁰R¹³¹ and SiR¹²²R¹²³R¹²⁴;
D is O or NR¹²⁵;
a is 0, 1 or 2;
when a is 1 or 2, R¹¹² is selected from hydrogen, alkyl, alkenyl, alkynyl, aryl, heteroaryl, heteroarylium, cycloalkyl, heterocyclyl, halo, pseudohalo, OR¹²⁵, SR¹²⁵ and NR¹³²R¹³³;
when a is 0, R¹¹² is selected from hydrogen, alkyl, alkenyl, alkynyl, aryl, heteroaryl, heteroarylium, cycloalkyl, heterocyclyl, SR¹²⁵ and C(A)R¹²⁹;
R¹¹⁵, R¹¹⁶ and R¹¹⁷ are each independently selected from (a) and (b) as follows:
(a) hydrogen, alkyl, alkenyl, alkynyl, aryl, heteroaryl, heteroarylium, cycloalkyl, heterocyclyl, C(A)R¹²⁹, OR¹²⁵ or NR¹³²R¹³³; or
(b) any two of R¹¹⁵, R¹¹⁶ and R¹¹⁷ together form alkylene, alkenylene, alkynylene, heteroalkylene, and the other is selected as in (a);
R¹²², R¹²³ and R¹²⁴ are selected as in (i) or (ii) as follows:
(i) R¹²², R¹²³ and R¹²⁴ are each independently hydrogen, alkyl, alkenyl, alkynyl, aryl, heteroaryl, heteroarylium, cycloalkyl, heterocyclyl, OR¹²⁵ or NR¹³²R¹³³; or
(ii) any two of R¹²², R¹²³ and R¹²⁴ together form alkylene, alkenylene, alkynylene, heteroalkylene; and the other is selected as in (i);
R¹²⁵ is hydrogen, alkyl, alkenyl, alkynyl, aryl, heteroaryl, heteroarylium, cycloalkyl or heterocyclyl;
R¹²⁶ is hydrogen, alkyl, alkenyl, alkynyl, aryl, heteroaryl, heteroarylium, cycloalkyl, heterocyclyl, OR¹²⁵ or NR¹³⁴R¹³⁵; where R¹³⁴ and R¹³⁵ are each independently hydrogen, alkyl, alkenyl, alkynyl, aryl, heteroaryl, heteroarylium, cycloalkyl, heterocyclyl, OR¹³⁶ or NR¹³²R¹³³, or R¹³⁴ and R¹³⁵ together form alkylene, alkenylene, alkynylene, heteroalkylene, where R¹³⁶ is hydrogen, alkyl, alkenyl, alkynyl, aryl, heteroaryl, heteroarylium, cycloalkyl or heterocyclyl;
R¹²⁷ and R¹²⁸ are selected as in (i) or (ii) as follows:
(i) R¹²⁷ and R¹²⁸ are each independently hydrogen, alkyl, alkenyl, alkynyl, aryl, heteroaryl, heteroarylium, cycloalkyl, heterocyclyl, OR¹²⁵, NR¹³⁷R¹³⁸ or C(A)R¹³⁹, where R¹³⁷ and R¹³⁸ are each independently hydrogen, alkyl, alkenyl, alkynyl, aryl, heteroaryl, heteroarylium, cycloalkyl or heterocyclyl, or together form alkylene, alkenylene, alkynylene, heteroalkylene; and R¹³⁹ is hydrogen, alkyl, alkenyl, alkynyl, aryl, heteroaryl, heteroarylium, cycloalkyl, heterocyclyl, OR¹⁴⁰ or NR¹³²R¹³³, where R¹⁴⁰ is alkyl, alkenyl, alkynyl, aryl, heteroaryl, heteroarylium, cycloalkyl, heterocyclyl; or
(ii) R¹²⁷ and R¹²⁸ together form alkylene, alkenylene, alkynylene, heteroalkylene;
R¹²⁹ is hydrogen, alkyl, alkenyl, alkynyl, aryl, heteroaryl, heteroarylium, cycloalkyl, heterocyclyl, OR¹⁴⁰ or NR¹³²R¹³³;
R¹³⁰ and R¹³¹ are each independently hydrogen, alkyl, alkenyl, alkynyl, aryl, heteroaryl, heteroarylium, cycloalkyl, heterocyclyl or C(A)R¹⁴¹, where R¹⁴¹ is alkyl, alkenyl, alkynyl, aryl, heteroaryl, heteroarylium, cycloalkyl, heterocyclyl, OR¹²⁵ or NR¹³²R¹³³; or R¹³⁰ and R¹³¹ together form alkylene, alkenylene, alkynylene, heteroalkylene;
R¹³² and R¹³³ are each independently hydrogen, alkyl, alkenyl, alkynyl, aryl, heteroaryl, heteroarylium, cycloalkyl, heterocyclyl, or R¹³² and R¹³³ together form alkylene, alkenylene, alkynylene, heteroalkylene; and
R¹⁰ is hydrogen, alkyl, alkenyl, alkynyl, cycloalkyl, heterocyclyl, aryl or heteroaryl;
where Ar¹, R⁷, R⁸, R⁹ and R¹⁰ are each independently unsubstituted or substituted with one or more, in one embodiment one, two or three substituents, each independently selected from Q¹, where Q¹ is halo, pseudohalo, hydroxy, oxo, thia, nitrile, nitro, formyl, mercapto, hydroxycarbonyl, hydroxycarbonylalkyl, hydroxycarbonylalkenyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, alkoxyxarbonylalkoxy, aryloxycarbonyl, aryloxycarbonylalkyl, aralkoxycarbonyl, aralkoxycarbonylalkyl, aralkoxycarbonylalkoxy, arylcarbonylalkyl, aminocarbonyl, aminocarbonylalkyl, aminocarbonylalkoxy, alkylaminocarbonyl, alkylaminocarbonylalkyl, alkylaminocarbonylalkoxy, dialkylaminocarbonyl, dialkylaminocarbonylalkyl, dialkylaminocarbonylalkoxy, arylaminocarbonyl, arylaminocarbonylalkyl, arylaminocarbonylalokoxy, diarylaminocarbonyl, diarylaminocarbonylalkyl, diarylaminocarbonyl alkoxy, arylalkylaminocarbonyl, arylalkylaminocarbonylalkyl, arylalkylaminocarbonylalkoxy, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R¹⁵¹R¹⁵²R¹⁵³, P(R¹⁵⁰)₂, P(═O)(R¹⁵⁰)₂, OP(═O)(R¹⁵⁰)₂, —NR¹⁶⁰C(═O)R¹⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; azido, tetrazolyl or two Q¹ groups, which substitute atoms in a 1,2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q¹ groups, which substitute the same atom, together form alkylene; and
each Q¹ is independently unsubstituted or substituted with one or more substituents, in one embodiment one, two or three substituents, each independently selected from Q²;
each Q² is independently halo, pseudohalo, hydroxy, oxo, thia, nitrile, nitro, formyl, mercapto, hydroxycarbonyl, hydroxycarbonylalkyl, hydroxycarbonylalkenyl alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R¹⁵¹R¹⁵²R¹⁵³, P(R¹⁵⁰)₂, P(═O)(R¹⁵⁰)₂, OP(═O)(R¹⁵⁰)₂, —NR¹⁶⁰C(═O)R¹⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q² groups, which substitute atoms in a 1,2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q² groups, which substitute the same atom, together form alkylene;
R¹⁵⁰ is hydroxy, alkoxy, aralkoxy, alkyl, heteroaryl, heterocyclyl, aryl or —NR¹⁷⁰R¹⁷¹, where R¹⁷⁰ and R¹⁷¹ are each independently hydrogen, alkyl, aralkyl, aryl, heteroaryl, heteroaralkyl or heterocyclyl, or R¹⁷⁰ and R¹⁷¹ together form alkylene, azaalkylene, oxaalkylene or thiaalkylene;
R¹⁵¹, R¹⁵² and R¹⁵³ are each independently hydrogen, alkyl, aryl, aralkyl, heteroaryl, heteroaralkyl, heterocyclyl or heterocyclylalkyl;
R¹⁶⁰ is hydrogen, alkyl, aryl, aralkyl, heteroaryl, heteroaralkyl, heterocyclyl or heterocyclylalkyl; and
R¹⁶³ is alkoxy, aralkoxy, alkyl, heteroaryl, heterocyclyl, aryl or —NR¹⁷⁰R¹⁷¹,
wherein the disorder is not a synucleinopathy.

29. The method of claim 27, wherein Ar¹ is aryl, heteroaryl, or cycloalkyl, and is unsubstituted or substituted with alkyl, alkenyl, alkynyl, heteroaryl, halo, pseudohalo, dialkylamino, aryloxy, aralkoxy, haloalkyl, alkoxy, haloalkoxy, cycloalkyl, or COOR, where R is hydrogen or alkyl;
R⁷is hydrogen or NRR, where R is hydrogen or alkyl;
R⁸ and R⁹ are each independently selected from (i) and (ii) as follows:
(i) R⁸ and R⁹ together with the atoms to which they are attached form a fused phenyl ring, which is unsubstituted or substituted with halo, pseudohalo, alkyl, alkoxy, cycloalkyl, fused cycloalkyl, fused heterocyclyl, fused heteroaryl, or fused aryl, which is unsubstituted or substituted with halo, pseudohalo, alkyl, alkoxy, aryl, cycloalkyl, heterocyclyl, fused aryl, fused heterocyclyl, and fused cycloalkyl; and
(ii) R⁸ is CN or COOR²⁰⁰, where R²⁰⁰ is hydrogen, alkyl, alkenyl, alkynyl, cycloalkyl, heterocyclyl, aryl or heteroaryl; and R⁹ is hydrogen, alkyl or alkylthio; and
R¹⁰ is hydrogen;
where Ar¹, R⁷, R⁸, R⁹ and R¹⁰ are each independently unsubstituted or substituted with one or more, in one embodiment one, two or three substituents, each independently selected from Q¹.

30-33. (canceled)

34. The method of claim 27, wherein R⁸ and R⁹ are each independently selected from (i) and (ii) as follows:
(i) R⁸ and R⁹ together with the atoms to which they are attached form a fused phenyl ring, which is unsubstituted or substituted with methyl, chloro, methoxy, cyclopentyl, fused cyclopentyl, or another fused phenyl ring, which is unsubstituted or substituted with bromo; and
(ii) R⁸ is CN or COOR²⁰⁰, where R²⁰⁰ is methyl, benzyl, ethyl, 4-methoxybenzyl or 2-phenylethyl; and R⁹ is methyl, methylthio or phenylaminocarbonylmethylthio.

35. The method of claim 27, wherein the compound is:

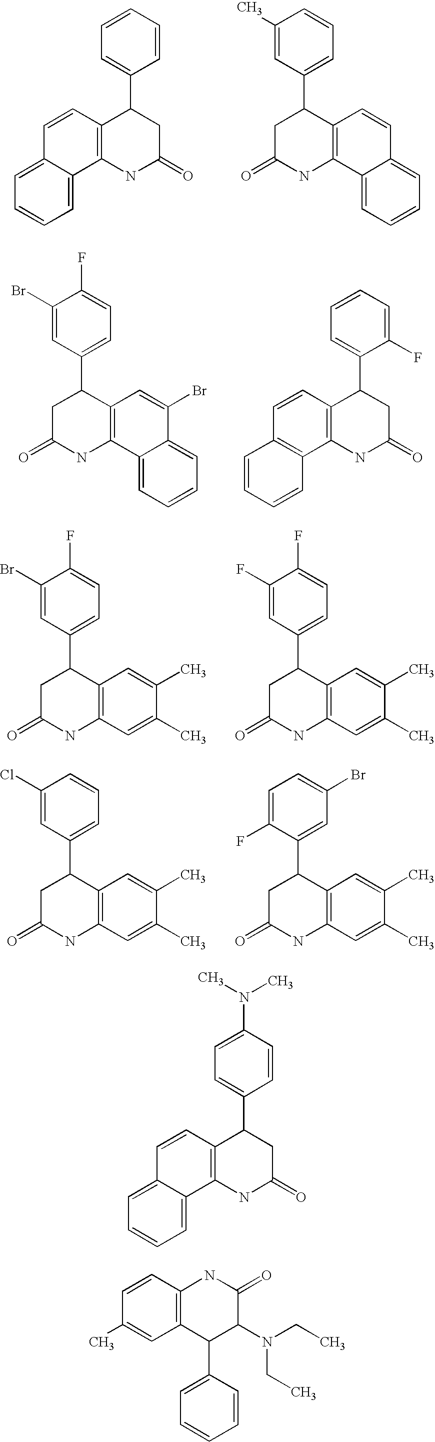

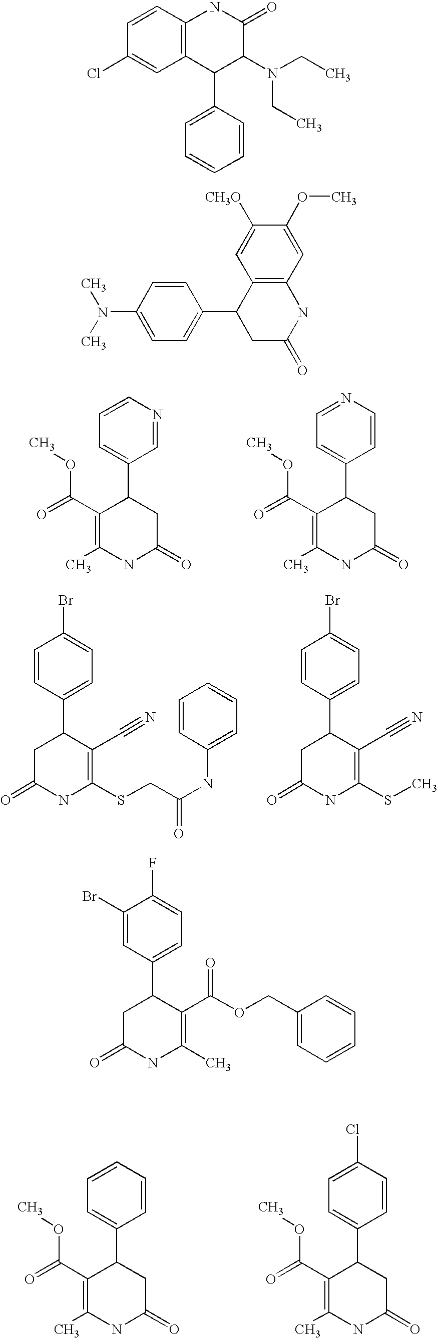

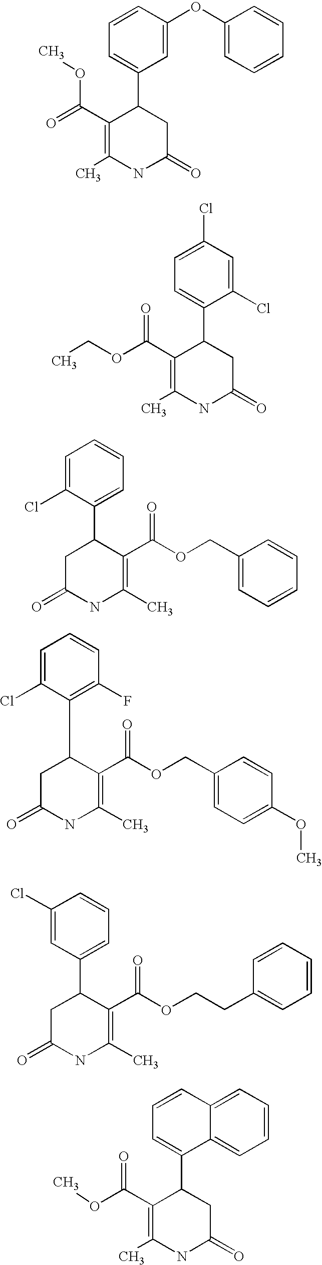

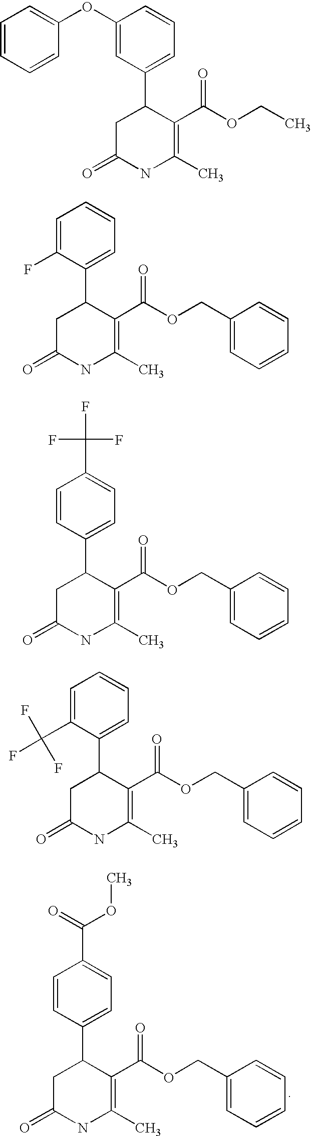

36. The method of claim 27, wherein the compound is:

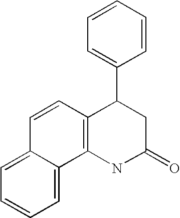

37. The method of claim 27, wherein the compound is:

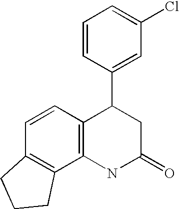

38. The method of claim 27, wherein the compound is represented by one of Formulas IIb-IIp: 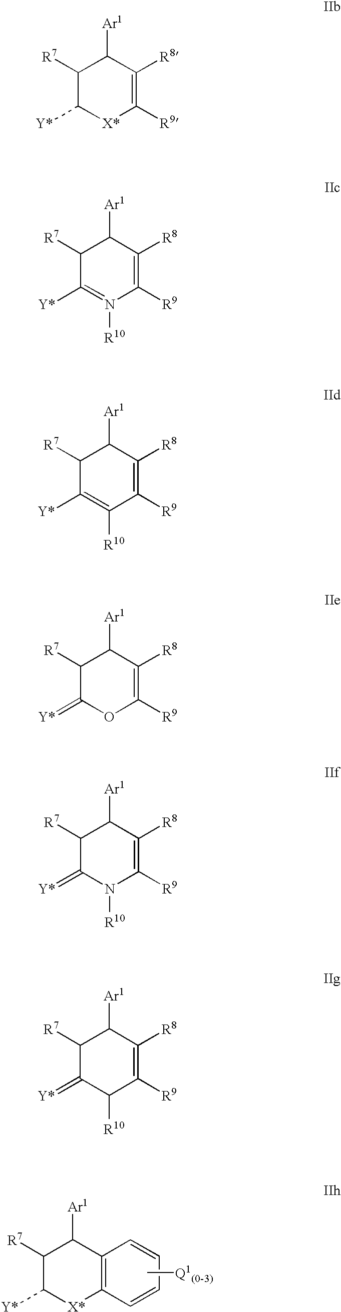 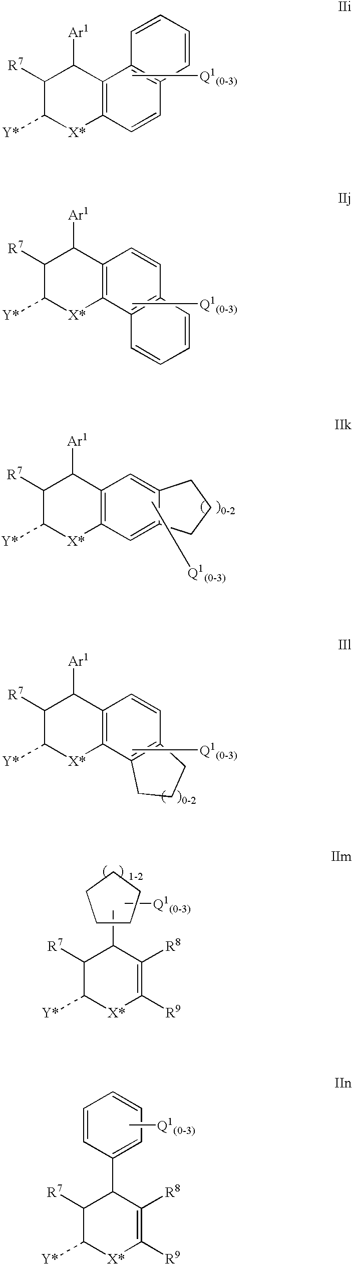 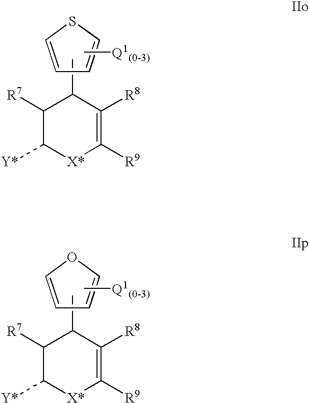
wherein X* and Y* are selected such that one of the dashed bonds (— — —) is a single bond and the other is a double bond; and
R⁸′ and R⁹′ are independently selected from hydrogen, alkyl, alkenyl, alkynyl, aryl, heteroaryl, heteroarylium, cycloalkyl, heterocyclyl, C(A)R¹¹⁰, halo, pseudohalo, OR¹¹¹, S(D)ₐR¹¹², NR¹¹⁵R¹¹⁶ or N⁺R¹¹⁵R¹¹⁶R¹¹⁷.

39. The method of claim 38, wherein the compound is represented by Formula Ib, wherein:
R^{8′} is CN or COOR²⁰⁰, where R²⁰⁰ is hydrogen, alkyl, alkenyl, alkynyl, cycloalkyl, heterocyclyl, aryl or heteroaryl; and
R^{9′} is hydrogen, alkyl or alkylthio.

40. The method of claim 39, wherein:
R^{8′} is CN or COOR²⁰⁰, where R²⁰⁰ is methyl, benzyl, ethyl, 4-methoxybenzyl or 2-phenylethyl; and
R^{9′} is methyl, methylthio or phenylaminocarbonylmethylthio.

41. The method of claim 38, wherein the compound is represented by one of Formulas IIh-IIp: 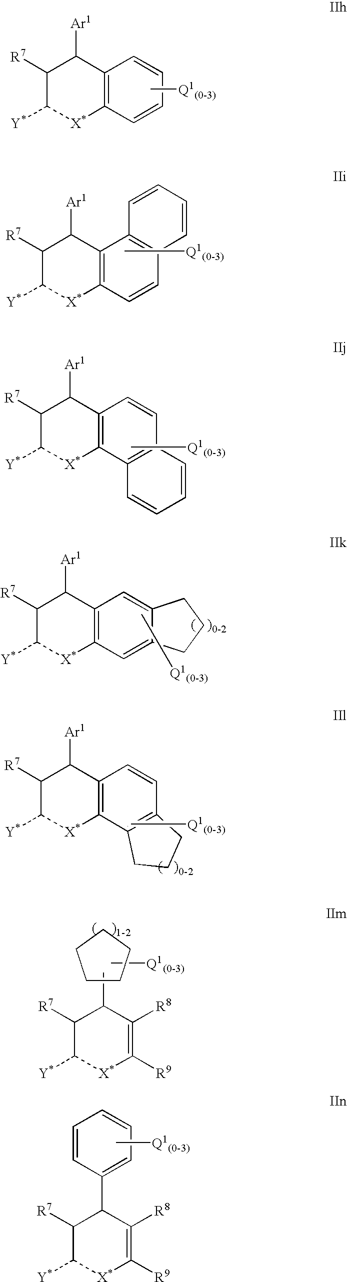 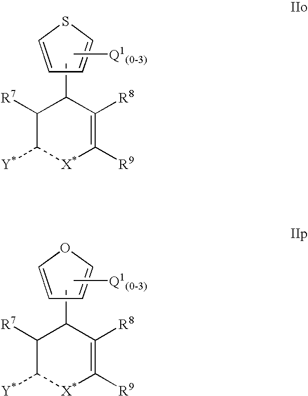
wherein each Q¹ is independently selected from halogen, alkyl, alkoxy, nitro, CN, N₃, aryl, aryloxy, arylalkyloxy, alkynyl, amino, alkylamino, heterocyclyl, heteroaryl, substituted carboxyl, haloalkyl, and haloalkoxy, or two adjacent Q¹, on the same phenyl or adjacent fused phenyl rings, together form a cycloalkyl or heterocyclyl ring fused with the phenyl or adjacent fused phenyl rings.

42. The method of claim 27, wherein the compound is represented by one of Formulas IIq, IIr, or IIs: 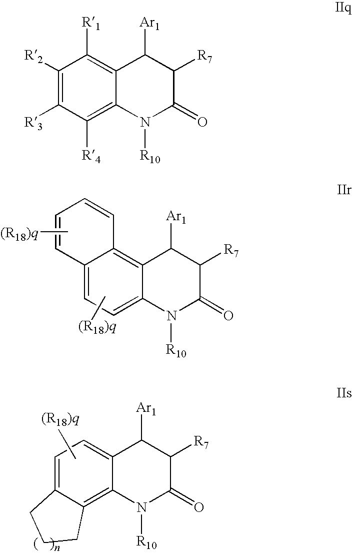
wherein
each q is independently 0, 1, or 2;
n is 0, 1 or 2;
R′1, R′2, R′3, R′4, and each R18 are independently selected from hydrogen, alkyl, alkenyl, alkynyl, aryl, heteroaryl, heteroarylium, cycloalkyl, heterocyclyl, C(A)R¹¹⁰, halo, pseudohalo, OR¹¹¹, S(D)ₐR¹¹², NR¹¹⁵R¹¹⁶ and N⁺R¹¹⁵R¹¹⁶R¹¹⁷.

43. (canceled)

44. A method of treating or preventing a disorder characterized by impaired protein trafficking, the method comprising administering to a subject a compound selected from the group consisting of doxorubicin, cycloheximide, hygromycin, novobiocin, aureobasidin, and tunicamycin.

45. The method of claim 1, wherein the disorder is a lysosomal storage disorder.

46. (canceled)

47. The method of claim 1, wherein the disorder is characterized by an impaired delivery of cargo to a cellular compartment.

48. (canceled)

49. The method of claim 48, wherein the disorder is Griscelli syndrome.

50. The method of claim 1, wherein the disorder is cystic fibrosis.

51. The method of claim 1, wherein the disorder is diabetes.

52. (canceled)

53. The method of claim 1, wherein the disorder is hereditary emphysema, hereditary hemochromatosis, oculocutaneous albinism, protein C deficiency, type I hereditary angioedema, congenital sucrase-isomaltase deficiency, Crigler-Najjar type II, Laron syndrome, hereditary Myeloperoxidase, primary hypothyroidism, congenital long QT syndrome, tyroxine binding globulin deficiency, familial hypercholesterolemia, familial chylomicronemia, abeta-lipoproteinema, low plasma lipoprotein a levels, hereditary emphysema with liver injury, congenital hypothyroidism, osteogenesis imperfecta, hereditary hypofibrinogenemia, alpha-lantichymotrypsin deficiency, nephrogenic diabetes insipidus, neurohypophyseal diabetes, insipidus, Charcot-Marie-Tooth syndrome, Pelizaeus Merzbacher disease, von Willebrand disease type IIA, combined factors V and VIII deficiency, spondylo-epiphyseal dysplasia tarda, choroideremia, I cell disease, Batten disease, ataxia telangiectasias, acute lymphoblastic leukemia, acute myeloid leukemia, myeloid leukemia, ADPKD-autosomal dominant polycystic kidney disease, microvillus inclusion disease, tuberous sclerosis, oculocerebro-renal syndrome of Lowe, amyotrophic lateral sclerosis, myelodysplastic syndrome, Bare lymphocyte syndrome, Tangier disease, familial intrahepatic cholestasis, X-linked adreno-leukodystrophy, Scott syndrome, Hermansky-Pudlak syndrome types 1 and 2, Zellweger syndrome, rhizomelic chondrodysplasia puncta, autosomal recessive primary hyperoxaluria, Mohr Tranebjaerg syndrome, spinal and bullar muscular atrophy, primary ciliary diskenesia (Kartagener's syndrome), Miller Dieker syndrome, lissencephaly, motor neuron disease, Usher's syndrome, Wiskott-Aldrich syndrome, Optiz syndrome, Huntington's disease, hereditary pancreatitis, anti-phospholipid syndrome, overlap connective tissue disease, Sjögren's syndrome, stiff-man syndrome, Brugada syndrome, congenital nephritic syndrome of the Finnish type, Dubin-Johnson syndrome, X-linked hypophosphosphatemia, Pendred syndrome, persistent hyperinsulinemic hypoglycemia of infancy, hereditary spherocytosis, aceruloplasminemia, infantile neuronal ceroid lipofuscinosis, pseudoachondroplasia and multiple epiphyseal, Stargardt-like macular dystrophy, X-linked Charcot-Marie-Tooth disease, autosomal dominant retinitis pigmentosa, Wolcott-Rallison syndrome, Cushing's disease, limb-girdle muscular dystrophy, mucoploy-saccharidosis type IV, hereditary familial amyloidosis of Finish, Anderson disease, sarcoma, chronic myelomonocytic leukemia, cardiomyopathy, faciogenital dysplasia, Torsion disease, Huntington and spinocerebellar ataxias, hereditary hyperhomosyteinemia, polyneuropathy, lower motor neuron disease, pigmented retinitis, seronegative polyarthritis, interstitial pulmonary fibrosis, Raynaud's phenomenon, Wegner's granulomatosis, preoteinuria, CDG-Ia, CDG-Ib, CDG-Ic, CDG-Id, CDG-Ie, CDG-If, CDG-IIa, CDG-IIb, CDG-IIc, CDG-IId, Ehlers-Danlos syndrome, multiple exostoses, Griscelli syndrome (type 1 or type 2), or X-linked non-specific mental retardation.

54. A method of identifying a compound that rescues impaired endoplasmic reticulum-mediated transport, the method comprising:
providing a cell that exhibits reduced expression or activity of a protein required for endoplasmic reticulum-mediated transport;
contacting the cell with a candidate agent; and
determining whether growth of the cell is enhanced in the presence of the candidate agent as compared to in the absence of the candidate agent,
wherein a compound that enhances growth is identified as a compound that rescues impaired endoplasmic reticulum-mediated transport.

55-56. (canceled)

57. A method of identifying a compound that enhances protein secretion, the method comprising:
providing a cell that exhibits reduced expression or activity of a protein required for endoplasmic reticulum-mediated transport;
contacting the cell with a candidate agent; and
determining whether protein secretion is enhanced in the presence of the candidate agent as compared to in the absence of the candidate agent,
wherein a compound that enhances growth is identified as a compound that enhances protein secretion.

58-67. (canceled)

68. A compound represented by Formula Ia: 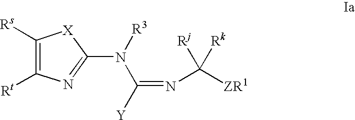
or a pharmaceutically acceptable derivative thereof, wherein:
R^{j} and R^{k} are independently selected from hydrogen, alkyl, alkenyl, alkynyl, cycloalkyl, heterocyclyl, aryl, heteroaryl or aralkyl; or, R^{j} and R^{k}, together with the carbon to which they are both bonded, are —C(═O)—, —CH(OR*)—, —C(═S)—, CH(SR*)—, —CH(NR*R*′)— or —C(═NR*)—;
R* and R*′ are independently hydrogen, alkyl, alkenyl, alkynyl, cycloalkyl, heterocyclyl, aryl, heteroaryl or aralkyl;
R^{s} and R^{t} are independently selected from hydrogen, alkyl, halo, pseudohalo, alkenyl, alkynyl, cycloalkyl, heterocyclyl, aryl, heteroaryl or aralkyl; or, R^{s} and R^{t}, together with the carbon-carbon double bond between them, form a 4-6 membered cycloalkenyl, aryl, heterocyclyl, or heteroaryl ring, wherein the ring formed by R^{s} and R^{t} is optionally substituted with 0-4 substituents R²;
X is O, S or NR, where R is hydrogen, alkyl, alkenyl, alkynyl, cycloalkyl, heterocyclyl, aryl, heteroaryl or aralkyl;
Y is NRR″, OR′, SR′, or CRR″; where R″ is hydrogen, alkyl, alkenyl, alkynyl, cycloalkyl, heterocyclyl, aryl, heteroaryl or aralkyl, or R″, together with R³ and the atoms therebetween, is a 4-6 membered heterocyclyl or heteroaryl ring; provided that when R^{j} and R^{k}, together with the carbon to which they are both bonded, are R″, together with R³ and the atoms therebetween, is a 4-6 membered heterocyclyl or heteroaryl ring;
Z is a direct bond or NR;
R¹ is hydrogen, alkyl, alkenyl, alkynyl, cycloalkyl, heterocyclyl, aryl, heteroaryl, aralkyl, aralkenyl, heteroaralkyl or heteroaralkenyl;
n is 0 to 4;
R² is selected from (i) or (ii) as follows:
(i) hydrogen, alkyl, alkenyl, alkynyl, aryl, heteroaryl, heteroarylium, cycloalkyl, heterocyclyl, C(A)R¹¹⁰, halo, pseudohalo, OR¹¹¹, S(D)ₐR¹¹², NR¹¹⁵R¹¹⁶ or N⁺R¹¹⁵R¹¹⁶R¹¹⁷; or
(ii) any two R² groups, which substitute adjacent atoms on the ring, together form alkylene, alkenylene, alkynylene or heteroalkylene;
A is O, S or NR¹²⁵;
R¹¹⁰ is hydrogen, alkyl, alkenyl, alkynyl, aryl, heteroaryl, heteroarylium, cycloalkyl, heterocyclyl, C(A)R¹²⁶, halo pseudohalo, OR¹²⁵, SR¹²⁵, NR¹²⁷R¹²⁸ and SiR¹²²R¹²³R¹²⁴;
R¹¹¹ is hydrogen, alkyl, alkenyl, alkynyl, aryl, heteroaryl, heteroarylium, cycloalkyl, heterocyclyl, C(A)R¹²⁹, NR¹³⁰R¹³¹ and SiR¹²²R¹²³R¹²⁴.
D is O or NR¹²⁵;
a is 0, 1 or 2;
when a is 1 or 2, R¹¹² is selected from hydrogen, alkyl, alkenyl, alkynyl, aryl, heteroaryl, heteroarylium, cycloalkyl, heterocyclyl, halo, pseudohalo, OR¹²⁵, SR¹²⁵ and NR¹³²R¹³³;
when a is 0, R¹¹² is selected from hydrogen, alkyl, alkenyl, alkynyl, aryl, heteroaryl, heteroarylium, cycloalkyl, heterocyclyl, SR¹²⁵ and C(A)R¹²⁹;
R¹¹⁵, R¹¹⁶ and R¹¹⁷ are each independently selected from (a) and (b) as follows:
(a) hydrogen, alkyl, alkenyl, alkynyl, aryl, heteroaryl, heteroarylium, cycloalkyl, heterocyclyl, C(A)R¹²⁹, OR¹²⁵ or NR¹³²R¹³³; or
(b) any two of R¹¹⁵, R¹¹⁶ and R¹¹⁷ together form alkylene, alkenylene, alkynylene, heteroalkylene, and the other is selected as in (a);
R¹²², R¹²³ and R¹²⁴ are selected as in (i) or (ii) as follows:
(i) R¹²², R¹²³ and R¹²⁴ are each independently hydrogen, alkyl, alkenyl, alkynyl, aryl, heteroaryl, heteroarylium, cycloalkyl, heterocyclyl, OR¹²⁵ or NR¹³²R¹³³; or
(ii) any two of R¹²², R¹²³ and R¹²⁴ together form alkylene, alkenylene, alkynylene, heteroalkylene; and the other is selected as in (i);
R¹²⁵ is hydrogen, alkyl, alkenyl, alkynyl, aryl, heteroaryl, heteroarylium, cycloalkyl or heterocyclyl;
R¹²⁶ is hydrogen, alkyl, alkenyl, alkynyl, aryl, heteroaryl, heteroarylium, cycloalkyl, heterocyclyl, OR¹²⁵ or NR¹³⁴R¹³⁵; where R¹³⁴ and R¹³⁵ are each independently hydrogen, alkyl, alkenyl, alkynyl, aryl, heteroaryl, heteroarylium, cycloalkyl, heterocyclyl, OR¹³⁶ or NR¹³²R¹³³, or R¹³⁴ and R¹³⁵ together form alkylene, alkenylene, alkynylene, heteroalkylene, where R¹³⁶ is hydrogen, alkyl, alkenyl, alkynyl, aryl, heteroaryl, heteroarylium, cycloalkyl or heterocyclyl;
R¹²⁷ and R¹²⁸ are selected as in (i) or (ii) as follows:
(i) R¹²⁷ and R¹²⁸ are each independently hydrogen, alkyl, alkenyl, alkynyl, aryl, heteroaryl, heteroarylium, cycloalkyl, heterocyclyl, OR¹²⁵, NR¹³⁷R¹³⁸ or C(A)R¹³⁹, where R¹³⁷ and R¹³⁸ are each independently hydrogen, alkyl, alkenyl, alkynyl, aryl, heteroaryl, heteroarylium, cycloalkyl or heterocyclyl, or together form alkylene, alkenylene, alkynylene, heteroalkylene; and R¹³⁹ is hydrogen, alkyl, alkenyl, alkynyl, aryl, heteroaryl, heteroarylium, cycloalkyl, heterocyclyl, OR¹⁴⁰ or NR¹³²R¹³³, where R¹⁴⁰ is alkyl, alkenyl, alkynyl, aryl, heteroaryl, heteroarylium, cycloalkyl, heterocyclyl; or
(ii) R¹²⁷ and R¹²⁸ together form alkylene, alkenylene, alkynylene, heteroalkylene;
R¹²⁹ is hydrogen, alkyl, alkenyl, alkynyl, aryl, heteroaryl, heteroarylium, cycloalkyl, heterocyclyl, OR¹⁴⁰ or NR¹³²R¹³³;
R¹³⁰ and R¹³¹ are each independently hydrogen, alkyl, alkenyl, alkynyl, aryl, heteroaryl, heteroarylium, cycloalkyl, heterocyclyl or C(A)R¹⁴¹, where R¹⁴¹ is alkyl, alkenyl, alkynyl, aryl, heteroaryl, heteroarylium, cycloalkyl, heterocyclyl, OR¹²⁵ or NR¹³²R¹³³; or R¹³⁰ and R¹³¹ together form alkylene, alkenylene, alkynylene, heteroalkylene;
R¹³² and R¹³³ are each independently hydrogen, alkyl, alkenyl, alkynyl, aryl, heteroaryl, heteroarylium, cycloalkyl, heterocyclyl, or R¹³² and R¹³³ together form alkylene, alkenylene, alkynylene, heteroalkylene; and
R³ is hydrogen, alkyl, alkenyl, alkynyl, cycloalkyl, heterocyclyl, aryl or heteroaryl;
wherein X, Y, Z, R¹, R² and R³ are each independently unsubstituted or substituted with one or more substituents, in one embodiment one, two or three substituents, each independently selected from Q¹, where Q¹ is halo, pseudohalo, hydroxy, oxo, thia, nitrile, nitro, formyl, mercapto, hydroxycarbonyl, hydroxycarbonylalkyl, hydroxycarbonylalkenyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, alkoxyxarbonylalkoxy, aryloxycarbonyl, aryloxycarbonylalkyl, aralkoxycarbonyl, aralkoxycarbonylalkyl, aralkoxycarbonylalkoxy, arylcarbonylalkyl, aminocarbonyl, aminocarbonylalkyl, aminocarbonylalkoxy, alkylaminocarbonyl, alkylaminocarbonylalkyl, alkylaminocarbonylalkoxy, dialkylaminocarbonyl, dialkylaminocarbonylalkyl, dialkylaminocarbonylalkoxy, arylaminocarbonyl, arylaminocarbonylalkyl, arylaminocarbonylalokoxy, diarylaminocarbonyl, diarylaminocarbonylalkyl, diarylaminocarbonyl alkoxy, arylalkylaminocarbonyl, arylalkylaminocarbonylalkyl, arylalkylaminocarbonylalkoxy, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R¹⁵¹R¹⁵²R¹⁵³, P(R¹⁵⁰)₂, P(═O)(R¹⁵⁰)₂, OP(═O)(R¹⁵⁰)₂, —NR¹⁶⁰C(═O)R¹⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; azido, tetrazolyl or two Q¹ groups, which substitute atoms in a 1,2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q¹ groups, which substitute the same atom, together form alkylene; and
each Q¹ is independently unsubstituted or substituted with one or more substituents, in one embodiment one, two or three substituents, each independently selected from Q²;
each Q² is independently halo, pseudohalo, hydroxy, oxo, thia, nitrile, nitro, formyl, mercapto, hydroxycarbonyl, hydroxycarbonylalkyl, hydroxycarbonylalkenyl alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, beteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R¹⁵¹R¹⁵²R¹⁵³, P(R¹⁵⁰)₂, P(═O)(R¹⁵⁰)₂, OP(═O)(R¹⁵⁰)₂, —NR¹⁶⁰C(═O)R¹⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q² groups, which substitute atoms in a 1,2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q² groups, which substitute the same atom, together form alkylene;
R¹⁵⁰ is hydroxy, alkoxy, aralkoxy, alkyl, heteroaryl, heterocyclyl, aryl or —NR¹⁷⁰R¹⁷¹, where R¹⁷⁰ and R¹⁷¹ are each independently hydrogen, alkyl, aralkyl, aryl, heteroaryl, heteroaralkyl or heterocyclyl, or R¹⁷⁰ and R¹⁷¹ together form alkylene, azaalkylene, oxaalkylene or thiaalkylene;
R¹⁵¹, R¹⁵² and R¹⁵³ are each independently hydrogen, alkyl, aryl, aralkyl, heteroaryl, heteroaralkyl, heterocyclyl or heterocyclylalkyl;
R¹⁶⁰ is hydrogen, alkyl, aryl, aralkyl, heteroaryl, heteroaralkyl, heterocyclyl or heterocyclylalkyl; and
R¹⁶³ is alkoxy, aralkoxy, alkyl, heteroaryl, heterocyclyl, aryl or —NR¹⁷⁰R¹⁷¹.

69. The compound of claim 68, wherein when R^{j} and R^{k}, together with the carbon to which they are both bonded, are —C(═O)—, —CH(OR*)—, —C(═S)—, —CH(SR*)—, —CH(NR*R*′)— or —C(═NR*)—, Y is NRR″ or CRR″ and R″, together with R³ and the atoms therebetween, is a 4-6 membered heterocyclyl or heteroaryl ring.

70. The compound of claim 68, wherein wherein:
X is O, S or NR, where R is hydrogen or alkyl;
Y is NRR′ or OH, where R is hydrogen or alkyl;
Z is a direct bond or NR;
R¹ is alkyl, alkenyl, alkynyl, cycloalkyl, aryl, heterocyclyl, heteroaryl, aralkyl, aralkenyl, heteroaralkyl, or heteroaralkenyl;
R² is halo, pseudohalo, alkoxy or alky;
n is 0 or 1;
R³ is hydrogen or alkyl;
wherein X, Y, Z, R¹, R² and R³ are each independently unsubstituted or substituted with one or more substituents, in one embodiment one, two or three substituents, each independently selected from Q¹.

71-81. (canceled)

82. The compound of claim 68, wherein the compound is:

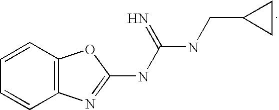

83. The compound of claim 68, wherein the compound is:

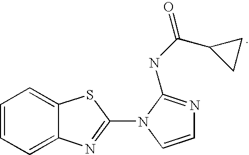

84. The compound of claim 68, wherein the compound is:

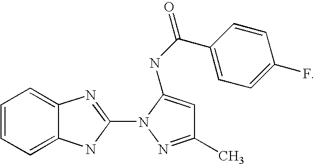

85. The compound of claim 68, wherein the compound is represented by one of Formulas Ib-Ie, Ig, or Ih-IIm: 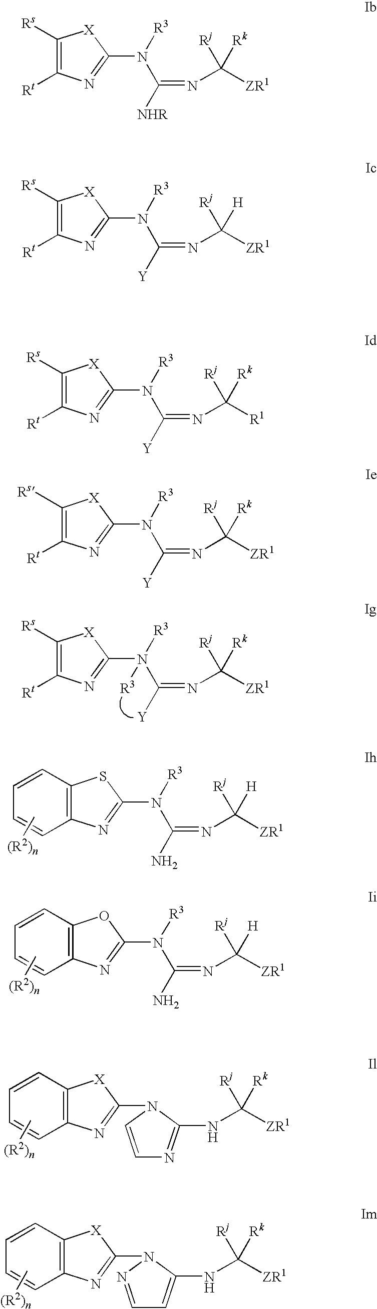
wherein
R¹ is hydrogen, alkyl, aryl, aralkyl, aralkenyl, alkynyl, heteroaryl, heteroaralkyl, heteroarylalkenyl, or cycloalkyl, each of which is substituted with 0, 1 or 2 groups selected from phenyl, alkyl, cycloalkyl, alkoxy, halo, pseudohalo, amino, alkylamino, or dialkylamino; and
R^{s}′ and R^{t}′ are independently selected from hydrogen, alkyl, halo, pseudohalo, alkenyl, alkynyl, cycloalkyl, heterocyclyl, aryl, heteroaryl and aralkyl.

86-89. (canceled)

90. The compound of claim 85, wherein the compound is represented by Formula Ie: 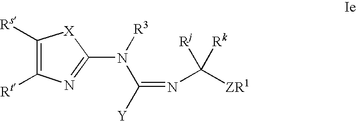
wherein R^{s}′ and R^{t}′ are independently selected from hydrogen, alkyl, and halo.

91. (canceled)

92. The compound of claim 85, wherein the compound is represented by one of Formulas Ih, Ii, Il or Im: 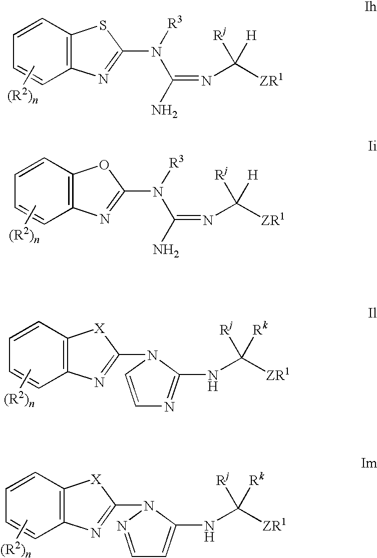
wherein
n is 0, 1 or 2; and
each R² is independently selected from halogen, alkyl, alkoxy, haloalkyl, and haloalkoxy.

93. The compound of claim 92, wherein each R² is independently selected from hydrogen, F, fluoroalkyl, and fluoroalkoxy.

94. A compound represented by Formula Ia: 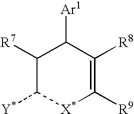
or a pharmaceutically acceptable derivative thereof, wherein:
X* is selected from the group consisting of —O—, ═N—, —N(R^{o})—, ═C(R^{o})— and —C(R^{o}R^{o}′)—;
Y* is selected from the group consisting of ═O, —OR^{o}, ═NR^{o}′, —NR^{o}R^{o}′, ═CR^{o}R^{o}′ and —CHR^{o}R^{o}′; where X* and Y* are selected such that both dashed bonds are single bonds, or one of the dashed bonds (— — —) is a single bond and the other is a double bond, provided that Y* is not ═O when X* is —N(H)—;
each R^{o}′ is independently selected from the group consisting of hydrogen, halogen, pseudohalo, amino, amido, carboxamido, sulfonamide, carboxyl, alkyl, alkenyl, alkynyl, cycloalkyl, heterocyclyl, aryl, heteroaryl, aralkyl, alkoxy, cycloalkoxy, heterocycloxy, aryloxy, heteroaryloxy, and aralkyloxy;
each R^{o} is selected from the group consisting of hydrogen, alkyl, alkenyl, alkynyl, cycloalkyl, heterocyclyl, aryl, heteroaryl and aralkyl;
Ar¹ is aryl, heteroaryl, or cycloalkyl;
R⁷ is hydrogen, alkyl, alkenyl, alkynyl, cycloalkyl, heterocyclyl, aryl, heteroaryl or NRR, where R is hydrogen or alkyl;
R¹⁰ is hydrogen, alkyl, alkenyl, alkynyl, cycloalkyl, heterocyclyl, aryl or heteroaryl;
R⁸ and R⁹ are each independently selected from (i) or (ii) as follows:
(i) hydrogen, alkyl, alkenyl, alkynyl, aryl, heteroaryl, heteroarylium, cycloalkyl, heterocyclyl, C(A)R¹¹⁰, halo, pseudohalo, OR¹¹¹, S(D)ₐR¹¹², NR¹¹⁵R¹¹⁶ or N⁺R¹¹⁵R¹¹⁶R¹¹⁷; or
(ii) any two R² groups, which substitute adjacent atoms on the ring, together form alkylene, alkenylene, alkynylene or heteroalkylene;
A is O, S or NR¹²⁵;
R¹¹⁰ is hydrogen, alkyl, alkenyl, alkynyl, aryl, heteroaryl, heteroarylium, cycloalkyl, heterocyclyl, C(A)R¹²⁶, halo pseudohalo, OR¹²⁵, SR¹²⁵ NR¹²⁷R¹²⁸ and SiR¹²²R¹²³R¹²⁴;
R¹¹¹ is hydrogen, alkyl, alkenyl, alkynyl, aryl, heteroaryl, heteroarylium, cycloalkyl, heterocyclyl, C(A)R¹²⁹, NR¹³⁰R¹³¹ and SiR¹²²R¹²³R¹²⁴;
D is O or NR¹²⁵;
a is 0, 1 or 2;
when a is 1 or 2, R¹¹² is selected from hydrogen, alkyl, alkenyl, alkynyl, aryl, heteroaryl, heteroarylium, cycloalkyl, heterocyclyl, halo, pseudohalo, OR¹²⁵, SR¹²⁵ and NR¹³²R¹³³;
when a is 0, R¹¹² is selected from hydrogen, alkyl, alkenyl, alkynyl, aryl, heteroaryl, heteroarylium, cycloalkyl, heterocyclyl, SR¹²⁵ and C(A)R¹²⁹;
R¹¹⁵, R¹¹⁶ and R¹¹⁷ are each independently selected from (a) and (b) as follows:
(a) hydrogen, alkyl, alkenyl, alkynyl, aryl, heteroaryl, heteroarylium, cycloalkyl, heterocyclyl, C(A)R¹²⁹, OR¹²⁵ or NR¹³²R¹³³; or
(b) any two of R¹¹⁵, R¹¹⁶ and R¹¹⁷ together form alkylene, alkenylene, alkynylene, heteroalkylene, and the other is selected as in (a);
R¹²², R¹²³ and R¹²⁴ are selected as in (i) or (ii) as follows:
(i) R¹²², R¹²³ and R¹²⁴ are each independently hydrogen, alkyl, alkenyl, alkynyl, aryl, heteroaryl, heteroarylium, cycloalkyl, heterocyclyl, OR¹²⁵ or NR¹³²R¹³³; or
(ii) any two of R¹²², R¹²³ and R¹²⁴ together form alkylene, alkenylene, alkynylene, heteroalkylene; and the other is selected as in (i);
R¹²⁵ is hydrogen, alkyl, alkenyl, alkynyl, aryl, heteroaryl, heteroarylium, cycloalkyl or heterocyclyl;
R¹²⁶ is hydrogen, alkyl, alkenyl, alkynyl, aryl, heteroaryl, heteroarylium, cycloalkyl, heterocyclyl, OR¹²⁵ or NR¹³⁴R¹³⁵; where R¹³⁴ and R¹³⁵ are each independently hydrogen, alkyl, alkenyl, alkynyl, aryl, heteroaryl, heteroarylium, cycloalkyl, heterocyclyl, OR¹³⁶ or NR¹³²R¹³³, or R¹³⁴ and R¹³⁵ together form alkylene, alkenylene, alkynylene, heteroalkylene, where R¹³⁶ is hydrogen, alkyl, alkenyl, alkynyl, aryl, heteroaryl, heteroarylium, cycloalkyl or heterocyclyl;
R¹²⁷ and R¹²⁸ are selected as in (i) or (ii) as follows:
(i) R¹²⁷ and R¹²⁸ are each independently hydrogen, alkyl, alkenyl, alkynyl, aryl, heteroaryl, heteroarylium, cycloalkyl, heterocyclyl, OR²⁵, NR¹³⁷R¹³⁸ or C(A)R¹³⁹, where R¹³⁷ and R¹³⁸ are each independently hydrogen, alkyl, alkenyl, alkynyl, aryl, heteroaryl, heteroarylium, cycloalkyl or heterocyclyl, or together form alkylene, alkenylene, alkynylene, heteroalkylene; and R¹³⁹ is hydrogen, alkyl, alkenyl, alkynyl, aryl, heteroaryl, heteroarylium, cycloalkyl, heterocyclyl, OR¹⁴⁰ or NR¹³²R¹³³, where R¹⁴⁰ is alkyl, alkenyl, alkynyl, aryl, heteroaryl, heteroarylium, cycloalkyl, heterocyclyl; or
(ii) R¹²⁷ and R¹²⁸ together form alkylene, alkenylene, alkynylene, heteroalkylene;
R¹²⁹ is hydrogen, alkyl, alkenyl, alkynyl, aryl, heteroaryl, heteroarylium, cycloalkyl, heterocyclyl, OR¹⁴⁰ or NR¹³²R¹³³;
R¹³⁰ and R¹³¹ are each independently hydrogen, alkyl, alkenyl, alkynyl, aryl, heteroaryl, heteroarylium, cycloalkyl, heterocyclyl or C(A)R¹⁴¹, where R¹⁴¹ is alkyl, alkenyl, alkynyl, aryl, heteroaryl, heteroarylium, cycloalkyl, heterocyclyl, OR¹²⁵ or NR¹³²R¹³³; or R¹³⁰ and R¹³¹ together form alkylene, alkenylene, alkynylene, heteroalkylene;
R¹³² and R¹³³ are each independently hydrogen, alkyl, alkenyl, alkynyl, aryl, heteroaryl, heteroarylium, cycloalkyl, heterocyclyl, or R¹³² and R¹³³ together form alkylene, alkenylene, alkynylene, heteroalkylene; and
R¹⁰ is hydrogen, alkyl, alkenyl, alkynyl, cycloalkyl, heterocyclyl, aryl or heteroaryl;
where Ar¹, R⁷, R⁸, R⁹ and R¹⁰ are each independently unsubstituted or substituted with one or more, in one embodiment one, two or three substituents, each independently selected from Q¹, where Q¹ is halo, pseudohalo, hydroxy, oxo, thia, nitrile, nitro, formyl, mercapto, hydroxycarbonyl, hydroxycarbonylalkyl, hydroxycarbonylalkenyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, alkoxyxarbonylalkoxy, aryloxycarbonyl, aryloxycarbonylalkyl, aralkoxycarbonyl, aralkoxycarbonylalkyl, aralkoxycarbonylalkoxy, arylcarbonylalkyl, aminocarbonyl, aminocarbonylalkyl, aminocarbonylalkoxy, alkylaminocarbonyl, alkylaminocarbonylalkyl, alkylaminocarbonylalkoxy, dialkylaminocarbonyl, dialkylaminocarbonylalkyl, dialkylaminocarbonylalkoxy, arylaminocarbonyl, arylaminocarbonylalkyl, arylaminocarbonylalokoxy, diarylaminocarbonyl, diarylaminocarbonylalkyl, diarylaminocarbonyl alkoxy, arylalkylaminocarbonyl, arylalkylaminocarbonylalkyl, arylalkylaminocarbonylalkoxy, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R¹⁵¹R¹⁵²R¹⁵³, P(R¹⁵⁰)₂, P(═O)(R¹⁵⁰)₂, OP(═O)(R¹⁵⁰)₂, —NR¹⁶⁰C(═O)R¹⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; azido, tetrazolyl or two Q¹ groups, which substitute atoms in a 1,2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q¹ groups, which substitute the same atom, together form alkylene; and
each Q¹ is independently unsubstituted or substituted with one or more substituents, in one embodiment one, two or three substituents, each independently selected from Q²;
each Q² is independently halo, pseudohalo, hydroxy, oxo, thia, nitrile, nitro, formyl, mercapto, hydroxycarbonyl, hydroxycarbonylalkyl, hydroxycarbonylalkenyl alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R¹⁵¹R¹⁵²R¹⁵³, P(R¹⁵⁰)₂, P(═O)(R¹⁵⁰)₂, OP(═O)(R¹⁵⁰)₂, —NR¹⁶⁰C(═O)R¹⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q² groups, which substitute atoms in a 1,2 or 1,3 arrangement, together form alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q² groups, which substitute the same atom, together form alkylene;
R¹⁵⁰ is hydroxy, alkoxy, aralkoxy, alkyl, heteroaryl, heterocyclyl, aryl or —NR¹⁷⁰R¹⁷¹, where R¹⁷⁰ and R¹⁷¹ are each independently hydrogen, alkyl, aralkyl, aryl, heteroaryl, heteroaralkyl or heterocyclyl, or R¹⁷⁰ and R¹⁷¹ together form alkylene, azaalkylene, oxaalkylene or thiaalkylene;
R¹⁵¹, R¹⁵² and R¹⁵³ are each independently hydrogen, alkyl, aryl, aralkyl, heteroaryl, heteroaralkyl, heterocyclyl or heterocyclylalkyl;
R¹⁶⁰ is hydrogen, alkyl, aryl, aralkyl, heteroaryl, heteroaralkyl, heterocyclyl or heterocyclylalkyl; and
R¹⁶³ is alkoxy, aralkoxy, alkyl, heteroaryl, heterocyclyl, aryl or —NR¹⁷⁰R¹⁷¹.

95-102. (canceled)

103. The compound of claim 94, wherein the compound is selected from the compounds in Table II.

104. The compound of claim 103, wherein wherein the compound is:

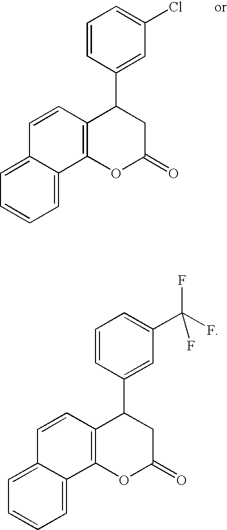

105. The compound of claim 103, wherein wherein the compound is:

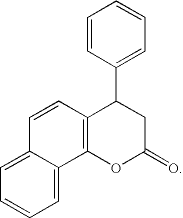

106. The compound of claim 103, wherein the compound is:

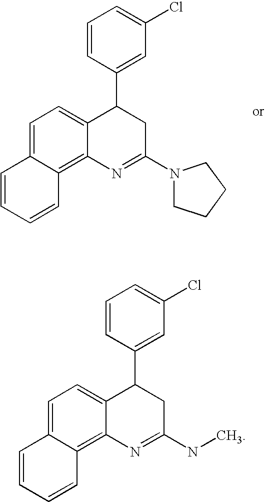

107. The compound of claim 94, wherein the compound is represented by one of Formulas IIb-IIp: 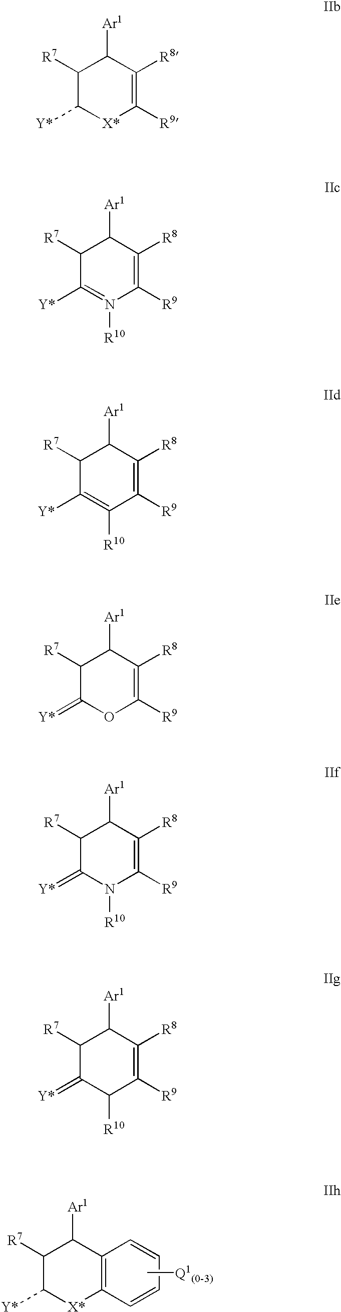 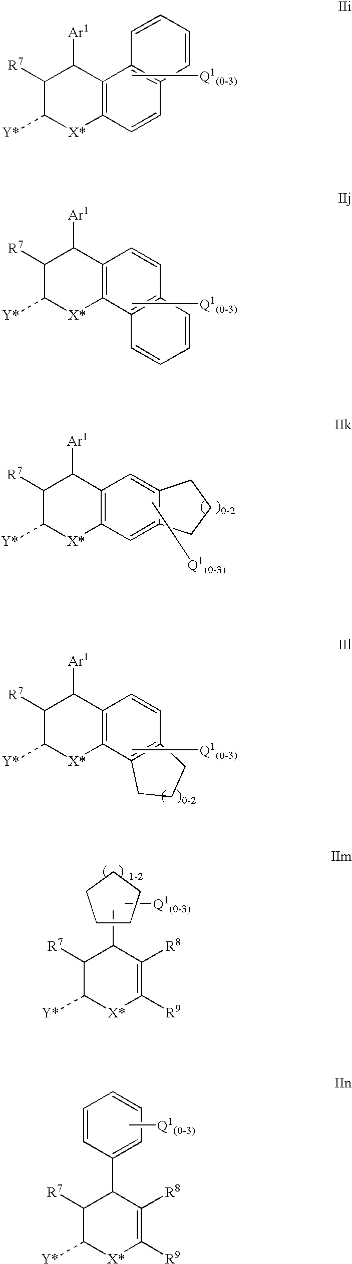 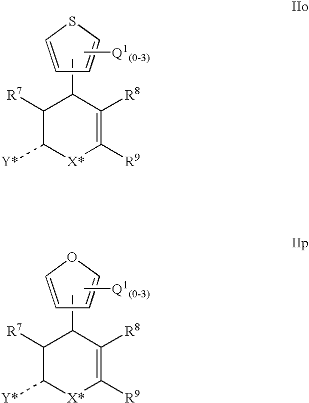
wherein R⁸′ and R⁹′ are independently selected from hydrogen, alkyl, alkenyl, alkynyl, aryl, heteroaryl, heteroarylium, cycloalkyl, heterocyclyl, C(A)R¹¹⁰, halo, pseudohalo, OR¹¹¹, S(D)ₐR¹¹², NR¹¹⁵R¹¹⁶ or N⁺R¹¹⁵R¹¹⁶R¹¹⁷.

108. The compound of claim 107, wherein the compound is represented by Formula IIb, wherein:
R^{8′} is CN or COOR²⁰⁰, where R²⁰⁰ is hydrogen, alkyl, alkenyl, alkynyl, cycloalkyl, heterocyclyl, aryl or heteroaryl; and
R^{9′} is hydrogen, alkyl or alkylthio.

109. The compound of claim 108, wherein:
R^{8′} is CN or COOR²⁰⁰, where R²⁰⁰ is methyl, benzyl, ethyl, 4-methoxybenzyl or 2-phenylethyl; and
R^{9′} is methyl, methylthio or phenylaminocarbonylmethylthio.

110. The compound of claim 107, wherein the compound is represented by one of Formulas IIh-IIp: 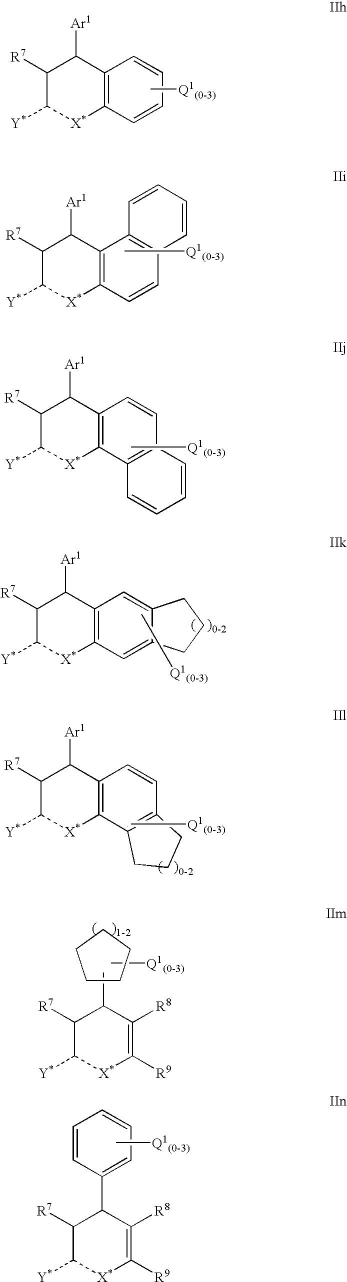 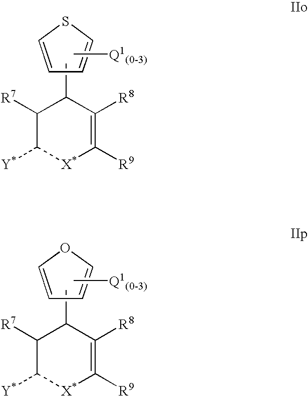
wherein each Q¹ is independently selected from halogen, alkyl, alkoxy, nitro, CN, N₃, aryl, aryloxy, arylalkyloxy, alkynyl, amino, alkylamino, heterocyclyl, heteroaryl, substituted carboxyl, haloalkyl, and haloalkoxy, or two adjacent Q¹, on the same phenyl or adjacent fused phenyl rings, together form a cycloalkyl or heterocyclyl ring fused with the phenyl or adjacent fused phenyl rings.

111-112. (canceled)

113. A method of identifying a compound that rescues impaired endoplasmic reticulum-mediated transport, the method comprising:
providing a cell lysate prepared from a cell that exhibits impaired endoplasmic reticulum-mediated transport;
contacting the cell lysate with a candidate agent; and
determining whether the candidate agent enhances endoplasmic reticulum-mediated transport in the cell lysate as compared to in the absence of the candidate agent, wherein a compound that enhances endoplasmic reticulum-mediated transport is identified as a compound that rescues impaired endoplasmic reticulum-mediated transport.

114-139. (canceled)

140. A method of identifying a compound that increases endoplasmic reticulum-mediated transport, the method comprising:
providing a cell that exhibits impaired endoplasmic reticulum-mediated transport;
contacting the cell with an agent that inhibits expression or activity of Bst1, Emp24, PGAP1, TMED2, TMED10, or TMED7; and
measuring endoplasmic reticulum-mediated transport in the cell in the presence of the agent,
wherein an increase in endoplasmic reticulum-mediated transport in the presence of the agent as compared to endoplasmic reticulum-mediated transport in the absence of the agent identifies the agent as a compound that increases endoplasmic reticulum-mediated transport.

141-143. (canceled)

144. A method of identifying a compound that increases endoplasmic reticulum-mediated transport, the method comprising:
providing a cell that exhibits impaired endoplasmic reticulum-mediated transport;
contacting the cell with an agent that enhances expression or activity of a protein selected from the group consisting of SEC12, Sec12, SED4, SEC16, HRD3, IRE1, STS1, SEC24, SEL1L, S20orf50, Ire1, Sec24A, Sec24B, Sec24C, and Sec24D; and
measuring cell viability in the presence of the agent,
wherein an increase in cell viability in the presence of the agent as compared to cell viability in the absence of the agent identifies the agent as a compound that increases endoplasmic reticulum-mediated transport.

145-153. (canceled)

154. A method of producing a protein, the method comprising:
culturing a cell in the presence of a compound described in claim 1 or in Table I or II; and
purifying a protein produced by the cell,
wherein the culturing of the cell in the presence of the compound results in enhanced production of the purified protein as compared to culture of the cell in the absence of the compound.

155. The method of claim 154, wherein the protein is a recombinant protein encoded by a heterologous nucleic acid.

156. The method of claim 154, wherein the protein is a secreted protein

157. The method of claim 154, wherein the protein is a glycosylated protein.

158. The method of claim 154, wherein the protein is a cytokine, a lymphokine, a growth factor, or an antibody.

159. The method of claim 154, wherein the cell is an insect cell, a mammalian cell, a fungal cell, or a bacterial cell.

160. The method of claim 159, wherein the cell is a Chinese Hamster Ovary (CHO) cell.
